(19) United States
(12) Patent Application Publication
(10) Pub. No.: US 2025/0376667 A1
(43) Pub. Date: Dec. 11, 2025

(54) ENGINEERED ALPHA KLOTHO POLYPEPTIDES AND USES THEREOF

(71) Applicant: Regeneron Pharmaceuticals, Inc., Tarrytown, NY (US)

(72) Inventors: Marc EGERMAN, Ossining, NY (US); Chauhongduc PHAN, Elmsford, NY (US); Nianshuang WANG, Greenwich, CT (US); Yang SHEN, Scarsdale, NY (US); Xin WANG, White Plains, NY (US); Kishor DEVALARAJA-NARASHIMHA, Yorktown Heights, NY (US); David GLASS, Briarcliff Manor, NY (US)

(73) Assignee: Regeneron Pharmaceuticals, Inc., Tarrytown, NY (US)

(21) Appl. No.: 19/231,084

(22) Filed: Jun. 6, 2025

Related U.S. Application Data
(60) Provisional application No. 63/657,229, filed on Jun. 7, 2024.

(51) Int. Cl. C12N 9/24; A61K 38/00; A61P 13/12

(52) U.S. Cl. CPC C12N 9/2402; A61P 13/12; A61K 38/00; C07K 2319/31; C07K 2319/50; C12Y 302/01031

(57) ABSTRACT

The present disclosure provides engineered alpha klotho polypeptides and methods of their production and use. Soluble alpha klotho polypeptides disclosed herein comprise an alpha klotho moiety having one or more mutations for enhancing activity, production yield, and/or stability relative to human alpha klotho. The disclosure further provides pharmaceutical compositions comprising the engineered alpha klotho polypeptides, and methods of use of the engineered alpha klotho polypeptides treatment methods, including treatment of age-related conditions and kidney disease. Also disclosed are nucleic acids encoding the engineered alpha klotho polypeptides, recombinant cells that express the engineered alpha klotho polypeptides, and methods of producing the engineered alpha klotho polypeptides.

### 1. CROSS-REFERENCE TO RELATED APPLICATIONS

This application claims the priority benefit of U.S. provisional application No. 63/657,229, filed Jun. 7, 2024, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Jun. 3, 2025, is named RGN-045US_SL.xml and is 483,020 bytes in size.

### 3. BACKGROUND

Alpha-Klotho, often referred to as Klotho (KL), is a single pass transmembrane protein located at the plasma membrane and expressed primarily in the kidney, parathyroid gland, and choroid plexus. The intracellular region of human KL (hKL) is relatively short and lacks any functional domains, whereas the extra cellular region comprises two primary domains, KL1 and KL2, which have amino-acid sequence homology to family 1 glycosidases.

KL can be proteolytically cleaved to produce a 130 kDa soluble form of KL comprising the KL extracellular region and released into the circulation. Despite its sequence homology to glycosidases, KL extracellular region does not exhibit glycosidase enzymatic activity. Instead, the extracellular portion of KL functions as an FGF23 coreceptor through FGF receptors and is involved in the regulation of Pᵢ and vitamin D metabolism, ion homeostasis, and oxidative stress.

KL has been associated with maintenance of kidney health. For instance, KL deficiency is implicated in human chronic kidney disease (Barker et al., 2014, Nephrol Dial Transplant 30(2):223-233), which affects more than 700 million individuals worldwide and is associated with more than one million deaths globally. KL also displays protective effects against aging-related conditions, and circulating levels of soluble KL decrease with age (Kim et al., 2015, J Lifestyle Med. 5(1):1-6).

Accordingly, there exists a need to develop KL polypeptides that exhibit effective bioactivity and can be produced efficiently at scale.

### 4. SUMMARY

The present disclosure relates to engineered alpha klotho polypeptides.

Typically, the engineered alpha klotho polypeptides of the disclosure are soluble, e.g., lack a transmembrane domain.

Engineered alpha klotho polypeptides of the present disclosure comprise an alpha klotho moiety. Exemplary alpha klotho moieties are described in Section 6.3.

In some embodiments, the engineered klotho polypeptides of the disclosure comprise a KL2 domain having an amino acid substitution at position 521 and a C-terminal tail region which does not comprise a cysteine at position 970, if present (numbering relative to human KL; SEQ ID NO:1). As described herein, these modifications produced a protein having surprisingly enhanced activity, production yield, and stability relative to human KL.

An engineered alpha klotho polypeptide may further comprise a stabilization moiety, optionally linked to the alpha klotho moiety via a linker. In certain embodiments, the linker is a protease-cleavable linker. Exemplary stabilization moieties are described in Section 6.4. Exemplary linkers are described in Section 6.5, with exemplary protease-cleavable linkers described in Section 6.5.2.

Exemplary engineered alpha klotho polypeptides of the disclosure are described in Section 6.2 and numbered embodiments 1 to 188.

The disclosure further provides nucleic acids encoding the engineered alpha klotho polypeptides of the disclosure. The disclosure further provides host cells and cell lines engineered to express the nucleic acids and engineered alpha klotho polypeptides of the disclosure. The disclosure further provides methods of producing an engineered alpha klotho polypeptide of the disclosure. Exemplary nucleic acids, host cells, and cell lines, and methods of producing an engineered alpha klotho polypeptide are described in Section 6.6 and numbered embodiments 189 to 195.

The disclosure further provides pharmaceutical compositions comprising the engineered alpha klotho polypeptides of the disclosure. Exemplary pharmaceutical compositions are described in Section 6.7 and numbered embodiment 196.

Further provided herein are methods of using the engineered alpha klotho polypeptides and the pharmaceutical compositions of the disclosure, e.g., for activating FGFR signaling, for treating age-related conditions, for treating kidney disease (e.g., acute kidney injury or chronic kidney disease), or for supplementing endogenous alpha klotho protein. Exemplary methods are described in Section 6.8 and numbered embodiments 197 to 212.

### 5. BRIEF DESCRIPTION OF THE FIGURES

FIG. 1 is a schematic illustration of an alpha klotho extracellular region, which comprises, from N-terminus to C-terminus: an alpha klotho-1 (KL1) domain (AAs 34-506), an interdomain region (AAs 507-514), an alpha klotho-2 (KL2) domain (AAs 515-950), and a C-terminal tail region (950-981). The amino acid numbering shown is relative to full length human alpha klotho (Genbank BAA23382.1; SEQ ID NO: 1 and UniProtKB Q9UEF7-1; SEQ ID NO: 2).

FIGS. 2A-2J are schematic illustrations of exemplary engineered alpha klotho polypeptides.

FIG. 2A shows an engineered alpha klotho polypeptide chain comprising, in N- to C-terminal orientation, a KL1 domain, an inter-domain region, a KL2 domain having a C521S mutation, and a C-terminal tail region. In some embodiments, an engineered alpha klotho polypeptide of the disclosure, e.g., as illustrated in FIG. 2A, is monomeric, consisting of only a single polypeptide chain, and may further comprise a signal sequence.

FIG. 2B shows an engineered alpha klotho polypeptide comprising, in N- to C-terminal orientation, a KL1 domain, an inter-domain region, a KL2 domain having a C521S mutation, a C-terminal tail region, a linker (L) which may be a protease cleavable linker such as those described in Section 6.5.2, and a stabilization moiety. In some embodiments, an engineered alpha klotho polypeptide of the disclosure, e.g., as illustrated in FIG. 2B, is monomeric, consisting of only a single polypeptide chain, and may further comprise a signal sequence.

FIG. 2C shows an engineered alpha klotho polypeptide comprising, in N- to C-terminal orientation, a KL1 domain having F352V and C370S mutations, an inter-domain region, a KL2 domain having a C521S mutation, and a C-terminal tail region. In some embodiments, an engineered alpha klotho polypeptide of the disclosure, e.g., as illustrated in FIG. 2C, is monomeric, consisting of only a single polypeptide chain, and may further comprise a signal sequence.

FIG. 2D shows an engineered alpha klotho polypeptide comprising, in N- to C-terminal orientation, a KL1 domain having F352V and C370S mutations, an inter-domain region, a KL2 domain having a C521S mutation, a C-terminal tail region, a linker (L) which may be a protease cleavable linker such as those described in Section 6.5.2, and a stabilization moiety. In some embodiments, an engineered alpha klotho polypeptide of the disclosure, e.g., as illustrated in FIG. 2D, is monomeric, consisting of only a single polypeptide chain, and may further comprise a signal sequence.

FIG. 2E shows an engineered alpha klotho polypeptide comprising, in N- to C-terminal orientation, a KL1 domain having an F352V mutation, an inter-domain region, a KL2 domain having a C521S mutation, and a C-terminal tail region. In some embodiments, an engineered alpha klotho polypeptide of the disclosure, e.g., as illustrated in FIG. 2E, is monomeric, consisting of only a single polypeptide chain, and may further comprise a signal sequence.

FIG. 2F shows an engineered alpha klotho polypeptide comprising, in N- to C-terminal orientation, a KL1 domain having an F352V mutation, an inter-domain region, a KL2 domain having a C521S mutation, a C-terminal tail region, a linker (L) which may be a protease cleavable linker such as those described in Section 6.5.2, and a stabilization moiety. In some embodiments, an engineered alpha klotho polypeptide of the disclosure, e.g., as illustrated in FIG. 2F, is monomeric, consisting of only a single polypeptide chain, and may further comprise a signal sequence.

FIG. 2G shows an engineered alpha klotho polypeptide comprising, in N- to C-terminal orientation, a KL1 domain having a C370S mutation, an inter-domain region, a KL2 domain having a C521S mutation, and a C-terminal tail region. In some embodiments, an engineered alpha klotho polypeptide of the disclosure, e.g., as illustrated in FIG. 2G, is monomeric, consisting of only a single polypeptide chain, and may further comprise a signal sequence.

FIG. 2H shows an engineered alpha klotho polypeptide comprising, in N- to C-terminal orientation, a KL1 domain having a C370S mutation, an inter-domain region, a KL2 domain having a C521S mutation, a C-terminal tail region, a linker (L) which may be a protease cleavable linker such as those described in Section 6.5.2, and a stabilization moiety. In some embodiments, an engineered alpha klotho polypeptide of the disclosure, e.g., as illustrated in FIG. 2H, is monomeric, consisting of only a single polypeptide chain, and may further comprise a signal sequence.

FIG. 2I shows an engineered alpha klotho polypeptide comprising, in N- to C-terminal orientation, a KL1 domain having an L111S mutation, an inter-domain region, a KL2 domain having a C521S mutation, and a C-terminal tail region. In some embodiments, an engineered alpha klotho polypeptide of the disclosure, e.g., as illustrated in FIG. 2I, is monomeric, consisting of only a single polypeptide chain, and may further comprise a signal sequence.

FIG. 2J shows an engineered alpha klotho polypeptide comprising, in N- to C-terminal orientation, a KL1 domain having an L111S mutation, an inter-domain region, a KL2 domain having a C521S mutation, a C-terminal tail region, a linker (L) which may be a protease cleavable linker such as those described in Section 6.5.2, and a stabilization moiety. In some embodiments, an engineered alpha klotho polypeptide of the disclosure, e.g., as illustrated in FIG. 2J, is monomeric, consisting of only a single polypeptide chain, and may further comprise a signal sequence.

For each of the constructs depicted in FIGS. 2A-2J, the C-terminal tail region may be a full-length C-terminal tail (optionally with one or more amino acid substitutions) or may be a truncated C-terminal tail region relative to human KL. In some embodiments, the C-terminal tail region of the constructs depicted in FIGS. 2A-2J comprises a C-terminal deletion of between 12 and 31 amino acids relative to human KL. All amino acid numbering is relative to full length human alpha klotho (Genbank BAA23382.1; SEQ ID NO:1 and UniProtKB Q9UEF7-1; SEQ ID NO:2).

FIG. 3 shows the amino acid sequence of full-length human klotho (UniProt KB Genbank BAA23382.1; SEQ ID NO:1). The first 33 amino acids underlined with the dotted line correspond to the native hKL signal sequence. Italicized amino acid residues correspond to the KL1 domain, bolded amino acid residues correspond to the KL2 domain, and the amino acid residues underlined with the solid line represent inter-domain region (the region located between the KL1 and KL2 domains). The amino acid residues presented inside the solid box show the C-terminal ADAM10/ADAM17 cleavage target sequence, while the dotted vertical line between the two bolded and underlined amino acid residues indicates the site of cleavage. The amino acid residues presented inside the dotted box correspond to the transmembrane region and the amino acid residues underlined with the double-lines correspond to the intracellular region of hKL. The amino acids between the KL2 domain and the transmembrane region correspond to the C-terminal tail region.

FIGS. 4A-4C show the effects of the C-terminal truncation and surface Cys residue engineering on the production and activity of engineered KL polypeptide constructs. FIG. 4A shows a graph displaying the post-purification yield and aggregation of His-tagged constructs REGN14416 (KL981 His), REGN14225 (KL958 His), REGN 14226 (KL958 C521S His), and REGN14227 (KL958 C521S C910S His), where the bars represent the production yield, and the line plot illustrates the post-size exclusion chromatography (postSEC) percentage of high molecular weight species (HMW %), which is used as a measure of aggregation. FIG. 4B is a bar graph depicting the fold change in phosphorylated extracellular signal-regulated kinase (PERK) levels in NIH3T3 cells as a result of engineered KL polypeptide and FGF23 co-treatment, relative to engineered KL polypeptide-alone treatment. FIG. 4C is a bar graph depicting the fold change in pERK levels in NHDF cells as a result of engineered KL polypeptide and FGF23 co-treatment, relative to engineered KL polypeptide-alone treatment.

FIGS. 5A-5C illustrate the effect of pH on the production yield and activity of the His and/or HSA-tagged KL958 constructs. FIG. 5A shows non-reduced (NR) and reduced (R) polyacrylamide gel electrophoresis (PAGE) images depicting the yield and aggregation differences when His- or HSA-tagged constructs were eluted either with a His column at pH 7 or with an HSA column at pH 3. The presence of a smear above 169 kDa in the NR condition indicates aggregate formation. FIG. 5B is a bar graph showing the results of a PERK homogeneous time-resolved fluorescence-based (HTRF) assay, which depicts pERK-associated fluorescence in untreated cells as well as in cells that were treated with FGF23, a positive control human alpha klotho protein (h aKL), or FGF23 together with h aKL. FIG. 5C is a bar graph showing pERK HTRF in cells treated with the construct, KL958 SS HSA His, eluted either with a His column at pH 7 or with an HSA column at pH 3, in the presence or absence of FGF23. Background level corresponds to the pERK HTRF level of the untreated cells in FIG. 5B.

FIGS. 6A-6D are cartoon depictions of engineered KL polypeptide comprising different stabilization moieties. FIG. 6A is a cartoon illustration of an engineered KL polypeptide with a C-terminal HSA stabilization moiety. FIG. 6B is a cartoon illustration of an engineered KL polypeptide dimer with a C-terminal Fc stabilization moiety. FIG. 6C is a cartoon illustration of an engineered KL polypeptide with a C-terminal Fc1.5 stabilization moiety. FIG. 6D is a cartoon illustration of an engineered KL polypeptide with a C-terminal monomeric Fc (moFc) stabilization moiety, where the moFc is a non-dimerizing Fc comprising one or more mutations on the hydrophobic surface of its CH3 region. Small ovals at the C-termini of the constructs shown in FIGS. 6A-6D represent optional additional tags (OATs) that can be used to facilitate purification of the constructs. Non-limiting examples of such additional tags include His tag (HHHHHH; SEQ ID NO: 186) and Twin-Strep-Tag® (WSHPQFEKGGGSGGGSGGSAWSHPQFEK; SEQ ID NO: 187). Such tags may be directly linked to the stabilization moiety, or connected via a linker.

FIGS. 7A-7E illustrate the production yield and purity of four distinct constructs, each comprising a different C-terminal tag linked to REGN 14226 (KL958 C521S). FIG. 7A is a polyacrylamide gel electrophoresis image showing the yields of reduced and non-reduced constructs comprising Fc, moFc, Fc1.5, and HSA stabilization moieties, respectively. All constructs also comprised a Twin-Strep-tag. The scissor icon above a lane indicates that the sample in that lane was treated with 3C protease to remove the Twin-Strep-tag prior to loading. FIG. 7B shows the size exclusion chromatography (SEC) purification profile of the Fc-tagged KL958 C521S polypeptide construct, KL958 (C521S)-Fc. FIG. 7C shows the size exclusion chromatography (SEC) purification profile of the moFc-tagged KL958 C521S polypeptide construct, KL958 (C521S)-MoFc. FIG. 7D shows the size exclusion chromatography (SEC) purification profile of the Fc1.5-tagged KL958 C521S polypeptide construct, KL958 (C521S)-Fc1.5. FIG. 7E shows the size exclusion chromatography (SEC) purification profile of the HSA-tagged KL958 C521S polypeptide construct, KL958 (C521S)-HSA. Striped peaks represent the high molecular weight (HMW) proteins and aggregates. Checker-patterned peaks correspond to monomeric KL958 C521S polypeptide constructs.

FIGS. 8A-8B show the results of modified high salt purification of the KL polypeptide construct KL958 (C521S)-HSA at neutral pH. FIG. 8A is a polyacrylamide gel electrophoresis image showing the yields of reduced and non-reduced KL958 (C521S)-HSA. Lane 1 was loaded with a molecular weight marker. Lanes 2 and 5 were loaded with non-reduced and reduced samples comprising aggregate and HMW proteins, respectively. Lanes 3 and 6 were loaded with non-reduced and reduced SEC elutes comprising the monomeric KL958 (C521S)-HSA constructs. FIG. 8B shows the SEC purification profile of KL958 (C521S)-HSA. Peak 1 corresponds to aggregated and HMW proteins, peak 2 corresponds to monomeric KL958 C521S polypeptide constructs, and peak 3 corresponds to low molecular weight peptides.

FIGS. 9A-9F show stepwise improvement of KL958 (C521S)-HSA purification with a three-step modified ion exchange chromatography (IEX) purification method. FIGS. 9A-9B show a polyacrylamide gel electrophoresis image of different fractions of proteins (FIG. 9A) and an SEC profile of the elute (FIG. 9B) obtained with the Q-Sepharose ion exchange step. FIGS. 9C-9D show a polyacrylamide gel electrophoresis image of different fractions of proteins (FIG. 9C) and an SEC profile of the elute (FIG. 9D) obtained with the HSA-affinity column purification step. FIGS. 9E-9F show a polyacrylamide gel electrophoresis image of the final elute (FIG. 9E) and an SEC profile of the final elute (FIG. 9F) obtained with the SD-200 purification step.

FIGS. 10A-10B show the effect of His and HSA tags on pharmacokinetic (PK) profiles of engineered KL polypeptide constructs. FIG. 10A is a graph displaying the average serum concentrations of KL polypeptide constructs over time. FIG. 10B is a graph displaying the dose-normalized PK profiles of KL polypeptide constructs.

FIGS. 11A-11C show the effect of His and HSA tags on engineered KL polypeptide-mediated proliferation of cultured 3T3 cells. FIG. 11A is a graph showing dose-dependent proliferation of cells treated with KL958 (C521S)-His in the presence or absence of FGF23. FIG. 11B is a graph showing dose-dependent proliferation of cells treated with KL958 (C521S)-HSA in the presence or absence of FGF23. FIG. 11C is a graph showing the differences in growth of cells treated with KL958 (C521S)-His and FGF23, KL958 (C521S)-HSA and FGF23, bFGF, or FGF23 alone, and untreated cells.

FIG. 12 is a graph showing the effect of different C-terminal truncations on the activity of certain engineered KL polypeptide constructs.

FIGS. 13A-13F illustrate exemplary engineered alpha klotho polypeptide constructs with the N-terminus to C-terminus configuration: [mROR1]-[KL1 domain]-[KL2 domain]-[G4S linker]-[HSA domain]. The first 33 amino acids of human KL, which correspond to the native KL signal sequence, are replaced with mROR1 signal sequence, and KL2 domains are truncated at residue 958. FIG. 13A shows a polypeptide construct comprising a C521S mutation in its KL2 domain. FIG. 13B shows a polypeptide construct comprising F352V and C370S mutations in its KL1 domain and a C521S mutation in its KL2 domain. FIG. 13C shows a polypeptide construct comprising an F352V mutation in its KL1 domain and a C521S mutation in its KL2 domain. FIG. 13D shows a polypeptide construct comprising a C370S mutation in its KL1 domain and a C521S mutation in its KL2 domain. FIG. 13E shows a polypeptide construct comprising an L111S mutation in its KL1 domain and a C521S mutation in its KL2 domain. FIG. 13F shows a polypeptide construct comprising a C521S mutation in its KL2 domain and a C34S mutation in its HSA domain. All constructs depicted in FIGS. 13A-13F contain a linker comprising the sequence “G4S” (SEQ ID NO: 188) between the KL2 domain and the HSA domain.

FIGS. 14A-14B illustrate exemplary engineered alpha klotho polypeptide constructs comprising a protease cleavable linker (PCL1) having the N-terminal to C-terminal configuration: [signal peptide]-[KL1 domain]-[KL2 domain]-[PCL1]-[HSA]. FIG. 14A shows a polypeptide construct having a C521S mutation in its KL2 domain. FIG. 14B shows a polypeptide construct having a C370S mutation in its KL1 domain and a C521S mutation in its KL2 domain.

FIG. 15 is a graph showing the activity of engineered KL polypeptide constructs comprising noncleavable (G4S; SEQ ID NO: 188) or protease cleavable (PCL1) linkers.

FIGS. 16A-16C show cleavage of KL polypeptide constructs comprising protease-cleavable linkers in murine and human sera over time. FIG. 16A shows cleavage of engineered KL polypeptide constructs in mouse serum between 0 and 48 hours, visualized with immunoblotting (IB) using an anti-KL antibody against the KL1 domain. FIG. 16B shows cleavage of engineered KL polypeptide constructs in human serum between 0 and 72 hours, visualized with IB using an anti-KL antibody against the KL1 domain. FIG. 16C shows cleavage of engineered KL polypeptide constructs in human serum between 0 and 72 hours, visualized with IB using an anti-KL antibody against the KL2 domain. K1: hKL958 (C521S)-G4S-HSA; K2: hKL958 (C521S)-PCL1-HSA; K3: KL958 (C521S C370S)-PCL1-HSA.

FIGS. 17A-17B show cleavage of a control peptide (PCL1 Pep) mediated by two different proteases, Kallikrein 2 (KLK2) and matrix metalloproteinase 7 (MMP7). The control peptide comprises the protease cleavable linker PCL1. FIG. 17A shows cleavage of PCL1 Pep by KLK2. FIG. 17B shows cleavage of PCL1 Pep by MMP7.

FIGS. 18A-18D show the effect of different buffers on MMP7-mediated cleavage of engineered KL polypeptide constructs comprising protease-cleavable linkers. FIG. 18A shows cleavage of engineered KL polypeptide constructs in reaction buffer (Rxn buffer), visualized with IB using an anti-KL antibody against the KL1 domain. FIG. 18B shows cleavage of engineered KL polypeptide constructs in Rxn buffer, visualized with IB using an anti-KL antibody against the KL2 domain. FIG. 18C shows cleavage of engineered KL polypeptide constructs in Rxn buffer, DMEM, or DMEM+FBS, visualized with IB using an anti-KL antibody against the KL1 domain. FIG. 18D shows cleavage of KL polypeptide constructs in Rxn buffer, DMEM, or DMEM+FBS, visualized with IB using an anti-KL antibody against the KL2 domain. K1: hKL958 (C521S)-G4S-HSA; K2: hKL958 (C521S)-PCL1-HSA; K3: KL958 (C521S C370S)-PCL1-HSA.

FIGS. 19A-19B are graphs showing the effect of MMP7-mediated cleavage and the buffer conditions of the MMP7-mediated cleavage reaction on the activity of engineered KL polypeptide constructs comprising noncleavable (G4S; SEQ ID NO:188) or protease cleavable (PCL1) linkers. FIG. 19A shows the change in activity of engineered KL polypeptide constructs upon cleavage with MMP7 in DMEM+10% FBS. FIG. 19B shows the change in activity of engineered KL polypeptide constructs upon cleavage with MMP7 in DMEM only.

FIG. 20 shows the effect of engineered KL polypeptide constructs on 3T3 cell proliferation and survival.

FIGS. 21A and 21B show the effect of engineered KL polypeptide constructs on serum levels of kidney function markers in an acute kidney injury model. FIG. 21A shows the serum levels of creatinine (CREA) in control (Veh) and cisplatin-induced kidney injury model (Cis) mice that were treated with an isotype (Iso) antibody or KL958 (C521S C370S)-PCL1-HSA (KL). FIG. 21B shows the serum levels of cystatin C (CysC) in serum of the same groups of mice in FIG. 21A.

### 6. DETAILED DESCRIPTION

### 6.1. Definitions

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, Or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

EC50: The term “EC50” refers to the half maximal effective concentration of a molecule, such as a polypeptide of the disclosure, which induces a response halfway between the baseline and maximum after a specified exposure time. The EC50 essentially represents the concentration of a polypeptide where 50% of its maximal effect is observed. In certain embodiments, the EC50 value equals the concentration of a polypeptide that gives half-maximal activation in a luciferase reporter assay as described in Section 9.1.5.

Fc Domain and Fc Region: The term “Fc domain” refers to a portion of a heavy chain that pairs with the corresponding portion of another heavy chain on a separate polypeptide chain. In some embodiments an Fc domain comprises a CH2 domain followed by a CH3 domain, with or without a hinge region N-terminal to the CH2 domain. In some embodiments an Fc domain is non-dimerizing. Optionally, a non-dimerizing Fc domain further comprises, in addition to a CH2 domain followed by a CH3 domain, an additional CH3 domain connected to the CH3 domain via a linker (e.g., an “Fc1.5 domain”). In some embodiments, the Fc domain comprises a CH3 domain incapable of dimerizing with another Fc domain (e.g., a “monomeric Fc”). The term “Fc region” refers to the region formed by association of two heavy chain Fc domains on separate polypeptide chains. The two Fc domains within the Fc region may be the same or different from one another.

Fibroblast Growth Factor Receptor or FGFR: The terms “fibroblast growth factor receptor” and “FGFR” as used herein refer to any one of FGFRs 1-4 from any vertebrate source, including mammals such as primates (e.g., humans, cynomolgus monkey (cyno)), dogs, and rodents (e.g., mice and rats), unless otherwise indicated, and includes naturally occurring variants of FGFR, e.g., splice variants or allelic variants (e.g., FGFR1c).

Host Cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

KL1 Domain or Alpha Klotho KL1 Domain: The terms “KL1 domain” and “alpha klotho KL1 domain,” as used herein, refer to an amino acid sequence corresponding to the KL1 domain of an alpha klotho protein (e.g., human alpha klotho or murine alpha klotho), as well as derivatives and variants thereof. Accordingly, a KL1 domain may be the amino acid sequence of a KL1 domain of an alpha klotho protein (e.g., human alpha klotho or murine alpha klotho) or a sequence having one, two, three, four, five, or more amino acid substitutions relative to the wild-type sequence. For example, in some embodiments, a KL1 domain of an alpha klotho moiety has L111S, F352V, and/or C370S substitutions, numbering relative to human alpha klotho (GenBank accession number BAA23382.1). In some embodiments, a KL1 domain is an amino acid sequence having at least 90%, at least 91%, at least 92%, at least 93%, at least 94%, at least 95%, at least 96%, at least 97%, at least 98%, at least 99%, at least 99.5% or 100% sequence identity to SEQ ID NO:9 or SEQ ID NO: 10. In some embodiments, a KL1 domain is an amino acid sequence having at least 90%, at least 91%, at least 92%, at least 93%, at least 94%, at least 95%, at least 96%, at least 97%, at least 98%, at least 99%, or at least 99.5% to SEQ ID NO:9 or SEQ ID NO: 10 and one, two or all three substitutions L111S, F352V, and/or C370S (e.g., L111S only, F352V only, C370S only, or both F352V and C370S).

KL2 Domain or Alpha Klotho KL2 Domain: The terms “KL2 domain” and “alpha klotho KL2 domain,” as used herein, refer to an amino acid sequence corresponding to the KL2 domain of an alpha klotho protein (e.g., human alpha klotho or murine alpha klotho), as well as derivatives and variants thereof. Accordingly, a KL2 domain may be the amino acid sequence of a KL2 domain of an alpha klotho protein (e.g., human alpha klotho or murine alpha klotho) or a sequence having one, two, three, four, five, or more amino acid substitutions relative to the wild-type sequence. For example, in some embodiments, a KL2 domain of an alpha klotho moiety has a C521S mutation, numbering relative to human alpha klotho (GenBank accession number BAA23382.1). In some embodiments, a KL2 domain is an amino acid sequence having at least 90%, at least 91%, at least 92%, at least 93%, at least 94%, at least 95%, at least 96%, at least 97%, at least 98%, at least 99%, at least 99.5%, or 100% sequence identity to SEQ ID NO:3 or SEQ ID NO:4. In particular embodiments, a KL2 domain is an amino acid sequence having at least 90%, at least 91%, at least 92%, at least 93%, at least 94%, at least 95%, at least 96%, at least 97%, at least 98%, at least 99%, or at least 99.5% sequence identity to SEQ ID NO:3 and comprising a C521S substitution, numbering relative to human alpha klotho (GenBank accession number BAA23382.1). In some embodiments, a KL2 domain is the amino acid sequence of SEQ ID NO: 4.

Klotho Moiety, Alpha Klotho Moiety, or KL Moiety: The terms “klotho moiety”, “alpha klotho moiety” and “KL moiety,” as used herein, refer to an amino acid sequence comprising at least one KL1 domain and/or at least one KL2 domain. In particular embodiments, an alpha klotho moiety comprises, in N- to C-terminal orientation, a KL1 domain and a KL2 domain. In some embodiments, the alpha klotho moiety further comprises an interdomain region between the KL1 domain and KL2 domain. In some embodiments, the interdomain region comprises the amino acid sequence of SEQ ID NO:21 or a variant thereof having 1, 2, 3, 4, or 5 amino acid substitutions relative to SEQ ID NO:21. In some embodiments, the alpha klotho moiety further comprises, at its C-terminus, the C-terminal tail of the KL extracellular domain or a portion thereof. In some embodiments, the C-terminal tail comprises amino acids 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, or 1-8 of SEQ ID NO: 7, or a variant thereof having 1, 2, 3, 4, or 5 amino acid substitutions relative to amino acids 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, or 1-8 of SEQ ID NO:7. In particular embodiments, the alpha klotho moiety comprises, in N- to C-terminal orientation: (i) a KL1 domain; (ii) an interdomain region; (iii) a KL2 domain; and (iv) a C-terminal tail region.

In some embodiments, the alpha klotho moiety comprises an amino acid sequence having at least 90%, at least 91%, at least 92%, at least 93%, at least 94%, at least 95%, at least 96%, at least 97%, at least 98%, at least 99%, at least 99.5%, or at least 99.8% sequence identity to SEQ ID NO:13. In some embodiments, the alpha klotho moiety comprises an amino acid sequence having at least 90%, at least 91%, at least 92%, at least 93%, at least 94%, at least 95%, at least 96%, at least 97%, at least 98%, at least 99%, at least 99.5%, at least 99.8%, or 100% sequence identity to SEQ ID NO:14. In some embodiments, the alpha klotho moiety comprises an amino acid sequence having at least 90%, at least 91%, at least 92%, at least 93%, at least 94%, at least 95%, at least 96%, at least 97%, at least 98%, at least 99%, at least 99.5%, or at least 99.8% sequence identity to SEQ ID NO: 15. In some embodiments, the alpha klotho moiety comprises an amino acid sequence having at least 90%, at least 91%, at least 92%, at least 93%, at least 94%, at least 95%, at least 96%, at least 97%, at least 98%, at least 99%, at least 99.5%, at least 99.8%, or 100% sequence identity to SEQ ID NO: 16.

Typically, the klotho moieties of the disclosure are soluble and lack a transmembrane domain and an intracellular domain.

Linker: The term “linker” as used herein refers to a connecting peptide between two moieties. For example, a linker can connect an alpha klotho moiety to a stabilization domain.

Operably Linked: The term “operably linked” as used herein refers to a functional relationship between two or more regions of a polypeptide chain in which the two or more regions are linked so as to produce a functional polypeptide, or two or more nucleic acid sequences, e.g., to produce an in-frame fusion of two polypeptide components or to link a regulatory sequence to a coding sequence.

Polypeptide, Peptide, and Protein: The terms “polypeptide”, “peptide” and “protein” are used interchangeably herein to refer to a polymer of amino acid residues.

Subject: The term “subject” includes human and non-human animals. Non-human animals include all vertebrates, e.g., mammals and non-mammals, such as non-human primates, sheep, dogs, cows, chickens, amphibians, and reptiles. Except when noted, the terms “patient” or “subject” are used herein interchangeably.

Treat, Treatment, Treating: As used herein, the terms “treat”, “treatment” and “treating” refer to the reduction or amelioration of the progression, severity and/or duration of a disorder as described herein, the amelioration of one or more symptoms (preferably, one or more discernible symptoms) of a condition or disorder as described herein, or prevention of a condition or disorder as described herein, e.g., kidney disease or an age-related condition or disorder, resulting from the administration of a molecule or composition (e.g., one or more alpha klotho polypeptides of the disclosure). In specific embodiments, the terms “treat”, “treatment” and “treating” refer to the amelioration of at least one measurable physical parameter of a disorder, e.g., kidney disease or an age-related disorder, not necessarily discernible by the patient. In other embodiments the terms “treat”, “treatment” and “treating” refer to the inhibition of the progression or onset of a disorder, either physically by, e.g., stabilization of a discernible symptom, physiologically by, e.g., stabilization of a physical parameter, or both.

### 6.2. Soluble Alpha Klotho Polypeptides

The present disclosure relates to engineered alpha klotho polypeptides (also “engineered KL polypeptides”) comprising an alpha klotho moiety. Typically, the alpha klotho moiety has a KL2 domain having an amino acid substitution at the position corresponding to amino acid C521 of full-length human alpha klotho (SEQ ID NO: 1) and also lacks a cysteine at the position corresponding to amino acid C970 of full-length human alpha klotho (SEQ ID NO: 1), if present.

Amino acid residues C521 and C970 of full-length human alpha klotho (SEQ ID NO: 1), without being bound by theory, are understood to be free cysteines which are not paired via a disulfide bond to any other cysteine in the natural protein. Although there are many other free cysteine residues in the alpha klotho protein, it was surprisingly discovered that modification of both of these cysteine residues particularly (e.g., by substitution of C521 and by deletion of a portion the alpha klotho protein comprising C970) results in an alpha klotho protein having significantly enhanced activity, production yield, and stability relative to wild-type. Accordingly, polypeptides of the present disclosure generally include an alpha klotho moiety comprising 1) a KL2 domain having an amino acid substitution at position 521, and 2) a C-terminal tail region which does not comprise a cysteine at position 970 (if present) or which comprises a C-terminal deletion of at least 12 amino acids such that position 970 is absent. Exemplary alpha klotho moieties are described in Section 6.3.

Soluble alpha klotho polypeptides of the present disclosure may, in some embodiments, further comprise a stabilization moiety, optionally linked to the alpha klotho moiety (e.g., N-terminally or C-terminally) via a linker (e.g., a protease cleavable linker). Exemplary stabilization moieties are described in Section 6.4, and include Fc domains (as described in Section 6.4.1), albumin moieties (as described in Section 6.4.2) and other stabilization moieties (e.g., as described in Section 6.4.3). Exemplary linkers are described in Section 6.5, with exemplary protease-cleavable linkers described in Section 6.5.2.

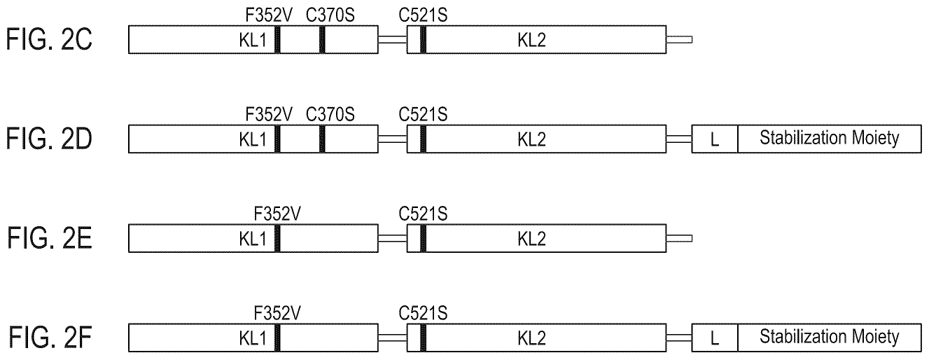

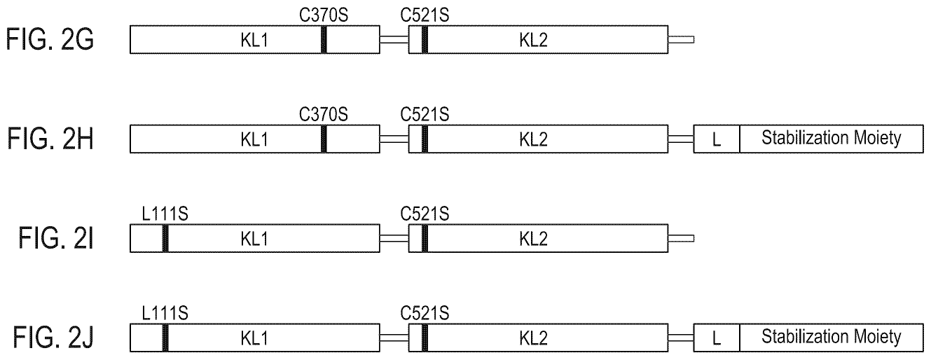

Exemplary engineered alpha klotho polypeptides are illustrated in FIGS. 2A-2J. Amino acid sequences of example engineered alpha klotho polypeptides are provided as SEQ ID NOs: 42, 44, 46, 48, 50, 52, 54, 56, 58, 60, 62, 64, 66, 68, and 70. Sequences of example nucleic acids encoding engineered alpha klotho polypeptides are provided as SEQ ID NOs: 41, 43, 45, 47, 49, 51, 53, 55, 57, 59, 61, 63, 65, 67, and 69.

### 6.3. Alpha Klotho Moieties

Naturally occurring alpha klotho (KL) is encoded by the α-klotho gene located on human chromosome 13. The product of the α-klotho gene is a single-pass transmembrane protein comprising, from N- to C-terminal direction, an extracellular region, a transmembrane region, and a cytoplasmic region. The extracellular region of KL has two domains (sometimes “subdomains”), termed KL1 and KL2. In addition, an interdomain amino acid sequence is located between the KL1 and KL2 domains and a C-terminal tail region is located C-terminal to the KL2 domain.

An amino acid sequence of human KL (GenBank accession number BAA23382.1) is reproduced below with the signal sequence italicized, the KL1 domain underlined with a straight line, the KL2 domain in bold, and the transmembrane region underlined with a dotted line.

(SEQ ID NO: 1)
MPASAPPRRPRPPPPSLSLLLVLLGLGGRRLRAEPGDGAQTWARVSRPPAPEAAGLF
QGTFPDGFLWAVGSAAYQTEGGWQQHGKGASIWDTFTHHPLAPPGDSRNASLPLGAP
SPLQPATGDVASDSYNNVFRDTEALRELGVTHYRFSISWARVLPNGSAGVPNREGLR
YYRRLLERLRELGVQPVVTLYHWDLPQRLQDAYGGWANRALADHFRDYAELCFRHFG
GQVKYWITIDNPYVVAWHGYATGRLAPGIRGSPRLGYLVAHNLLLAHAKVWHLYNTS
FRPTQGGQVSIALSSHWINPRRMTDHSIKECQKSLDFVLGWFAKPVFIDGDYPESMK
NNLSSILPDFTESEKKFIKGTADFFALCFGPTLSFQLLDPHMKFRQLESPNLRQLLS
WIDLEFNHPQIFIVENGWFVSGTTKRDDAKYMYYLKKFIMETLKAIKLDGVDVIGYT
AWSLMDGFEWHRGYSIRRGLFYVDFLSQDKMLLPKSSALFYQKLIEKNGFPPLPENQ
PLEGTFPCDFAWGVVDNYIQVDTTLSQFTDLNVYLWDVHHSKRLIKVDGVVTKKRKS
YCVDFAAIQPQIALLQEMHVTHFRFSLDWALILPLGNQSQVNHTILQYYRCMASELV
RVNITPVVALWQPMAPNQGLPRLLARQGAWENPYTALAFAEYARLCFQELGHHVKLW
ITMNEPYTRNMTYSAGHNLLKAHALAWHVYNEKFRHAQNGKISIALQADWIEPACPF
SQKDKEVAERVLEFDIGWLAEPIFGSGDYPWVMRDWLNQRNNFLLPYFTEDEKKLIQ
GTFDFLALSHYTTILVDSEKEDPIKYNDYLEVQEMTDITWLNSPSQVAVVPWGLRKV
LNWLKFKYGDLPMYIISNGIDDGLHAEDDQLRVYYMQNYINEALKAHILDGINLCGY
FAYSFNDRTAPRFGLYRYAADQFEPKASMKHYRKIIDSNGFPGPETLERFCPEEFTV

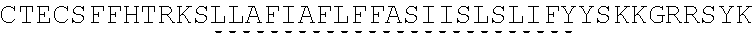

An alternative amino acid sequence of human KL (UniProtKB accession number Q9UEF7-1), having a single amino acid substitution relative to SEQ ID NO:1, is reproduced below with the signal sequence italicized, the KL1 domain underlined with a straight line, the KL2 domain in bold, and the transmembrane region underlined with a dotted line.

(SEQ ID NO: 2)
MPASAPPRRPRPPPPSLSLLLVLLGLGGRRLRAEPGDGAQTWARVSRPPAPEAAGLF
QGTFPDGFLWAVGSAAYQTEGGWQQHGKGASIWDTFTHHPLAPPGDSRNASLPLGAP
SPLQPATGDVASDSYNNVFRDTEALRELGVTHYRFSISWARVLPNGSAGVPNREGLR
YYRRLLERLRELGVQPVVTLYHWDLPQRLQDAYGGWANRALADHFRDYAELCFRHFG
GQVKYWITIDNPYVVAWHGYATGRLAPGIRGSPRLGYLVAHNLLLAHAKVWHLYNTS
FRPTQGGQVSIALSSHWINPRRMTDHSIKECQKSLDFVLGWFAKPVFIDGDYPESMK
NNLSSILPDFTESEKKFIKGTADFFALCFGPTLSFQLLDPHMKFRQLESPNLRQLLS
WIDLEFNHPQIFIVENGWFVSGTTKRDDAKYMYYLKKFIMETLKAIKLDGVDVIGYT
AWSLMDGFEWHRGYSIRRGLFYVDFLSQDKMLLPKSSALFYQKLIEKNGFPPLPENQ
PLEGTFPCDFAWGVVDNYIQVDTTLSQFTDLNVYLWDVHHSKRLIKVDGVVTKKRKS
YCVDFAAIQPQIALLQEMHVTHFRFSLDWALILPLGNQSQVNHTILQYYRCMASELV
RVNITPVVALWQPMAPNQGLPRLLARQGAWENPYTALAFAEYARLCFQELGHHVKLW
ITMNEPYTRNMTYSAGHNLLKAHALAWHVYNEKFRHAQNGKISIALQADWIEPACPF
SQKDKEVAERVLEFDIGWLAEPIFGSGDYPWVMRDWLNQRNNFLLPYFTEDEKKLIQ
GTFDFLALSHYTTILVDSEKEDPIKYNDYLEVQEMTDITWLNSPSQVAVVPWGLRKV
LNWLKFKYGDLPMYIISNGIDDGLHAEDDQLRVYYMQNYINEALKAHILDGINLCGY
FAYSFNDRTAPRFGLYRYAADQFEPKASMKHYRKIIDSNGFPGPETLERFCPEEFTV

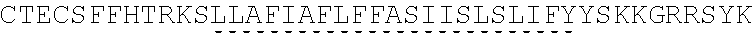

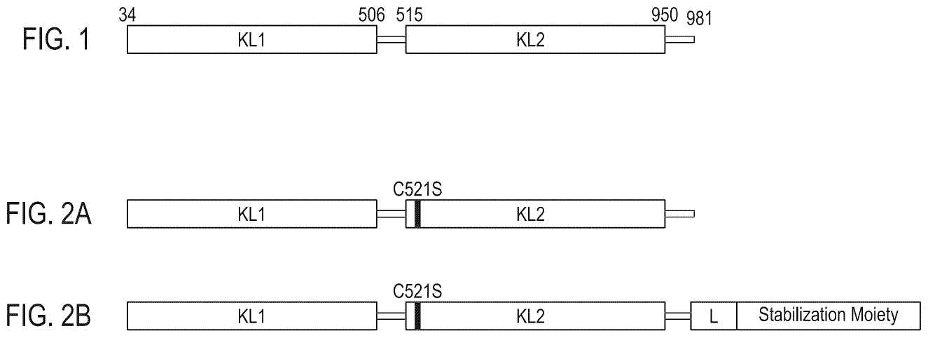

The extracellular region of human KL (FIG. 1) consists of amino acids 34 through 981 and, as discussed above, includes a KL1 domain (amino acids 34-506; SEQ ID NO:9), an interdomain region (amino acids 507-514; SEQ ID NO:21), a KL2 domain (amino acids 515-950; SEQ ID NO:3), and a C-terminal tail region (amino acids 951-981; SEQ ID NO:7). An alpha klotho moiety, as discussed herein in the context of an engineered alpha klotho polypeptide, comprises at least a portion of the extracellular region of human KL or a variant or derivative thereof.

In certain aspects, the alpha klotho moiety comprises an alpha klotho KL2 domain. In some embodiments, the alpha klotho KL2 domain has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or at least about 99.8% sequence identity to the KL2 domain of human KL (SEQ ID NO:3). The alpha klotho KL2 domain may comprise 1, 2, 3, 4, 5, or more amino acid mutations (e.g., deletions, additions, or substitutions) relative to the KL2 domain of human KL. In some embodiments, the alpha klotho KL2 domain has an amino acid substitution at the position corresponding to C521 of full-length human KL (SEQ ID NO:1). In some embodiments, this amino acid substitution is a cysteine to serine substitution. In other embodiments, this amino acid substitution is a substitution of cysteine with a different amino acid that is not a serine.

In some embodiments, the alpha klotho moiety lacks a cysteine at the position corresponding to C970 of full-length human KL (SEQ ID NO:1), if present. Accordingly, in some aspects, the alpha klotho moiety comprises a position corresponding to C970 of full-length human KL (SEQ ID NO:1), where the position is not a cysteine. For example, the alpha klotho moiety may comprise an amino acid substitution at the position corresponding to C970, which substitution may be, for example, a cysteine to serine substitution. Alternatively, in some aspects, the alpha klotho moiety lacks a position corresponding to C970 of full-length human KL (SEQ ID NO: 1) entirely. For example, the alpha klotho moiety may comprise only a portion of the C-terminal tail region of the extracellular region of human KL (SEQ ID NO:7), which portion lacks the position corresponding to C970 of full-length human KL. Thus, in some embodiments, the alpha klotho moiety lacks the C-terminal 12, 13, 14, 15, 16, 17, 18, 19, 20, 21, 22, 23, 24, 25, 26, 27, 28, 29, or 30 amino acids of the C-terminal tail of full-length human KL (SEQ ID NO:7). In some embodiments, the alpha klotho moiety lacks the C-terminal 23 amino acids of the C-terminal tail of full-length human KL (SEQ ID NO:7). Without being bound by theory, it is believed that an alpha klotho moiety having both a C521 mutation (e.g., C521S) and lacking a cysteine at position 970 (or lacking position 970 entirely), in the context of an engineered alpha klotho polypeptide, provides for a significant increase in both production yield and bioactivity compared with an alpha klotho moiety comprising a wild type human KL2 domain and C-terminal tail region. Accordingly, in some embodiments, the alpha klotho moiety (1) comprises a KL2 domain having a C521S mutation (numbering relative to SEQ ID NO:1) and (2) lacks the C-terminal 12, 13, 14, 15, 16, 17, 18, 19, 20, 21, 22, 23, 24, 25, 26, 27, 28, 29, or 30 amino acids of the C-terminal tail of full-length human KL (SEQ ID NO:7). In some embodiments, the alpha klotho moiety (1) comprises a KL2 domain having a C521S mutation (numbering relative to SEQ ID NO:1) and (2) lacks an amino acid sequence having 80%, 85%, 90%, 91%, 92%, 93%, 94%, 95%, 96%, 97%, 98%, 99%, or 100% sequence identity to SEQ ID NO:8.

In certain aspects, the alpha klotho moiety further comprises an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or 100% sequence identity to the KL1 domain of human KL (SEQ ID NO:9 or SEQ ID NO: 10). The alpha klotho KL1 domain may comprise 1, 2, 3, 4, 5, or more amino acid mutations (e.g., deletions, additions, or substitutions) relative to the KL1 domain of human KL. In some embodiments, the alpha klotho KL1 domain has an amino acid substitution at the position corresponding to L111 of full-length human KL (SEQ ID NO: 1). In some embodiments, this amino acid substitution is a lysine to serine substitution. In some embodiments, the alpha klotho KL1 domain has an amino acid substitution at the position corresponding to F352 of full-length human KL (SEQ ID NO: 1). In some embodiments, this amino acid substitution is a phenylalanine to valine substitution. In some embodiments, the alpha klotho KL1 domain has an amino acid substitution at the position corresponding to C370 of full-length human KL (SEQ ID NO: 1). In some embodiments, this amino acid substitution is a cysteine to serine substitution.

Sequences of certain example alpha klotho moieties of the present disclosure are provided in Table 1, below. In some embodiments, the alpha klotho moiety has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5% sequence identity, or 100% sequence identity to a sequence of Table 1.

TABLE 1
Example Alpha Klotho Moieties

| | | SEQ ID |
|---|---|---|
| Name | Sequence | NO: |
| hKL ECD | EPGDGAQTWARFSRPPAPEAAGLFQGTFPDGFLWAVGSAAYQTEGG | 13 |
| | WQQHGKGASIWDTFTHHPLAPPGDSRNASLPLGAPSPLQPATGDVAS | |
| | DSYNNVFRDTEALRELGVTHYRFSISWARVLPNGSAGVPNREGLRYY | |
| | RRLLERLRELGVQPVVTLYHWDLPQRLQDAYGGWANRALADHFRDY | |
| | AELCFRHFGGQVKYWITIDNPYVVAWHGYATGRLAPGIRGSPRLGYLV | |
| | AHNLLLAHAKVWHLYNTSFRPTQGGQVSIALSSHWINPRRMTDHSIKE | |
| | CQKSLDFVLGWFAKPVFIDGDYPESMKNNLSSILPDFTESEKKFIKGTA | |
| | DFFALCFGPTLSFQLLDPHMKFRQLESPNLRQLLSWIDLEFNHPQIFIV | |
| | ENGWFVSGTTKRDDAKYMYYLKKFIMETLKAIKLDGVDVIGYTAWSLM | |
| | DGFEWHRGYSIRRGLFYVDFLSQDKMLLPKSSALFYQKLIEKNGFPPL | |
| | PENQPLEGTFPCDFAWGVVDNYIQVDTTLSQFTDLNVYLWDVHHSKR | |
| | LIKVDGVVTKKRKSYCVDFAAIQPQIALLQEMHVTHFRFSLDWALILPLG | |
| | NQSQVNHTILQYYRCMASELVRVNITPVVALWQPMAPNQGLPRLLAR | |
| | QGAWENPYTALAFAEYARLCFQELGHHVKLWITMNEPYTRNMTYSAG | |
| | HNLLKAHALAWHVYNEKFRHAQNGKISIALQADWIEPACPFSQKDKEV | |
| | AERVLEFDIGWLAEPIFGSGDYPWVMRDWLNQRNNFLLPYFTEDEKK | |
| | LIQGTFDFLALSHYTTILVDSEKEDPIKYNDYLEVQEMTDITWLNSPSQV | |
| | AVVPWGLRKVLNWLKFKYGDLPMYIISNGIDDGLHAEDDQLRVYYMQ | |
| | NYINEALKAHILDGINLCGYFAYSFNDRTAPRFGLYRYAADQFEPKASM | |
| | KHYRKIIDSNGFPGPETLERFCPEEFTVCTECSFFHTRKS | |
| hKL ECD - | EPGDGAQTWARFSRPPAPEAAGLFQGTFPDGFLWAVGSAAYQTEGG | 14 |
| C521S | WQQHGKGASIWDTFTHHPLAPPGDSRNASLPLGAPSPLQPATGDVAS | |
| | DSYNNVFRDTEALRELGVTHYRFSISWARVLPNGSAGVPNREGLRYY | |
| | RRLLERLRELGVQPVVTLYHWDLPQRLQDAYGGWANRALADHFRDY | |
| | AELCFRHFGGQVKYWITIDNPYVVAWHGYATGRLAPGIRGSPRLGYLV | |
| | AHNLLLAHAKVWHLYNTSFRPTQGGQVSIALSSHWINPRRMTDHSIKE | |
| | CQKSLDFVLGWFAKPVFIDGDYPESMKNNLSSILPDFTESEKKFIKGTA | |
| | DFFALCFGPTLSFQLLDPHMKFRQLESPNLRQLLSWIDLEFNHPQIFIV | |
| | ENGWFVSGTTKRDDAKYMYYLKKFIMETLKAIKLDGVDVIGYTAWSLM | |
| | DGFEWHRGYSIRRGLFYVDFLSQDKMLLPKSSALFYQKLIEKNGFPPL | |
| | PENQPLEGTFPSDFAWGVVDNYIQVDTTLSQFTDLNVYLWDVHHSKR | |
| | LIKVDGVVTKKRKSYCVDFAAIQPQIALLQEMHVTHFRFSLDWALILPLG | |
| | NQSQVNHTILQYYRCMASELVRVNITPVVALWQPMAPNQGLPRLLAR | |
| | QGAWENPYTALAFAEYARLCFQELGHHVKLWITMNEPYTRNMTYSAG | |
| | HNLLKAHALAWHVYNEKFRHAQNGKISIALQADWIEPACPFSQKDKEV | |
| | AERVLEFDIGWLAEPIFGSGDYPWVMRDWLNQRNNFLLPYFTEDEKK | |
| | LIQGTFDFLALSHYTTILVDSEKEDPIKYNDYLEVQEMTDITWLNSPSQV | |
| | AVVPWGLRKVLNWLKFKYGDLPMYIISNGIDDGLHAEDDQLRVYYMQ | |
| | NYINEALKAHILDGINLCGYFAYSFNDRTAPRFGLYRYAADQFEPKASM | |
| | KHYRKIIDSNGFPGPETLERFCPEEFTVCTECSFFHTRKS | |
| hKL ECD - | EPGDGAQTWARFSRPPAPEAAGLFQGTFPDGFLWAVGSAAYQTEGG | 15 |
| 23aa | WQQHGKGASIWDTFTHHPLAPPGDSRNASLPLGAPSPLQPATGDVAS | |
| truncation | DSYNNVFRDTEALRELGVTHYRFSISWARVLPNGSAGVPNREGLRYY | |
| (wt) | RRLLERLRELGVQPVVTLYHWDLPQRLQDAYGGWANRALADHFRDY | |
| | AELCFRHFGGQVKYWITIDNPYVVAWHGYATGRLAPGIRGSPRLGYLV | |
| | AHNLLLAHAKVWHLYNTSFRPTQGGQVSIALSSHWINPRRMTDHSIKE | |
| | CQKSLDFVLGWFAKPVFIDGDYPESMKNNLSSILPDFTESEKKFIKGTA | |
| | DFFALCFGPTLSFQLLDPHMKFRQLESPNLRQLLSWIDLEFNHPQIFIV | |
| | ENGWFVSGTTKRDDAKYMYYLKKFIMETLKAIKLDGVDVIGYTAWSLM | |
| | DGFEWHRGYSIRRGLFYVDFLSQDKMLLPKSSALFYQKLIEKNGFPPL | |
| | PENQPLEGTFPCDFAWGVVDNYIQVDTTLSQFTDLNVYLWDVHHSKR | |
| | LIKVDGVVTKKRKSYCVDFAAIQPQIALLQEMHVTHFRFSLDWALILPLG | |
| | NQSQVNHTILQYYRCMASELVRVNITPVVALWQPMAPNQGLPRLLAR | |
| | QGAWENPYTALAFAEYARLCFQELGHHVKLWITMNEPYTRNMTYSAG | |
| | HNLLKAHALAWHVYNEKFRHAQNGKISIALQADWIEPACPFSQKDKEV | |
| | AERVLEFDIGWLAEPIFGSGDYPWVMRDWLNQRNNFLLPYFTEDEKK | |
| | LIQGTFDFLALSHYTTILVDSEKEDPIKYNDYLEVQEMTDITWLNSPSQV | |
| | AVVPWGLRKVLNWLKFKYGDLPMYIISNGIDDGLHAEDDQLRVYYMQ | |
| | NYINEALKAHILDGINLCGYFAYSFNDRTAPRFGLYRYAADQFEPKASM | |
| | KHYRKIIDSNGFPGPET | |
| hKL ECD - | EPGDGAQTWARFSRPPAPEAAGLFQGTFPDGFLWAVGSAAYQTEGG | 16 |
| 23aa | WQQHGKGASIWDTFTHHPLAPPGDSRNASLPLGAPSPLQPATGDVAS | |
| truncation | DSYNNVFRDTEALRELGVTHYRFSISWARVLPNGSAGVPNREGLRYY | |
| (C521S) | RRLLERLRELGVQPVVTLYHWDLPQRLQDAYGGWANRALADHFRDY | |
| | AELCFRHFGGQVKYWITIDNPYVVAWHGYATGRLAPGIRGSPRLGYLV | |
| | AHNLLLAHAKVWHLYNTSFRPTQGGQVSIALSSHWINPRRMTDHSIKE | |
| | CQKSLDFVLGWFAKPVFIDGDYPESMKNNLSSILPDFTESEKKFIKGTA | |
| | DFFALCFGPTLSFQLLDPHMKFRQLESPNLRQLLSWIDLEFNHPQIFIV | |
| | ENGWFVSGTTKRDDAKYMYYLKKFIMETLKAIKLDGVDVIGYTAWSLM | |
| | DGFEWHRGYSIRRGLFYVDFLSQDKMLLPKSSALFYQKLIEKNGFPPL | |
| | PENQPLEGTFPSDFAWGVVDNYIQVDTTLSQFTDLNVYLWDVHHSKR | |
| | LIKVDGVVTKKRKSYCVDFAAIQPQIALLQEMHVTHFRFSLDWALILPLG | |
| | NQSQVNHTILQYYRCMASELVRVNITPVVALWQPMAPNQGLPRLLAR | |
| | QGAWENPYTALAFAEYARLCFQELGHHVKLWITMNEPYTRNMTYSAG | |
| | HNLLKAHALAWHVYNEKFRHAQNGKISIALQADWIEPACPFSQKDKEV | |
| | AERVLEFDIGWLAEPIFGSGDYPWVMRDWLNQRNNFLLPYFTEDEKK | |
| | LIQGTFDFLALSHYTTILVDSEKEDPIKYNDYLEVQEMTDITWLNSPSQV | |
| | AVVPWGLRKVLNWLKFKYGDLPMYIISNGIDDGLHAEDDQLRVYYMQ | |
| | NYINEALKAHILDGINLCGYFAYSFNDRTAPRFGLYRYAADQFEPKASM | |
| | KHYRKIIDSNGFPGPET | |
| hKL ECD - | EPGDGAQTWARFSRPPAPEAAGLFQGTFPDGFLWAVGSAAYQTEGG | 76 |
| 23aa | WQQHGKGASIWDTFTHHPLAPPGDSRNASLPLGAPSPLQPATGDVAS | |
| truncation | DSYNNVFRDTEALRELGVTHYRFSISWARVLPNGSAGVPNREGLRYY | |
| (C521S + | RRLLERLRELGVQPVVTLYHWDLPQRLQDAYGGWANRALADHFRDY | |
| C370S) | AELCFRHFGGQVKYWITIDNPYVVAWHGYATGRLAPGIRGSPRLGYLV | |
| | AHNLLLAHAKVWHLYNTSFRPTQGGQVSIALSSHWINPRRMTDHSIKE | |
| | CQKSLDFVLGWFAKPVFIDGDYPESMKNNLSSILPDFTESEKKFIKGTA | |
| | DFFALSFGPTLSFQLLDPHMKFRQLESPNLRQLLSWIDLEFNHPQIFIV | |
| | ENGWFVSGTTKRDDAKYMYYLKKFIMETLKAIKLDGVDVIGYTAWSLM | |
| | DGFEWHRGYSIRRGLFYVDFLSQDKMLLPKSSALFYQKLIEKNGFPPL | |
| | PENQPLEGTFPSDFAWGVVDNYIQVDTTLSQFTDLNVYLWDVHHSKR | |
| | LIKVDGVVTKKRKSYCVDFAAIQPQIALLQEMHVTHFRFSLDWALILPLG | |
| | NQSQVNHTILQYYRCMASELVRVNITPVVALWQPMAPNQGLPRLLAR | |
| | QGAWENPYTALAFAEYARLCFQELGHHVKLWITMNEPYTRNMTYSAG | |
| | HNLLKAHALAWHVYNEKFRHAQNGKISIALQADWIEPACPFSQKDKEV | |
| | AERVLEFDIGWLAEPIFGSGDYPWVMRDWLNQRNNFLLPYFTEDEKK | |
| | LIQGTFDFLALSHYTTILVDSEKEDPIKYNDYLEVQEMTDITWLNSPSQV | |
| | AVVPWGLRKVLNWLKFKYGDLPMYIISNGIDDGLHAEDDQLRVYYMQ | |
| | NYINEALKAHILDGINLCGYFAYSFNDRTAPRFGLYRYAADQFEPKASM | |
| | KHYRKIIDSNGFPGPET | |

### 6.4. Stabilization Moieties

The engineered alpha klotho polypeptides of the disclosure can comprise a stabilization moiety that can extend the molecule's serum half-life in vivo. Serum half-life is often divided into an alpha phase and a beta phase. Either or both phases may be improved significantly by addition of an appropriate stabilization moiety. For example, the stabilization moiety can increase the serum half-life of the engineered alpha klotho polypeptide by more than 5, 10, 20, 30, 40, 50, 60, 70, 80, 90, 100, 120, 150, 200, 400, 600, 800, 1000% or more relative to a corresponding stabilization alpha klotho polypeptide not containing the stabilization moiety. For the purpose of this disclosure, serum half-life can refer to the half-life in humans or other mammals (e.g., mice or non-human primates).

Stabilization moieties include but are not limited to polyoxyalkylene moieties (e.g., polyethylene glycol), sugars (e.g., sialic acid), and well-tolerated protein moieties (e.g., Fc and fragments and variants thereof, transferrin, and serum albumin). In some embodiments, the stabilization moiety is human serum albumin (or a variant thereof having 1, 2, 3, 4, 5, or more amino acid substitutions relative to human serum albumin). In some embodiments, the stabilization moiety is an Fc domain.

Other stabilization moieties that can be used in the engineered alpha klotho polypeptides of the disclosure include those described in Kontermann et al., 2011, Current Opinion in Biotechnology 22:868-76. Such stabilization moieties include, but are not limited to, human serum albumin, human serum albumin binders (e.g., Adnectin PKE, AlbudAb, ABD), XTEN®, PAS (recombinant PEG mimetics based on the three amino acids proline, alanine, and serine), carbohydrates (e.g., hydroxyethyl starch (HES)), glycosylation, polysialic acid, and fatty acids.

In some embodiments, the engineered alpha klotho polypeptide comprising a stabilization moiety will preferably retain at least about 25%, 50%, 60%, 70%, 80%, 85%, 90%, 95% or 100% of the biological activity associated with the unmodified engineered alpha klotho polypeptide. In some embodiments, biological activity refers to its ability to bind to FGFR, or both FGFR and FGF23, as assessed by Kp, Kon, or Koff.

The stabilization moiety can be connected to one or more other components of the engineered alpha klotho polypeptides of the disclosure (e.g., the alpha klotho moiety) via a linker, for example as described in Section 6.5. In some embodiments, the stabilization moiety is C-terminal to the alpha klotho moiety. In some embodiments, the stabilization moiety is N-terminal to the alpha klotho moiety.

In some embodiments, the stabilization moiety is an Fc domain or a variant of fragment thereof, as described in Section 6.4.1. In some embodiments, the stabilization moiety is a non-dimerizing Fc domain, as described in Section 6.4.1.1. In some embodiments, the stabilization moiety is an Fc1.5 domain, as described in Section 6.4.1.1.1.

In other embodiments, the stabilization moiety is a serum albumin or a variant or fragment thereof, as described in Section 6.4.2.

Additional stabilization moieties are also contemplated herein, including a polyethylene glycol moiety or other polymer, as described in Section 6.4.3.

### 6.4.1. Fc Domains

In some embodiments, the engineered alpha klotho polypeptides of the disclosure comprise one or more Fc domains as a stabilization moiety.

The Fc domains can be derived from any suitable species. In one embodiment the Fc domain is derived from a human Fc domain. In some embodiments, the alpha klotho moiety is fused to an IgG Fc domain. The alpha klotho moiety may be fused to the N-terminus or the C-terminus of the IgG Fc domain.

The Fc domains can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

Exemplary sequences of Fc domains from IgG1, IgG2, IgG3, and IgG4 are provided in Table Y-1, below.

TABLE Y-1
Fc Sequences

| | | SEQ |
|---|---|---|
| Fc | Sequence | ID NO |
| hIgG1 Fc (amino | APELLGGPSVFLFPPKPKDTLMISRTPEVTCVVVDVSHEDPEVKFNWYV | 23 |
| acids 114-330 of | DGVEVHNAKTKPREEQYNSTYRVVSVLTVLHQDWLNGKEYKCKVSNK | |
| UniprotKB | ALPAPIEKTISKAKGQPREPQVYTLPPSRDELTKNQVSLTCLVKGFYPSD | |
| P01857-1) | IAVEWESNGQPENNYKTTPPVLDSDGSFFLYSKLTVDKSRWQQGNVFS | |
| | CSVMHEALHNHYTQKSLSLSPGK | |
| hIgG2 Fc (amino | APPVAGPSVFLFPPKPKDTLMISRTPEVTCVVVDVSHEDPEVQFNWYV | 24 |
| acids 111-326 of | DGVEVHNAKTKPREEQFNSTFRVVSVLTVVHQDWLNGKEYKCKVSNK | |
| UniprotKB | GLPAPIEKTISKTKGQPREPQVYTLPPSREEMTKNQVSLTCLVKGFYPS | |
| P01859-1) | DISVEWESNGQPENNYKTTPPMLDSDGSFFLYSKLTVDKSRWQQGNV | |
| | FSCSVMHEALHNHYTQKSLSLSPGK | |
| hIgG3 Fc (amino | APELLGGPSVFLFPPKPKDTLMISRTPEVTCVVVDVSHEDPEVQFKWYV | 25 |
| acids 161-377 of | DGVEVHNAKTKPREEQYNSTFRVVSVLTVLHQDWLNGKEYKCKVSNK | |
| UniprotKB | ALPAPIEKTISKTKGQPREPQVYTLPPSREEMTKNQVSLTCLVKGFYPS | |
| P01860-1) | DIAVEWESSGQPENNYNTTPPMLDSDGSFFLYSKLTVDKSRWQQGNIF | |
| | SCSVMHEALHNRFTQKSLSLSPGK | |
| hIgG4 Fc (amino | APEFLGGPSVFLFPPKPKDTLMISRTPEVTCVVVDVSQEDPEVQFNWY | 26 |
| acids 111-327 of | VDGVEVHNAKTKPREEQFNSTYRVVSVLTVLHQDWLNGKEYKCKVSN | |
| UniprotKB | KGLPSSIEKTISKAKGQPREPQVYTLPPSQEEMTKNQVSLTCLVKGFYP | |
| P01861-1) | SDIAVEWESNGQPENNYKTTPPVLDSDGSFFLYSRLTVDKSRWQEGN | |
| | VFSCSVMHEALHNHYTQKSLSLSLGK | |

In some embodiments, the Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:23. In cases where an Fc domain comprises at least 90% sequence identity and less than 100% sequence identity to SEQ ID NO:23 (e.g., between 90% and 99% sequence identity to SEQ ID NO:23), an Fc domain may also comprise one or more amino acid substitutions described herein, for example one or more substitutions that prevent dimerization (e.g., as described in Section 6.4.1.1) and/or one or more substitutions that alter effector function (e.g., as described in Section 6.4.1.2).

In some embodiments, the Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:24. In cases where an Fc domain comprises at least 90% sequence identity and less than 100% sequence identity to SEQ ID NO:24 (e.g., between 90% and 99% sequence identity to SEQ ID NO:24), an Fc domain may also comprise one or more amino acid substitutions described herein, for example one or more substitutions that prevent dimerization (e.g., as described in Section 6.4.1.1) and/or one or more substitutions that alter effector function (e.g., as described in Section 6.4.1.2).

In some embodiments, the Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:25. In cases where an Fc domain comprises at least 90% sequence identity and less than 100% sequence identity to SEQ ID NO:25 (e.g., between 90% and 99% sequence identity to SEQ ID NO:25), an Fc domain may also comprise one or more amino acid substitutions described herein, for example one or more substitutions that prevent dimerization (e.g., as described in Section 6.4.1.1) and/or one or more substitutions that alter effector function (e.g., as described in Section 6.4.1.2).

In some embodiments, the Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:26. In cases where an Fc domain comprises at least 90% sequence identity and less than 100% sequence identity to SEQ ID NO:26 (e.g., between 90% and 99% sequence identity to SEQ ID NO:26), an Fc domain may also comprise one or more amino acid substitutions described herein, for example one or more substitutions that prevent dimerization (e.g., as described in Section 6.4.1.1) and/or one or more substitutions that alter effector function (e.g., as described in Section 6.4.1.2).

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region. The alpha klotho polypeptides of the present disclosure can comprise Fc domains comprising heavy chain constant domains from one or more different classes of antibody, for example one, two or three different classes.

In some other embodiments, the stabilization moiety comprises two Fc domains forming a dimer. The two Fc domains can be the same or different from one another. In certain embodiments, the two Fc domains are identical. However, the Fc domains that allow for heterodimerization can be used to manufacture engineered alpha klotho polypeptides comprising different polypeptide components. An engineered alpha klotho polypeptide with different polypeptide components may comprise, for example, an alpha klotho moiety and another polypeptide, or a first alpha klotho moiety fused to another polypeptide and a second alpha klotho moiety that is not fused to another polypeptide.

### 6.4.1.1. Non-Dimerizing Fc Domains

In some embodiments, the Fc domain is a non-dimerizing (or “monomeric”) Fc domain, which refers to an Fc domain that has a reduced ability to self-associate relative to a wild type Fc domain, or which lacks the ability to self-associate entirely, e.g., as described in Helm et al., 1996, J. Biol. Chem. 271:7494-7500 or Ying et al., 2012, J Biol Chem. 287 (23): 19399-19408. An example non-dimerizing Fc domain comprises amino acid substitutions in the positions corresponding to T366 and/or Y407 in CH3 (numberings according to Kabat EU index), as described in U.S. Patent Publication No. 2019/0367611, incorporated herein by reference. Particular amino acid substitutions which may be included in a non-dimerizing Fc domain include, for example, L351S, T366R, L368H, P395K, L242C, K334C, L351S, P343C, A431C, L351Y, T366Y, L368A, P395R, F405R, Y407M, K409A, F405E, Y407K, L351K, T366S, P395V, Y407A, and K409Y (numberings according to Kabat EU index). A non-dimerizing Fc domain of the present disclosure may include any 1, 2, 3, 4, 5, 6, 7, 8, 9, or 10 of the abovementioned substitutions, or more.

Exemplary sequences of non-dimerizing Fc domains are provided in Table Y-2, below. Bolded residues show locations of amino acid substitutions relative to wild type IgG sequence.

TABLE Y-2
Non-dimerizing Fc Sequences

| | | SEQ |
|---|---|---|
| Non-dimerizing Fc | Sequence | ID NO |
| IgG1 mFc (L351S, | APELLGGPSVFLFPPKPKDTLMISRTPEVTCVVVDVSHEDPEVKF | 27 |
| T366R, L368H, P395K; | NWYVDGVEVHNAKTKPREEQYNSTYRVVSVLTVLHQDWLNGKE | |
| Kabat EU numbering) | YKCKVSNKALPAPIEKTISKAKGQPREPQVYTSPPSRDELTKNQV | |
| | SLRCHVKGFYPSDIAVEWESNGQPENNYKTTKPVLDSDGSFFLY | |
| | SKLTVDKSRWQQGNVFSCSVMHEALHNHYTQKSLSLSPGK | |
| IgG1 smFc (L242C, | APELLGGPSVFCFPPKPKDTLMISRTPEVTCVVVDVSHEDPEVKF | 28 |
| K334C, L351S, T366R, | NWYVDGVEVHNAKTKPREEQYNSTYRVVSVLTVLHQDWLNGKE | |
| L368H, P395K; Kabat | YKCKVSNKALPAPIECTISKAKGQPREPQVYTSPPSRDELTKNQV | |
| EU numbering) | SLRCHVKGFYPSDIAVEWESNGQPENNYKTTKPVLDSDGSFFLY | |
| | SKLTVDKSRWQQGNVFSCSVMHEALHNHYTQKSLSLSPGK | |
| IgG1 ssmFc (L242C, | APELLGGPSVFCFPPKPKDTLMISRTPEVTCVVVDVSHEDPEVKF | 29 |
| K334C, P343C, L351S, | NWYVDGVEVHNAKTKPREEQYNSTYRVVSVLTVLHQDWLNGKE | |
| T366R, L368H, P395K, | YKCKVSNKALPAPIECTISKAKGQCREPQVYTSPPSRDELTKNQV | |
| A431C; Kabat EU | SLRCHVKGFYPSDIAVEWESNGQPENNYKTTKPVLDSDGSFFLY | |
| numbering) | SKLTVDKSRWQQGNVFSCSVMHECLHNHYTQKSLSLSPGK | |
| IgG1 mFc.1 (L351Y, | APELLGGPSVFLFPPKPKDTLMISRTPEVTCVVVDVSHEDPEVKF | 30 |
| T366Y, L368A, P395R, | NWYVDGVEVHNAKTKPREEQYNSTYRVVSVLTVLHQDWLNGKE | |
| F405R, Y407M, K409A; | YKCKVSNKALPAPIEKTISKAKGQPREPQVYTYPPSRDELTKNQV | |
| Kabat EU numbering) | SLYCAVKGFYPSDIAVEWESNGQPENNYKTTRPVLDSDGSFRLM | |
| | SALTVDKSRWQQGNVFSCSVMHEALHNHYTQKSLSLSPGK | |
| IgG1 mFc.23 (L351S, | APELLGGPSVFLFPPKPKDTLMISRTPEVTCVVVDVSHEDPEVKF | 31 |
| T366R, L368H, P395K, | NWYVDGVEVHNAKTKPREEQYNSTYRVVSVLTVLHQDWLNGKE | |
| F405E, Y407K, K409A; | YKCKVSNKALPAPIEKTISKAKGQPREPQVYTSPPSRDELTKNQV | |
| Kabat EU numbering) | SLRCHVKGFYPSDIAVEWESNGQPENNYKTTKPVLDSDGSFELK | |
| | SALTVDKSRWQQGNVFSCSVMHEALHNHYTQKSLSLSPGK | |
| IgG1 mFc.67 (L351K, | APELLGGPSVFLFPPKPKDTLMISRTPEVTCVVVDVSHEDPEVKF | 32 |
| T366S, P395V, F405R, | NWYVDGVEVHNAKTKPREEQYNSTYRVVSVLTVLHQDWLNGKE | |
| Y407A, K409Y; Kabat | YKCKVSNKALPAPIEKTISKAKGQPREPQVYTKPPSRDELTKNQV | |
| EU numbering) | SLSCLVKGFYPSDIAVEWESNGQPENNYKTTVPVLDSDGSFRLA | |
| | SYLTVDKSRWQQGNVFSCSVMHEALHNHYTQKSLSLSPGK | |

In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:27. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:28. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:29. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:30. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO: 31. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:32.

### 6.4.1.1.1. Fc1.5 Domains

In some embodiments, the Fc domain further comprises, in addition to a CH2 and CH3 domain, an additional CH3 domain connected to the first CH3 domain via a linker (e.g., a linker as described in Section 6.5). An Fc domain comprising such a configuration (CH2-CH3-linker-CH3) is sometimes referred to herein as an “Fc1.5 domain” or simply “Fc1.5” for convenience. The linker between the first and second CH3 domains of an Fc1.5 domain is preferably of sufficient length and flexibility so as to permit dimerization of the first CH3 domain with the second CH3 domain. Thus, in some embodiments, the stabilization moiety is an Fc1.5 domain comprising a linker of at least 5, at least 10, at least 15, or at least 20 amino acids in length connecting the first and second CH3 domains.

Exemplary sequences of Fc1.5 domains are provided in Table Y-3, below.

TABLE Y-3
Fc1.5 Sequences

| | | SEQ |
|---|---|---|
| Fc1.5 | Sequence | ID NO |
| Fc1.5 (IgG1; (G₄S)₈) | GGPSVFLFPPKPKDTLMISRTPEVTCVVVDVSHEDPEVKFNWYV | 33 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGG | |
| | GGSGGGGSGGGGSGGGGSGGGGSGGGGSGGGGSGQPREPQ | |
| | VYTLPPSRDELTKNQVSLTCLVKGFYPSDIAVEWESNGQPENNY | |
| | KTTPPVLDSDGSFFLYSKLTVDKSRWQQGNVFSCSVMHEALHN | |
| | HYTQKSLSLSPGK | |
| Fc1.5 (IgG1; (G₄S)₇) | GGPSVFLFPPKPKDTLMISRTPEVTCVVVDVSHEDPEVKFNWYV | 34 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGG | |
| | GGSGGGGSGGGGSGGGGSGGGGSGGGGSGQPREPQVYTLP | |
| | PSRDELTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPGK | |
| Fc1.5 (IgG1; (G₄S)₆) | GGPSVFLFPPKPKDTLMISRTPEVTCVVVDVSHEDPEVKFNWYV | 35 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGG | |
| | GGSGGGGSGGGGSGGGGSGGGGSGQPREPQVYTLPPSRDEL | |
| | TKNQVSLTCLVKGFYPSDIAVEWESNGQPENNYKTTPPVLDSDG | |
| | SFFLYSKLTVDKSRWQQGNVFSCSVMHEALHNHYTQKSLSLSPG | |
| | K | |
| Fc1.5 (IgG1; (G₄S)₅) | GGPSVFLFPPKPKDTLMISRTPEVTCVVVDVSHEDPEVKFNWYV | 36 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGG | |
| | GGSGGGGSGGGGSGGGGSGQPREPQVYTLPPSRDELTKNQVS | |
| | LTCLVKGFYPSDIAVEWESNGQPENNYKTTPPVLDSDGSFFLYSK | |
| | LTVDKSRWQQGNVFSCSVMHEALHNHYTQKSLSLSPGK | |
| Fc1.5 (IgG1; (G₄S)₄) | GGPSVFLFPPKPKDTLMISRTPEVTCVVVDVSHEDPEVKFNWYV | 37 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGG | |
| | GGSGGGGSGGGGSGQPREPQVYTLPPSRDELTKNQVSLTCLVK | |
| | GFYPSDIAVEWESNGQPENNYKTTPPVLDSDGSFFLYSKLTVDK | |
| | SRWQQGNVFSCSVMHEALHNHYTQKSLSLSPGK | |
| Fc1.5 (IgG1; (G₄S)₃) | GGPSVFLFPPKPKDTLMISRTPEVTCVVVDVSHEDPEVKFNWYV | 38 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGG | |
| | GGSGGGGSGQPREPQVYTLPPSRDELTKNQVSLTCLVKGFYPS | |
| | DIAVEWESNGQPENNYKTTPPVLDSDGSFFLYSKLTVDKSRWQQ | |
| | GNVFSCSVMHEALHNHYTQKSLSLSPGK | |
| Fc1.5 (IgG1; (G₄S)₂) | GGPSVFLFPPKPKDTLMISRTPEVTCVVVDVSHEDPEVKFNWYV | 39 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGG | |
| | GGSGQPREPQVYTLPPSRDELTKNQVSLTCLVKGFYPSDIAVEW | |
| | ESNGQPENNYKTTPPVLDSDGSFFLYSKLTVDKSRWQQGNVFS | |
| | CSVMHEALHNHYTQKSLSLSPGK | |
| Fc1.5 (IgG1; (G₄S)) | GGPSVFLFPPKPKDTLMISRTPEVTCVVVDVSHEDPEVKFNWYV | 40 |
| | DGVEVHNAKTKPREEQYNSTYRVVSVLTVLHQDWLNGKEYKCK | |
| | VSNKALPAPIEKTISKAKGQPREPQVYTLPPSRDELTKNQVSLTCL | |
| | VKGFYPSDIAVEWESNGQPENNYKTTPPVLDSDGSFFLYSKLTV | |
| | DKSRWQQGNVFSCSVMHEALHNHYTQKSLSLSPGKGGGGSGQ | |
| | PREPQVYTLPPSRDELTKNQVSLTCLVKGFYPSDIAVEWESNGQ | |
| | PENNYKTTPPVLDSDGSFFLYSKLTVDKSRWQQGNVFSCSVMH | |
| | EALHNHYTQKSLSLSPGK | |

In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:33. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:34. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO: 35. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:36. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:37. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO: 38. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:39. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:40.

6.4.1.2. Fc Domains with Altered Effector Function

In some embodiments, the Fc domain comprises one or more amino acid substitutions that reduce binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain comprises an amino acid substitution at a position selected from the group of E233, L234, L235, N297, P331 and P329 (numberings according to Kabat EU index). In a more specific embodiment, the Fc domain comprises an amino acid substitution at a position selected from the group of L234, L235 and P329 (numberings according to Kabat EU index). In some embodiments, the Fc domain comprises the amino acid substitutions L234A and L235A (numberings according to Kabat EU index). In one such embodiment, the Fc domain is an Igd Fc domain, particularly a human Igd Fc domain. In one embodiment, the Fc domain comprises an amino acid substitution at position P329. In a more specific embodiment, the amino acid substitution is P329A or P329G, particularly P329G (numberings according to Kabat EU index). In one embodiment, the Fc domain comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table F below. In some embodiments, the Fc domain includes only the bolded portion of the sequences shown below:

TABLE F

| Fc Domain | Sequence |
|---|---|
| SEQ ID NO: 1 of | DKRVESKYGP PCPPCPAPPV AGPSVFLFPP KPKDTLMISR |
| WO2014/121087 | TPEVTCVVVD VSQEDPEVQF NWYVDGVEVH NAKTKPREEQ |
| | FNSTYRVVSV LTVLHQDWLN GKEYKCKVSN KGLPSSIEKT |
| | ISKAKGQPRE PQVYTLPPSQ EEMTKNQVSL TCLVKGFYPS |
| | DIAVEWESNG QPENNYKTTP PVLDSDGSFF LYSRLTVDKS |
| | RWQEGNVFSC SVMHEALHNH YTQKSLSLSL GK (SEQ ID |
| | NO: 439) |
| SEQ ID NO: 2 of | DKKVEPKSCD KTHTCPPCPA PPVAGPSVFL FPPKPKDTLM |
| WO2014/121087 | ISRTPEVTCV VVDVSQEDPE VQFNWYVDGV EVHNAKTKPR |
| | EEQFNSTYRV VSVLTVLHQD WINGKEYKCK VSNKGLPSSI |
| | EKTISKAKGQ PREPQVYTLP PSRDELTKNQ VSLTCLVKGF |
| | YPSDIAVEWE SNGQPENNYK TTPPVLDSDG SFFLYSKLTV |
| | DKSRWQQGNV FSCSVMHEAL HNHYTQKSLS LSPGK (SEQ |
| | ID NO: 440) |
| SEQ ID NO: 30 of | ASTKGPSVFP LAPSSKSTSG GTAALGCLVK DYFPEPVTVS |
| WO2014/121087 | WNSGALTSGV HTFPAVLQSS GLYSLSSVVT VPSSSLGTQT |
| | YICNVNHKPS NTKVDKKVEP KSCDKTHTCP PCPAPPVAGP |
| | SVFLFPPKPK DTLMISRTPE VTCVVVDVSQ EDPEVQFNWY |
| | VDGVEVHNAK TKPREEQFNS TYRVVSVLTV LHQDWLNGKE |
| | YKCKVSNKGL PSSIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGK (SEQ ID NO: 196) |
| SEQ ID NO: 31 of | ASTKGPSVFP LAPCSRSTSE STAALGCLVK DYFPEPVTVS |
| WO2014/121087 | WNSGALTSGV HTFPAVLQSS GLYSLSSVVT VPSSSLGTKT |
| | YTCNVDHKPS NTKVDKRVES KYGPPCPPCP APPVAGPSVF |
| | LFPPKPKDTL MISRTPEVTC VVVDVSQEDP EVQFNWYVDG |
| | VEVHNAKTKP REEQFNSTYR VVSVLTVLHQ DWLNGKEYKC |
| | KVSNKGLPSS IEKTISKAKG QPREPQVYTL PPSQEEMTKN |
| | QVSLTCLVKG FYPSDIAVEW ESNGQPENNY KTTPPVLDSD |
| | GSFFLYSRLT VDKSRWQEGN VFSCSVMHEA LHNHYTQKSL |
| | SLSLGK (SEQ ID NO: 197) |
| SEQ ID NO: 37 of | ASTKGPSVFP LAPSSKSTSG GTAALGCLVK DYFPEPVTVS |
| WO2014/121087 | WNSGALTSGV HTFPAVLQSS GLYSLSSVVT VPSSSLGTQT |
| | YICNVNHKPS NTKVDKKVEP KSCDKTHTCP PCPAPPVAGP |
| | SVFLFPPKPK DTLMISRTPE VTCVVVDVSQ EDPEVQFNWY |
| | VDGVEVHNAK TKPREEQFNS TYRVVSVLTV LHQDWLNGKE |
| | YKCKVSNKGL PSSIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNRFTQ |
| | KSLSLSPGK (SEQ ID NO: 198) |
| SEQ ID NO: 38 of | ASTKGPSVFP LAPCSRSTSE STAALGCLVK DYFPEPVTVS |
| WO2014/121087 | WNSGALTSGV HTFPAVLQSS GLYSLSSVVT VPSSSLGTKT |
| | YTCNVDHKPS NTKVDKRVES KYGPPCPPCP APPVAGPSVF |
| | LFPPKPKDTL MISRTPEVTC VVVDVSQEDP EVQFNWYVDG |
| | VEVHNAKTKP REEQFNSTYR VVSVLTVLHQ DWLNGKEYKC |
| | KVSNKGLPSS IEKTISKAKG QPREPQVYTL PPSQEEMTKN |
| | QVSLTCLVKG FYPSDIAVEW ESNGQPENNY KTTPPVLDSD |
| | GSFFLYSRLT VDKSRWQEGN VFSCSVMHEA LHNRFTQKSL |
| | SLSLGK (SEQ ID NO: 199) |

In a particular embodiment, the IgG4 with reduced effector function comprises the bolded portion of the amino acid sequence of SEQ ID NO:31 of WO2014/121087, sometimes referred to herein as IgG4s or hIgG4s.

### 6.4.2. Albumin Moieties

Human serum albumin (HSA) has a long average half-life of approximately 19 days. HSA is the most abundant protein in human plasma and serves a variety of functions including maintenance of plasma pH, transport of fatty acids and other metabolites, and maintenance of blood pressure, among others. The amino acid sequence of mature HSA (lacking the signal sequence and propeptide) is shown below.

(SEQ ID NO: 19)
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL

An albumin moiety of an engineered alpha klotho polypeptide of the disclosure may be HSA, a portion thereof, or a variant thereof. Thus, in some embodiments, the albumin moiety comprises the sequence of wild-type, mature HSA. In some embodiments, the albumin moiety comprises an amino acid sequence having 1, 2, 3, 4, 5, or more amino acid substitutions relative to wild-type, mature HSA. For example, in some embodiments, the albumin moiety comprises an amino acid sequence having a C34S mutation relative to wild-type, mature HSA. Without being bound by theory, a C34S in HSA is believed to improve stability by virtue of elimination of a free cysteine. Additional HSA variants are recognized in the art and contemplated herein including, for example, K573P mutant HSA. In some embodiments, the albumin moiety comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO: 19. In some embodiments, the albumin moiety comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:20.

### 6.4.3. Other Stabilization Moieties

In some embodiments, the engineered alpha klotho polypeptide comprises polyethylene glycol (PEG) or another hydrophilic polymer as a stabilization moiety, for example a copolymer of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), dextran or poly(n-vinyl pyrrolidone) polyethylene glycol, a propropylene glycol homopolymer, a prolypropylene oxide/ethylene oxide co-polymer, a polyoxyethylated polyol (e.g., glycerol), polyvinyl alcohol, and mixtures thereof. The polymer may be of any molecular weight, and may be branched or unbranched.

Serum albumin can be engaged in half-life extension through modules with the capacity to non-covalently interact with albumin. Accordingly, the engineered alpha klotho polypeptides of the disclosure can include as a stabilization moiety an albumin-binding protein. The albumin-binding protein can be either conjugated or genetically fused to one or more other components of the engineered alpha klotho polypeptides of the disclosure. Proteins with albumin-binding activity are known from certain bacteria. For example, streptococcal protein G contains several small albumin-binding domains composed of roughly 50 amino acid residues (6 kDa). Additional examples of serum albumin binding proteins such as those described in U.S. Publication Nos. 2007/0178082 and 2007/0269422. Fusion of an albumin binding domain to a protein results in a strongly extended half-life (see Kontermann et al., 2011, Current Opinion in Biotechnology 22:868-76).

### 6.5. Linkers

In certain aspects, the present disclosure provides engineered alpha klotho polypeptides in which two or more components of are connected to one another by a peptide linker. By way of example and not limitation, linkers can be used to connect (a) an alpha klotho moiety and a stabilization moiety and/or (b) a stabilization moiety and another moiety, e.g., a purification tag moiety.

Linkers in the engineered alpha klotho polypeptides can be peptides linkers that are protease-cleavable linkers (PCLs) or non-cleavable linkers (NCLs). In some embodiments, the linker is a protease-cleavable linker.

### 6.5.1. Non-Cleavable Linkers

Typically, a non-cleavable peptide linker is a linker that is not cleaved by a protease. A non-cleavable linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a non-cleavable linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the non-cleavable linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible non-cleavable linkers are particularly preferred.

Examples of flexible non-cleavable linkers that can be used in the engineered alpha klotho polypeptides of the disclosure include those disclosed by Chen et al., 2013, Adv Drug Deliv Rev. 65 (10): 1357-1369 and Klein et al., 2014, Protein Engineering, Design & Selection 27 (10): 325-330. Particularly useful flexible linkers are or comprise repeats of glycines and serines, e.g., a monomer or multimer of GₙS (SEQ ID NO: 200) or SGₙ (SEQ ID NO: 201), where n is an integer from 1 to 10, e.g., 12, 3, 4, 5, 6, 7, 8, 9 or 10. In one embodiment, the linker is or comprises a monomer or multimer of repeat of G4S (SEQ ID NO: 188) e.g., (GGGGS), (SEQ ID NO: 188).

Polyglycine non-cleavable linkers can suitably be used in the engineered alpha klotho polypeptides of the disclosure. In some embodiments, a peptide non-cleavable linker comprises two consecutive glycines (2Gly), three consecutive glycines (3Gly), four consecutive glycines (4Gly) (SEQ ID NO: 202), five consecutive glycines (5Gly) (SEQ ID NO: 203), six consecutive glycines (6Gly) (SEQ ID NO: 204), seven consecutive glycines (7Gly) (SEQ ID NO: 205), eight consecutive glycines (8Gly) (SEQ ID NO: 206) or nine consecutive glycines (9Gly) (SEQ ID NO: 207).

Exemplary non-cleavable linker sequences are set forth in Table L below. An engineered alpha klotho polypeptide of the disclosure may comprise one or more linkers of Table L. For example, in some embodiments, an engineered alpha klotho polypeptide of the disclosure comprises an alpha klotho moiety and a stabilization moiety connected by a linker of Table L.

TABLE L
Non-Cleavable Linker Sequences

| | | SEQ ID |
|---|---|---|
| Linker Sequence | Designation | NO: |
| (GGGGS)ₙ | L1 | 188 |
| (GGGS)ₙ | L2 | 208 |
| (GGS)ₙ | L3 | N/A |
| (GS)ₙ | L4 | N/A |
| (GSGGS)ₙ | L5 | 209 |
| AAAGG | L6 | 210 |
| ADAAP | L7 | 211 |
| ADAAPTVSIFP | L8 | 212 |
| ADAAPTVSIFPP | L9 | 213 |
| AKTTAP | L10 | 214 |
| AKTTAPSVYPLAP | L11 | 215 |
| AKTTPKLEEGEFSEARV | L12 | 216 |
| AKTTPKLGG | L13 | 217 |
| AKTTPP | L14 | 218 |
| AKTTPPSVTPLAP | L15 | 219 |
| ASTKGP | L16 | 220 |
| ASTKGPSVFPLAPASTKGPSVFPLAP | L17 | 221 |
| EGKSSGSGSESKST | L18 | 222 |
| GEGESGEGESGEGES | L19 | 223 |
| GEGESGEGESGEGESGEGES | L20 | 224 |
| GEGGSGEGGSGEGGS | L21 | 225 |
| GENKVEYAPALMALS | L22 | 226 |
| GGEGSGGEGSGGEGS | L23 | 227 |
| GGGESGGEGSGEGGS | L24 | 228 |
| GGGESGGGESGGGES | L25 | 229 |
| GGGGSGGGGS | L26 | 195 |
| GGGGGGGGSGGGGS | L27 | 194 |
| GGGGSGGGGSGGGGSGGGGS | L28 | 193 |
| GGGKSGGGKSGGGKS | L29 | 230 |
| GGGKSGGKGSGKGGS | L30 | 231 |
| GGGS | L31 | 208 |
| GGGSG | L32 | 232 |
| GGKGSGGKGSGGKGS | L33 | 233 |
| GGS | L34 | N/A |
| GGSG | L35 | 234 |
| GGSGG | L36 | 235 |
| GGSGG | L37 | 235 |
| GGSGGGGSG | L38 | 236 |
| GGSGGGGSGGGGS | L39 | 237 |
| GHEAAAVMQVQYPAS | L40 | 238 |
| GKGGSGKGGSGKGGS | L41 | 239 |
| GKGKSGKGKSGKGKS | L42 | 240 |
| GKGKSGKGKSGKGKSGKGKS | L43 | 241 |
| GKPGSGKPGSGKPGS | L44 | 242 |
| GKPGSGKPGSGKPGSGKPSGS | L45 | 243 |
| GPAKELTPLKEAKVS | L46 | 244 |
| GSAGSAAGSGEF | L47 | 245 |
| GSGGG | L48 | 246 |
| GSGSG | L49 | 247 |
| GSS | L50 | N/A |
| GSSG | L51 | 248 |
| GSSGGSGGSG | L52 | 249 |
| GSSGGSGGSGG | L53 | 250 |
| GSSGGSGGSGGS | L54 | 251 |
| GSSGGSGGSGGSG | L55 | 252 |
| GSSGGSGGSGGSGGGS | L56 | 253 |
| GSSGGSGGSGS | L57 | 254 |
| GSSGT | L58 | 255 |
| GSSSG | L59 | 256 |
| GSTSGSGKPGSGEGSTKG | L60 | 257 |
| GTAAAGAGAAGGAAAGAAG | L61 | 258 |
| GTSGSSGSGSGGSGSGGGG | L62 | 259 |
| IRPRAIGGSKPRVA | L63 | 260 |
| KESGSVSSEQLAQFRSLD | L64 | 261 |
| KTTPKLEEGEFSEAR | L65 | 262 |
| PRGASKSGSASQTGSAPGS | L66 | 263 |
| QPKAAP | L67 | 264 |
| QPKAAPSVTLFPP | L68 | 265 |
| RADAAAA(G4S)₄ | L69 | 266 |
| RADAAAAGGPGS | L70 | 267 |
| RADAAP | L71 | 268 |
| RADAAPTVS | L72 | 269 |
| SAKTTP | L73 | 270 |
| SAKTTPKLEEGEFSEARV | L74 | 271 |
| SAKTTPKLGG | L75 | 272 |
| STAGDTHLGGEDFD | L76 | 273 |
| TVAAP | L77 | 274 |
| TVAAPSVFIFPP | L78 | 275 |
| TVAAPSVFIFPPTVAAPSVFIFPP | L79 | 276 |
| GGGGSLALGPGGGGGSLALGPGGGGGSLALGPGGS | L80 | 277 |
| AGSGNSSGSGGSGGSGNSSGSGGSPVPSTPPTPSPST | L81 | 278 |
| PPTPSPSAS | | |
| GGGGSAS | L82 | 279 |
| GGGGSGGGGSAS | L83 | 280 |
| GGGGSGGGGGGGGSAS | L84 | 281 |
| GGGGSGGGGSGGGGSGGGGSGGGGSAS | L85 | 282 |
| GGGGSGGGGSGGGGSGGGGSGGGGSGGGGSAS | L86 | 283 |
| AGGGSGGGGSGGGGSGGGGSGGGGSGGGGGGGGS | L87 | 284 |
| AS | | |
| AGGGSGGGGSGGGGSGGGGSGGGGSGGGGSGGGGS | L88 | 285 |
| GGGGSAS | | |
| AGGGSGGGGSGGGGSGGGGSGGGGSGGGGSGGGGS | L89 | 286 |
| GGGGSGGGGSAS | | |

In some embodiments, the engineered KL polypeptide comprises linker L1. In some embodiments, the engineered KL polypeptide comprises linker L2. In some embodiments, the engineered KL polypeptide comprises linker L3. In some embodiments, the engineered KL polypeptide comprises linker L4. In some embodiments, the engineered KL polypeptide comprises linker L5. In some embodiments, the engineered KL polypeptide comprises linker L6. In some embodiments, the engineered KL polypeptide comprises linker L7. In some embodiments, the engineered KL polypeptide comprises linker L8. In some embodiments, the engineered KL polypeptide comprises linker L9. In some embodiments, the engineered KL polypeptide comprises linker L10. In some embodiments, the engineered KL polypeptide comprises linker L11. In some embodiments, the engineered KL polypeptide comprises linker L12. In some embodiments, the engineered KL polypeptide comprises linker L13. In some embodiments, the engineered KL polypeptide comprises linker L14. In some embodiments, the engineered KL polypeptide comprises linker L15. In some embodiments, the engineered KL polypeptide comprises linker L16. In some embodiments, the engineered KL polypeptide comprises linker L17. In some embodiments, the engineered KL polypeptide comprises linker L18. In some embodiments, the engineered KL polypeptide comprises linker L19. In some embodiments, the engineered KL polypeptide comprises linker L20. In some embodiments, the engineered KL polypeptide comprises linker L21. In some embodiments, the engineered KL polypeptide comprises linker L22. In some embodiments, the engineered KL polypeptide comprises linker L23. In some embodiments, the engineered KL polypeptide comprises linker L24. In some embodiments, the engineered KL polypeptide comprises linker L25. In some embodiments, the engineered KL polypeptide comprises linker L26. In some embodiments, the engineered KL polypeptide comprises linker L27. In some embodiments, the engineered KL polypeptide comprises linker L28. In some embodiments, the engineered KL polypeptide comprises linker L29. In some embodiments, the engineered KL polypeptide comprises linker L30. In some embodiments, the engineered KL polypeptide comprises linker L31. In some embodiments, the engineered KL polypeptide comprises linker L32. In some embodiments, the engineered KL polypeptide comprises linker L33. In some embodiments, the engineered KL polypeptide comprises linker L34. In some embodiments, the engineered KL polypeptide comprises linker L35. In some embodiments, the engineered KL polypeptide comprises linker L36. In some embodiments, the engineered KL polypeptide comprises linker L37. In some embodiments, the engineered KL polypeptide comprises linker L38. In some embodiments, the engineered KL polypeptide comprises linker L39. In some embodiments, the engineered KL polypeptide comprises linker L40. In some embodiments, the engineered KL polypeptide comprises linker L41. In some embodiments, the engineered KL polypeptide comprises linker L42. In some embodiments, the engineered KL polypeptide comprises linker L43. In some embodiments, the engineered KL polypeptide comprises linker L44. In some embodiments, the engineered KL polypeptide comprises linker L45. In some embodiments, the engineered KL polypeptide comprises linker L46. In some embodiments, the engineered KL polypeptide comprises linker L47. In some embodiments, the engineered KL polypeptide comprises linker L48. In some embodiments, the engineered KL polypeptide comprises linker L49. In some embodiments, the engineered KL polypeptide comprises linker L50. In some embodiments, the engineered KL polypeptide comprises linker L51. In some embodiments, the engineered KL polypeptide comprises linker L52. In some embodiments, the engineered KL polypeptide comprises linker L53. In some embodiments, the engineered KL polypeptide comprises linker L54. In some embodiments, the engineered KL polypeptide comprises linker L55. In some embodiments, the engineered KL polypeptide comprises linker L56. In some embodiments, the engineered KL polypeptide comprises linker L57. In some embodiments, the engineered KL polypeptide comprises linker L58. In some embodiments, the engineered KL polypeptide comprises linker L59. In some embodiments, the engineered KL polypeptide comprises linker L60. In some embodiments, the engineered KL polypeptide comprises linker L61. In some embodiments, the engineered KL polypeptide comprises linker L62. In some embodiments, the engineered KL polypeptide comprises linker L63. In some embodiments, the engineered KL polypeptide comprises linker L64. In some embodiments, the engineered KL polypeptide comprises linker L65. In some embodiments, the engineered KL polypeptide comprises linker L66. In some embodiments, the engineered KL polypeptide comprises linker L67. In some embodiments, the engineered KL polypeptide comprises linker L68. In some embodiments, the engineered KL polypeptide comprises linker L69. In some embodiments, the engineered KL polypeptide comprises linker L70. In some embodiments, the engineered KL polypeptide comprises linker L71. In some embodiments, the engineered KL polypeptide comprises linker L72. In some embodiments, the engineered KL polypeptide comprises linker L73. In some embodiments, the engineered KL polypeptide comprises linker L74. In some embodiments, the engineered KL polypeptide comprises linker L75. In some embodiments, the engineered KL polypeptide comprises linker L76. In some embodiments, the engineered KL polypeptide comprises linker L77. In some embodiments, the engineered KL polypeptide comprises linker L78. In some embodiments, the engineered KL polypeptide comprises linker L79. In some embodiments, the engineered KL polypeptide comprises linker L80. In some embodiments, the engineered KL polypeptide comprises linker L81. In some embodiments, the engineered KL polypeptide comprises linker L82. In some embodiments, the engineered KL polypeptide comprises linker L83. In some embodiments, the engineered KL polypeptide comprises linker L84. In some embodiments, the engineered KL polypeptide comprises linker L85. In some embodiments, the engineered KL polypeptide comprises linker L86. In some embodiments, the engineered KL polypeptide comprises linker L87. In some embodiments, the engineered KL polypeptide comprises linker L88. In some embodiments, the engineered KL polypeptide comprises linker L89.

### 6.5.2. Protease-Cleavable Linkers

Protease-cleavable linkers are peptide linkers that can be cleaved by a protease. The engineered alpha klotho polypeptides optionally include protease-cleavable linkers (PCLs). Incorporating a PCL into an alpha klotho polypeptide may lead to, for example, release of the alpha klotho moiety from the stabilization moiety upon cleavage of the PCL.

A protease-cleavable linker can range from 8 amino acids to 100 or more amino acids. In various embodiments, the protease-cleavable linker ranges from 8 amino acids to 15 amino acids, from 10 amino acids to 20 amino acids, 20 amino acids to 80, and in certain aspects a non-cleavable peptide linker ranges from 20 amino acids to 60 amino acids, 20 amino acids to 40 amino acids, from 30 amino acids to 50 amino acids, from 20 amino acids to 80 amino acids, or from 30 amino acids to 70 amino acids in length.

The protease-cleavable linkers comprise one or more substrate sequences for one or more proteases, for example one or more of the proteases set forth in Section 6.5.2.1. The one or more substrate sequences, e.g., one or more of the substrate sequences set forth in Section 6.5.2.2, are typically (but not necessarily) flanked by one or more spacer sequences, e.g., spacer sequences as described in Section 6.5.2.3. Each protease-cleavable linker can include one, two, three or more substrate sequences. The spacer sequences can be adjoining, overlapping, or separated by spacer sequences. Preferably, the C- and N-termini of the protease-cleavable linkers contain spacer sequences. Exemplary protease-cleavable linker sequences are set forth in Section 6.5.2.4.

### 6.5.2.1. Proteases

Exemplary proteases whose substrate sequences can be incorporated into the protease-cleavable linkers are set forth in Table A below.

TABLE A
Exemplary Proteases for Substrate Cleavage

| | | |
|---|---|---|
| ADAMS, ADAMTS, e.g. | Caspases, e.g., | MMP24 |
| ADAM8 | Caspase 1 | MMP26 |
| ADAM9 | Caspase 2 | MMP27 |
| ADAM10 | Caspase 3 | Cysteine proteinases, e.g., |
| ADAM12 | Caspase 4 | Cruzipain |
| ADAM15 | Caspase 5 | Legumain |
| ADAM17/TACE | Caspase 6 | Otubain-2 |
| ADAMDEC1 | Caspase 7 | KLKs, e.g., |
| ADAMTS1 | Caspase 8 | KLK4 |
| ADAMTS4 | Caspase 9 | KLK5 |
| ADAMTS5 | Caspase 10 | KLK6 |
| Aspartate proteases, e.g., | Caspase 14 | KLK7 |
| BACE | Cysteine cathepsins, e.g., | KLK8 |
| Renin | Cathepsin B | KLK10 |
| Aspartic cathepsins, e.g., | Cathepsin C | KLK11 |
| Cathepsin D | Cathepsin K | KLK13 |
| Cathepsin E | Cathepsin L | KLK14 |
| NS3/4A | Cathepsin S | Metallo proteinases, e.g., |
| PACE4 | Cathepsin V/L2 | Meprin |
| Plasmin | Cathepsin X/Z/P | Neprilysin |
| PSA | MMPs, e.g., | PSMA |
| tPA | MMP1 | BMP-1 |
| Thrombin | MMP2 | Serine proteases, e.g., |
| Tryptase | MMP3 | activated protein C |
| uPA | MMP7 | Cathepsin A |
| Type II Transmembrane | MMP8 | Cathepsin G |
| Serine Proteases (TTSPs), | MMP9 | Chymase |
| e.g., | MMP10 | coagulation factor proteases |
| DESC1 | MMP11 | (e.g., FVIIa, FIXa, FXa, |
| DPP-4 | MMP12 | FXIa, FXIIa) |
| FAP | MMP13 | Human Neutrophil Elastase |
| Hepsin | MMP14 | Lactoferrin |
| Matriptase-2 | MMP15 | |
| MT/SP1/Matriptase | MMP16 | |
| TMPRSS2 | MMP17 | |
| TMPRSS3 | MMP19 | |
| TMPRSS4 | MMP20 | |
| | MMP23 | |

In particular embodiments, the protease is matrix metalloprotease (MMP)-2, MMP-9, legumain asparaginyl endopeptidase, thrombin, fibroblast activation protease (FAP), MMP-1, MMP-3, MMP-7, MMP-8, MMP-12, MMP-13, MMP-14, membrane type 1 matrix metalloprotease (MT1-MMP), plasmin, transmembrane protease, serine (TMPRSS-3/4), cathepsin A, cathepsin B, cathepsin D, cathepsin E, cathepsin F, cathepsin H, cathepsin K, cathepsin L, cathepsin L2, cathepsin O, cathepsin S, caspase 1, caspase 2, caspase 3, caspase 4, caspase 5, caspase 6, caspase 7, caspase 8, caspase 9, caspase 10, caspase 11, caspase 12, caspase 13, caspase 14, human neutrophil elastase, urokinase/urokinase-type plasminogen activator (uPA), a disintegrin and metalloprotease (ADAM) 10, ADAM12, ADAM17, ADAM with thrombospondin motifs (ADAMTS), ADAMTS5, beta secretase (BACE), granzyme A, granzyme B, guanidinobenzoatase, hepsin, matriptase, matriptase 2, meprin, neprilysin, prostate-specific membrane antigen (PSMA), tumor necrosis factor-converting enzyme (TACE), kallikrein-related peptidase (KLK) 3, KLK2, KLK5, KLK7, KLK11, NS3/4 protease of hepatitis C virus (HCV-NS3/4), tissue plasminogen activator (tPA), calpain, calpain 2, glutamate carboxypeptidase II, plasma kallikrein, AMSH-like protease, AMSH, γ-secretase component, antiplasmin cleaving enzyme (APCE), decysin 1, apoptosis-related cysteine peptidase, or N-acetylated alpha-linked acidic dipeptidase-like 1.

### 6.5.2.2. Substrates

Exemplary substrate sequences that are cleavable by a protease and can be incorporated into the protease-cleavable linkers are set forth in Table B below.

TABLE B
Substrate Sequences for Protease-Cleavable Linkers

| Substrate Sequence | Designation | Cleaving Protease | SEQ ID NO: |
|---|---|---|---|
| (DE)₈RPLALWRS(DR)₈ | SU1 | MMP7 | 287 |
| AARGPAIH | SU2 | | 288 |
| AAYHLVSQ | SU3 | Collagenase | 289 |
| AGLGISST | SU4 | Collagenase | 290 |
| AGLGVVER | SU5 | Collagenase | 291 |
| ALAL | SU6 | Lysosomal Enzyme | 292 |
| ALFFSSPP | SU7 | | 293 |
| ALFKSSFP | SU8 | | 294 |
| ALLLALL | SU9 | TOP | 295 |
| AQFVLTEG | SU10 | Collagenase | 296 |
| AQNLLGMV | SU11 | | 297 |
| AVGLLAPP | SU12 | Serine protease | 298 |
| DAFK | SU13 | Urokinase plasminogen | 299 |
| | | activator (uPA) | |
| DEVD | SU14 | Caspase-3 | 300 |
| DEVDP | SU15 | Caspase-3 | 301 |
| DPRSFL | SU16 | Thrombin | 302 |
| DVAQFVLT | SU17 | Collagenase | 303 |
| DVLK | SU18 | Plasmin | 304 |
| DWLYWPGI | SU19 | | 305 |
| EDDDDKA | SU20 | Enterokinase | 306 |
| EP(Cit)G(Hof)YL | SU21 | MMP2, MMP9, MMP14 | 307 |
| EPQALAMS | SU22 | Collagenase | 308 |
| ESLPVVAV | SU23 | Collagenase | 309 |
| ESPAYYTA | SU24 | MMP | 310 |
| F(Pip)RS | SU25 | Thrombin | N/A |
| FK | SU26 | Lysosomal Enzyme | N/A |
| FPRPLGITGL | SU27 | | 311 |
| FRLLDWQW | SU28 | | 312 |
| GFLG | SU29 | Lysosomal Enzyme | 313 |
| GGAANLVRGG | SU30 | MMP11 | 314 |
| GGGRR | SU31 | Urokinase plasminogen | 315 |
| | | activator (uPA) | |
| GGPRGLPG | SU32 | Cathepsin K | 316 |
| GGQPSGMWGW | SU33 | | 317 |
| GGSIDGR | SU34 | Factor Xa | 318 |
| GGWHTGRN | SU35 | | 319 |
| GIAGQ | SU36 | Collagenase | 320 |
| GKAFRR | SU37 | Kallikrein 2 | 321 |
| GPAGLYAQ | SU38 | | 322 |
| GPAGMKGL | SU39 | | 323 |
| GPEGLRVG | SU40 | Collagenase | 324 |
| GPLGIAGI | SU41 | Collagenase | 325 |
| GPLGVRG | SU42 | | 326 |
| GPQGIAGQ | SU43 | Collagenase | 168 |
| GPQGLLGA | SU44 | Collagenase | 327 |
| GPRSFG | SU45 | | 328 |
| GPRSFGL | SU46 | | 329 |
| GPSHLVLT | SU47 | | 330 |
| GVSQNYPIVG | SU48 | HIV Protease | 331 |
| GVVQASCRLA | SU49 | CMV Protease | 332 |
| GWEHDG | SU50 | Interleukin 1β converting | 333 |
| | | enzyme | |
| HSSKLQ | SU51 | Prostate Specific | 334 |
| | | Antigen | |
| HSSKLQEDA | SU52 | Prostate Specific | 335 |
| | | Antigen | |
| HSSKLQL | SU53 | Prostate Specific | 336 |
| | | Antigen | |
| HTGRSGAL | SU54 | | 337 |
| IDGR | SU55 | Factor Xa | 338 |
| IEGR | SU56 | Factor Xa | 339 |
| ILPRSPAF | SU57 | | 340 |
| IPVSLRSG | SU58 | MMP | 170 |
| ISSGL | SU59 | MMP | 341 |
| ISSGLL | SU60 | MMP | 342 |
| ISSGLLS | SU61 | MMP | 343 |
| ISSGLLSS | SU62 | MMP | 344 |
| ISSGLSS | SU63 | MMP | 345 |
| KGSGDVEG | SU64 | Caspase-3 | 346 |
| KQEQNPGST | SU65 | FAP | 347 |
| KRALGLPG | SU66 | MMP7 | 348 |
| LAAPLGLL | SU67 | | 349 |
| LAPLGLQRR | SU68 | | 350 |
| LAQKLKSS | SU69 | | 351 |
| LAQRLRSS | SU70 | | 352 |
| LEATA | SU71 | MMP9 | 353 |
| LKAAPRWA | SU72 | | 354 |
| LLAPSHRA | SU73 | | 355 |
| LPGGLSPW | SU74 | | 356 |
| LSGRSANI | SU75 | Serine protease | 357 |
| LSGRSANP | SU76 | Serine protease | 358 |
| LSGRSDDH | SU77 | Serine protease | 359 |
| LSGRSDIH | SU78 | Serine protease | 360 |
| LSGRSDNH | SU79 | Serine protease | 361 |
| LSGRSDNI | SU80 | Serine protease | 362 |
| LSGRSDNP | SU81 | Serine protease | 363 |
| LSGRSDQG | SU82 | Serine protease | 364 |
| LSGRSDQH | SU83 | Serine protease | 365 |
| LSGRSDTH | SU84 | Serine protease | 366 |
| LSGRSDYH | SU85 | Serine protease | 367 |
| LSGRSGNH | SU86 | Serine protease | 368 |
| LVLASSSFGY | SU87 | Herpes Simplex Virus | 369 |
| | | Protease | |
| MDAFLESS | SU88 | Collagenase | 370 |
| MGLFSEAG | SU89 | | 371 |
| MIAPVAYR | SU90 | | 372 |
| MVLGRSLL | SU91 | | 373 |
| NLL | SU92 | Cathepsin B | N/A |
| NTLSGRSENHSG | SU93 | | 374 |
| NTLSGRSGNHGS | SU94 | | 375 |
| PAGLWLDP | SU95 | | 376 |
| PGGPAGIG | SU96 | | 377 |
| PIC(Et)FF | SU97 | Cathepsin D | 378 |
| PLGC(me)AG | SU98 | MMP | 379 |
| PLGL | SU99 | | 380 |
| PLGLAG | SU100 | MMP | 381 |
| PLGLAX | SU101 | MMP | 382 |
| PLGLWA | SU102 | MMP | 383 |
| PLGLWSQ | SU103 | MMP | 384 |
| PLTGRSGG | SU104 | | 385 |
| PMAKK | SU105 | | 386 |
| PPRSFL | SU106 | Thrombin | 387 |
| PR(S/T)(L/I)(S/T) | SU107 | MMP9 | N/A |
| PRFRIIGG | SU108 | Plasmin | 388 |
| PVGYTSSL | SU109 | | 389 |
| PVQPIGPQ | SU110 | Collagenase | 390 |
| QALAMSAI | SU111 | Collagenase | 391 |
| QGRAITFI | SU112 | | 392 |
| QNQALRMA | SU113 | | 393 |
| RGPA | SU114 | | 394 |
| RGPAFNPM | SU115 | | 395 |
| RGPATPIM | SU116 | | 396 |
| RKSSIIIRMRDVVL | SU117 | Plasmin | 397 |
| RLQLKAC | SU118 | MMP | 398 |
| RLQLKL | SU119 | MMP | 399 |
| RMHLRSLG | SU120 | | 400 |
| RPSPMWAY | SU121 | | 401 |
| RQARVVNG | SU122 | Matriptase | 402 |
| SAGFSLPA | SU123 | | 403 |
| SAPAVESE | SU124 | Collagenase | 404 |
| SARGPSRW | SU125 | | 405 |
| SGEPAYYTA | SU126 | | 406 |
| SGGPLGVR | SU127 | | 407 |
| SGRIGFLRTA | SU128 | MMP14 | 408 |
| SGRSA | SU129 | Urokinase plasminogen | 409 |
| | | activator (uPA) | |
| SGRSANPRG | SU130 | | 410 |
| SMLRSMPL | SU131 | | 411 |
| SPLPLRVP | SU132 | | 412 |
| SPLTGRSG | SU133 | | 413 |
| SPRSIMLA | SU134 | | 414 |
| SSRGPAYL | SU135 | | 415 |
| SSRHRRALD | SU136 | Plasmin | 416 |
| SSSFDKGKYKKGDDA | SU137 | Plasmin | 417 |
| SSSFDKGKYKRGDDA | SU138 | Plasmin | 418 |
| STFPFGMF | SU139 | | 419 |
| TARGPSFK | SU140 | | 420 |
| TGRGPSWV | SU141 | | 421 |
| TSGRSANP | SU142 | | 422 |
| TSTSGRSANPRG | SU143 | | 423 |
| VAGRSMRP | SU144 | | 424 |
| VAQFVLTE | SU145 | Collagenase | 425 |
| VHMPLGFLGP | SU146 | | 426 |
| VPLSLYSG | SU147 | MMP9 | 173 |
| VVPEGRRS | SU148 | | 427 |
| WATPRPMR | SU149 | | 428 |
| YGAGLGVV | SU150 | Collagenase | 429 |
| HPVGLLAR | SU151 | | 167 |

### 6.5.2.3. Spacers

Exemplary spacer sequences that can be incorporated into the protease-cleavable linkers are set forth in Table C below. In addition to the spacer sequences set forth in Table C, any of the non-cleavable linker sequences described in Section 6.5.1, e.g., the non-cleavable linker sequences set forth in Table L, or portions thereof can be used as spacer sequences. In some embodiments, spacer sequences are absent entirely from the protease-cleavable linkers.

TABLE C
Spacer Sequences for Protease-Cleavable Linkers

| Spacer Sequence | Designation | SEQ ID NO: |
|---|---|---|
| (GGGGS)ₙ | SP1 | 188 |
| (GGGS)ₙ | SP2 | 208 |
| (GGS)ₙ | SP3 | N/A |
| (GS)ₙ | SP4 | N/A |
| (GSGGS)ₙ | SP5 | 209 |
| GGGGSGGGGS | SP6 | 195 |
| GGGGSGGGGSGGGGS | SP7 | 194 |
| GGGGSGGGGSGGGGSGGGGS | SP8 | 193 |
| GGGKSGGGKSGGGKS | SP9 | 230 |
| GGGKSGGKGSGKGGS | SP10 | 231 |
| GGGS | SP11 | 208 |
| GGGSG | SP12 | 232 |
| GGKGSGGKGSGGKGS | SP13 | 233 |
| GGSGGGGSGGGGS | SP14 | 237 |
| GGSGGS | SP15 | 430 |
| GGSGGSGGSGS | SP16 | 431 |
| GSGGG | SP17 | 246 |
| GSGSG | SP18 | 247 |
| GSS | SP19 | N/A |
| GSSG | SP20 | 248 |
| GSSGGSGGSG | SP21 | 249 |
| GSSGGSGGSGG | SP22 | 250 |
| GSSGGSGGSGGS | SP23 | 251 |
| GSSGGSGGSGGSG | SP24 | 252 |
| GSSGGSGGSGGSGGGS | SP25 | 253 |
| GSSGGSGGSGS | SP26 | 254 |
| GSSGT | SP27 | 255 |
| GSSSG | SP28 | 256 |
| QGQSGQ | SP29 | 432 |
| QGQSGQG | SP30 | 433 |
| QGQSGS | SP31 | 434 |
| QSGQ | SP32 | 435 |
| QSGQG | SP33 | 436 |
| QSGS | SP34 | 437 |
| SGQ | SP35 | N/A |
| SGQG | SP36 | 438 |
| SGS | SP37 | N/A |
| (G)ₙ | SP38 | N/A |

In some embodiments, as used in Table C above, n is an integer from 1 to 10, e.g., 1, 2, 3, 4, 5, 6, 7, 8, 9 or 10.

### 6.5.2.4. Exemplary Protease-Cleavable Linkers

Exemplary protease-cleavable linkers comprising one or more substrate sequences as well as spacer sequences are set forth in Table D below.

TABLE D
Protease-Cleavable Linker Sequences

| | | | SEQ ID |
|---|---|---|---|
| Linker Sequence | Designation | Cleaving Protease(s) | NO: |
| SRLRAYLLPAPPAPGNASESEED | PCL1 | MMP7, KLK2, MMP3 | 90 |
| RSAGSVESPSVSSTHRVSDPKFH | | | |
| PLHSKIIIIKKGHAKDSQRYKVDYE | | | |
| S | | | |
| GGGISSGLLSGRSDNHGGGISSG | PCL2 | | 91 |
| LLSGRSDNHGGS | | | |
| GGGISSGLLSGRSDNHGGGISSG | PCL3 | | 92 |
| LLSGRSDNHGGS | | | |
| GGGISSGLLSGRSDNHGGGISSG | | | |
| LLSGRSDNHGGS | | | |
| GGSGGSIPVSLRSGGGISSGLLS | PCL4 | | 93 |
| GRSDNHGGSGGS | | | |
| GGSGGSVPLSLYSGGGISSGLLS | PCL5 | | 94 |
| GRSDNHGGSGGS | | | |
| GGSHPVGLLARGGGHPVGLLAR | PCL6 | | 95 |
| GGGHPVGLLARGS | | | |
| GGSHPVGLLARGGGHPVGLLAR | PCL7 | | 96 |
| GGSGRSAGGSGRSA | | | |
| AVGLLAPPGGLSGRSANI | PCL8 | ADAM17_2, FAPa_1, | 97 |
| | | CTSL1_1 | |
| AVGLLAPPGGLSGRSANP | PCL9 | FAPa_1, ADAM17_2, | 98 |
| | | CTSL1_1 | |
| AVGLLAPPGGLSGRSDDH | PCL10 | MMP14_1, MMP14_1, | 99 |
| | | MMP14_1 | |
| AVGLLAPPGGLSGRSDIH | PCL11 | MMP14_1, MMP14_1, | 100 |
| | | MMP14_1 | |
| AVGLLAPPGGLSGRSDNH | PCL12 | MMP14_1, MMP14_1 | 101 |
| AVGLLAPPGGLSGRSDNI | PCL13 | MMP14_1, CTSL1_1, | 102 |
| | | ADAM17_2 | |
| AVGLLAPPGGLSGRSDNP | PCL14 | CTSL1_1, ADAM17_2, | 103 |
| | | FAPa_1 | |
| AVGLLAPPGGLSGRSDQH | PCL15 | | 104 |
| AVGLLAPPGGLSGRSDTH | PCL16 | FAPa_1, CTSL1_1, | 105 |
| | | ADAM17_2 | |
| AVGLLAPPGGLSGRSDYH | PCL17 | | 106 |
| AVGLLAPPGGTSTSGRSANPRG | PCL18 | | 107 |
| AVGLLAPPSGRSANPRG | PCL19 | | 108 |
| AVGLLAPPTSGRSANPRG | PCL20 | | 109 |
| GGALFKSSFPGPAGLYAQPLAQK | PCL21 | CTSL1_1, MMP14_1, | 110 |
| LKSSGGK | | ADAM17_2 | |
| GGGGGGGGSGGGGSFVGGTG | PCL22 | | 111 |
| GGGSGGGGSGGS | | | |
| GGGGSGGGGSGGGGSISSGLLS | PCL23 | | 112 |
| GRSDNHGGSGGS | | | |
| GGGGSGGGGSGGGGSVPLSLYS | PCL24 | | 113 |
| GGGSGGSGGSGS | | | |
| GGGGSGGGGSGPLGLWSQGGG | PCL25 | | 114 |
| GSGGGGSGGGGSGG | | | |
| GGGGSGGGGSKKAAPGGGGSG | PCL26 | | 115 |
| GGGSGGGGSGGS | | | |
| GGGGSGGGGSKKAAPVNGGGG | PCL27 | | 116 |
| GSGGGGSGGGGS | | | |
| GGGGSGGGGSPMAKKGGGGSG | PCL28 | | 117 |
| GGGSGGGGSGGS | | | |
| GGGGSGGGGSPMAKKVNGGGG | PCL29 | | 118 |
| GSGGGGSGGGGS | | | |
| GGGGSGGGGSQARAKGGGGSG | PCL30 | | 119 |
| GGGSGGGGSGGS | | | |
| GGGGSGGGGSQARAKVNGGGG | PCL31 | | 120 |
| GSGGGGSGGGGS | | | |
| GGGGSGGGGSRQARVVNGGGG | PCL32 | | 121 |
| GSGGGGSGGGGS | | | |
| GGGGSGGGGSRQARVVNGGGG | PCL33 | | 122 |
| GSVPLSLYSGGGGGSGGGGS | | | |
| GGGGSGGGGSRQARVVNSVPLS | PCL34 | | 123 |
| LYSGGGGGSGGGGS | | | |
| GGGGSGGGGSVHMPLGFLGPG | PCL35 | | 124 |
| GGGSGGGGSGGS | | | |
| GGGGSVHMPLGFLGPGRSRGSF | PCL36 | | 125 |
| PGGGGS | | | |
| GGGGSVHMPLGFLGPPMAKKGG | PCL37 | | 126 |
| GGSGGGGSGGS | | | |
| GGGGSVHMPLGFLGPRQARVVN | PCL38 | | 127 |
| GGGGSGGGGS | | | |
| GGGGSVHMPLGFLGPRQARVVN | PCL39 | | 128 |
| GGGGSGGGGSGG | | | |
| GGPLAQKLKSSALFKSSFPGPAG | PCL40 | ADAM17_2, CTSL1_1, | 129 |
| LYAQGGR | | MMP14_1 | |
| GLSGRSDNHGGAVGLLAPP | PCL41 | | 130 |
| GLSGRSDNHGGVHMPLGFLGP | PCL42 | | 131 |
| ISSGLLSGRSANI | PCL43 | MMP, Serine protease | 132 |
| ISSGLLSGRSANP | PCL44 | MMP, Serine protease | 133 |
| ISSGLLSGRSANPRG | PCL45 | MMP, Serine protease | 134 |
| ISSGLLSGRSDDH | PCL46 | MMP, Serine protease | 135 |
| ISSGLLSGRSDIH | PCL47 | MMP, Serine protease | 136 |
| ISSGLLSGRSDNH | PCL48 | MMP, Serine protease | 137 |
| ISSGLLSGRSDNI | PCL49 | CTSL1_1, MMP14_1 | 138 |
| ISSGLLSGRSDNP | PCL50 | MMP, Serine protease | 139 |
| ISSGLLSGRSDQH | PCL51 | MMP, Serine protease | 140 |
| ISSGLLSGRSDTH | PCL52 | MMP, Serine protease | 141 |
| ISSGLLSGRSDYH | PCL53 | MMP, Serine protease | 142 |
| ISSGLLSGRSGNH | PCL54 | MMP, Serine protease | 143 |
| ISSGLLSSGGSGGSLSGRSDNH | PCL55 | | 144 |
| ISSGLLSSGGSGGSLSGRSGNH | PCL56 | | 145 |
| KGGPGGPAGIGPLAQRLRSSALF | PCL57 | FAPa_1, ADAM17_1, | 146 |
| KSSFPGR | | CTSL1_1 | |
| KSGPGGPAGIGALFFSSPPLAQKL | PCL58 | FAPa_1, CTSL1_2, | 147 |
| KSSGGR | | ADAM17_2 | |
| LSGRSDNHGGAVGLLAPP | PCL59 | | 148 |
| LSGRSDNHGGSGGSISSGLLSS | PCL60 | | 149 |
| LSGRSDNHGGSGGSQNQALRMA | PCL61 | | 150 |
| LSGRSDNHGGVHMPLGFLGP | PCL62 | | 151 |
| LSGRSGNHGGSGGSISSGLLSS | PCL63 | | 152 |
| LSGRSGNHGGSGGSQNQALRMA | PCL64 | | 153 |
| QNQALRMAGGSGGSLSGRSDNH | PCL65 | | 154 |
| QNQALRMAGGSGGSLSGRSGNH | PCL66 | | 155 |
| RGGALFKSSFPLAQKLKSSGPAG | PCL67 | CTSL1_1, ADAM17_2, | 156 |
| LYAQGGK | | MMP14_1 | |
| RGGGPAGLYAQPLAQKLKSSALF | PCL68 | MMP14_1, ADAM17_2, | 157 |
| KSSFPGG | | CTSL1_1 | |
| SGGFPRSGGSFNPRTFGSKRKR | PCL69 | thrombin, factor Xa, | 158 |
| RGSRGGGG | | hepsin | |
| SGPLAQKLKSSGPAGLYAQALFK | PCL70 | ADAM17_2, MMP14_1, | 159 |
| SSFPGSK | | CTSL1_1 | |
| TSTSGRSANPRGGGAVGLLAPP | PCL71 | | 160 |
| TSTSGRSANPRGGGVHMPLGFL | PCL72 | | 161 |
| GP | | | |
| VHMPLGFLGPGGLSGRSDNH | PCL73 | | 162 |
| VHMPLGFLGPGGTSTSGRSANP | PCL74 | | 163 |
| RG | | | |
| SGRSAGGGSGRSAGGGSGRSA | PCL75 | uPA | 164 |
| HPVGLLARGGGHPVGLLARGGG | PCL76 | MPA (MMP-2 and uPA) | 165 |
| SGRSAGGGSGRSA | | | |
| GPLGVRGK | PCL77 | MMP-2 | 166 |
| HPVGLLAR | PCL78 | MMP-2 | 167 |
| GPQGIAGQ | PCL79 | MMP-2, MMP-9, and to | 168 |
| | | some degree MT1-MMP | |
| VPMSMRGG | PCL80 | MMP-9 and MMP-2 | 169 |
| IPVSLRSG | PCL81 | MMP-2, and to some | 170 |
| | | degree MMP-9 or MMP-7 | |
| RPFSMIMG | PCL82 | MMP-9 and MMP-7, to | 171 |
| | | some degree MMP-3 | |
| VPLSLTMG | PCL83 | MMP-7, to some degree | 172 |
| | | MMP-9, MMP-2, MPT-1- | |
| | | MMP | |
| VPLSLYSG | PCL84 | MMP-2, MMP-9, MMP-7 | 173 |
| IPESLRAG | PCL85 | MMP-2, MMP-7, MMP-9, | 174 |
| | | to some degree MPT-1- | |
| | | MMP | |
| VPLSLYSGGGISSGLLSGRSDNH | PCL86 | | 175 |
| GGGISSGLLSGRSDNHGGGS | PCL87 | | 176 |
| GGGHPVGLLARGGGS | PCL88 | | 177 |
| GGGSGGGSGGGGISSGLLSGRS | PCL89 | | 178 |
| DNHGGGSGGGSGGS | | | |
| GGGGISSGLLSGRSDNHGGGISS | PCL90 | | 179 |
| GLLSGRSDNHGGS | | | |
| GGGSGGSIPVSLRSGGGISSGLL | PCL91 | | 180 |
| SGRSDNHGGSGGS | | | |
| GGGSGGSVPLSLYSGGGISSGLL | PCL92 | | 181 |
| SGRSDNHGGSGGS | | | |
| GGGSHPVGLLARGGGHPVGLLA | PCL93 | | 182 |
| RGGGHPVGLLARGS | | | |
| GGGSHPVGLLARGGGHPVGLLA | PCL94 | | 183 |
| RGGSGRSAGGSGRS | | | |
| GISSGLLSGRSDNHG | PCL95 | | 184 |
| GGGSISSGLLSGRSDNHGGGS | PCL96 | | 185 |

In certain aspects, the protease-cleavable linker comprises an amino acid sequence having up to 5, up to 4, up to 3, up to 2 or up to 1 amino acid substitution(s) as compared to the sequence set forth in Table D. Thus, in some embodiments, the protease-cleavable linker comprises or consists of any amino acid sequence in Table D with 1-5 amino acid substitutions as compared to the sequence set forth in Table D.

In some embodiments, the engineered KL polypeptide comprises linker PCL1. In some embodiments, the engineered KL polypeptide comprises linker PCL2. In some embodiments, the engineered KL polypeptide comprises linker PCL3. In some embodiments, the engineered KL polypeptide comprises linker PCL4. In some embodiments, the engineered KL polypeptide comprises linker PCL5. In some embodiments, the engineered KL polypeptide comprises linker PCL6. In some embodiments, the engineered KL polypeptide comprises linker PCL7. In some embodiments, the engineered KL polypeptide comprises linker PCL8. In some embodiments, the engineered KL polypeptide comprises linker PCL9. In some embodiments, the engineered KL polypeptide comprises linker PCL10. In some embodiments, the engineered KL polypeptide comprises linker PCL11. In some embodiments, the engineered KL polypeptide comprises linker PCL12. In some embodiments, the engineered KL polypeptide comprises linker PCL13. In some embodiments, the engineered KL polypeptide comprises linker PCL14. In some embodiments, the engineered KL polypeptide comprises linker PCL15. In some embodiments, the engineered KL polypeptide comprises linker PCL16. In some embodiments, the engineered KL polypeptide comprises linker PCL17. In some embodiments, the engineered KL polypeptide comprises linker PCL18. In some embodiments, the engineered KL polypeptide comprises linker PCL19. In some embodiments, the engineered KL polypeptide comprises linker PCL20. In some embodiments, the engineered KL polypeptide comprises linker PCL21. In some embodiments, the engineered KL polypeptide comprises linker PCL22. In some embodiments, the engineered KL polypeptide comprises linker PCL23. In some embodiments, the engineered KL polypeptide comprises linker PCL24. In some embodiments, the engineered KL polypeptide comprises linker PCL25. In some embodiments, the engineered KL polypeptide comprises linker PCL26. In some embodiments, the engineered KL polypeptide comprises linker PCL27. In some embodiments, the engineered KL polypeptide comprises linker PCL28. In some embodiments, the engineered KL polypeptide comprises linker PCL29. In some embodiments, the engineered KL polypeptide comprises linker PCL30. In some embodiments, the engineered KL polypeptide comprises linker PCL31. In some embodiments, the engineered KL polypeptide comprises linker PCL32. In some embodiments, the engineered KL polypeptide comprises linker PCL33. In some embodiments, the engineered KL polypeptide comprises linker PCL34. In some embodiments, the engineered KL polypeptide comprises linker PCL35. In some embodiments, the engineered KL polypeptide comprises linker PCL36. In some embodiments, the engineered KL polypeptide comprises linker PCL37. In some embodiments, the engineered KL polypeptide comprises linker PCL38. In some embodiments, the engineered KL polypeptide comprises linker PCL39. In some embodiments, the engineered KL polypeptide comprises linker PCL40. In some embodiments, the engineered KL polypeptide comprises linker PCL41. In some embodiments, the engineered KL polypeptide comprises linker PCL42. In some embodiments, the engineered KL polypeptide comprises linker PCL43. In some embodiments, the engineered KL polypeptide comprises linker PCL44. In some embodiments, the engineered KL polypeptide comprises linker PCL45. In some embodiments, the engineered KL polypeptide comprises linker PCL46. In some embodiments, the engineered KL polypeptide comprises linker PCL47. In some embodiments, the engineered KL polypeptide comprises linker PCL48. In some embodiments, the engineered KL polypeptide comprises linker PCL49. In some embodiments, the engineered KL polypeptide comprises linker PCL50. In some embodiments, the engineered KL polypeptide comprises linker PCL51. In some embodiments, the engineered KL polypeptide comprises linker PCL52. In some embodiments, the engineered KL polypeptide comprises linker PCL53. In some embodiments, the engineered KL polypeptide comprises linker PCL54. In some embodiments, the engineered KL polypeptide comprises linker PCL55. In some embodiments, the engineered KL polypeptide comprises linker PCL56. In some embodiments, the engineered KL polypeptide comprises linker PCL57. In some embodiments, the engineered KL polypeptide comprises linker PCL58. In some embodiments, the engineered KL polypeptide comprises linker PCL59. In some embodiments, the engineered KL polypeptide comprises linker PCL60. In some embodiments, the engineered KL polypeptide comprises linker PCL61. In some embodiments, the engineered KL polypeptide comprises linker PCL62. In some embodiments, the engineered KL polypeptide comprises linker PCL63. In some embodiments, the engineered KL polypeptide comprises linker PCL64. In some embodiments, the engineered KL polypeptide comprises linker PCL65. In some embodiments, the engineered KL polypeptide comprises linker PCL66. In some embodiments, the engineered KL polypeptide comprises linker PCL67. In some embodiments, the engineered KL polypeptide comprises linker PCL68. In some embodiments, the engineered KL polypeptide comprises linker PCL69. In some embodiments, the engineered KL polypeptide comprises linker PCL70. In some embodiments, the engineered KL polypeptide comprises linker PCL71. In some embodiments, the engineered KL polypeptide comprises linker PCL72. In some embodiments, the engineered KL polypeptide comprises linker PCL73. In some embodiments, the engineered KL polypeptide comprises linker PCL74. In some embodiments, the engineered KL polypeptide comprises linker PCL75. In some embodiments, the engineered KL polypeptide comprises linker PCL76. In some embodiments, the engineered KL polypeptide comprises linker PCL77. In some embodiments, the engineered KL polypeptide comprises linker PCL78. In some embodiments, the engineered KL polypeptide comprises linker PCL79. In some embodiments, the engineered KL polypeptide comprises linker PCL80. In some embodiments, the engineered KL polypeptide comprises linker PCL81. In some embodiments, the engineered KL polypeptide comprises linker PCL82. In some embodiments, the engineered KL polypeptide comprises linker PCL83. In some embodiments, the engineered KL polypeptide comprises linker PCL84. In some embodiments, the engineered KL polypeptide comprises linker PCL85. In some embodiments, the engineered KL polypeptide comprises linker PCL86. In some embodiments, the engineered KL polypeptide comprises linker PCL87. In some embodiments, the engineered KL polypeptide comprises linker PCL88. In some embodiments, the engineered KL polypeptide comprises linker PCL89. In some embodiments, the engineered KL polypeptide comprises linker PCL90. In some embodiments, the engineered KL polypeptide comprises linker PCL91. In some embodiments, the engineered KL polypeptide comprises linker PCL92. In some embodiments, the engineered KL polypeptide comprises linker PCL93. In some embodiments, the engineered KL polypeptide comprises linker PCL94. In some embodiments, the engineered KL polypeptide comprises linker PCL95. In some embodiments, the engineered KL polypeptide comprises linker PCL96.

### 6.6. Nucleic Acids and Host Cells

In another aspect, the disclosure provides nucleic acids encoding the engineered alpha klotho polypeptides of the disclosure. The nucleic acids of the disclosure can be DNA (e.g., plasmid) or RNA (e.g., mRNA).

In some embodiments, a nucleic acid of the present disclosure comprises a nucleic acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or 100% sequence identity to any one of SEQ ID NOs: 41, 43, 45, 47, 49, 51, 53, 55, 57, 59, 61, 63, 65, 67, and 69. In some embodiments, the nucleic acid comprises a nucleic acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or 100% sequence identity to SEQ ID NO:67. In some embodiments, the nucleic acid comprises a nucleic acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or 100% sequence identity to SEQ ID NO:69.

In some aspects, the disclosure provides host cells and vectors containing the nucleic acids of the disclosure. The nucleic acids may be present in a single vector or separate vectors present in the same host cell or separate host cell, as described in more detail herein below.

### 6.6.1. Vectors

The disclosure provides vectors comprising nucleotide sequences encoding an alpha klotho polypeptide or a component thereof described herein, for example a polypeptide chains of an alpha klotho polypeptide. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection, or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.6.2. Host Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein. The cell can be, but is not limited to, a eukaryotic cell, a bacterial cell, an insect cell, or a human cell. Suitable eukaryotic cells include, but are not limited to, Vero cells, Hela cells, COS cells, CHO cells, HEK293 cells, BHK cells and MDCKII cells. Suitable insect cells include, but are not limited to, Sf9 cells.

### 6.7. Pharmaceutical Compositions

The engineered alpha klotho polypeptides of the disclosure may be in the form of compositions comprising the engineered alpha klotho polypeptide and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the engineered alpha klotho polypeptide and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically, or locally. The most suitable route for administration in any given case will depend on the particular alpha klotho polypeptide, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an engineered alpha klotho polypeptide of the disclosure per dose. The quantity of engineered alpha klotho polypeptide included in a unit dose will depend on the disease being treated, as well as other factors as are well known in the art. Such unit dosages may be in the form of a lyophilized dry powder containing an amount of engineered alpha klotho polypeptide suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of alpha klotho polypeptide suitable for a single administration.

The pharmaceutical compositions may also be supplied in bulk from containing quantities of alpha klotho polypeptide suitable for multiple administrations.

Pharmaceutical compositions may be prepared for storage as lyophilized formulations or aqueous solutions by mixing an alpha klotho polypeptide having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of alpha klotho polypeptide.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The engineered alpha klotho polypeptides of the disclosure can be formulated as pharmaceutical compositions comprising the engineered alpha klotho polypeptides, for example containing one or more pharmaceutically acceptable excipients or carriers. To prepare pharmaceutical or sterile compositions comprising the engineered alpha klotho polypeptides of the present disclosure, an engineered alpha klotho polypeptide preparation can be combined with one or more pharmaceutically acceptable excipient or carrier.

For example, formulations of engineered alpha klotho polypeptides can be prepared by mixing engineered alpha klotho polypeptides with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

An effective amount for a particular subject may vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

A composition of the present disclosure may also be administered via one or more routes of administration using one or more of a variety of methods known in the art. As will be appreciated by the skilled artisan, the route and/or mode of administration will vary depending upon the desired results. Selected routes of administration for engineered alpha klotho polypeptides include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal, or mucosal route of administration, for example, intranasally, orally, vaginally, rectally, sublingually, or topically. In one embodiment, the engineered alpha klotho polypeptides are administered by infusion. In another embodiment, an engineered alpha klotho polypeptide of the disclosure is administered subcutaneously.

### 6.8. Therapeutic Indications and Methods of Use

The present disclosure provides methods for using and applications for the engineered alpha klotho polypeptides of the disclosure.

The engineered alpha klotho polypeptides of the disclosure can be used in therapeutic methods for treatment of a variety of disorders including age-related conditions, metabolic disorders, and kidney disease.

In certain aspects, the disclosure provides a method of treating an age-related condition comprising administering to a subject in need thereof an engineered alpha klotho polypeptide as described herein. Age-related conditions include, but are not limited to, hearing loss, cataracts and refractive errors, osteoarthritis, chronic obstructive pulmonary disease, diabetes, and dementia. An engineered alpha klotho polypeptide of the disclosure may be administered to a subject suffering from or diagnosed with an age-related condition. An engineered alpha klotho polypeptide may also be administered to a subject at risk for developing an age-related condition. For example, a subject having an increased risk for developing an age-related condition may be administered an engineered alpha klotho polypeptide prophylactically, thereby reducing the risk of developing the age-related condition.

In certain aspects, the disclosure provides a method of treating kidney disease comprising administering to a subject in need thereof an engineered alpha klotho polypeptide as described herein. Kidney disease includes both acute kidney injury and chronic kidney disease. Accordingly, an engineered alpha klotho polypeptide may be administered to a subject following acute kidney injury or following diagnosis of chronic kidney disease. In some embodiments, the engineered alpha klotho polypeptide is administered to the subject following acute kidney injury. An engineered alpha klotho polypeptide may also be administered to a subject at risk for developing a kidney disease. For example, a subject having an increased risk for chronic kidney disease may be administered an engineered alpha klotho polypeptide prophylactically, thereby reducing the risk of developing chronic kidney disease.

Also disclosed are methods of supplementing loss of endogenous alpha klotho comprising administering to a subject in need thereof an engineered alpha klotho polypeptide as described herein. Endogenous levels of alpha klotho protein decrease with age, and engineered alpha klotho polypeptides may be used to supplement this loss. Accordingly, in some embodiments, an engineered alpha klotho polypeptide of the disclosure is administered to a subject having reduced alpha klotho protein levels relative to prior levels in the same subject (e.g., reduced by at least about 5%, 10%, 15%, 20%, 25%, or 30% relative to alpha klotho proteins levels in the same subject from at least 6 months prior).

Methods of activating FGFR signaling are also disclosed herein using engineered alpha klotho polypeptides. Exemplary engineered alpha klotho polypeptides of the present disclosure can interact with FGF23 and activate FGFR signaling in a cell. Accordingly, disclosed is a method of activating FGFR signaling in a cell comprising contacting the cell with an engineered alpha klotho polypeptide of the disclosure. The cell may be any cell expressing an FGFR on its surface. In some embodiments, the cell is a kidney cell.

### 7. SEQUENCES

Certain sequences of the disclosure are provided in Table S below.

TABLE S
Sequences

| | | SEQ ID |
|---|---|---|
| Name | Sequence | NO: |
| Human alpha | MPASAPPRRPRPPPPSLSLLLVLLGLGGRRLRAEPGDGAQTWAR | 1 |
| klotho | VSRPPAPEAAGLFQGTFPDGFLWAVGSAAYQTEGGWQQHGKGA | |
| (BAA23382.1) | SIWDTFTHHPLAPPGDSRNASLPLGAPSPLQPATGDVASDSYNNV | |
| | FRDTEALRELGVTHYRFSISWARVLPNGSAGVPNREGLRYYRRLL | |
| | ERLRELGVQPVVTLYHWDLPQRLQDAYGGWANRALADHFRDYA | |
| | ELCFRHFGGQVKYWITIDNPYVVAWHGYATGRLAPGIRGSPRLGY | |
| | LVAHNLLLAHAKVWHLYNTSFRPTQGGQVSIALSSHWINPRRMTD | |
| | HSIKECQKSLDFVLGWFAKPVFIDGDYPESMKNNLSSILPDFTESE | |
| | KKFIKGTADFFALCFGPTLSFQLLDPHMKFRQLESPNLRQLLSWID | |
| | LEFNHPQIFIVENGWFVSGTTKRDDAKYMYYLKKFIMETLKAIKLDG | |
| | VDVIGYTAWSLMDGFEWHRGYSIRRGLFYVDFLSQDKMLLPKSSA | |
| | LFYQKLIEKNGFPPLPENQPLEGTFPCDFAWGVVDNYIQVDTTLSQ | |
| | FTDLNVYLWDVHHSKRLIKVDGVVTKKRKSYCVDFAAIQPQIALLQ | |
| | EMHVTHFRFSLDWALILPLGNQSQVNHTILQYYRCMASELVRVNIT | |
| | PWVALWQPMAPNQGLPRLLARQGAWENPYTALAFAEYARLCFQE | |
| | LGHHVKLWITMNEPYTRNMTYSAGHNLLKAHALAWHVYNEKFRH | |
| | AQNGKISIALQADWIEPACPFSQKDKEVAERVLEFDIGWLAEPIFG | |
| | SGDYPWVMRDWLNQRNNFLLPYFTEDEKKLIQGTFDFLALSHYTT | |
| | ILVDSEKEDPIKYNDYLEVQEMTDITWLNSPSQVAVVPWGLRKVLN | |
| | WLKFKYGDLPMYIISNGIDDGLHAEDDQLRVYYMQNYINEALKAHI | |
| | LDGINLCGYFAYSFNDRTAPRFGLYRYAADQFEPKASMKHYRKIID | |
| | SNGFPGPETLERFCPEEFTVCTECSFFHTRKSLLAFIAFLFFASIISL | |
| | SLIFYYSKKGRRSYK | |
| Human alpha | MPASAPPRRPRPPPPSLSLLLVLLGLGGRRLRAEPGDGAQTWAR | 2 |
| klotho | FSRPPAPEAAGLFQGTFPDGFLWAVGSAAYQTEGGWQQHGKGA | |
| (Q9UEF7-1) | SIWDTFTHHPLAPPGDSRNASLPLGAPSPLQPATGDVASDSYNNV | |
| | FRDTEALRELGVTHYRFSISWARVLPNGSAGVPNREGLRYYRRLL | |
| | ERLRELGVQPVVTLYHWDLPQRLQDAYGGWANRALADHFRDYA | |
| | ELCFRHFGGQVKYWITIDNPYVVAWHGYATGRLAPGIRGSPRLGY | |
| | LVAHNLLLAHAKVWHLYNTSFRPTQGGQVSIALSSHWINPRRMTD | |
| | HSIKECQKSLDFVLGWFAKPVFIDGDYPESMKNNLSSILPDFTESE | |
| | KKFIKGTADFFALCFGPTLSFQLLDPHMKFRQLESPNLRQLLSWID | |
| | LEFNHPQIFIVENGWFVSGTTKRDDAKYMYYLKKFIMETLKAIKLDG | |
| | VDVIGYTAWSLMDGFEWHRGYSIRRGLFYVDFLSQDKMLLPKSSA | |
| | LFYQKLIEKNGFPPLPENQPLEGTFPCDFAWGVVDNYIQVDTTLSQ | |
| | FTDLNVYLWDVHHSKRLIKVDGVVTKKRKSYCVDFAAIQPQIALLQ | |
| | EMHVTHFRFSLDWALILPLGNQSQVNHTILQYYRCMASELVRVNIT | |
| | PWVALWQPMAPNQGLPRLLARQGAWENPYTALAFAEYARLCFQE | |
| | LGHHVKLWITMNEPYTRNMTYSAGHNLLKAHALAWHVYNEKFRH | |
| | AQNGKISIALQADWIEPACPFSQKDKEVAERVLEFDIGWLAEPIFG | |
| | SGDYPWVMRDWLNQRNNFLLPYFTEDEKKLIQGTFDFLALSHYTT | |
| | ILVDSEKEDPIKYNDYLEVQEMTDITWLNSPSQVAVVPWGLRKVLN | |
| | WLKFKYGDLPMYIISNGIDDGLHAEDDQLRVYYMQNYINEALKAHI | |
| | LDGINLCGYFAYSFNDRTAPRFGLYRYAADQFEPKASMKHYRKIID | |
| | SNGFPGPETLERFCPEEFTVCTECSFFHTRKSLLAFIAFLFFASIISL | |
| | SLIFYYSKKGRRSYK | |
| KL2 (wt) | LEGTFPCDFAWGVVDNYIQVDTTLSQFTDLNVYLWDVHHSKRLIK | 3 |
| | VDGVVTKKRKSYCVDFAAIQPQIALLQEMHVTHFRFSLDWALILPL | |
| | GNQSQVNHTILQYYRCMASELVRVNITPVVALWQPMAPNQGLPR | |
| | LLARQGAWENPYTALAFAEYARLCFQELGHHVKLWITMNEPYTRN | |
| | MTYSAGHNLLKAHALAWHVYNEKFRHAQNGKISIALQADWIEPAC | |
| | PFSQKDKEVAERVLEFDIGWLAEPIFGSGDYPWVMRDWLNQRNN | |
| | FLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGID | |
| | DGLHAEDDQLRVYYMQNYINEALKAHILDGINLCGYFAYSFNDRTA | |
| | PRFGLYRYAADQFEPKASMKHYRKIIDS | |
| KL2 (C521S) | LEGTFPSDFAWGVVDNYIQVDTTLSQFTDLNVYLWDVHHSKRLIK | 4 |
| | VDGVVTKKRKSYCVDFAAIQPQIALLQEMHVTHFRFSLDWALILPL | |
| | GNQSQVNHTILQYYRCMASELVRVNITPVVALWQPMAPNQGLPR | |
| | LLARQGAWENPYTALAFAEYARLCFQELGHHVKLWITMNEPYTRN | |
| | MTYSAGHNLLKAHALAWHVYNEKFRHAQNGKISIALQADWIEPAC | |
| | PFSQKDKEVAERVLEFDIGWLAEPIFGSGDYPWVMRDWLNQRNN | |
| | FLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGID | |
| | DGLHAEDDQLRVYYMQNYINEALKAHILDGINLCGYFAYSFNDRTA | |
| | PRFGLYRYAADQFEPKASMKHYRKIIDS | |
| KL2 + c-term | LEGTFPCDFAWGVVDNYIQVDTTLSQFTDLNVYLWDVHHSKRLIK | 5 |
| loop (wt) | VDGVVTKKRKSYCVDFAAIQPQIALLQEMHVTHFRFSLDWALILPL | |
| | GNQSQVNHTILQYYRCMASELVRVNITPVVALWQPMAPNQGLPR | |
| | LLARQGAWENPYTALAFAEYARLCFQELGHHVKLWITMNEPYTRN | |
| | MTYSAGHNLLKAHALAWHVYNEKFRHAQNGKISIALQADWIEPAC | |
| | PFSQKDKEVAERVLEFDIGWLAEPIFGSGDYPWVMRDWLNQRNN | |
| | FLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGID | |
| | DGLHAEDDQLRVYYMQNYINEALKAHILDGINLCGYFAYSFNDRTA | |
| | PRFGLYRYAADQFEPKASMKHYRKIIDSNGFPGPETLERFCPEEFT | |
| | VCTECSFFHTRKS | |
| KL2 + c-term | LEGTFPSDFAWGVVDNYIQVDTTLSQFTDLNVYLWDVHHSKRLIK | 6 |
| loop (C521S) | VDGVVTKKRKSYCVDFAAIQPQIALLQEMHVTHFRFSLDWALILPL | |
| | GNQSQVNHTILQYYRCMASELVRVNITPVVALWQPMAPNQGLPR | |
| | LLARQGAWENPYTALAFAEYARLCFQELGHHVKLWITMNEPYTRN | |
| | MTYSAGHNLLKAHALAWHVYNEKFRHAQNGKISIALQADWIEPAC | |
| | PFSQKDKEVAERVLEFDIGWLAEPIFGSGDYPWVMRDWLNQRNN | |
| | FLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGID | |
| | DGLHAEDDQLRVYYMQNYINEALKAHILDGINLCGYFAYSFNDRTA | |
| | PRFGLYRYAADQFEPKASMKHYRKIIDSNGFPGPETLERFCPEEFT | |
| | VCTECSFFHTRKS | |
| C-term tail | NGFPGPETLERFCPEEFTVCTECSFFHTRKS | 7 |
| region | | |
| C-terminal 23AA | LERFCPEEFTVCTECSFFHTRKS | 8 |
| of C-term tail | | |
| region | | |
| KL1 | EPGDGAQTWARVSRPPAPEAAGLFQGTFPDGFLWAVGSAAYQT | 9 |
| (BAA23382.1) | EGGWQQHGKGASIWDTFTHHPLAPPGDSRNASLPLGAPSPLQPA | |
| | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGF | |
| KL1 (Q9UEF7-1) | EPGDGAQTWARFSRPPAPEAAGLFQGTFPDGFLWAVGSAAYQT | 10 |
| | EGGWQQHGKGASIWDTFTHHPLAPPGDSRNASLPLGAPSPLQPA | |
| | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGF | |
| KL1 | EPGDGAQTWARVSRPPAPEAAGLFQGTFPDGFLWAVGSAAYQT | 71 |
| (BAA23382.1) | EGGWQQHGKGASIWDTFTHHPLAPPGDSRNASLPLGAPSPLQPA | |
| (C370S) | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALSFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGF | |
| KL1 (Q9UEF7-1) | EPGDGAQTWARFSRPPAPEAAGLFQGTFPDGFLWAVGSAAYQT | 72 |
| (C370S) | EGGWQQHGKGASIWDTFTHHPLAPPGDSRNASLPLGAPSPLQPA | |
| | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALSFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGF | |
| KL1 through | EPGDGAQTWARFSRPPAPEAAGLFQGTFPDGFLWAVGSAAYQT | 11 |
| KL2 (wt - | EGGWQQHGKGASIWDTFTHHPLAPPGDSRNASLPLGAPSPLQPA | |
| Q9UEF7-1) | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPCDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDS | |
| KL1 through | EPGDGAQTWARFSRPPAPEAAGLFQGTFPDGFLWAVGSAAYQT | 73 |
| KL2 (C521S - | EGGWQQHGKGASIWDTFTHHPLAPPGDSRNASLPLGAPSPLQPA | |
| Q9UEF7-1) | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPSDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDS | |
| KL1 through | EPGDGAQTWARFSRPPAPEAAGLFQGTFPDGFLWAVGSAAYQT | 12 |
| KL2 (C521S - | EGGWQQHGKGASIWDTFTHHPLAPPGDSRNASLPLGAPSPLQPA | |
| BAA23382.1) | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYWVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPSDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDS | |
| KL1 through | EPGDGAQTWARFSRPPAPEAAGLFQGTFPDGFLWAVGSAAYQT | 74 |
| KL2 (C521S + | EGGWQQHGKGASIWDTFTHHPLAPPGDSRNASLPLGAPSPLQPA | |
| C370S - | TGDVASDSYNNVFRDTEALRELGVTHYRFSISWARVLPNGSAGVP | |
| BAA23382.1 ) | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALSFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPSDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDS | |
| hKL ECD - wt | EPGDGAQTWARFSRPPAPEAAGLFQGTFPDGFLWAVGSAAYQT | 13 |
| | EGGWQQHGKGASIWDTFTHHPLAPPGDSRNASLPLGAPSPLQPA | |
| | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPCDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDSNGFPGPETLERFCPEEFTVCTECSFFHTRKS | |
| hKL ECD - | EPGDGAQTWARFSRPPAPEAAGLFQGTFPDGFLWAVGSAAYQT | 14 |
| C521S | EGGWQQHGKGASIWDTFTHHPLAPPGDSRNASLPLGAPSPLQPA | |
| | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPSDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDSNGFPGPETLERFCPEEFTVCTECSFFHTRKS | |
| hKL ECD - | EPGDGAQTWARFSRPPAPEAAGLFQGTFPDGFLWAVGSAAYQT | 75 |
| C370S + C521S | EGGWQQHGKGASIWDTFTHHPLAPPGDSRNASLPLGAPSPLQPA | |
| | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALSFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPSDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDSNGFPGPETLERFCPEEFTVCTECSFFHTRKS | |
| hKL ECD - | EPGDGAQTWARFSRPPAPEAAGLFQGTFPDGFLWAVGSAAYQT | 15 |
| 23aa truncation | EGGWQQHGKGASIWDTFTHHPLAPPGDSRNASLPLGAPSPLQPA | |
| (wt) | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPCDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDSNGFPGPET | |
| hKL ECD - | EPGDGAQTWARFSRPPAPEAAGLFQGTFPDGFLWAVGSAAYQT | 16 |
| 23aa truncation | EGGWQQHGKGASIWDTFTHHPLAPPGDSRNASLPLGAPSPLQPA | |
| (C521S) | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALCFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPSDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDSNGFPGPET | |
| hKL ECD - | EPGDGAQTWARFSRPPAPEAAGLFQGTFPDGFLWAVGSAAYQT | 76 |
| 23aa truncation | EGGWQQHGKGASIWDTFTHHPLAPPGDSRNASLPLGAPSPLQPA | |
| (C521S + C370S) | TGDVASDSYNNVFRDTEALRELGVTHYRFSISWARVLPNGSAGVP | |
| | NREGLRYYRRLLERLRELGVQPVVTLYHWDLPQRLQDAYGGWAN | |
| | RALADHFRDYAELCFRHFGGQVKYWITIDNPYVVAWHGYATGRLA | |
| | PGIRGSPRLGYLVAHNLLLAHAKVWHLYNTSFRPTQGGQVSIALS | |
| | SHWINPRRMTDHSIKECQKSLDFVLGWFAKPVFIDGDYPESMKNN | |
| | LSSILPDFTESEKKFIKGTADFFALSFGPTLSFQLLDPHMKFRQLES | |
| | PNLRQLLSWIDLEFNHPQIFIVENGWFVSGTTKRDDAKYMYYLKKF | |
| | IMETLKAIKLDGVDVIGYTAWSLMDGFEWHRGYSIRRGLFYVDFLS | |
| | QDKMLLPKSSALFYQKLIEKNGFPPLPENQPLEGTFPSDFAWGVV | |
| | DNYIQVDTTLSQFTDLNVYLWDVHHSKRLIKVDGVVTKKRKSYCV | |
| | DFAAIQPQIALLQEMHVTHFRFSLDWALILPLGNQSQVNHTILQYY | |
| | RCMASELVRVNITPVVALWQPMAPNQGLPRLLARQGAWENPYTA | |
| | LAFAEYARLCFQELGHHVKLWITMNEPYTRNMTYSAGHNLLKAHA | |
| | LAWHVYNEKFRHAQNGKISIALQADWIEPACPFSQKDKEVAERVL | |
| | EFDIGWLAEPIFGSGDYPWVMRDWLNQRNNFLLPYFTEDEKKLIQ | |
| | GTFDFLALSHYTTILVDSEKEDPIKYNDYLEVQEMTDITWLNSPSQ | |
| | VAVVPWGLRKVLNWLKFKYGDLPMYIISNGIDDGLHAEDDQLRVY | |
| | YMQNYINEALKAHILDGINLCGYFAYSFNDRTAPRFGLYRYAADQF | |
| | EPKASMKHYRKIIDSNGFPGPET | |
| hKL | LLAFIAFLFFASIISLSLIFY | 17 |
| Transmembrane | | |
| domain | | |
| hKL Intracellular | YSKKGRRSYK | 18 |
| domain | | |
| Human Serum | DAHKSEVAHRFKDLGEENFKALVLIAFAQYLQQCPFEDHVKLVNE | 19 |
| Albumin (wt) | VTEFAKTCVADESAENCDKSLHTLFGDKLCTVATLRETYGEMADC | |
| | CAKQEPERNECFLQHKDDNPNLPRLVRPEVDVMCTAFHDNEETF | |
| | LKKYLYEIARRHPYFYAPELLFFAKRYKAAFTECCQAADKAACLLP | |
| | KLDELRDEGKASSAKQRLKCASLQKFGERAFKAWAVARLSQRFP | |
| | KAEFAEVSKLVTDLTKVHTECCHGDLLECADDRADLAKYICENQD | |
| | SISSKLKECCEKPLLEKSHCIAEVENDEMPADLPSLAADFVESKDV | |
| | CKNYAEAKDVFLGMFLYEYARRHPDYSVVLLLRLAKTYETTLEKC | |
| | CAAADPHECYAKVFDEFKPLVEEPQNLIKQNCELFEQLGEYKFQN | |
| | ALLVRYTKKVPQVSTPTLVEVSRNLGKVGSKCCKHPEAKRMPCAE | |
| | DYLSVVLNQLCVLHEKTPVSDRVTKCCTESLVNRRPCFSALEVDE | |
| | TYVPKEFNAETFTFHADICTLSEKERQIKKQTALVELVKHKPKATKE | |
| | QLKAVMDDFAAFVEKCCKADDKETCFAEEGKKLVAASQAALGL | |
| Human Serum | DAHKSEVAHRFKDLGEENFKALVLIAFAQYLQQSPFEDHVKLVNE | 20 |
| Albumin (C34S) | VTEFAKTCVADESAENCDKSLHTLFGDKLCTVATLRETYGEMADC | |
| | CAKQEPERNECFLQHKDDNPNLPRLVRPEVDVMCTAFHDNEETF | |
| | LKKYLYEIARRHPYFYAPELLFFAKRYKAAFTECCQAADKAACLLP | |
| | KLDELRDEGKASSAKQRLKCASLQKFGERAFKAWAVARLSQRFP | |
| | KAEFAEVSKLVTDLTKVHTECCHGDLLECADDRADLAKYICENQD | |
| | SISSKLKECCEKPLLEKSHCIAEVENDEMPADLPSLAADFVESKDV | |
| | CKNYAEAKDVFLGMFLYEYARRHPDYSVVLLLRLAKTYETTLEKC | |
| | CAAADPHECYAKVFDEFKPLVEEPQNLIKQNCELFEQLGEYKFQN | |
| | ALLVRYTKKVPQVSTPTLVEVSRNLGKVGSKCCKHPEAKRMPCAE | |
| | DYLSVVLNQLCVLHEKTPVSDRVTKCCTESLVNRRPCFSALEVDE | |
| | TYVPKEFNAETFTFHADICTLSEKERQIKKQTALVELVKHKPKATKE | |
| | QLKAVMDDFAAFVEKCCKADDKETCFAEEGKKLVAASQAALGL | |
| Human KL | PPLPENQP | 21 |
| interdomain | | |
| region | | |
| Human KL C- | NGFPGPET | 22 |
| terminal tail | | |
| region | | |
| (truncated by | | |
| 23AA) | | |

### 8. SPECIFIC EMBODIMENTS

While various specific embodiments have been illustrated and described, it will be appreciated that various changes can be made without departing from the spirit and scope of the disclosure(s). The present disclosure is exemplified by the numbered embodiments set forth below.

In the numbered embodiments that follow, the alpha klotho moieties are preferably derived from a mammalian alpha klotho, the albumin moieties are preferably derived from a mammalian albumin, Fc domains are preferably derived from a mammalian antibody, and the subjects are preferably mammals. More preferably, the mammal is human.

1. A polypeptide comprising an alpha klotho moiety (e.g., a soluble alpha klotho moiety having a variant alpha klotho KL1 and/or alpha klotho KL2 domain) operably linked to a stabilization moiety via a protease cleavable linker.

2. The polypeptide of embodiment 1, wherein the alpha klotho moiety:
(a) comprises an alpha klotho KL2 domain having (i) at least about 80% sequence identity to SEQ ID NO:3 and (ii) an amino acid substitution at the position corresponding to amino acid C521 of SEQ ID NO:1; and
(b) lacks a cysteine at the amino acid corresponding to amino acid C970 of SEQ ID NO: 1, if present.

3. The polypeptide of embodiment 2, wherein the alpha klotho KL2 domain has at least about 85% sequence identity to SEQ ID NO:3.

4. The polypeptide of embodiment 2, wherein the alpha klotho KL2 domain has at least about 90% sequence identity to SEQ ID NO:3.

5. The polypeptide of embodiment 2, wherein the alpha klotho KL2 domain has at least about 95% sequence identity to SEQ ID NO:3.

6. The polypeptide of embodiment 2, wherein the alpha klotho KL2 domain has at least about 99% sequence identity to SEQ ID NO:3.

7. The polypeptide of embodiment 2, wherein the alpha klotho KL2 domain has at least about 99.5% sequence identity to SEQ ID NO:3.

8. The polypeptide of embodiment 2, wherein the alpha klotho KL2 domain has at least about 85% sequence identity to SEQ ID NO:4.

9. The polypeptide of embodiment 2, wherein the alpha klotho KL2 domain has at least about 90% sequence identity to SEQ ID NO:4.

10. The polypeptide of embodiment 2, wherein the alpha klotho KL2 domain has at least about 95% sequence identity to SEQ ID NO:4.

11. The polypeptide of embodiment 2, wherein the alpha klotho KL2 domain has at least about 99% sequence identity to SEQ ID NO:4.

12. The polypeptide of embodiment 2, wherein the alpha klotho KL2 domain has at least about 99.5% sequence identity to SEQ ID NO:4.

13. The polypeptide of embodiment 2, wherein the alpha klotho KL2 domain comprises the amino acid sequence of SEQ ID NO:4.

14. The polypeptide of any one of embodiments 2 to 13, wherein the alpha klotho moiety further comprises an alpha klotho KL1 domain having at least about 80% sequence identity to SEQ ID NO:9.

15. The polypeptide of embodiment 14, wherein the alpha klotho KL1 domain has at least about 90% sequence identity to the amino acid sequence of SEQ ID NO:9.

16. The polypeptide of embodiment 14, wherein the alpha klotho KL1 domain has at least about 95% sequence identity to the amino acid sequence of SEQ ID NO:9.

17. The polypeptide of embodiment 14, wherein the alpha klotho KL1 domain has at least about 99% sequence identity to the amino acid sequence of SEQ ID NO:9.

18. The polypeptide of embodiment 14, wherein the alpha klotho KL1 domain comprises the amino acid sequence of SEQ ID NO:9.

19. The polypeptide of embodiment 14, wherein the alpha klotho KL1 domain has at least about 90% sequence identity to the amino acid sequence of SEQ ID NO: 10.

20. The polypeptide of embodiment 14, wherein the alpha klotho KL1 domain has at least about 95% sequence identity to the amino acid sequence of SEQ ID NO: 10.

21. The polypeptide of embodiment 14, wherein the alpha klotho KL1 domain has at least about 99% sequence identity to the amino acid sequence of SEQ ID NO: 10.

22. The polypeptide of embodiment 14, wherein the alpha klotho KL1 domain comprises the amino acid sequence of SEQ ID NO: 10.

23. The polypeptide of embodiment 14, wherein the alpha klotho KL1 domain has at least about 90% sequence identity to the amino acid sequence of SEQ ID NO:71.

24. The polypeptide of embodiment 14, wherein the alpha klotho KL1 domain has at least about 95% sequence identity to the amino acid sequence of SEQ ID NO:71.

25. The polypeptide of embodiment 14, wherein the alpha klotho KL1 domain has at least about 99% sequence identity to the amino acid sequence of SEQ ID NO:71.

26. The polypeptide of embodiment 14, wherein the alpha klotho KL1 domain comprises the amino acid sequence of SEQ ID NO:71.

27. The polypeptide of embodiment 14, wherein the alpha klotho KL1 domain comprises an amino acid substitution at the position corresponding to position C370 of SEQ ID NO: 1.

28. The polypeptide of embodiment 27, wherein the amino acid substitution at the position corresponding to position C370 of SEQ ID NO:1 is a cysteine to serine substitution.

29. The polypeptide of any one of embodiments 1 to 28, wherein the amino acid substitution at the position corresponding to amino acid C521 of SEQ ID NO: 1 is a cysteine to serine mutation.

30. The polypeptide of any one of embodiments 1 to 29, wherein the alpha klotho moiety lacks or wherein the polypeptide lacks an amino acid corresponding to amino acid C970 of SEQ ID NO:1.

31. The polypeptide of any one of embodiments 1 to 30, wherein the alpha klotho moiety lacks or wherein the polypeptide lacks a cysteine at the amino acid corresponding to C973 of SEQ ID NO: 1, if present.

32. The polypeptide of embodiment 31, wherein the alpha klotho moiety lacks or wherein the polypeptide lacks an amino acid corresponding to amino acid C973 of SEQ ID NO: 1.

33. The polypeptide of any one of embodiments 1 to 32, wherein the alpha klotho moiety lacks or wherein the polypeptide lacks a cysteine at the amino acid corresponding to C963 of SEQ ID NO:1, if present.

34. The polypeptide of embodiment 33, wherein the alpha klotho moiety lacks or wherein the polypeptide lacks an amino acid corresponding to amino acid C963 of SEQ ID NO: 1.

35. The polypeptide of any one of embodiments 1 to 34, wherein the alpha klotho moiety comprises a C-terminal deletion of at least 12 amino acids as compared to the amino acid sequence of SEQ ID NO: 13.

36. The polypeptide of any one of embodiments 1 to 34, wherein the alpha klotho moiety comprises a C-terminal deletion of at least 13 amino acids as compared to the amino acid sequence of SEQ ID NO: 13.

37. The polypeptide of any one of embodiments 1 to 34, wherein the alpha klotho moiety comprises a C-terminal deletion of at least 14 amino acids as compared to the amino acid sequence of SEQ ID NO: 13.

38. The polypeptide of any one of embodiments 1 to 34, wherein the alpha klotho moiety comprises a C-terminal deletion of at least 15 amino acids as compared to the amino acid sequence of SEQ ID NO:13.

39. The polypeptide of any one of embodiments 1 to 34, wherein the alpha klotho moiety comprises a C-terminal deletion of at least 16 amino acids as compared to the amino acid sequence of SEQ ID NO: 13.

40. The polypeptide of any one of embodiments 1 to 34, wherein the alpha klotho moiety comprises a C-terminal deletion of at least 17 amino acids as compared to the amino acid sequence of SEQ ID NO:13.

41. The polypeptide of any one of embodiments 1 to 34, wherein the alpha klotho moiety comprises a C-terminal deletion of at least 18 amino acids as compared to the amino acid sequence of SEQ ID NO:13.

42. The polypeptide of any one of embodiments 1 to 34, wherein the alpha klotho moiety comprises a C-terminal deletion of at least 19 amino acids as compared to the amino acid sequence of SEQ ID NO: 13.

43. The polypeptide of any one of embodiments 1 to 34, wherein the alpha klotho moiety comprises a C-terminal deletion of at least 20 amino acids as compared to the amino acid sequence of SEQ ID NO:13.

44. The polypeptide of any one of embodiments 1 to 34, wherein the alpha klotho moiety comprises a C-terminal deletion of at least 21 amino acids as compared to the amino acid sequence of SEQ ID NO: 13

45. The polypeptide of any one of embodiments 1 to 34, wherein the alpha klotho moiety comprises a C-terminal deletion of at least 22 amino acids as compared to the amino acid sequence of SEQ ID NO: 13.

46. The polypeptide of any one of embodiments 1 to 34, wherein the alpha klotho moiety comprises a C-terminal deletion of at least 23 amino acids as compared to the amino acid sequence of SEQ ID NO:13.

47. The polypeptide of any one of embodiments 1 to 34, wherein the alpha klotho moiety comprises a C-terminal deletion of at least 24 amino acids as compared to the amino acid sequence of SEQ ID NO: 13.

48. The polypeptide of any one of embodiments 1 to 34, wherein the alpha klotho moiety comprises a C-terminal deletion of at least 25 amino acids as compared to the amino acid sequence of SEQ ID NO:13.

49. The polypeptide of any one of embodiments 1 to 34, wherein the alpha klotho moiety comprises a C-terminal deletion of at least 26 amino acids as compared to the amino acid sequence of SEQ ID NO: 13.

50. The polypeptide of any one of embodiments 1 to 34, wherein the alpha klotho moiety comprises a C-terminal deletion of at least 27 amino acids as compared to the amino acid sequence of SEQ ID NO:13.

51. The polypeptide of any one of embodiments 1 to 34, wherein the alpha klotho moiety comprises a C-terminal deletion of at least 28 amino acids as compared to the amino acid sequence of SEQ ID NO:13.

52. The polypeptide of any one of embodiments 1 to 34, wherein the alpha klotho moiety comprises a C-terminal deletion of at least 29 amino acids as compared to the amino acid sequence of SEQ ID NO: 13.

53. The polypeptide of any one of embodiments 1 to 34, wherein the alpha klotho moiety comprises a C-terminal deletion of at least 30 amino acids as compared to the amino acid sequence of SEQ ID NO: 13.

54. The polypeptide of any one of embodiments 1 to 34, wherein the alpha klotho moiety comprises a C-terminal deletion of at least 31 amino acids as compared to the amino acid sequence of SEQ ID NO:13.

55. The polypeptide of any one of embodiments 1 to 34, wherein the alpha klotho moiety comprises a C-terminal deletion of between 12 and 31 amino acids as compared to the amino acid sequence of SEQ ID NO: 13.

56. The polypeptide of any one of embodiments 1 to 34, wherein the alpha klotho moiety comprises a C-terminal deletion of between 13 and 31 amino acids as compared to the amino acid sequence of SEQ ID NO: 13.

57. The polypeptide of any one of embodiments 1 to 34, wherein the alpha klotho moiety comprises a C-terminal deletion of between 14 and 31 amino acids as compared to the amino acid sequence of SEQ ID NO: 13.

58. The polypeptide of any one of embodiments 1 to 34, wherein the alpha klotho moiety comprises a C-terminal deletion of between 15 and 31 amino acids as compared to the amino acid sequence of SEQ ID NO:13.

59. The polypeptide of any one of embodiments 1 to 34, wherein the alpha klotho moiety comprises a C-terminal deletion of between 16 and 31 amino acids as compared to the amino acid sequence of SEQ ID NO: 13.

60. The polypeptide of any one of embodiments 1 to 34, wherein the alpha klotho moiety comprises a C-terminal deletion of between 17 and 31 amino acids as compared to the amino acid sequence of SEQ ID NO:13.

61. The polypeptide of any one of embodiments 1 to 34, wherein the alpha klotho moiety comprises a C-terminal deletion of between 18 and 31 amino acids as compared to the amino acid sequence of SEQ ID NO: 13.

62. The polypeptide of any one of embodiments 1 to 34, wherein the alpha klotho moiety comprises a C-terminal deletion of between 19 and 31 amino acids as compared to the amino acid sequence of SEQ ID NO:13.

63. The polypeptide of any one of embodiments 1 to 34, wherein the alpha klotho moiety comprises a C-terminal deletion of between 20 and 31 amino acids as compared to the amino acid sequence of SEQ ID NO:13.

64. The polypeptide of any one of embodiments 1 to 34, wherein the alpha klotho moiety comprises a C-terminal deletion of between 21 and 31 amino acids as compared to the amino acid sequence of SEQ ID NO:13.

65. The polypeptide of any one of embodiments 1 to 34, wherein the alpha klotho moiety comprises a C-terminal deletion of between 22 and 31 amino acids as compared to the amino acid sequence of SEQ ID NO: 13.

66. The polypeptide of any one of embodiments 1 to 34, wherein the alpha klotho moiety comprises a C-terminal deletion of between 23 and 31 amino acids as compared to the amino acid sequence of SEQ ID NO:13.

67. The polypeptide of any one of embodiments 1 to 34, wherein the alpha klotho moiety comprises a C-terminal deletion of between 12 and 23 amino acids as compared to the amino acid sequence of SEQ ID NO: 13.

68. The polypeptide of any one of embodiments 1 to 34, wherein the alpha klotho moiety comprises a C-terminal deletion of between 13 and 23 amino acids as compared to the amino acid sequence of SEQ ID NO:13.

69. The polypeptide of any one of embodiments 1 to 34, wherein the alpha klotho moiety comprises a C-terminal deletion of between 14 and 23 amino acids as compared to the amino acid sequence of SEQ ID NO:13.

70. The polypeptide of any one of embodiments 1 to 34, wherein the alpha klotho moiety comprises a C-terminal deletion of between 15 and 23 amino acids as compared to the amino acid sequence of SEQ ID NO:13.

71. The polypeptide of any one of embodiments 1 to 34, which comprises a C-terminal deletion of between 16 and 23 amino acids as compared to the amino acid sequence of SEQ ID NO: 13.

72. The polypeptide of any one of embodiments 1 to 34, wherein the alpha klotho moiety comprises a C-terminal deletion of between 17 and 23 amino acids as compared to the amino acid sequence of SEQ ID NO:13.

73. The polypeptide of any one of embodiments 1 to 34, wherein the alpha klotho moiety comprises a C-terminal deletion of between 18 and 23 amino acids as compared to the amino acid sequence of SEQ ID NO: 13.

74. The polypeptide of any one of embodiments 1 to 34, wherein the alpha klotho moiety comprises a C-terminal deletion of between 19 and 23 amino acids as compared to the amino acid sequence of SEQ ID NO: 13.

75. The polypeptide of any one of embodiments 1 to 34, wherein the alpha klotho moiety comprises a C-terminal deletion of between 20 and 23 amino acids as compared to the amino acid sequence of SEQ ID NO: 13 76. The polypeptide of any one of embodiments 1 to 34, wherein the alpha klotho moiety comprises a C-terminal deletion of between 21 and 23 amino acids as compared to the amino acid sequence of SEQ ID NO: 13.

77. The polypeptide of any one of embodiments 1 to 34, wherein the alpha klotho moiety comprises a C-terminal deletion of between 22 and 23 amino acids as compared to the amino acid sequence of SEQ ID NO:13.

78. The polypeptide of any one of embodiments 1 to 77, (a) which comprises an amino acid sequence having at least about 95% sequence identity to SEQ ID NO:11 or (b) wherein the alpha klotho moiety comprises or consists of an amino acid sequence having at least 95% sequence identity to SEQ ID NO: 11.

79. The polypeptide of any one of embodiments 1 to 77, (a) which comprises an amino acid sequence having at least about 98% sequence identity to SEQ ID NO: 11 or (b) wherein the alpha klotho moiety comprises or consists of an amino acid sequence having at least 98% sequence identity to SEQ ID NO: 11.

80. The polypeptide of any one of embodiments 1 to 77, (a) which comprises an amino acid sequence having at least about 99% sequence identity to SEQ ID NO:11 or (b) wherein the alpha klotho moiety comprises or consists of an amino acid sequence having at least 99% sequence identity to SEQ ID NO:11.

81. The polypeptide of any one of embodiments 1 to 77, (a) which comprises an amino acid sequence having at least about 95% sequence identity to SEQ ID NO: 12 or (b) wherein the alpha klotho moiety comprises or consists of an amino acid sequence having at least 95% sequence identity to SEQ ID NO: 12.

82. The polypeptide of any one of embodiments 1 to 77, (a) which comprises an amino acid sequence having at least about 98% sequence identity to SEQ ID NO:12 or (b) wherein the alpha klotho moiety comprises or consists of an amino acid sequence having at least 98% sequence identity to SEQ ID NO:12.

83. The polypeptide of any one of embodiments 1 to 77, (a) which comprises an amino acid sequence having at least about 99% sequence identity to SEQ ID NO: 12 or (b) wherein the alpha klotho moiety comprises or consists of an amino acid sequence having at least 99% sequence identity to SEQ ID NO: 12.

84. The polypeptide of any one of embodiments 1 to 77, (a) which comprises an amino acid sequence having at least about 99.5% sequence identity to SEQ ID NO: 12 or (b) wherein the alpha klotho moiety comprises or consists of an amino acid sequence having at least 99.5% sequence identity to SEQ ID NO: 12.

85. The polypeptide of any one of embodiments 81 to 84, (a) which comprises an amino acid sequence having one amino acid substitution relative to SEQ ID NO: 12 or (b) wherein the alpha klotho moiety comprises or consists of an amino acid sequence having one amino acid substitution relative to SEQ ID NO: 12.

86. The polypeptide of any one of embodiments 81 to 84, (a) which comprises an amino acid sequence having two amino acid substitutions relative to SEQ ID NO: 12 or (b) wherein the alpha klotho moiety comprises or consists of an amino acid sequence having two amino acid substitutions relative to SEQ ID NO: 12.

87. The polypeptide of any one of embodiments 81 to 84, (a) which comprises an amino acid sequence having three amino acid substitutions relative to SEQ ID NO: 12 or (b) wherein the alpha klotho moiety comprises or consists of an amino acid sequence having threeamino acid substitutions relative to SEQ ID NO: 12.

88. The polypeptide of any one of embodiments 81 to 84, (a) which comprises an amino acid sequence having four amino acid substitutions relative to SEQ ID NO: 12 or (b) wherein the alpha klotho moiety comprises or consists of an amino acid sequence having four amino acid substitutions relative to SEQ ID NO: 12.

89. The polypeptide of any one of embodiments 81 to 84, (a) which comprises an amino acid sequence having five amino acid substitutions relative to SEQ ID NO: 12 or (b) wherein the alpha klotho moiety comprises or consists of an amino acid sequence having five amino acid substitutions relative to SEQ ID NO: 12.

90. The polypeptide of any one of embodiments 81 to 84, (a) which comprises an amino acid sequence having 1, 2, 3, 4, 5, 6, 7, 8, 9, or 10 amino acid substitutions relative to SEQ ID NO: 12 or (b) wherein the alpha klotho moiety comprises or consists of an amino acid sequence having 1, 2, 3, 4, 5, 6, 7, 8, 9, or 10 amino acid substitutions relative to SEQ ID NO: 12.

91. The polypeptide of any one of embodiments 81 to 84, (a) which comprises an amino acid sequence having 1, 2, 3, 4, 5, 6, 7, 8, 9, or 10 amino acid variations (substitutions, deletions, or insertions) relative to SEQ ID NO: 12 or (b) wherein the alpha klotho moiety comprises or consists of an amino acid sequence having 1, 2, 3, 4, 5, 6, 7, 8, 9, or 10 amino acid variations (substitutions, deletions, or insertions) relative to SEQ ID NO: 12.

92. The polypeptide of any one of embodiments 1 to 77, (a) which comprises an amino acid sequence having at least about 95% sequence identity to SEQ ID NO:73 or (b) wherein the alpha klotho moiety comprises or consists of an amino acid sequence having at least 95% sequence identity to SEQ ID NO:73.

93. The polypeptide of any one of embodiments 1 to 77, (a) which comprises an amino acid sequence having at least about 98% sequence identity to SEQ ID NO:73 or (b) wherein the alpha klotho moiety comprises or consists of an amino acid sequence having at least 98% sequence identity to SEQ ID NO:73.

94. The polypeptide of any one of embodiments 1 to 77, (a) which comprises an amino acid sequence having at least about 99% sequence identity to SEQ ID NO:73 or (b) wherein the alpha klotho moiety comprises or consists of an amino acid sequence having at least 99% sequence identity to SEQ ID NO:73.

95. The polypeptide of any one of embodiments 1 to 77, (a) which comprises an amino acid sequence having at least about 99.5% sequence identity to SEQ ID NO:73 or (b) wherein the alpha klotho moiety comprises or consists of an amino acid sequence having at least 99.5% sequence identity to SEQ ID NO:73.

96. The polypeptide of any one of embodiments 1 to 77, (a) which comprises the amino acid sequence of SEQ ID NO:73 or (b) wherein the alpha klotho moiety comprises or consists of the amino acid sequence of SEQ ID NO:73.

97. The polypeptide of any one of embodiments 1 to 77, (a) which comprises an amino acid sequence having at least about 95% sequence identity to SEQ ID NO:74 or (b) wherein the alpha klotho moiety comprises or consists of an amino acid sequence having at least 95% sequence identity to SEQ ID NO:74.

98. The polypeptide of any one of embodiments 1 to 77, (a) which comprises an amino acid sequence having at least about 98% sequence identity to SEQ ID NO:74 or (b) wherein the alpha klotho moiety comprises or consists of an amino acid sequence having at least 98% sequence identity to SEQ ID NO:74.

99. The polypeptide of any one of embodiments 1 to 77, (a) which comprises an amino acid sequence having at least about 99% sequence identity to SEQ ID NO:74 or (b) wherein the alpha klotho moiety comprises or consists of an amino acid sequence having at least 99% sequence identity to SEQ ID NO:74.

100. The polypeptide of any one of embodiments 1 to 77, (a) which comprises an amino acid sequence having at least about 99.5% sequence identity to SEQ ID NO:74 or (b) wherein the alpha klotho moiety comprises or consists of an amino acid sequence having at least 99.5% sequence identity to SEQ ID NO:74.

101. The polypeptide of any one of embodiments 1 to 77, (a) which comprises the amino acid sequence of SEQ ID NO:74 or (b) wherein the alpha klotho moiety comprises or consists of the amino acid sequence of SEQ ID NO:74.

102. The polypeptide of any one of embodiments 1 to 101, (a) which comprises an amino acid sequence having at least about 90% sequence identity to SEQ ID NO: 15 or (b) wherein the alpha klotho moiety comprises or consists of an amino acid sequence having at least 90% sequence identity to SEQ ID NO: 15.

103. The polypeptide of any one of embodiments 1 to 101, (a) which comprises an amino acid sequence having at least about 95% sequence identity to SEQ ID NO: 15 or (b) wherein the alpha klotho moiety comprises or consists of an amino acid sequence having at least 95% sequence identity to SEQ ID NO: 15.

104. The polypeptide of any one of embodiments 1 to 101, (a) which comprises an amino acid sequence having at least about 90% sequence identity to SEQ ID NO: 16 or (b) wherein the alpha klotho moiety comprises or consists of an amino acid sequence having at least 90% sequence identity to SEQ ID NO: 16.

105. The polypeptide of any one of embodiments 1 to 101, (a) which comprises an amino acid sequence having at least about 95% sequence identity to SEQ ID NO: 16 or (b) wherein the alpha klotho moiety comprises or consists of an amino acid sequence having at least 95% sequence identity to SEQ ID NO: 16.

106. The polypeptide of any one of embodiments 1 to 101, (a) which comprises an amino acid sequence having at least about 90% sequence identity to SEQ ID NO:76 or (b) wherein the alpha klotho moiety comprises or consists of an amino acid sequence having at least 90% sequence identity to SEQ ID NO:76.

107. The polypeptide of any one of embodiments 1 to 101, (a) which comprises an amino acid sequence having at least about 95% sequence identity to SEQ ID NO:76 or (b) wherein the alpha klotho moiety comprises or consists of an amino acid sequence having at least 95% sequence identity to SEQ ID NO:76.

108. The polypeptide of any one of embodiments 102 to 107, wherein the amino acid sequence is between 850 and 950 amino acids in length.

109. The polypeptide of any one of embodiments 102 to 107, wherein the amino acid sequence is between 900 and 925 amino acids in length.

110. The polypeptide of any one of embodiments 102 to 107, wherein the amino acid sequence is between 910 and 920 amino acids in length.

111. The polypeptide of any one of embodiments 102 to 107, wherein the amino acid sequence is between 915 and 920 amino acids in length.

112. The polypeptide of any one of embodiments 1 to 111, (a) which comprises the amino acid sequence of SEQ ID NO: 16 or (b) wherein the alpha klotho moiety comprises or consists of the amino acid sequence of SEQ ID NO: 16.

113. The polypeptide of any one of embodiments 1 to 111, (a) which comprises the amino acid sequence of SEQ ID NO:76 or (b) wherein the alpha klotho moiety comprises or consists of the amino acid sequence of SEQ ID NO: 16.

114. The polypeptide of any one of embodiments 1 to 113, (a) which lacks an amino acid sequence having at least 95% sequence identity to SEQ ID NO:17 or (b) wherein the alpha klotho moiety lacks an amino acid sequence having at least 95% sequence identity to SEQ ID NO: 17.

115. The polypeptide of any one of embodiments 1 to 113, (a) which lacks an amino acid sequence having at least 98% sequence identity to SEQ ID NO: 17 or (b) wherein the alpha klotho moiety lacks an amino acid sequence having at least 98% sequence identity to SEQ ID NO: 17.

116. The polypeptide of any one of embodiments 1 to 113, (a) which lacks the amino acid sequence of SEQ ID NO: 17 or (b) wherein the alpha klotho moiety lacks the amino acid sequence of SEQ ID NO: 17.

117. The polypeptide of any one of embodiments 1 to 116, wherein the stabilization moiety is C-terminal to the alpha klotho moiety.

118. The polypeptide of embodiment 117, wherein the polypeptide comprises, in N- to C-terminal order: the alpha klotho moiety, the protease cleavable linker, and the stabilization moiety.

119. The polypeptide of any one of embodiments 1 to 116, wherein the stabilization moiety is N-terminal to the alpha klotho moiety.

120. The polypeptide of embodiment 119, wherein the polypeptide comprises, in N- to C-terminal order: the stabilization moiety, the protease cleavable linker, and the alpha klotho moiety.

121. The polypeptide of any one of embodiments 1 to 120, wherein the stabilization moiety is an albumin moiety.

122. The polypeptide of embodiment 121, wherein the albumin moiety is human serum albumin.

123. The polypeptide of embodiment 121, wherein the albumin moiety is a human serum albumin variant.

124. The polypeptide of embodiment 123, wherein the human serum albumin variant has the amino acid substitution C34S.

125. The polypeptide of embodiment 123 or 124, wherein the albumin moiety comprises an amino acid sequence having at least about 90% sequence identity to SEQ ID NO: 20.

126. The polypeptide of embodiment 123 or 124, wherein the albumin moiety comprises an amino acid sequence having at least about 95% sequence identity to SEQ ID NO: 20.

127. The polypeptide of embodiment 123 or 124, wherein the albumin moiety comprises an amino acid sequence having at least about 98% sequence identity to SEQ ID NO: 20.

128. The polypeptide of embodiment 123 or 124, wherein the albumin moiety comprises the amino acid sequence of SEQ ID NO:20.

129. A polypeptide, optionally a polypeptide of any one of embodiments 1 to 116, comprising:
(a) an amino acid sequence having at least 80% sequence identity to SEQ ID NO: 16, wherein the position corresponding to amino acid 488 of SEQ ID NO: 16 is not a cysteine, and which lacks an amino acid sequence having at least 80% sequence identity to SEQ ID NO:8;
(b) a protease cleavable linker; and
(c) a stabilization moiety.

130. The polypeptide of embodiment 129, wherein the position corresponding to amino acid 488 of SEQ ID NO: 16 is a serine.

131. The polypeptide of embodiment 129 or 130, wherein the amino acid sequence has at least 85% sequence identity to SEQ ID NO: 16.

132. The polypeptide of embodiment 129 or 130, wherein the amino acid sequence has at least 90% sequence identity to SEQ ID NO: 16.

133. The polypeptide of embodiment 129 or 130, wherein the amino acid sequence has at least 95% sequence identity to SEQ ID NO: 16.

134. The polypeptide of embodiment 129 or 130, wherein the amino acid sequence has at least 96% sequence identity to SEQ ID NO: 16.

135. The polypeptide of embodiment 129 or 130, wherein the amino acid sequence has at least 97% sequence identity to SEQ ID NO: 16.

136. The polypeptide of embodiment 129 or 130, wherein the amino acid sequence has at least 98% sequence identity to SEQ ID NO: 16.

137. The polypeptide of embodiment 129 or 130, wherein the amino acid sequence has at least 99% sequence identity to SEQ ID NO: 16.

138. The polypeptide of embodiment 129 or 130, wherein the amino acid sequence comprises the amino acid sequence of SEQ ID NO: 16.

139. The polypeptide of any one of embodiments 129 to 138, wherein the amino acid sequence lacks an amino acid sequence having at least 90% sequence identity to SEQ ID NO: 8.

140. The polypeptide of any one of embodiments 129 to 138, wherein the amino acid sequence lacks an amino acid sequence having at least 95% sequence identity to SEQ ID NO: 8.

141. The polypeptide of any one of embodiments 129 to 138, wherein the amino acid sequence lacks the amino acid sequence of SEQ ID NO:8.

142. The polypeptide of any one of embodiments 1 to 141, which comprises an amino acid sequence having at least 90% sequence identity to SEQ ID NO:64.

143. The polypeptide of embodiment 142, which comprises an amino acid sequence having at least 95% sequence identity to SEQ ID NO:64.

144. The polypeptide of embodiment 142, which comprises an amino acid sequence having at least 96% sequence identity to SEQ ID NO:64.

145. The polypeptide of embodiment 142, which comprises an amino acid sequence having at least 97% sequence identity to SEQ ID NO:64.

146. The polypeptide of embodiment 142, which comprises an amino acid sequence having at least 98% sequence identity to SEQ ID NO:64.

147. The polypeptide of embodiment 142, which comprises an amino acid sequence having at least 99% sequence identity to SEQ ID NO:64.

148. The polypeptide of embodiment 142, which comprises an amino acid sequence having at least 99.5% sequence identity to SEQ ID NO:64.

149. The polypeptide of embodiment 142, which comprises the amino acid sequence of SEQ ID NO:64.

150. The polypeptide of any one of embodiments 1 to 141, which comprises an amino acid sequence having at least 90% sequence identity to SEQ ID NO:66.

151. The polypeptide of embodiment 150, which comprises an amino acid sequence having at least 95% sequence identity to SEQ ID NO:66.

152. The polypeptide of embodiment 150, which comprises an amino acid sequence having at least 96% sequence identity to SEQ ID NO:66.

153. The polypeptide of embodiment 150, which comprises an amino acid sequence having at least 97% sequence identity to SEQ ID NO:66.

154. The polypeptide of embodiment 150, which comprises an amino acid sequence having at least 98% sequence identity to SEQ ID NO:66.

155. The polypeptide of embodiment 150, which comprises an amino acid sequence having at least 99% sequence identity to SEQ ID NO:66.

156. The polypeptide of embodiment 150, which comprises an amino acid sequence having at least 99.5% sequence identity to SEQ ID NO:66.

157. The polypeptide of embodiment 150, which comprises the amino acid sequence of SEQ ID NO:66.

158. The polypeptide of any one of embodiments 1 to 141, which comprises an amino acid sequence having at least 90% sequence identity to SEQ ID NO:68.

159. The polypeptide of embodiment 158, which comprises an amino acid sequence having at least 95% sequence identity to SEQ ID NO:68.

160. The polypeptide of embodiment 158, which comprises an amino acid sequence having at least 96% sequence identity to SEQ ID NO:68.

161. The polypeptide of embodiment 158, which comprises an amino acid sequence having at least 97% sequence identity to SEQ ID NO:68.

162. The polypeptide of embodiment 158, which comprises an amino acid sequence having at least 98% sequence identity to SEQ ID NO:68.

163. The polypeptide of embodiment 158, which comprises an amino acid sequence having at least 99% sequence identity to SEQ ID NO:68.

164. The polypeptide of embodiment 158, which comprises an amino acid sequence having at least 99.5% sequence identity to SEQ ID NO:68.

165. The polypeptide of embodiment 158, which comprises the amino acid sequence of SEQ ID NO:68.

166. The polypeptide of any one of embodiments 1 to 141, which comprises an amino acid sequence having at least 90% sequence identity to SEQ ID NO:70.

167. The polypeptide of embodiment 166, which comprises an amino acid sequence having at least 95% sequence identity to SEQ ID NO:70.

168. The polypeptide of embodiment 166, which comprises an amino acid sequence having at least 96% sequence identity to SEQ ID NO:70.

169. The polypeptide of embodiment 166, which comprises an amino acid sequence having at least 97% sequence identity to SEQ ID NO:70.

170. The polypeptide of embodiment 166, which comprises an amino acid sequence having at least 98% sequence identity to SEQ ID NO:70.

171. The polypeptide of embodiment 166, which comprises an amino acid sequence having at least 99% sequence identity to SEQ ID NO:70.

172. The polypeptide of embodiment 166, which comprises an amino acid sequence having at least 99.5% sequence identity to SEQ ID NO:70.

173. The polypeptide of embodiment 166, which comprises the amino acid sequence of SEQ ID NO:70.

174. The polypeptide of any one of embodiments 1 to 173, wherein the protease cleavable linker is a linker set forth in Table D.

175. The polypeptide of any one of embodiments 1 to 174, wherein the protease cleavable linker comprises an amino acid sequence having at least 80% sequence identity to SEQ ID NO:90.

176. The polypeptide of any one of embodiments 1 to 174, wherein the protease cleavable linker comprises an amino acid sequence having at least 85% sequence identity to SEQ ID NO:90.

177. The polypeptide of any one of embodiments 1 to 174, wherein the protease cleavable linker comprises an amino acid sequence having at least 90% sequence identity to SEQ ID NO:90.

178. The polypeptide of any one of embodiments 1 to 174, wherein the protease cleavable linker comprises an amino acid sequence having at least 95% sequence identity to SEQ ID NO:90.

179. The polypeptide of any one of embodiments 1 to 174, wherein the protease cleavable linker comprises an amino acid sequence having at least 98% sequence identity to SEQ ID NO:90.

180. The polypeptide of any one of embodiments 1 to 174, wherein the protease cleavable linker comprises an amino acid sequence having at least 99% sequence identity to SEQ ID NO:90.

181. The polypeptide of any one of embodiments 1 to 174, wherein the protease cleavable linker comprises the amino acid sequence of SEQ ID NO:90.

182. The polypeptide of any one of embodiments 1 to 181, further comprising a signal peptide.

183. The polypeptide of embodiment 182, wherein the signal peptide is an alpha klotho signal peptide.

184. The polypeptide of embodiment 182, wherein the signal peptide is not an alpha klotho signal peptide.

185. The polypeptide of embodiment 184, wherein the signal peptide is a serum albumin (SA) signal peptide.

186. The polypeptide of embodiment 184, wherein the signal peptide is an azurocidin (AZ) signal peptide.

187. The polypeptide of embodiment 184, wherein the signal peptide is an SP1 signal peptide.

188. The polypeptide of any one of embodiments 1 to 181, which lacks a signal peptide.

189. A nucleic acid encoding the polypeptide of any one of embodiments 1 to 188.

190. A host cell engineered to express the polypeptide of any one of embodiments 1 to 188 or the nucleic acid of embodiment 189.

191. A method of producing the polypeptide of any one of embodiments 1 to 188, comprising culturing the host cell of embodiment 190 and recovering the polypeptide expressed thereby.

192. The method of embodiment 191, further comprising purifying the polypeptide.

193. The method of embodiment 192, wherein the polypeptide is purified at a pH between 6.5 and 7.5.

194. The method of embodiment 192 or 193, wherein the polypeptide is purified at a pH between 6.9 and 7.1.

195. The method of any one of embodiments 192 or 194, wherein the polypeptide is purified at a pH of about 7.0.

196. A pharmaceutical composition comprising the polypeptide of any one of embodiments 1 to 188 and an excipient.

197. A method of activating FGFR signaling in a cell, the method comprising contacting the cell with the polypeptide of any one of embodiments 1 to 188.

198. The method of embodiment 197, wherein the cell is a kidney cell.

199. The method of embodiment 197 or 198, wherein the method is an in vitro method.

200. The method of embodiment 197 or 198, wherein the method is an in vivo method.

201. The method of embodiment 200, wherein the method comprises administering the polypeptide to a subject in need thereof.

202. The method of embodiment 201, wherein the subject is at risk for developing an age-related condition.

203. The method of embodiment 201, wherein the subject is a patient suffering from an age-related condition.

204. The method of embodiment 201, wherein the subject is at risk for developing kidney disease.

205. The method of embodiment 204, wherein the kidney disease is acute kidney injury.

206. The method of embodiment 204, wherein the kidney disease is chronic kidney disease.

207. A method of treating a subject suffering from an age-related condition comprising administering to the subject the polypeptide of any one of embodiments 1 to 188 or the pharmaceutical composition of embodiment 196.

208. A method of preventing an age-related condition comprising administering to a subject in need thereof the polypeptide of any one of embodiments 1 to 188 or the pharmaceutical composition of embodiment 196.

209. A method of treating a subject suffering from kidney disease comprising administering to the subject the polypeptide of any one of embodiments 1 to 188 or the pharmaceutical composition of embodiment 196.

210. A method of preventing kidney disease comprising administering to a subject in need thereof the polypeptide of any one of embodiments 1 to 188 or the pharmaceutical composition of embodiment 196.

211. The method of embodiment 209 or 210, wherein the kidney disease is acute kidney injury.

212. The method of embodiment 209 or 210, wherein the kidney disease is chronic kidney disease.

### 9. EXAMPLES

### 9.1. Materials and Methods

### 9.1.1. Design and Production of KL Polypeptide Constructs

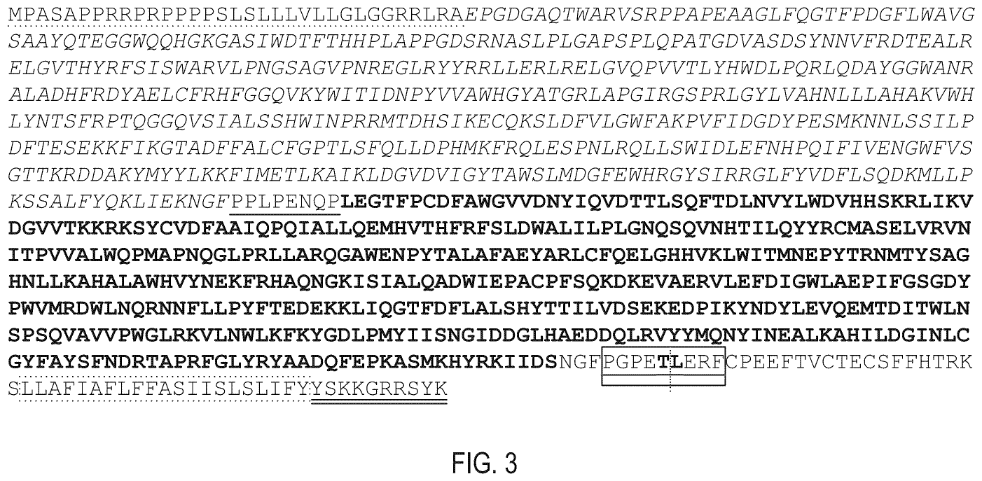

KL polypeptide constructs were designed using the wildtype human alpha-Klotho (hKL) amino acid sequence (UniProtKB Accession Number Q9UEF7-1; FIG. 3). Where applicable, 29-amino acid signal sequence from murine inactive tyrosine-protein kinase transmembrane receptor ROR1 (mROR1) was used to replace the native KL signal sequence to increase production yield; the hKL amino acid sequence was truncated at the C-terminus to remove the C-terminal protease cleavage site; one or more Cys amino acids were mutated to Ser to remove free surface Cys residues; and one or more stabilization moieties were added via a linker to the N- or C-termini of constructs. Exemplary KL polypeptide constructs are shown in FIGS. 2A-2J.

Constructs encoding KL polypeptides were generated in standard mammalian protein expression DNA vectors (pcDNA3.4 or similar) suitable for high yield protein production and containing standard elements such as promoter sequence, polyA sequence, regulatory elements, and resistance genes. Where applicable, sequences were codon optimized. The KL polypeptide constructs were expressed in suitable cells (e.g., Expi293 or CHO cells) by transient transfection. Proteins in cellular supernatants were purified using one of the methods described in Section 9.1.2, neutralized, dialyzed into a final buffer of phosphate buffered saline (PBS) with 5% glycerol, aliquoted, and stored at −80° C. Samples were further analyzed by size exclusion chromatography (SEC) to determine the presence of high or low molecular weight species relative to the species of interest as described in Section 9.1.2. Exemplary sequences are set forth in Table E1.

TABLE E1

| | | SEQ |
|---|---|---|
| | | ID |
| Name | Sequence | NO: |
| REGN14416 | GAGCCGGGCGACGGCGCGCAGACCTGGGCCCGTTTCTCGCGGCCTCCTGCCCCCGAG | 41 |
| (KL981-His) | GCCGCGGGCCTCTTCCAGGGCACCTTCCCCGACGGCTTCCTCTGGGCCGTGGGCAGC | |
| nucleotide | GCCGCCTACCAGACCGAGGGCGGCTGGCAGCAGCACGGCAAGGGTGCGTCCATCTGG | |
| sequence | GATACGTTCACCCACCACCCCCTGGCACCCCCGGGAGACTCCCGGAACGCCAGTCTG | |
| | CCGTTGGGCGCCCCGTCGCCGCTGCAGCCCGCCACCGGGGACGTAGCCAGCGACAGC | |
| | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCTGTGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTCTGGAAAGATTTTGTCCA | |
| | GAAGAATTCACCGTGTGTACTGAGTGCAGTTTTTTTCACACCCGAAAGTCTCACCAT | |
| | CACCATCACCATTGA | |
| REGN14416 | EPGDGAQTWARFSRPPAPEAAGLFQGTFPDGFLWAVGSAAYQTEGGWQQHGKGASIW | 42 |
| (KL981-His) | DTFTHHPLAPPGDSRNASLPLGAPSPLQPATGDVASDSYNNVFRDTEALRELGVTHY | |
| Amino acid | RFSISWARVLPNGSAGVPNREGLRYYRRLLERLRELGVQPVVTLYHWDLPQRLQDAY | |
| sequence | GGWANRALADHFRDYAELCFRHFGGQVKYWITIDNPYVVAWHGYATGRLAPGIRGSP | |
| | RLGYLVAHNLLLAHAKVWHLYNTSFRPTQGGQVSIALSSHWINPRRMTDHSIKECQK | |
| | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPCDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETLERFCPEEFTVCTECSFFHTRKSHHHHHH | |
| REGN14225 | GAGCCGGGCGACGGCGCGCAGACCTGGGCCCGTTTCTCGCGGCCTCCTGCCCCCGAG | 43 |
| (KL958-His) | GCCGCGGGCCTCTTCCAGGGCACCTTCCCCGACGGCTTCCTCTGGGCCGTGGGCAGC | |
| Nucleotide | GCCGCCTACCAGACCGAGGGCGGCTGGCAGCAGCACGGCAAGGGTGCGTCCATCTGG | |
| sequence | GATACGTTCACCCACCACCCCCTGGCACCCCCGGGAGACTCCCGGAACGCCAGTCTG | |
| | CCGTTGGGCGCCCCGTCGCCGCTGCAGCCCGCCACCGGGGACGTAGCCAGCGACAGC | |
| | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCTGTGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTCACCATCACCATCACCAT | |
| | TGA | |
| REGN14225 | EPGDGAQTWARFSRPPAPEAAGLFQGTFPDGFLWAVGSAAYQTEGGWQQHGKGASIW | 44 |
| (KL958-His) | DTFTHHPLAPPGDSRNASLPLGAPSPLQPATGDVASDSYNNVFRDTEALRELGVTHY | |
| Amino acid | RFSISWARVLPNGSAGVPNREGLRYYRRLLERLRELGVQPVVTLYHWDLPQRLQDAY | |
| sequence | GGWANRALADHFRDYAELCFRHFGGQVKYWITIDNPYVVAWHGYATGRLAPGIRGSP | |
| | RLGYLVAHNLLLAHAKVWHLYNTSFRPTQGGQVSIALSSHWINPRRMTDHSIKECQK | |
| | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPCDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNOGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETHHHHHH | |
| REGN14226 | GAGCCGGGCGACGGCGCGCAGACCTGGGCCCGTTTCTCGCGGCCTCCTGCCCCCGAG | 45 |
| (KL958 | GCCGCGGGCCTCTTCCAGGGCACCTTCCCCGACGGCTTCCTCTGGGCCGTGGGCAGC | |
| (C521S)-His) | GCCGCCTACCAGACCGAGGGCGGCTGGCAGCAGCACGGCAAGGGTGCGTCCATCTGG | |
| Nucleotide | GATACGTTCACCCACCACCCCCTGGCACCCCCGGGAGACTCCCGGAACGCCAGTCTG | |
| sequence | CCGTTGGGCGCCCCGTCGCCGCTGCAGCCCGCCACCGGGGACGTAGCCAGCGACAGC | |
| | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTCACCATCACCATCACCAT | |
| | TGA | |
| REGN14226 | EPGDGAQTWARFSRPPAPEAAGLFQGTFPDGFLWAVGSAAYQTEGGWQQHGKGASIW | 46 |
| (KL958 | DTFTHHPLAPPGDSRNASLPLGAPSPLQPATGDVASDSYNNVFRDTEALRELGVTHY | |
| (C521S)-His) | RFSISWARVLPNGSAGVPNREGLRYYRRLLERLRELGVQPVVTLYHWDLPQRLQDAY | |
| Amino acid | GGWANRALADHFRDYAELCFRHFGGQVKYWITIDNPYVVAWHGYATGRLAPGIRGSP | |
| sequence | RLGYLVAHNLLLAHAKVWHLYNTSFRPTQGGQVSIALSSHWINPRRMTDHSIKECQK | |
| | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNOGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETHHHHHH | |
| REGN14227 | GAGCCGGGCGACGGCGCGCAGACCTGGGCCCGTTTCTCGCGGCCTCCTGCCCCCGAG | 47 |
| (KL958 | GCCGCGGGCCTCTTCCAGGGCACCTTCCCCGACGGCTTCCTCTGGGCCGTGGGCAGC | |
| (C521S | GCCGCCTACCAGACCGAGGGCGGCTGGCAGCAGCACGGCAAGGGTGCGTCCATCTGG | |
| C910S)-His) | GATACGTTCACCCACCACCCCCTGGCACCCCCGGGAGACTCCCGGAACGCCAGTCTG | |
| Nucleotide | CCGTTGGGCGCCCCGTCGCCGCTGCAGCCCGCCACCGGGGACGTAGCCAGCGACAGC | |
| sequence | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTAGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTCACCATCACCATCACCAT | |
| | TGA | |
| REGN14227 | EPGDGAQTWARFSRPPAPEAAGLFQGTFPDGFLWAVGSAAYQTEGGWQQHGKGASIW | 48 |
| (KL958 | DTFTHHPLAPPGDSRNASLPLGAPSPLQPATGDVASDSYNNVFRDTEALRELGVTHY | |
| (C521S | RFSISWARVLPNGSAGVPNREGLRYYRRLLERLRELGVQPVVTLYHWDLPQRLQDAY | |
| C910S)-His) | GGWANRALADHFRDYAELCFRHFGGQVKYWITIDNPYVVAWHGYATGRLAPGIRGSP | |
| Amino acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNOGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLSGYFAYSFNDRTAPRFGLYRYAADQFEPKASMKHYR | |
| | KIIDSNGFPGPETHHHHHH | |
| REGN15823 | GAGCCGGGCGACGGCGCGCAGACCTGGGCCCGTTTCTCGCGGCCTCCTGCCCCCGAG | 49 |
| (KL981-G4S- | GCCGCGGGCCTCTTCCAGGGCACCTTCCCCGACGGCTTCCTCTGGGCCGTGGGCAGC | |
| HSA) | GCCGCCTACCAGACCGAGGGCGGCTGGCAGCAGCACGGCAAGGGTGCGTCCATCTGG | |
| nucleotide | GATACGTTCACCCACCACCCCCTGGCACCCCCGGGAGACTCCCGGAACGCCAGTCTG | |
| sequence | CCGTTGGGCGCCCCGTCGCCGCTGCAGCCCGCCACCGGGGACGTAGCCAGCGACAGC | |
| | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCTGTGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTCTGGAAAGATTTTGTCCA | |
| | GAAGAATTTACCGTGTGTACTGAGTGCAGTTTTTTTCACACCCGAAAGTCTGGTGGA | |
| | GGAGGTAGTGATGCACACAAGAGTGAGGTTGCTCATCGGTTTAAAGATTTGGGAGAA | |
| | GAAAATTTCAAAGCCTTGGTGTTGATTGCCTTTGCTCAGTATCTTCAGCAGTGTCCA | |
| | TTTGAAGATCATGTAAAATTAGTGAATGAAGTAACTGAATTTGCAAAAACATGTGTT | |
| | GCTGATGAGTCAGCTGAAAATTGTGACAAATCACTTCATACCCTTTTTGGAGACAAA | |
| | TTATGCACAGTTGCAACTCTTCGTGAAACCTATGGTGAAATGGCTGACTGCTGTGCA | |
| | AAACAAGAACCTGAGAGAAATGAATGCTTCTTGCAACACAAAGATGACAACCCAAAC | |
| | CTCCCCCGATTGGTGAGACCAGAGGTTGATGTGATGTGCACTGCTTTTCATGACAAT | |
| | GAAGAGACATTTTTGAAAAAATACTTATATGAAATTGCCAGAAGACATCCTTACTTT | |
| | TATGCCCCGGAACTCCTTTTCTTTGCTAAAAGGTATAAAGCTGCTTTTACAGAATGT | |
| | TGCCAAGCTGCTGATAAAGCTGCCTGCCTGTTGCCAAAGCTCGATGAACTTCGGGAT | |
| | GAAGGGAAGGCTTCGTCTGCCAAACAGAGACTCAAGTGTGCCAGTCTCCAAAAATTT | |
| | GGAGAAAGAGCTTTCAAAGCATGGGCAGTAGCTCGCCTGAGCCAGAGATTTCCCAAA | |
| | GCTGAGTTTGCAGAAGTTTCCAAGTTAGTGACAGATCTTACCAAAGTCCACACGGAA | |
| | TGCTGCCATGGAGATCTGCTTGAATGTGCTGATGACAGGGCGGACCTTGCCAAGTAT | |
| | ATCTGTGAAAATCAAGATTCGATCTCCAGTAAACTGAAGGAATGCTGTGAAAAACCT | |
| | CTGTTGGAAAAATCCCACTGCATTGCCGAAGTGGAAAATGATGAGATGCCTGCTGAC | |
| | TTGCCTTCATTAGCTGCTGATTTTGTTGAAAGTAAGGATGTTTGCAAAAACTATGCT | |
| | GAGGCAAAGGATGTCTTCCTGGGCATGTTTTTGTATGAATATGCAAGAAGGCATCCT | |
| | GATTACTCTGTCGTGCTGCTGCTGAGACTTGCCAAGACATATGAAACCACTCTAGAG | |
| | AAGTGCTGTGCCGCTGCAGATCCTCATGAATGCTATGCCAAAGTGTTCGATGAATTT | |
| | AAACCTCTTGTGGAAGAGCCTCAGAATTTAATCAAACAAAATTGTGAGCTTTTTGAG | |
| | CAGCTTGGAGAGTACAAATTCCAGAATGCGCTATTAGTTCGTTACACCAAGAAAGTA | |
| | CCCCAAGTGTCAACTCCAACTCTTGTAGAGGTCTCAAGAAACCTAGGAAAAGTGGGC | |
| | AGCAAATGTTGTAAACATCCTGAAGCAAAAAGAATGCCCTGTGCAGAAGACTATCTA | |
| | TCCGTGGTCCTGAACCAGTTATGTGTGTTGCATGAGAAAACGCCAGTAAGTGACAGA | |
| | GTCACCAAATGCTGCACAGAATCCTTGGTGAACAGGCGACCATGCTTTTCAGCTCTG | |
| | GAAGTCGATGAAACATACGTTCCCAAAGAGTTTAATGCTGAAACATTCACCTTCCAT | |
| | GCAGATATATGCACACTTTCTGAGAAGGAGAGACAAATCAAGAAACAAACTGCACTT | |
| | GTTGAGCTCGTGAAACACAAGCCCAAGGCAACAAAAGAGCAACTGAAAGCTGTTATG | |
| | GATGATTTCGCAGCTTTTGTAGAGAAGTGCTGCAAGGCTGACGATAAGGAGACCTGC | |
| | TTTGCCGAGGAGGGTAAAAAACTTGTTGCTGCAAGTCAAGCTGCCTTAGGCTTATGA | |
| REGN15823 | EPGDGAQTWARFSRPPAPEAAGLFQGTFPDGFLWAVGSAAYQTEGGWQQHGKGASIW | 50 |
| (KL981-G4S- | DTFTHHPLAPPGDSRNASLPLGAPSPLQPATGDVASDSYNNVFRDTEALRELGVTHY | |
| HSA) amino | RFSISWARVLPNGSAGVPNREGLRYYRRLLERLRELGVQPVVTLYHWDLPQRLQDAY | |
| acid | GGWANRALADHFRDYAELCFRHFGGQVKYWITIDNPYVVAWHGYATGRLAPGIRGSP | |
| sequence | RLGYLVAHNLLLAHAKVWHLYNTSFRPTQGGQVSIALSSHWINPRRMTDHSIKECQK | |
| | SLDFVLGWFAKPVFIDGDYPESMKNNLSSILPDFTESEKKFIKGTADFFALCFGPTL | |
| | SFQLLDPHMKFRQLESPNLRQLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPCDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETLERFCPEEFTVCTECSFFHTRKSGGGGSDAHKSEVAHRFKDLGE | |
| | ENFKALVLIAFAQYLQQCPFEDHVKLVNEVTEFAKTCVADESAENCDKSLHTLFGDK | |
| | LCTVATLRETYGEMADCCAKQEPERNECFLQHKDDNPNLPRLVRPEVDVMCTAFHDN | |
| | EETFLKKYLYEIARRHPYFYAPELLFFAKRYKAAFTECCQAADKAACLLPKLDELRD | |
| | EGKASSAKQRLKCASLQKFGERAFKAWAVARLSQRFPKAEFAEVSKLVTDLTKVHTE | |
| | CCHGDLLECADDRADLAKYICENQDSISSKLKECCEKPLLEKSHCIAEVENDEMPAD | |
| | LPSLAADFVESKDVCKNYAEAKDVFLGMFLYEYARRHPDYSVVLLLRLAKTYETTLE | |
| | KCCAAADPHECYAKVFDEFKPLVEEPQNLIKQNCELFEQLGEYKFQNALLVRYTKKV | |
| | PQVSTPTLVEVSRNLGKVGSKCCKHPEAKRMPCAEDYLSVVLNQLCVLHEKTPVSDR | |
| | VTKCCTESLVNRRPCFSALEVDETYVPKEFNAETFTFHADICTLSEKERQIKKQTAL | |
| | VELVKHKPKATKEQLKAVMDDFAAFVEKCCKADDKETCFAEEGKKLVAASQAALGL | |
| REGN15548 | GAGCCGGGCGACGGCGCGCAGACCTGGGCCCGTTTCTCGCGGCCTCCTGCCCCCGAG | 51 |
| (KL958 | GCCGCGGGCCTCTTCCAGGGCACCTTCCCCGACGGCTTCCTCTGGGCCGTGGGCAGC | |
| (C521S)- | GCCGCCTACCAGACCGAGGGCGGCTGGCAGCAGCACGGCAAGGGTGCGTCCATCTGG | |
| G4S-HSA) | GATACGTTCACCCACCACCCCCTGGCACCCCCGGGAGACTCCCGGAACGCCAGTCTG | |
| Nucleotide | CCGTTGGGCGCCCCGTCGCCGCTGCAGCCCGCCACCGGGGACGTAGCCAGCGACAGC | |
| sequence | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTGGTGGAGGAGGTAGTGAT | |
| | GCACACAAGAGTGAGGTTGCTCATCGGTTTAAAGATTTGGGAGAAGAAAATTTCAAA | |
| | GCCTTGGTGTTGATTGCCTTTGCTCAGTATCTTCAGCAGTGTCCATTTGAAGATCAT | |
| | GTAAAATTAGTGAATGAAGTAACTGAATTTGCAAAAACATGTGTTGCTGATGAGTCA | |
| | GCTGAAAATTGTGACAAATCACTTCATACCCTTTTTGGAGACAAATTATGCACAGTT | |
| | GCAACTCTTCGTGAAACCTATGGTGAAATGGCTGACTGCTGTGCAAAACAAGAACCT | |
| | GAGAGAAATGAATGCTTCTTGCAACACAAAGATGACAACCCAAACCTCCCCCGATTG | |
| | GTGAGACCAGAGGTTGATGTGATGTGCACTGCTTTTCATGACAATGAAGAGACATTT | |
| | TTGAAAAAATACTTATATGAAATTGCCAGAAGACATCCTTACTTTTATGCCCCGGAA | |
| | CTCCTTTTCTTTGCTAAAAGGTATAAAGCTGCTTTTACAGAATGTTGCCAAGCTGCT | |
| | GATAAAGCTGCCTGCCTGTTGCCAAAGCTCGATGAACTTCGGGATGAAGGGAAGGCT | |
| | TCGTCTGCCAAACAGAGACTCAAGTGTGCCAGTCTCCAAAAATTTGGAGAAAGAGCT | |
| | TTCAAAGCATGGGCAGTAGCTCGCCTGAGCCAGAGATTTCCCAAAGCTGAGTTTGCA | |
| | GAAGTTTCCAAGTTAGTGACAGATCTTACCAAAGTCCACACGGAATGCTGCCATGGA | |
| | GATCTGCTTGAATGTGCTGATGACAGGGCGGACCTTGCCAAGTATATCTGTGAAAAT | |
| | CAAGATTCGATCTCCAGTAAACTGAAGGAATGCTGTGAAAAACCTCTGTTGGAAAAA | |
| | TCCCACTGCATTGCCGAAGTGGAAAATGATGAGATGCCTGCTGACTTGCCTTCATTA | |
| | GCTGCTGATTTTGTTGAAAGTAAGGATGTTTGCAAAAACTATGCTGAGGCAAAGGAT | |
| | GTCTTCCTGGGCATGTTTTTGTATGAATATGCAAGAAGGCATCCTGATTACTCTGTC | |
| | GTGCTGCTGCTGAGACTTGCCAAGACATATGAAACCACTCTAGAGAAGTGCTGTGCC | |
| | GCTGCAGATCCTCATGAATGCTATGCCAAAGTGTTCGATGAATTTAAACCTCTTGTG | |
| | GAAGAGCCTCAGAATTTAATCAAACAAAATTGTGAGCTTTTTGAGCAGCTTGGAGAG | |
| | TACAAATTCCAGAATGCGCTATTAGTTCGTTACACCAAGAAAGTACCCCAAGTGTCA | |
| | ACTCCAACTCTTGTAGAGGTCTCAAGAAACCTAGGAAAAGTGGGCAGCAAATGTTGT | |
| | AAACATCCTGAAGCAAAAAGAATGCCCTGTGCAGAAGACTATCTATCCGTGGTCCTG | |
| | AACCAGTTATGTGTGTTGCATGAGAAAACGCCAGTAAGTGACAGAGTCACCAAATGC | |
| | TGCACAGAATCCTTGGTGAACAGGCGACCATGCTTTTCAGCTCTGGAAGTCGATGAA | |
| | ACATACGTTCCCAAAGAGTTTAATGCTGAAACATTCACCTTCCATGCAGATATATGC | |
| | ACACTTTCTGAGAAGGAGAGACAAATCAAGAAACAAACTGCACTTGTTGAGCTCGTG | |
| | AAACACAAGCCCAAGGCAACAAAAGAGCAACTGAAAGCTGTTATGGATGATTTCGCA | |
| | GCTTTTGTAGAGAAGTGCTGCAAGGCTGACGATAAGGAGACCTGCTTTGCCGAGGAG | |
| | GGTAAAAAACTTGTTGCTGCAAGTCAAGCTGCCTTAGGCTTATGA | |
| REGN15548 | EPGDGAQTWARFSRPPAPEAAGLFQGTFPDGFLWAVGSAAYQTEGGWQQHGKGASIW | 52 |
| (KL958 | DTFTHHPLAPPGDSRNASLPLGAPSPLQPATGDVASDSYNNVFRDTEALRELGVTHY | |
| (C521S)- | RFSISWARVLPNGSAGVPNREGLRYYRRLLERLRELGVQPVVTLYHWDLPQRLQDAY | |
| G4S-HSA) | GGWANRALADHFRDYAELCFRHFGGQVKYWITIDNPYVVAWHGYATGRLAPGIRGSP | |
| Amino acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNOGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETGGGGSDAHKSEVAHRFKDLGEENFKALVLIAFAQYLQQCPFEDH | |
| | VKLVNEVTEFAKTCVADESAENCDKSLHTLFGDKLCTVATLRETYGEMADCCAKQEP | |
| | ERNECFLQHKDDNPNLPRLVRPEVDVMCTAFHDNEETFLKKYLYEIARRHPYFYAPE | |
| | LLFFAKRYKAAFTECCQAADKAACLLPKLDELRDEGKASSAKQRLKCASLQKFGERA | |
| | FKAWAVARLSQRFPKAEFAEVSKLVTDLTKVHTECCHGDLLECADDRADLAKYICEN | |
| | QDSISSKLKECCEKPLLEKSHCIAEVENDEMPADLPSLAADFVESKDVCKNYAEAKD | |
| | VFLGMFLYEYARRHPDYSVVLLLRLAKTYETTLEKCCAAADPHECYAKVFDEFKPLV | |
| | EEPQNLIKQNCELFEQLGEYKFQNALLVRYTKKVPQVSTPTLVEVSRNLGKVGSKCC | |
| | KHPEAKRMPCAEDYLSVVLNQLCVLHEKTPVSDRVTKCCTESLVNRRPCFSALEVDE | |
| | TYVPKEFNAETFTFHADICTLSEKERQIKKQTALVELVKHKPKATKEQLKAVMDDFA | |
| | AFVEKCCKADDKETCFAEEGKKLVAASQAALGL | |
| KL958 | GAGCCTGGGGACGGCGCTCAGACCTGGGCTCGTTTTTCTCGTCCTCCTGCTCCTGAG | 53 |
| ((C521S | GCTGCTGGCCTCTTCCAGGGCACGTTCCCAGACGGCTTTTTGTGGGCGGTGGGTTCT | |
| C910S)- | GCGGCCTACCAGACGGAGGGGGGTTGGCAGCAGCATGGCAAGGGCGCCTCCATCTGG | |
| HSA-His) | GATACTTTCACGCACCACCCCCTGGCTCCGCCTGGCGATTCCCGCAACGCCTCCCTG | |
| nucleotide | CCCTTAGGAGCCCCCTCTCCCCTGCAGCCGGCCACCGGCGATGTAGCGTCCGATAGC | |
| sequence | TACAATAACGTGTTCCGCGACACCGAGGCTTTGAGAGAGTTGGGCGTTACTCATTAC | |
| | CGCTTCTCTATCAGTTGGGCCCGCGTACTGCCGAACGGTTCTGCCGGGGTGCCAAAC | |
| | AGGGAGGGGCTTCGCTACTACCGCCGCCTGCTGGAGCGTTTACGGGAGCTGGGAGTG | |
| | CAGCCTGTCGTTACTCTGTACCACTGGGACCTCCCGCAGAGGCTGCAGGACGCCTAT | |
| | GGAGGCTGGGCCAACCGCGCCCTTGCGGACCATTTTCGGGATTACGCGGAACTGTGC | |
| | TTCCGCCACTTCGGTGGCCAGGTGAAGTATTGGATCACCATTGACAATCCGTATGTG | |
| | GTGGCTTGGCATGGATACGCGACCGGTAGGCTGGCCCCCGGCATCCGCGGGAGCCCT | |
| | CGGTTGGGCTACCTGGTGGCGCACAACCTGCTGTTGGCCCACGCTAAGGTGTGGCAC | |
| | CTGTACAACACCTCCTTTCGCCCCACCCAGGGCGGTCAGGTGTCCATCGCCCTCAGC | |
| | TCCCACTGGATTAACCCCCGGCGCATGACCGACCACTCCATCAAAGAATGTCAGAAG | |
| | TCCCTCGACTTTGTGCTGGGCTGGTTCGCCAAGCCCGTGTTTATTGACGGTGACTAC | |
| | CCCGAGTCTATGAAGAACAACCTAAGCTCGATCCTGCCCGACTTCACAGAGTCTGAG | |
| | AAAAAATTTATTAAGGGCACCGCGGACTTCTTCGCACTCTGCTTTGGCCCTACCCTG | |
| | TCCTTTCAGCTTCTGGACCCCCACATGAAATTCAGACAGCTGGAGTCACCCAACCTG | |
| | AGGCAGCTGCTGAGTTGGATCGATCTGGAATTCAATCACCCTCAGATCTTCATCGTG | |
| | GAGAATGGATGGTTCGTCTCAGGCACCACCAAGCGCGACGATGCCAAGTACATGTAC | |
| | TATCTGAAGAAATTTATCATGGAGACACTCAAGGCGATCAAGCTGGACGGCGTCGAT | |
| | GTGATCGGTTACACCGCTTGGTCTTTGATGGATGGCTTCGAATGGCACCGCGGCTAC | |
| | TCCATCCGTCGCGGACTGTTCTACGTGGATTTCCTGTCTCAGGACAAAATGCTCCTG | |
| | CCAAAGTCCAGTGCACTGTTCTATCAGAAGCTAATCGAGAAAAATGGCTTCCCACCT | |
| | CTGCCTGAGAACCAGCCCCTCGAAGGCACCTTCCCCTCGGACTTCGCCTGGGGCGTC | |
| | GTGGACAACTATATACAAGTGGACACCACACTTTCTCAGTTTACTGACCTGAACGTG | |
| | TACCTGTGGGACGTGCACCACTCTAAGCGACTTATCAAGGTGGATGGCGTGGTTACT | |
| | AAGAAGCGCAAATCATATTGCGTGGATTTTGCGGCCATCCAGCCTCAGATCGCCCTG | |
| | CTGCAGGAAATGCACGTGACCCACTTTCGATTCTCTCTCGACTGGGCACTGATTCTG | |
| | CCACTTGGCAACCAGAGCCAGGTGAACCATACAATCCTGCAATATTACAGGTGTATG | |
| | GCCAGCGAGCTGGTGCGCGTGAACATCACTCCGGTGGTCGCTCTCTGGCAACCCATG | |
| | GCCCCTAACCAAGGCCTGCCTCGCCTGCTGGCTCGCCAGGGTGCGTGGGAAAACCCG | |
| | TACACGGCCCTGGCCTTCGCCGAGTACGCACGTCTGTGCTTCCAGGAGCTCGGCCAC | |
| | CACGTCAAGTTGTGGATCACCATGAACGAGCCCTACACCCGTAATATGACCTACAGC | |
| | GCAGGCCACAACCTGTTGAAGGCCCATGCCCTGGCTTGGCACGTGTACAACGAGAAG | |
| | TTCCGCCACGCACAGAACGGTAAGATCTCCATCGCTCTTCAGGCGGACTGGATTGAG | |
| | CCAGCCTGCCCATTCTCGCAGAAGGACAAAGAGGTGGCCGAACGCGTCCTCGAATTC | |
| | GACATCGGTTGGCTCGCTGAGCCCATCTTCGGCTCCGGCGACTACCCGTGGGTGATG | |
| | CGTGACTGGCTGAACCAGCGCAACAACTTCCTGCTGCCCTATTTCACCGAGGACGAG | |
| | AAGAAACTGATACAGGGAACCTTCGACTTTCTAGCACTGTCGCACTACACGACAATC | |
| | CTGGTGGACAGCGAGAAGGAGGACCCGATCAAGTACAACGACTACCTGGAGGTGCAG | |
| | GAGATGACCGACATCACCTGGCTGAACTCGCCCAGCCAGGTCGCCGTGGTTCCGTGG | |
| | GGCCTGCGCAAGGTACTGAACTGGCTGAAGTTCAAGTACGGCGACCTGCCCATGTAT | |
| | ATCATCTCCAACGGGATTGACGATGGGCTACATGCTGAGGATGACCAGCTGCGGGTG | |
| | TACTACATGCAGAACTACATCAATGAGGCTCTTAAGGCTCACATTCTAGACGGGATT | |
| | AACTTATCGGGGTACTTCGCGTACTCCTTCAACGACCGCACGGCCCCGCGCTTTGGG | |
| | CTCTACCGCTACGCTGCGGACCAGTTCGAGCCAAAAGCGTCCATGAAGCACTACCGG | |
| | AAGATAATCGACAGCAATGGCTTCCCTGGACCCGAGACTGACGCACATAAGTCCGAG | |
| | GTCGCCCACCGCTTCAAGGACCTGGGCGAGGAGAATTTCAAGGCCTTGGTCCTCATC | |
| | GCCTTCGCCCAGTATCTGCAGCAGTGCCCATTTGAAGATCACGTGAAGCTGGTCAAC | |
| | GAGGTGACGGAGTTCGCCAAGACCTGCGTGGCTGATGAGAGCGCCGAAAACTGCGAT | |
| | AAGAGCCTTCACACACTCTTCGGTGATAAGCTGTGCACCGTGGCCACCCTGCGCGAG | |
| | ACGTACGGCGAGATGGCAGACTGTTGTGCCAAGCAGGAGCCCGAGAGAAACGAGTGC | |
| | TTTCTGCAGCACAAAGACGATAATCCAAACTTGCCGCGCCTGGTGCGTCCCGAGGTA | |
| | GATGTGATGTGCACTGCGTTCCATGACAACGAGGAGACTTTCCTGAAGAAGTATCTC | |
| | TATGAGATCGCCCGCCGCCACCCCTATTTCTACGCGCCGGAGCTACTTTTTTTCGCC | |
| | AAGCGTTACAAGGCGGCCTTCACCGAATGCTGTCAGGCTGCCGACAAAGCTGCCTGC | |
| | CTACTGCCTAAGCTGGACGAGCTCAGGGACGAGGGCAAAGCGAGTTCTGCTAAGCAG | |
| | CGCCTGAAGTGCGCCTCCCTGCAGAAGTTTGGGGAGCGCGCTTTCAAGGCCTGGGCG | |
| | GTGGCGCGCCTGTCGCAGCGTTTTCCCAAAGCTGAGTTCGCGGAGGTGAGCAAATTG | |
| | GTAACCGACCTGACCAAGGTGCACACTGAATGTTGTCATGGCGACCTGCTGGAGTGC | |
| | GCCGATGACCGCGCCGACTTAGCCAAGTACATCTGCGAAAATCAGGACTCTATCTCT | |
| | TCTAAGCTCAAGGAATGTTGCGAGAAACCCCTGCTAGAGAAGTCCCACTGTATTGCT | |
| | GAGGTCGAGAACGACGAGATGCCCGCCGACCTGCCCTCTCTGGCCGCCGACTTTGTC | |
| | GAGAGCAAGGACGTGTGCAAGAACTACGCGGAGGCTAAGGACGTGTTCCTCGGCATG | |
| | TTCCTGTACGAGTACGCCCGACGGCACCCCGACTACTCCGTAGTGCTTTTGCTGCGT | |
| | CTGGCTAAAACCTACGAGACTACCCTTGAGAAATGTTGTGCAGCTGCAGACCCGCAT | |
| | GAGTGTTACGCCAAAGTCTTCGACGAGTTCAAACCTCTGGTCGAAGAGCCTCAGAAC | |
| | CTGATCAAGCAGAACTGCGAGCTGTTCGAGCAGCTAGGAGAGTACAAGTTCCAAAAT | |
| | GCTCTGCTGGTGCGCTACACCAAGAAGGTCCCTCAGGTGTCCACCCCAACCCTGGTG | |
| | GAGGTGTCGCGCAACCTGGGTAAGGTGGGCTCCAAGTGTTGCAAGCACCCGGAGGCC | |
| | AAGCGGATGCCTTGTGCTGAGGACTACCTGTCCGTGGTGTTGAACCAGCTGTGCGTG | |
| | TTGCACGAGAAGACTCCGGTGTCCGATCGTGTGACGAAATGCTGCACTGAGAGTCTT | |
| | GTGAACCGCAGGCCTTGCTTTTCAGCCCTGGAGGTAGACGAGACTTACGTGCCCAAG | |
| | GAGTTCAACGCTGAGACTTTCACATTTCACGCCGACATCTGCACTCTTAGCGAGAAG | |
| | GAGCGGCAAATTAAGAAGCAGACGGCGCTGGTTGAATTAGTTAAGCACAAGCCGAAG | |
| | GCTACCAAGGAACAGCTGAAGGCGGTGATGGATGACTTCGCTGCATTCGTCGAGAAA | |
| | TGTTGCAAAGCGGACGACAAGGAGACTTGCTTCGCAGAGGAGGGCAAGAAGCTGGTG | |
| | GCTGCTTCCCAGGCCGCCCTGGGGCTCCATCACCATCATCACCACTGA | |
| KL958 | EPGDGAQTWARFSRPPAPEAAGLFQGTFPDGFLWAVGSAAYQTEGGWQQHGKGASIW | 54 |
| ((C521S | DTFTHHPLAPPGDSRNASLPLGAPSPLQPATGDVASDSYNNVFRDTEALRELGVTHY | |
| C910S)- | RFSISWARVLPNGSAGVPNREGLRYYRRLLERLRELGVQPVVTLYHWDLPQRLQDAY | |
| HSA-His) | GGWANRALADHFRDYAELCFRHFGGQVKYWITIDNPYVVAWHGYATGRLAPGIRGSP | |
| amino acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNOGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLSGYFAYSFNDRTAPRFGLYRYAADQFEPKASMKHYR | |
| | KIIDSNGFPGPETDAHKSEVAHRFKDLGEENFKALVLIAFAQYLQQCPFEDHVKLVN | |
| | EVTEFAKTCVADESAENCDKSLHTLFGDKLCTVATLRETYGEMADCCAKQEPERNEC | |
| | FLQHKDDNPNLPRLVRPEVDVMCTAFHDNEETFLKKYLYEIARRHPYFYAPELLFFA | |
| | KRYKAAFTECCQAADKAACLLPKLDELRDEGKASSAKORLKCASLQKFGERAFKAWA | |
| | VARLSQRFPKAEFAEVSKLVTDLTKVHTECCHGDLLECADDRADLAKYICENQDSIS | |
| | SKLKECCEKPLLEKSHCIAEVENDEMPADLPSLAADFVESKDVCKNYAEAKDVFLGM | |
| | FLYEYARRHPDYSVVLLLRLAKTYETTLEKCCAAADPHECYAKVFDEFKPLVEEPQN | |
| | LIKQNCELFEQLGEYKFQNALLVRYTKKVPQVSTPTLVEVSRNLGKVGSKCCKHPEA | |
| | KRMPCAEDYLSVVLNQLCVLHEKTPVSDRVTKCCTESLVNRRPCFSALEVDETYVPK | |
| | EFNAETFTFHADICTLSEKERQIKKQTALVELVKHKPKATKEQLKAVMDDFAAFVEK | |
| | CCKADDKETCFAEEGKKLVAASQAALGLHHHHHH | |
| KL958 | GAGCCTGGGGACGGCGCTCAGACCTGGGCTCGTTTTTCTCGTCCTCCTGCTCCTGAG | 55 |
| ((C521S)-Fc- | GCTGCTGGCCTCTTCCAGGGCACGTTCCCAGACGGCTTTTTGTGGGCGGTGGGTTCT | |
| Twin-Strep) | GCGGCCTACCAGACGGAGGGGGGTTGGCAGCAGCATGGCAAGGGCGCCTCCATCTGG | |
| nucleotide | GATACTTTCACGCACCACCCCCTGGCTCCGCCTGGCGATTCCCGCAACGCCTCCCTG | |
| sequence | CCCTTAGGAGCCCCCTCTCCCCTGCAGCCGGCCACCGGCGATGTAGCGTCCGATAGC | |
| | TACAATAACGTGTTCCGCGACACCGAGGCTTTGAGAGAGTTGGGCGTTACTCATTAC | |
| | CGCTTCTCTATCAGTTGGGCCCGCGTACTGCCGAACGGTTCTGCCGGGGTGCCAAAC | |
| | AGGGAGGGGCTTCGCTACTACCGCCGCCTGCTGGAGCGTTTACGGGAGCTGGGAGTG | |
| | CAGCCTGTCGTTACTCTGTACCACTGGGACCTCCCGCAGAGGCTGCAGGACGCCTAT | |
| | GGAGGCTGGGCCAACCGCGCCCTTGCGGACCATTTTCGGGATTACGCGGAACTGTGC | |
| | TTCCGCCACTTCGGTGGCCAGGTGAAGTATTGGATCACCATTGACAATCCGTATGTG | |
| | GTGGCTTGGCATGGATACGCGACCGGTAGGCTGGCCCCCGGCATCCGCGGGAGCCCT | |
| | CGGTTGGGCTACCTGGTGGCGCACAACCTGCTGTTGGCCCACGCTAAGGTGTGGCAC | |
| | CTGTACAACACCTCCTTTCGCCCCACCCAGGGCGGTCAGGTGTCCATCGCCCTCAGC | |
| | TCCCACTGGATTAACCCCCGGCGCATGACCGACCACTCCATCAAAGAATGTCAGAAG | |
| | TCCCTCGACTTTGTGCTGGGCTGGTTCGCCAAGCCCGTGTTTATTGACGGTGACTAC | |
| | CCCGAGTCTATGAAGAACAACCTAAGCTCGATCCTGCCCGACTTCACAGAGTCTGAG | |
| | AAAAAATTTATTAAGGGCACCGCGGACTTCTTCGCACTCTGCTTTGGCCCTACCCTG | |
| | TCCTTTCAGCTTCTGGACCCCCACATGAAATTCAGACAGCTGGAGTCACCCAACCTG | |
| | AGGCAGCTGCTGAGTTGGATCGATCTGGAATTCAATCACCCTCAGATCTTCATCGTG | |
| | GAGAATGGATGGTTCGTCTCAGGCACCACCAAGCGCGACGATGCCAAGTACATGTAC | |
| | TATCTGAAGAAATTTATCATGGAGACACTCAAGGCGATCAAGCTGGACGGCGTCGAT | |
| | GTGATCGGTTACACCGCTTGGTCTTTGATGGATGGCTTCGAATGGCACCGCGGCTAC | |
| | TCCATCCGTCGCGGACTGTTCTACGTGGATTTCCTGTCTCAGGACAAAATGCTCCTG | |
| | CCAAAGTCCAGTGCACTGTTCTATCAGAAGCTAATCGAGAAAAATGGCTTCCCACCT | |
| | CTGCCTGAGAACCAGCCCCTCGAAGGCACCTTCCCCTCGGACTTCGCCTGGGGCGTC | |
| | GTGGACAACTATATACAAGTGGACACCACACTTTCTCAGTTTACTGACCTGAACGTG | |
| | TACCTGTGGGACGTGCACCACTCTAAGCGACTTATCAAGGTGGATGGCGTGGTTACT | |
| | AAGAAGCGCAAATCATATTGCGTGGATTTTGCGGCCATCCAGCCTCAGATCGCCCTG | |
| | CTGCAGGAAATGCACGTGACCCACTTTCGATTCTCTCTCGACTGGGCACTGATTCTG | |
| | CCACTTGGCAACCAGAGCCAGGTGAACCATACAATCCTGCAATATTACAGGTGTATG | |
| | GCCAGCGAGCTGGTGCGCGTGAACATCACTCCGGTGGTCGCTCTCTGGCAACCCATG | |
| | GCCCCTAACCAAGGCCTGCCTCGCCTGCTGGCTCGCCAGGGTGCGTGGGAAAACCCG | |
| | TACACGGCCCTGGCCTTCGCCGAGTACGCACGTCTGTGCTTCCAGGAGCTCGGCCAC | |
| | CACGTCAAGTTGTGGATCACCATGAACGAGCCCTACACCCGTAATATGACCTACAGC | |
| | GCAGGCCACAACCTGTTGAAGGCCCATGCCCTGGCTTGGCACGTGTACAACGAGAAG | |
| | TTCCGCCACGCACAGAACGGTAAGATCTCCATCGCTCTTCAGGCGGACTGGATTGAG | |
| | CCAGCCTGCCCATTCTCGCAGAAGGACAAAGAGGTGGCCGAACGCGTCCTCGAATTT | |
| | GACATCGGTTGGCTCGCTGAGCCCATCTTCGGCTCCGGCGACTACCCGTGGGTGATG | |
| | CGTGACTGGCTGAACCAGCGCAACAACTTCCTGCTGCCCTATTTCACCGAGGACGAG | |
| | AAGAAACTGATACAGGGAACCTTCGACTTTCTAGCACTGTCGCACTACACGACAATC | |
| | CTGGTGGACAGCGAGAAGGAGGACCCGATCAAGTACAACGACTACCTGGAGGTGCAG | |
| | GAGATGACCGACATCACCTGGCTGAACTCGCCCAGCCAGGTCGCCGTGGTTCCGTGG | |
| | GGCCTGCGCAAGGTACTGAACTGGCTGAAGTTCAAGTACGGCGACCTGCCCATGTAT | |
| | ATCATCTCCAACGGGATTGACGATGGGCTACATGCTGAGGATGACCAGCTGCGGGTG | |
| | TACTACATGCAGAACTACATCAATGAGGCTCTTAAGGCTCACATTCTAGACGGGATT | |
| | AACTTATGCGGGTACTTCGCGTACTCCTTCAACGACCGCACGGCCCCGCGCTTTGGG | |
| | CTCTACCGCTACGCTGCGGACCAGTTCGAGCCAAAAGCGTCCATGAAGCACTACCGG | |
| | AAGATAATCGACAGCAATGGCTTCCCTGGACCCGAGACTGGATCCGGAGGCGGTTCG | |
| | GCCCCCGAGGCCGCTGGCGGCCCTTCCGTGTTCCTGTTCCCCCCAAAGCCCAAGGAC | |
| | ACCCTGATGATCTCTCGCACCCCTGAGGTGACCTGCGTGGTCGTTGATGTGAGCCAC | |
| | GAAGATCCAGAGGTTAAGTTCAACTGGTACGTGGACGGCGTCGAAGTCCACAATGCC | |
| | AAGACCAAGCCGAGGGAGGAGCAGTACAACTCCACGTACCGCGTGGTGTCCGTTCTA | |
| | ACCGTGCTTCACCAGGACTGGCTAAACGGCAAGGAGTACAAGTGTAAGGTGTCAAAC | |
| | AAGGCCCTCCCTGCCCCCATTGAGAAGACCATCTCGAAGGCGAAGGGACAGCCCCGC | |
| | GAGCCTCAGGTCTACACGCTGCCCCCATCCCGCGACGAGCTCACTAAGAACCAGGTC | |
| | AGCCTTACTTGCCTGGTGAAGGGCTTTTACCCGTCCGACATCGCGGTAGAGTGGGAG | |
| | AGCAACGGTCAGCCAGAGAATAACTACAAGACGACTCCCCCGGTGCTGGACAGTGAC | |
| | GGCTCCTTCTTTCTGTATTCTAAGCTGACAGTCGACAAAAGCCGCTGGCAACAGGGT | |
| | AACGTGTTCTCGTGTTCTGTAATGCATGAGGCTCTGCACAACCATTACACCCAGAAG | |
| | AGCCTGTCCCTGTCTCCCGGCAAAGGTTCCCTGGAGGTGCTGTTCCAGGGGCCAGGC | |
| | AGCGCCTGGTCCCACCCCCAGTTCGAGAAGGGCGGCGGTAGTGGAGGGGGCGGATCT | |
| | GGCGGCTCAGCTTGGAGCCACCCCCAGTTCGAAAAGTGA | |
| KL958 | EPGDGAQTWARFSRPPAPEAAGLFQGTFPDGFLWAVGSAAYQTEGGWQQHGKGASIW | 56 |
| ((C521S)-Fc- | DTFTHHPLAPPGDSRNASLPLGAPSPLQPATGDVASDSYNNVFRDTEALRELGVTHY | |
| Twin-Strep) | RFSISWARVLPNGSAGVPNREGLRYYRRLLERLRELGVQPVVTLYHWDLPQRLQDAY | |
| amino acid | GGWANRALADHFRDYAELCFRHFGGQVKYWITIDNPYVVAWHGYATGRLAPGIRGSP | |
| sequence | RLGYLVAHNLLLAHAKVWHLYNTSFRPTQGGQVSIALSSHWINPRRMTDHSIKECQK | |
| | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETGSGGGSAPEAAGGPSVFLFPPKPKDTLMISRTPEVTCVVVDVSH | |
| | EDPEVKFNWYVDGVEVHNAKTKPREEQYNSTYRVVSVLTVLHQDWLNGKEYKCKVSN | |
| | KALPAPIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHYTQK | |
| | SLSLSPGKGSLEVLFQGPGSAWSHPQFEKGGGSGGGGSGGSAWSHPQFEK | |
| KL958 | GAGCCTGGGGACGGCGCTCAGACCTGGGCTCGTTTTTCTCGTCCTCCTGCTCCTGAG | 57 |
| ((C521S)- | GCTGCTGGCCTCTTCCAGGGCACGTTCCCAGACGGCTTTTTGTGGGCGGTGGGTTCT | |
| monoFc- | GCGGCCTACCAGACGGAGGGGGGTTGGCAGCAGCATGGCAAGGGCGCCTCCATCTGG | |
| Twin-Strep) | GATACTTTCACGCACCACCCCCTGGCTCCGCCTGGCGATTCCCGCAACGCCTCCCTG | |
| Nucleotide | CCCTTAGGAGCCCCCTCTCCCCTGCAGCCGGCCACCGGCGATGTAGCGTCCGATAGC | |
| sequence | TACAATAACGTGTTCCGCGACACCGAGGCTTTGAGAGAGTTGGGCGTTACTCATTAC | |
| | CGCTTCTCTATCAGTTGGGCCCGCGTACTGCCGAACGGTTCTGCCGGGGTGCCAAAC | |
| | AGGGAGGGGCTTCGCTACTACCGCCGCCTGCTGGAGCGTTTACGGGAGCTGGGAGTG | |
| | CAGCCTGTCGTTACTCTGTACCACTGGGACCTCCCGCAGAGGCTGCAGGACGCCTAT | |
| | GGAGGCTGGGCCAACCGCGCCCTTGCGGACCATTTTCGGGATTACGCGGAACTGTGC | |
| | TTCCGCCACTTCGGTGGCCAGGTGAAGTATTGGATCACCATTGACAATCCGTATGTG | |
| | GTGGCTTGGCATGGATACGCGACCGGTAGGCTGGCCCCCGGCATCCGCGGGAGCCCT | |
| | CGGTTGGGCTACCTGGTGGCGCACAACCTGCTGTTGGCCCACGCTAAGGTGTGGCAC | |
| | CTGTACAACACCTCCTTTCGCCCCACCCAGGGCGGTCAGGTGTCCATCGCCCTCAGC | |
| | TCCCACTGGATTAACCCCCGGCGCATGACCGACCACTCCATCAAAGAATGTCAGAAG | |
| | TCCCTCGACTTTGTGCTGGGCTGGTTCGCCAAGCCCGTGTTTATTGACGGTGACTAC | |
| | CCCGAGTCTATGAAGAACAACCTAAGCTCGATCCTGCCCGACTTCACAGAGTCTGAG | |
| | AAAAAATTTATTAAGGGCACCGCGGACTTCTTCGCACTCTGCTTTGGCCCTACCCTG | |
| | TCCTTTCAGCTTCTGGACCCCCACATGAAATTCAGACAGCTGGAGTCACCCAACCTG | |
| | AGGCAGCTGCTGAGTTGGATCGATCTGGAATTCAATCACCCTCAGATCTTCATCGTG | |
| | GAGAATGGATGGTTCGTCTCAGGCACCACCAAGCGCGACGATGCCAAGTACATGTAC | |
| | TATCTGAAGAAATTTATCATGGAGACACTCAAGGCGATCAAGCTGGACGGCGTCGAT | |
| | GTGATCGGTTACACCGCTTGGTCTTTGATGGATGGCTTCGAATGGCACCGCGGCTAC | |
| | TCCATCCGTCGCGGACTGTTCTACGTGGATTTCCTGTCTCAGGACAAAATGCTCCTG | |
| | CCAAAGTCCAGTGCACTGTTCTATCAGAAGCTAATCGAGAAAAATGGCTTCCCACCT | |
| | CTGCCTGAGAACCAGCCCCTCGAAGGCACCTTCCCCTCGGACTTCGCCTGGGGCGTC | |
| | GTGGACAACTATATACAAGTGGACACCACACTTTCTCAGTTTACTGACCTGAACGTG | |
| | TACCTGTGGGACGTGCACCACTCTAAGCGACTTATCAAGGTGGATGGCGTGGTTACT | |
| | AAGAAGCGCAAATCATATTGCGTGGATTTTGCGGCCATCCAGCCTCAGATCGCCCTG | |
| | CTGCAGGAAATGCACGTGACCCACTTTCGATTCTCTCTCGACTGGGCACTGATTCTG | |
| | CCACTTGGCAACCAGAGCCAGGTGAACCATACAATCCTGCAATATTACAGGTGTATG | |
| | GCCAGCGAGCTGGTGCGCGTGAACATCACTCCGGTGGTCGCTCTCTGGCAACCCATG | |
| | GCCCCTAACCAAGGCCTGCCTCGCCTGCTGGCTCGCCAGGGTGCGTGGGAAAACCCG | |
| | TACACGGCCCTGGCCTTCGCCGAGTACGCACGTCTGTGCTTCCAGGAGCTCGGCCAC | |
| | CACGTCAAGTTGTGGATCACCATGAACGAGCCCTACACCCGTAATATGACCTACAGC | |
| | GCAGGCCACAACCTGTTGAAGGCCCATGCCCTGGCTTGGCACGTGTACAACGAGAAG | |
| | TTCCGCCACGCACAGAACGGTAAGATCTCCATCGCTCTTCAGGCGGACTGGATTGAG | |
| | CCAGCCTGCCCATTCTCGCAGAAGGACAAAGAGGTGGCCGAACGCGTCCTCGAATTT | |
| | GACATCGGTTGGCTCGCTGAGCCCATCTTCGGCTCCGGCGACTACCCGTGGGTGATG | |
| | CGTGACTGGCTGAACCAGCGCAACAACTTCCTGCTGCCCTATTTCACCGAGGACGAG | |
| | AAGAAACTGATACAGGGAACCTTCGACTTTCTAGCACTGTCGCACTACACGACAATC | |
| | CTGGTGGACAGCGAGAAGGAGGACCCGATCAAGTACAACGACTACCTGGAGGTGCAG | |
| | GAGATGACCGACATCACCTGGCTGAACTCGCCCAGCCAGGTCGCCGTGGTTCCGTGG | |
| | GGCCTGCGCAAGGTACTGAACTGGCTGAAGTTCAAGTACGGCGACCTGCCCATGTAT | |
| | ATCATCTCCAACGGGATTGACGATGGGCTACATGCTGAGGATGACCAGCTGCGGGTG | |
| | TACTACATGCAGAACTACATCAATGAGGCTCTTAAGGCTCACATTCTAGACGGGATT | |
| | AACTTATGCGGGTACTTCGCGTACTCCTTCAACGACCGCACGGCCCCGCGCTTTGGG | |
| | CTCTACCGCTACGCTGCGGACCAGTTCGAGCCAAAAGCGTCCATGAAGCACTACCGG | |
| | AAGATAATCGACAGCAATGGCTTCCCTGGACCCGAGACTGGATCCGGAGGCGGTTCG | |
| | GCACCTGAGGCCGCCGGGGGTCCCTCCGTGTTCCTGTTCCCACCCAAGCCTAAGGAC | |
| | ACCCTGATGATCTCCCGCACTCCTGAAGTCACTTGCGTGGTGGTTGATGTAAGTCAT | |
| | GAGGACCCGGAGGTGAAATTTAATTGGTACGTGGACGGCGTGGAGGTGCACAACGCC | |
| | AAGACTAAGCCCCGCGAGGAGCAGTACAACTCCACCTACCGGGTCGTGTCGGTTCTT | |
| | ACGGTGCTCCACCAGGACTGGCTCAACGGTAAGGAGTACAAGTGCAAAGTATCTAAC | |
| | AAGGCGCTGCCCGCCCCCATTGAGAAAACCATCTCTAAGGCTAAGGGACAGCCGCGC | |
| | GAACCTCAGGTGTACACGAGCCCTCCCAGCCGCGACGAGCTGACCAAGAACCAGGTC | |
| | AGCCTGAGGTGTCACGTGAAGGGCTTCTATCCATCCGACATCGCGGTGGAATGGGAG | |
| | AGCAATGGCCAGCCAGAGAACAACTACAAGACCACAAAGCCGGTGCTAGATTCTGAT | |
| | GGCTCCTTCTTCCTGTATTCCAAGCTGACTGTGGACAAGTCCCGTTGGCAACAGGGC | |
| | AACGTCTTTTCATGCTCTGTGATGCACGAGGCTTTGCACAACCATTACACCCAGAAG | |
| | AGCCTTTCGCTGTCCCCCGGCAAAGGCAGCCTGGAAGTGCTGTTCCAGGGACCTGGC | |
| | AGCGCCTGGAGCCACCCTCAGTTTGAGAAGGGCGGCGGAAGCGGCGGAGGAGGCTCT | |
| | GGCGGCAGCGCCTGGTCCCACCCCCAGTTCGAGAAGTGA | |
| KL958 | EPGDGAQTWARFSRPPAPEAAGLFQGTFPDGFLWAVGSAAYQTEGGWQQHGKGASIW | 58 |
| ((C521S)- | DTFTHHPLAPPGDSRNASLPLGAPSPLQPATGDVASDSYNNVFRDTEALRELGVTHY | |
| monoFc- | RFSISWARVLPNGSAGVPNREGLRYYRRLLERLRELGVQPVVTLYHWDLPQRLQDAY | |
| Twin-Strep) | GGWANRALADHFRDYAELCFRHFGGQVKYWITIDNPYVVAWHGYATGRLAPGIRGSP | |
| Amino acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETGSGGGSAPEAAGGPSVFLFPPKPKDTLMISRTPEVTCVVVDVSH | |
| | EDPEVKFNWYVDGVEVHNAKTKPREEQYNSTYRVVSVLTVLHQDWLNGKEYKCKVSN | |
| | KALPAPIEKTISKAKGQPREPQVYTSPPSRDELTKNQVSLRCHVKGFYPSDIAVEWE | |
| | SNGQPENNYKTTKPVLDSDGSFFLYSKLTVDKSRWQQGNVFSCSVMHEALHNHYTQK | |
| | SLSLSPGKGSLEVLFQGPGSAWSHPQFEKGGGSGGGGSGGSAWSHPQFEK | |
| KL958 | GAGCCTGGGGACGGCGCTCAGACCTGGGCTCGTTTTTCTCGTCCTCCTGCTCCTGAG | 59 |
| ((C521S)- | GCTGCTGGCCTCTTCCAGGGCACGTTCCCAGACGGCTTTTTGTGGGCGGTGGGTTCT | |
| Fc1.5-Twin | GCGGCCTACCAGACGGAGGGGGGTTGGCAGCAGCATGGCAAGGGCGCCTCCATCTGG | |
| Strep) | GATACTTTCACGCACCACCCCCTGGCTCCGCCTGGCGATTCCCGCAACGCCTCCCTG | |
| nucleotide | CCCTTAGGAGCCCCCTCTCCCCTGCAGCCGGCCACCGGCGATGTAGCGTCCGATAGC | |
| sequence | TACAATAACGTGTTCCGCGACACCGAGGCTTTGAGAGAGTTGGGCGTTACTCATTAC | |
| | CGCTTCTCTATCAGTTGGGCCCGCGTACTGCCGAACGGTTCTGCCGGGGTGCCAAAC | |
| | AGGGAGGGGCTTCGCTACTACCGCCGCCTGCTGGAGCGTTTACGGGAGCTGGGAGTG | |
| | CAGCCTGTCGTTACTCTGTACCACTGGGACCTCCCGCAGAGGCTGCAGGACGCCTAT | |
| | GGAGGCTGGGCCAACCGCGCCCTTGCGGACCATTTTCGGGATTACGCGGAACTGTGC | |
| | TTCCGCCACTTCGGTGGCCAGGTGAAGTATTGGATCACCATTGACAATCCGTATGTG | |
| | GTGGCTTGGCATGGATACGCGACCGGTAGGCTGGCCCCCGGCATCCGCGGGAGCCCT | |
| | CGGTTGGGCTACCTGGTGGCGCACAACCTGCTGTTGGCCCACGCTAAGGTGTGGCAC | |
| | CTGTACAACACCTCCTTTCGCCCCACCCAGGGCGGTCAGGTGTCCATCGCCCTCAGC | |
| | TCCCACTGGATTAACCCCCGGCGCATGACCGACCACTCCATCAAAGAATGTCAGAAG | |
| | TCCCTCGACTTTGTGCTGGGCTGGTTCGCCAAGCCCGTGTTTATTGACGGTGACTAC | |
| | CCCGAGTCTATGAAGAACAACCTAAGCTCGATCCTGCCCGACTTCACAGAGTCTGAG | |
| | AAAAAATTTATTAAGGGCACCGCGGACTTCTTCGCACTCTGCTTTGGCCCTACCCTG | |
| | TCCTTTCAGCTTCTGGACCCCCACATGAAATTCAGACAGCTGGAGTCACCCAACCTG | |
| | AGGCAGCTGCTGAGTTGGATCGATCTGGAATTCAATCACCCTCAGATCTTCATCGTG | |
| | GAGAATGGATGGTTCGTCTCAGGCACCACCAAGCGCGACGATGCCAAGTACATGTAC | |
| | TATCTGAAGAAATTTATCATGGAGACACTCAAGGCGATCAAGCTGGACGGCGTCGAT | |
| | GTGATCGGTTACACCGCTTGGTCTTTGATGGATGGCTTCGAATGGCACCGCGGCTAC | |
| | TCCATCCGTCGCGGACTGTTCTACGTGGATTTCCTGTCTCAGGACAAAATGCTCCTG | |
| | CCAAAGTCCAGTGCACTGTTCTATCAGAAGCTAATCGAGAAAAATGGCTTCCCACCT | |
| | CTGCCTGAGAACCAGCCCCTCGAAGGCACCTTCCCCTCGGACTTCGCCTGGGGCGTC | |
| | GTGGACAACTATATACAAGTGGACACCACACTTTCTCAGTTTACTGACCTGAACGTG | |
| | TACCTGTGGGACGTGCACCACTCTAAGCGACTTATCAAGGTGGATGGCGTGGTTACT | |
| | AAGAAGCGCAAATCATATTGCGTGGATTTTGCGGCCATCCAGCCTCAGATCGCCCTG | |
| | CTGCAGGAAATGCACGTGACCCACTTTCGATTCTCTCTCGACTGGGCACTGATTCTG | |
| | CCACTTGGCAACCAGAGCCAGGTGAACCATACAATCCTGCAATATTACAGGTGTATG | |
| | GCCAGCGAGCTGGTGCGCGTGAACATCACTCCGGTGGTCGCTCTCTGGCAACCCATG | |
| | GCCCCTAACCAAGGCCTGCCTCGCCTGCTGGCTCGCCAGGGTGCGTGGGAAAACCCG | |
| | TACACGGCCCTGGCCTTCGCCGAGTACGCACGTCTGTGCTTCCAGGAGCTCGGCCAC | |
| | CACGTCAAGTTGTGGATCACCATGAACGAGCCCTACACCCGTAATATGACCTACAGC | |
| | GCAGGCCACAACCTGTTGAAGGCCCATGCCCTGGCTTGGCACGTGTACAACGAGAAG | |
| | TTCCGCCACGCACAGAACGGTAAGATCTCCATCGCTCTTCAGGCGGACTGGATTGAG | |
| | CCAGCCTGCCCATTCTCGCAGAAGGACAAAGAGGTGGCCGAACGCGTCCTCGAATTT | |
| | GACATCGGTTGGCTCGCTGAGCCCATCTTCGGCTCCGGCGACTACCCGTGGGTGATG | |
| | CGTGACTGGCTGAACCAGCGCAACAACTTCCTGCTGCCCTATTTCACCGAGGACGAG | |
| | AAGAAACTGATACAGGGAACCTTCGACTTTCTAGCACTGTCGCACTACACGACAATC | |
| | CTGGTGGACAGCGAGAAGGAGGACCCGATCAAGTACAACGACTACCTGGAGGTGCAG | |
| | GAGATGACCGACATCACCTGGCTGAACTCGCCCAGCCAGGTCGCCGTGGTTCCGTGG | |
| | GGCCTGCGCAAGGTACTGAACTGGCTGAAGTTCAAGTACGGCGACCTGCCCATGTAT | |
| | ATCATCTCCAACGGGATTGACGATGGGCTACATGCTGAGGATGACCAGCTGCGGGTG | |
| | TACTACATGCAGAACTACATCAATGAGGCTCTTAAGGCTCACATTCTAGACGGGATT | |
| | AACTTATGCGGGTACTTCGCGTACTCCTTCAACGACCGCACGGCCCCGCGCTTTGGG | |
| | CTCTACCGCTACGCTGCGGACCAGTTCGAGCCAAAAGCGTCCATGAAGCACTACCGG | |
| | AAGATAATCGACAGCAATGGCTTCCCTGGACCCGAGACTGGATCCGGAGGCGGTTCG | |
| | GCCCCCGAGGCCGCTGGCGGCCCTTCCGTGTTCCTGTTCCCCCCAAAGCCCAAGGAC | |
| | ACCCTGATGATCTCTCGCACCCCTGAGGTGACCTGCGTGGTCGTTGATGTGAGCCAC | |
| | GAAGATCCAGAGGTTAAGTTCAACTGGTACGTGGACGGCGTCGAAGTCCACAATGCC | |
| | AAGACCAAGCCGAGGGAGGAGCAGTACAACTCCACGTACCGCGTGGTGTCCGTTCTA | |
| | ACCGTGCTTCACCAGGACTGGCTAAACGGCAAGGAGTACAAGTGTAAGGTGTCAAAC | |
| | AAGGCCCTCCCTGCCCCCATTGAGAAGACCATCTCGAAGGCGAAGGGACAGCCCCGC | |
| | GAGCCTCAGGTCTACACGCTGCCCCCATCCCGCGACGAGCTCACTAAGAACCAGGTC | |
| | AGCCTTACTTGCCTGGTGAAGGGCTTTTACCCGTCCGACATCGCGGTAGAGTGGGAG | |
| | AGCAACGGTCAGCCAGAGAATAACTACAAGACGACTCCCCCGGTGCTGGACAGTGAC | |
| | GGCTCCTTCTTTCTGTATTCTAAGCTGACAGTCGACAAAAGCCGCTGGCAACAGGGT | |
| | AACGTGTTCTCGTGTTCTGTAATGCATGAGGCTCTGCACAACCATTACACCCAGAAG | |
| | AGCCTGTCCCTGTCTCCCGGCAAAGGAGGCGGCGGGTCCGGGGGCGGCGGTTCGGGC | |
| | GGTGGTGGCTCCGGGGGTGGAGGTTCTGGACAGCCGCGTGAACCTCAGGTGTACACC | |
| | CTTCCCCCCTCTCGGGACGAGCTCACCAAAAACCAGGTGTCCCTGACCTGCCTGGTA | |
| | AAGGGCTTCTATCCGTCCGACATCGCCGTGGAGTGGGAGAGTAATGGGCAGCCGGAG | |
| | AACAACTATAAAACCACACCTCCCGTGCTGGACAGCGATGGCTCCTTCTTCCTGTAC | |
| | TCCAAGTTGACTGTCGATAAATCGCGATGGCAGCAGGGCAACGTGTTTTCTTGCTCT | |
| | GTGATGCACGAAGCACTGCACAACCACTACACCCAGAAGTCTTTGTCCCTGAGCCCG | |
| | GGGAAAGGTTCCCTGGAGGTGCTGTTCCAGGGGCCAGGCAGCGCCTGGTCCCACCCC | |
| | CAGTTCGAGAAGGGCGGCGGTAGTGGAGGGGGCGGATCTGGCGGCTCAGCTTGGAGC | |
| | CACCCCCAGTTCGAAAAGTGA | |
| KL958 | EPGDGAQTWARFSRPPAPEAAGLFQGTFPDGFLWAVGSAAYQTEGGWQQHGKGASIW | 60 |
| ((C521S)- | DTFTHHPLAPPGDSRNASLPLGAPSPLQPATGDVASDSYNNVFRDTEALRELGVTHY | |
| Fc1.5-Twin- | RFSISWARVLPNGSAGVPNREGLRYYRRLLERLRELGVQPVVTLYHWDLPQRLQDAY | |
| Strep) amino | GGWANRALADHFRDYAELCFRHFGGQVKYWITIDNPYVVAWHGYATGRLAPGIRGSP | |
| acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNOGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETGSGGGSAPEAAGGPSVFLFPPKPKDTLMISRTPEVTCVVVDVSH | |
| | EDPEVKFNWYVDGVEVHNAKTKPREEQYNSTYRVVSVLTVLHQDWLNGKEYKCKVSN | |
| | KALPAPIEKTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEALHNHYTQK | |
| | SLSLSPGKGGGGSGGGGSGGGGSGGGGSGQPREPQVYTLPPSRDELTKNQVSLTCLV | |
| | KGFYPSDIAVEWESNGQPENNYKTTPPVLDSDGSFFLYSKLTVDKSRWQQGNVFSCS | |
| | VMHEALHNHYTQKSLSLSPGKGSLEVLFQGPGSAWSHPQFEKGGGSGGGGSGGSAWS | |
| | HPQFEK | |
| KL958 | GAGCCGGGCGACGGCGCGCAGACCTGGGCCCGTTTCTCGCGGCCTCCTGCCCCCGAG | 61 |
| ((C521S)- | GCCGCGGGCCTCTTCCAGGGCACCTTCCCCGACGGCTTCCTCTGGGCCGTGGGCAGC | |
| HSA-Twin- | GCCGCCTACCAGACCGAGGGCGGCTGGCAGCAGCACGGCAAGGGTGCGTCCATCTGG | |
| Strep) | GATACGTTCACCCACCACCCCCTGGCACCCCCGGGAGACTCCCGGAACGCCAGTCTG | |
| nucleotide | CCGTTGGGCGCCCCGTCGCCGCTGCAGCCCGCCACCGGGGACGTAGCCAGCGACAGC | |
| sequence | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTGGTGGAGGAGGTAGTGAT | |
| | GCACACAAGAGTGAGGTTGCTCATCGGTTTAAAGATTTGGGAGAAGAAAATTTCAAA | |
| | GCCTTGGTGTTGATTGCCTTTGCTCAGTATCTTCAGCAGTGTCCATTTGAAGATCAT | |
| | GTAAAATTAGTGAATGAAGTAACTGAATTTGCAAAAACATGTGTTGCTGATGAGTCA | |
| | GCTGAAAATTGTGACAAATCACTTCATACCCTTTTTGGAGACAAATTATGCACAGTT | |
| | GCAACTCTTCGTGAAACCTATGGTGAAATGGCTGACTGCTGTGCAAAACAAGAACCT | |
| | GAGAGAAATGAATGCTTCTTGCAACACAAAGATGACAACCCAAACCTCCCCCGATTG | |
| | GTGAGACCAGAGGTTGATGTGATGTGCACTGCTTTTCATGACAATGAAGAGACATTT | |
| | TTGAAAAAATACTTATATGAAATTGCCAGAAGACATCCTTACTTTTATGCCCCGGAA | |
| | CTCCTTTTCTTTGCTAAAAGGTATAAAGCTGCTTTTACAGAATGTTGCCAAGCTGCT | |
| | GATAAAGCTGCCTGCCTGTTGCCAAAGCTCGATGAACTTCGGGATGAAGGGAAGGCT | |
| | TCGTCTGCCAAACAGAGACTCAAGTGTGCCAGTCTCCAAAAATTTGGAGAAAGAGCT | |
| | TTCAAAGCATGGGCAGTAGCTCGCCTGAGCCAGAGATTTCCCAAAGCTGAGTTTGCA | |
| | GAAGTTTCCAAGTTAGTGACAGATCTTACCAAAGTCCACACGGAATGCTGCCATGGA | |
| | GATCTGCTTGAATGTGCTGATGACAGGGCGGACCTTGCCAAGTATATCTGTGAAAAT | |
| | CAAGATTCGATCTCCAGTAAACTGAAGGAATGCTGTGAAAAACCTCTGTTGGAAAAA | |
| | TCCCACTGCATTGCCGAAGTGGAAAATGATGAGATGCCTGCTGACTTGCCTTCATTA | |
| | GCTGCTGATTTTGTTGAAAGTAAGGATGTTTGCAAAAACTATGCTGAGGCAAAGGAT | |
| | GTCTTCCTGGGCATGTTTTTGTATGAATATGCAAGAAGGCATCCTGATTACTCTGTC | |
| | GTGCTGCTGCTGAGACTTGCCAAGACATATGAAACCACTCTAGAGAAGTGCTGTGCC | |
| | GCTGCAGATCCTCATGAATGCTATGCCAAAGTGTTCGATGAATTTAAACCTCTTGTG | |
| | GAAGAGCCTCAGAATTTAATCAAACAAAATTGTGAGCTTTTTGAGCAGCTTGGAGAG | |
| | TACAAATTCCAGAATGCGCTATGGAGCCGGGCGACGGCGCGCAGACCTGGGCCCGTT | |
| | TCTCGCGGCCTCCTGCCCCCGAGGCCGCGGGCCTCTTCCAGGGCACCTTCCCCGACG | |
| | GCTTCCTCTGGGCCGTGGGCAGCGCCGCCTACCAGACCGAGGGCGGCTGGCAGCAGC | |
| | ACGGCAAGGGTGCGTCCATCTGGGATACGTTCACCCACCACCCCCTGGCACCCCCGG | |
| | GAGACTCCCGGAACGCCAGTCTGCCGTTGGGCGCCCCGTCGCCGCTGCAGCCCGCCA | |
| | CCGGGGACGTAGCCAGCGACAGCTACAACAACGTCTTCCGCGACACGGAGGCGCTGC | |
| | GCGAGCTCGGGGTCACTCACTACCGCTTCTCCATCTCGTGGGCGCGAGTGCTCCCCA | |
| | ATGGCAGCGCGGGCGTCCCCAACCGCGAGGGGCTGCGCTACTACCGGCGCCTGCTGG | |
| | AGCGGCTGCGGGAGCTGGGCGTGCAGCCCGTGGTCACCCTGTACCACTGGGACCTGC | |
| | CCCAGCGCCTGCAGGACGCCTACGGCGGCTGGGCCAACCGCGCCCTGGCCGACCACT | |
| | TCAGGGATTACGCGGAGCTCTGCTTCCGCCACTTCGGCGGTCAGGTCAAGTACTGGA | |
| | TCACCATCGACAACCCCTACGTGGTGGCCTGGCACGGCTACGCCACCGGGCGCCTGG | |
| | CCCCCGGCATCCGGGGCAGCCCGCGGCTCGGGTACCTGGTGGCGCACAACCTCCTCC | |
| | TGGCTCATGCCAAAGTCTGGCATCTCTACAATACTTCTTTCCGTCCCACTCAGGGAG | |
| | GTCAGGTGTCCATTGCCCTAAGCTCTCACTGGATCAATCCTCGAAGAATGACCGACC | |
| | ACAGCATCAAAGAATGTCAAAAATCTCTGGACTTTGTACTAGGTTGGTTTGCCAAAC | |
| | CCGTATTTATTGATGGTGACTATCCCGAGAGCATGAAGAATAACCTTTCATCTATTC | |
| | TGCCTGATTTTACTGAATCTGAGAAAAAGTTCATCAAAGGAACTGCTGACTTTTTTG | |
| | CTCTTTGCTTTGGACCCACCTTGAGTTTTCAACTTTTGGACCCTCACATGAAGTTCC | |
| | GCCAATTGGAATCTCCCAACCTGAGGCAACTGCTTTCCTGGATTGACCTTGAATTTA | |
| | ACCATCCTCAAATATTTATTGTGGAAAATGGCTGGTTTGTCTCAGGGACCACCAAGA | |
| | GAGATGATGCCAAATATATGTATTACCTCAAAAAGTTCATCATGGAAACCTTAAAAG | |
| | CCATCAAGCTGGATGGGGTGGATGTCATCGGGTATACCGCATGGTCCCTCATGGATG | |
| | GTTTCGAGTGGCACAGAGGTTACAGCATCAGGCGTGGACTCTTCTATGTTGACTTTC | |
| | TAAGCCAGGACAAGATGTTGTTGCCAAAGTCTTCAGCCTTGTTCTACCAAAAGCTGA | |
| | TAGAGAAAAATGGCTTCCCTCCTTTACCTGAAAATCAGCCCCTAGAAGGGACATTTC | |
| | CCAGCGACTTTGCTTGGGGAGTTGTTGACAACTACATTCAAGTAGATACCACTCTGT | |
| | CTCAGTTTACCGACCTGAATGTTTACCTGTGGGATGTCCACCACAGTAAAAGGCTTA | |
| | TTAAAGTGGATGGGGTTGTGACCAAGAAGAGGAAATCCTACTGTGTTGACTTTGCTG | |
| | CCATCCAGCCCCAGATCGCTTTACTCCAGGAAATGCACGTTACACATTTTCGCTTCT | |
| | CCCTGGACTGGGCCCTGATTCTCCCTCTGGGTAACCAGTCCCAGGTGAACCACACCA | |
| | TCCTGCAGTACTATCGCTGCATGGCCAGCGAGCTTGTCCGTGTCAACATCACCCCAG | |
| | TGGTGGCCCTGTGGCAGCCTATGGCCCCGAACCAAGGACTGCCGCGCCTCCTGGCCA | |
| | GGCAGGGCGCCTGGGAGAACCCCTACACTGCCCTGGCCTTTGCAGAGTATGCCCGAC | |
| | TGTGCTTTCAAGAGCTCGGCCATCACGTCAAGCTTTGGATAACGATGAATGAGCCGT | |
| | ATACAAGGAATATGACATACAGTGCTGGCCACAACCTTCTGAAGGCCCATGCCCTGG | |
| | CTTGGCATGTGTACAATGAAAAGTTTAGGCATGCTCAGAATGGGAAAATATCCATAG | |
| | CCTTGCAGGCTGATTGGATAGAACCTGCCTGCCCTTTCTCCCAAAAGGACAAAGAGG | |
| | TGGCTGAGAGAGTTTTGGAATTTGACATTGGCTGGCTGGCTGAGCCCATTTTCGGCT | |
| | CTGGAGATTATCCATGGGTGATGAGGGACTGGCTGAACCAAAGAAACAATTTTCTTC | |
| | TTCCTTATTTCACTGAAGATGAAAAAAAGCTAATCCAGGGTACCTTTGACTTTTTGG | |
| | CTTTAAGCCATTATACCACCATCCTTGTAGACTCAGAAAAAGAAGATCCAATAAAAT | |
| | ACAATGATTACCTAGAAGTGCAAGAAATGACCGACATCACGTGGCTCAACTCCCCCA | |
| | GTCAGGTGGCGGTAGTGCCCTGGGGGTTGCGCAAAGTGCTGAACTGGCTGAAGTTCA | |
| | AGTACGGAGACCTCCCCATGTACATAATATCCAATGGAATCGATGACGGGCTGCATG | |
| | CTGAGGACGACCAGCTGAGGGTGTATTATATGCAGAATTACATAAACGAAGCTCTCA | |
| | AAGCCCACATACTGGATGGTATCAATCTTTGCGGATACTTTGCTTATTCGTTTAACG | |
| | ACCGCACAGCTCCGAGGTTTGGCCTCTATCGTTATGCTGCAGATCAGTTTGAGCCCA | |
| | AGGCATCCATGAAACATTACAGGAAAATTATTGACAGCAATGGTTTCCCGGGCCCAG | |
| | AAACTGGTGGAGGAGGTAGTGATGCACACAAGAGTGAGGTTGCTCATCGGTTTAAAG | |
| | ATTTGGGAGAAGAAAATTTCAAAGCCTTGGTGTTGATTGCCTTTGCTCAGTATCTTC | |
| | AGCAGTGTCCATTTGAAGATCATGTAAAATTAGTGAATGAAGTAACTGAATTTGCAA | |
| | AAACATGTGTTGCTGATGAGTCAGCTGAAAATTGTGACAAATCACTTCATACCCTTT | |
| | TTGGAGACAAATTATGCACAGTTGCAACTCTTCGTGAAACCTATGGTGAAATGGCTG | |
| | ACTGCTGTGCAAAACAAGAACCTGAGAGAAATGAATGCTTCTTGCAACACAAAGATG | |
| | ACAACCCAAACCTCCCCCGATTGGTGAGACCAGAGGTTGATGTGATGTGCACTGCTT | |
| | TTCATGACAATGAAGAGACATTTTTGAAAAAATACTTATATGAAATTGCCAGAAGAC | |
| | ATCCTTACTTTTATGCCCCGGAACTCCTTTTCTTTGCTAAAAGGTATAAAGCTGCTT | |
| | TTACAGAATGTTGCCAAGCTGCTGATAAAGCTGCCTGCCTGTTGCCAAAGCTCGATG | |
| | AACTTCGGGATGAAGGGAAGGCTTCGTCTGCCAAACAGAGACTCAAGTGTGCCAGTC | |
| | TCCAAAAATTTGGAGAAAGAGCTTTCAAAGCATGGGCAGTAGCTCGCCTGAGCCAGA | |
| | GATTTCCCAAAGCTGAGTTTGCAGAAGTTTCCAAGTTAGTGACAGATCTTACCAAAG | |
| | TCCACACGGAATGCTGCCATGGAGATCTGCTTGAATGTGCTGATGACAGGGCGGACC | |
| | TTGCCAAGTATATCTGTGAAAATCAAGATTCGATCTCCAGTAAACTGAAGGAATGCT | |
| | GTGAAAAACCTCTGTTGGAAAAATCCCACTGCATTGCCGAAGTGGAAAATGATGAGA | |
| | TGCCTGCTGACTTGCCTTCATTAGCTGCTGATTTTGTTGAAAGTAAGGATGTTTGCA | |
| | AAAACTATGCTGAGGCAAAGGATGTCTTCCTGGGCATGTTTTTGTATGAATATGCAA | |
| | GAAGGCATCCTGATTACTCTGTCGTGCTGCTGCTGAGACTTGCCAAGACATATGAAA | |
| | CCACTCTAGAGAAGTGCTGTGCCGCTGCAGATCCTCATGAATGCTATGCCAAAGTGT | |
| | TCGATGAATTTAAACCTCTTGTGGAAGAGCCTCAGAATTTAATCAAACAAAATTGTG | |
| | AGCTTTTTGAGCAGCTTGGAGAGTACAAATTCCAGAATGCGCTATTAGTTCGTTACA | |
| | CCAAGAAAGTACCCCAAGTGTCAACTCCAACTCTTGTAGAGGTCTCAAGAAACCTAG | |
| | GAAAAGTGGGCAGCAAATGTTGTAAACATCCTGAAGCAAAAAGAATGCCCTGTGCAG | |
| | AAGACTATCTATCCGTGGTCCTGAACCAGTTATGTGTGTTGCATGAGAAAACGCCAG | |
| | TAAGTGACAGAGTCACCAAATGCTGCACAGAATCCTTGGTGAACAGGCGACCATGCT | |
| | TTTCAGCTCTGGAAGTCGATGAAACATACGTTCCCAAAGAGTTTAATGCTGAAACAT | |
| | TCACCTTCCATGCAGATATATGCACACTTTCTGAGAAGGAGAGACAAATCAAGAAAC | |
| | AAACTGCACTTGTTGAGCTCGTGAAACACAAGCCCAAGGCAACAAAAGAGCAACTGA | |
| | AAGCTGTTATGGATGATTTCGCAGCTTTTGTAGAGAAGTGCTGCAAGGCTGACGATA | |
| | AGGAGACCTGCTTTGCCGAGGAGGGTAAAAAACTTGTTGCTGCAAGTCAAGCTGCCT | |
| | TAGGCTTACTGGAAGTGCTGTTCCAGGGACCTGGCAGCGCCTGGAGCCACCCTCAGT | |
| | TTGAGAAGGGCGGCGGAAGCGGCGGAGGAGGCTCTGGCGGCAGCGCCTGGTCCCACC | |
| | CCCAGTTCGAGAAGTGA | |
| KL958 | EPGDGAQTWARFSRPPAPEAAGLFQGTFPDGFLWAVGSAAYQTEGGWQQHGKGASIW | 62 |
| ((C521S)- | DTFTHHPLAPPGDSRNASLPLGAPSPLQPATGDVASDSYNNVFRDTEALRELGVTHY | |
| HSA-Twin- | RFSISWARVLPNGSAGVPNREGLRYYRRLLERLRELGVQPVVTLYHWDLPQRLQDAY | |
| Strep) amino | GGWANRALADHFRDYAELCFRHFGGQVKYWITIDNPYVVAWHGYATGRLAPGIRGSP | |
| acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALCFGPTL | |
| | SFQLLDPHMKFRQLESPNLROLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETGSGGGSDAHKSEVAHRFKDLGEENFKALVLIAFAQYLQQCPFED | |
| | HVKLVNEVTEFAKTCVADESAENCDKSLHTLFGDKLCTVATLRETYGEMADCCAKQE | |
| | PERNECFLQHKDDNPNLPRLVRPEVDVMCTAFHDNEETFLKKYLYEIARRHPYFYAP | |
| | ELLFFAKRYKAAFTECCQAADKAACLLPKLDELRDEGKASSAKQRLKCASLQKFGER | |
| | AFKAWAVARLSQRFPKAEFAEVSKLVTDLTKVHTECCHGDLLECADDRADLAKYICE | |
| | NQDSISSKLKECCEKPLLEKSHCIAEVENDEMPADLPSLAADFVESKDVCKNYAEAK | |
| | DVFLGMFLYEYARRHPDYSVVLLLRLAKTYETTLEKCCAAADPHECYAKVFDEFKPL | |
| | VEEPQNLIKONCELFEQLGEYKFQNALLVRYTKKVPQVSTPTLVEVSRNLGKVGSKC | |
| | CKHPEAKRMPCAEDYLSVVLNQLCVLHEKTPVSDRVTKCCTESLVNRRPCFSALEVD | |
| | ETYVPKEFNAETFTFHADICTLSEKERQIKKQTALVELVKHKPKATKEQLKAVMDDF | |
| | AAFVEKCCKADDKETCFAEEGKKLVAASQAALGLGSLEVLFQGPGSAWSHPQFEKGG | |
| | GSGGGGSGGSAWSHPQFEK | |
| KL958 | GAGCCGGGCGACGGCGCGCAGACCTGGGCCCGTTTCTCGCGGCCTCCTGCCCCCGAG | 63 |
| (C521S)- | GCCGCGGGCCTCTTCCAGGGCACCTTCCCCGACGGCTTCCTCTGGGCCGTGGGCAGC | |
| G4S-HSA | GCCGCCTACCAGACCGAGGGGGGCTGGCAGCAGCACGGCAAGGGTGCGTCCATCTGG | |
| nucleotide | GATACGTTCACCCACCACCCCCTGGCACCCCCGGGAGACTCCCGGAACGCCAGTCTG | |
| sequence | CCGTTGGGCGCCCCGTCGCCGOTGCAGCCCGCCACCGGGGACGTAGCCAGCGACAGC | |
| | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCOTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTOTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTOCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTOCTGGCCAGGCAGGGCGCCTGGGAGAACCCC | |
| | TACACTGCCCTGGCCTTTGCAGAGTATGCCCGACTGTGOTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGGGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGOTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTGGTGGAGGAGGTAGTGAT | |
| | GCACACAAGAGTGAGGTTGCTCATCGGTTTAAAGATTTGGGAGAAGAAAATTTCAAA | |
| | GCCTTGGTGTTGATTGCCTTTGCTCAGTATOTTCAGCAGTGTCCATTTGAAGATCAT | |
| | GTAAAATTAGTGAATGAAGTAACTGAATTTGCAAAAACATGTGTTGCTGATGAGTCA | |
| | GCTGAAAATTGTGACAAATCACTTCATACCCTTTTTGGAGACAAATTATGCACAGTT | |
| | GCAACTCTTCGTGAAACCTATGGTGAAATGGCTGACTGCTGTGCAAAACAAGAACCT | |
| | GAGAGAAATGAATGCTTCTTGCAACACAAAGATGACAACCCAAACCTCCCCCGATTG | |
| | GTGAGACCAGAGGTTGATGTGATGTGCACTGCTTTTCATGACAATGAAGAGACATTT | |
| | TTGAAAAAATACTTATATGAAATTGCCAGAAGACATCCTTACTTTTATGCCCCGGAA | |
| | CTCCTTTTCTTTGCTAAAAGGTATAAAGCTGCTTTTACAGAATGTTGCCAAGCTGCT | |
| | GATAAAGCTGCCTGCCTGTTGCCAAAGCTCGATGAACTTCGGGATGAAGGGAAGGCT | |
| | TCGTCTGCCAAACAGAGACTCAAGTGTGCCAGTCTCCAAAAATTTGGAGAAAGAGCT | |
| | TTCAAAGCATGGGCAGTAGCTCGCCTGAGCCAGAGATTTCCCAAAGCTGAGTTTGCA | |
| | GAAGTTTCCAAGTTAGTGACAGATOTTACCAAAGTCCACACGGAATGCTGCCATGGA | |
| | GATCTGCTTGAATGTGCTGATGACAGGGGGGACCTTGCCAAGTATATCTGTGAAAAT | |
| | CAAGATTCGATCTCCAGTAAACTGAAGGAATGCTGTGAAAAACCTCTGTTGGAAAAA | |
| | TCCCACTGCATTGCCGAAGTGGAAAATGATGAGATGCCTGCTGACTTGCCTTCATTA | |
| | GCTGCTGATTTTGTTGAAAGTAAGGATGTTTGCAAAAACTATGCTGAGGCAAAGGAT | |
| | GTCTTCCTGGGCATGTTTTTGTATGAATATGCAAGAAGGCATCCTGATTACTCTGTC | |
| | GTGCTGCTGCTGAGACTTGCCAAGACATATGAAACCACTCTAGAGAAGTGOTGTGCC | |
| | GCTGCAGATCCTCATGAATGCTATGCCAAAGTGTTCGATGAATTTAAACCTCTTGTG | |
| | GAAGAGCCTCAGAATTTAATCAAACAAAATTGTGAGCTTTTTGAGCAGCTTGGAGAG | |
| | TACAAATTCCAGAATGCGCTATTAGTTCGTTACACCAAGAAAGTACCCCAAGTGTCA | |
| | ACTCCAACTCTTGTAGAGGTCTCAAGAAACCTAGGAAAAGTGGGCAGCAAATGTTGT | |
| | AAACATCCTGAAGCAAAAAGAATGCCCTGTGCAGAAGACTATCTATCCGTGGTCCTG | |
| | AACCAGTTATGTGTGTTGCATGAGAAAACGCCAGTAAGIGACAGAGTCACCAAATGC | |
| | TGCACAGAATCCTTGGTGAACAGGCGACCATGOTTTTCAGCTOTGGAAGTCGATGAA | |
| | ACATACGTTCCCAAAGAGTTTAATGCTGAAACATTCACCTTCCATGCAGATATATGC | |
| | ACACTTTCTGAGAAGGAGAGACAAATCAAGAAACAAACTGCACTTGTTGAGCTCGTG | |
| | AAACACAAGCCCAAGGCAACAAAAGAGCAACTGAAAGCTGTTATGGATGATTTOGCA | |
| | GCTTTTGTAGAGAAGTGCTGCAAGGCTGACGATAAGGAGACCTGCTTTGCCGAGGAG | |
| | GGTAAAAAACTTGTTGCTGCAAGTCAAGCTGCCTTAGGCTTATGA | |
| KL958 | EPGDGAQTWARFSRPPAPEAAGLFQGTFPDGFLWAVGSAAYQTEGGWQQHGKGASIW | 64 |
| (C521S)- | DTFTHHPLAPPGDSRNASLPLGAPSPLQPATGDVASDSYNNVERDTEALRELGVTHY | |
| G4S-HSA | RFSISWARVIPNGSAGVPNREGLRYYRRLLERIRELGVQPVVTLYHWDLPQRLQDAY | |
| amino acid | GGWANRALADHFRDYAELCFRHFGGQVKYWITIDNPYVVAWHGYATGRLAPGIRGSP | |
| sequence | RIGYLVAHNLLLAHAKVWHLYNTSFRPTQGGQVSIALSSHWINPRRMTDHSIKECQK | |
| | SLDFVLGWFAKPVFIDGDYPESMKNNLSSILPDFTESEKKFIKGTADFFALCFGPTL | |
| | SFQLLDPHMKFRQLESPNLRQLLSWIDLEFNHPQIFIVENGWFVSGTTKRDDAKYMY | |
| | YIKKFIMETIKAIKLDGVDVIGYTAWSIMDGFEWHRGYSIRRGIFYVDFLSQDKMLL | |
| | PKSSALFYQKLIEKNGFPPLPENQPLEGTFPSDFAWGVVDNYIQVDTTLSQFTDLNV | |
| | YIWDVHHSKRLIKVDGVVTKKRKSYCVDFAAIQPQIALLQEMHVTHERFSLDWALIL | |
| | PLGNQSQVNHTILQYYRQMASELVRVNITPVVALWQPMAPNQGLPRLLARQGAWENP | |
| | YTALAFARYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWINSPSQVAVVPWGLRKVINWLKPKYGDLPMYIISNGIDDGLHAEDDQLRV | |
| | YYMQNYINEALKAHILDGINLCGYFAYSENDRTAPRFGLYRYAADQFEPKASMKHYR | |
| | KIIDSNGFPGPETGGGGSDAHKSEVAHRFKDLGEENFKALVLIAPAQYLQQCPFEDH | |
| | VKLVNEVTEFAKTCVADESAENCDKSLHTLFGDKLCTVATLRETYGEMADCCAKQEP | |
| | ERNECFLQHKDDNPNLPRLVRPEVDVMCTAFHDNEETFLKKYLYEIARRHPYFYAPE | |
| | LLFFAKRYKAAFTECCQAADKAACLLPKLDELRDEGKASSAKQRLKCASLQKFGERA | |
| | FKAWAVARLSQRFPKAEFAEVSKLVTDLTKVHTECCHGDLLECADDRADLAKYICEN | |
| | QDSISSKLKECCEKPLLEKSHCIAEVENDEMPADLPSLAADFVESKDVCKNYAEAKD | |
| | VFLGMFLYEYARRHPDYSVVLLLRLAKTYETTLEKCCAAADPHECYAKVFDEFKPLV | |
| | EEPQNLIKQNCELFEQLGEYKFQNALLVRYTKKVPQVSTPTIVEVSRNIGKVGSKCC | |
| | KHPEAKRMPCAEDYLSVVLNQLCVLHEKTPVSDRVTKCCTESLVNRRPCFSALEVDE | |
| | TYVPKEFNAETFTFHADICTLSEKERQIKKQTALVELVKHKPKATKEQLKAVMDDFA | |
| | AFVEKCCKADDKETCFAEEGKKIVAASQAALGL | |
| KL958 | GAGCCGGGCGACGGCGCGCAGACCTGGGCCCGTTTCTCGCGGCCTCCTGCCCCCGAG | 65 |
| (C521S | GCCGCGGGCCTCTTCCAGGGCACCTTCCCCGACGGCTTCCTCTGGGCCGTGGGCAGC | |
| C370S)-G4S- | GCCGCCTACCAGACCGAGGGCGGCTGGCAGCAGCACGGCAAGGGTGCGTCCATCTGG | |
| HSA | GATACGTTCACCCACCACCCCCTGGCACCCCCGGGAGACTCCCGGAACGCCAGTCTG | |
| nucleotide | CCGTTGGGCGCCCCGTCGCCGCTGCAGCCCGCCACCGGGGACGTAGCCAGCGACAGC | |
| sequence | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTCCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTGGTGGAGGAGGTAGTGAT | |
| | GCACACAAGAGTGAGGTTGCTCATCGGTTTAAAGATTTGGGAGAAGAAAATTTCAAA | |
| | GCCTTGGTGTTGATTGCCTTTGCTCAGTATCTTCAGCAGTGTCCATTTGAAGATCAT | |
| | GTAAAATTAGTGAATGAAGTAACTGAATTTGCAAAAACATGTGTTGCTGATGAGTCA | |
| | GCTGAAAATTGTGACAAATCACTTCATACCCTTTTTGGAGACAAATTATGCACAGTT | |
| | GCAACTCTTCGTGAAACCTATGGTGAAATGGCTGACTGCTGTGCAAAACAAGAACCT | |
| | GAGAGAAATGAATGCTTCTTGCAACACAAAGATGACAACCCAAACCTCCCCCGATTG | |
| | GTGAGACCAGAGGTTGATGTGATGTGCACTGCTTTTCATGACAATGAAGAGACATTT | |
| | TTGAAAAAATACTTATATGAAATTGCCAGAAGACATCCTTACTTTTATGCCCCGGAA | |
| | CTCCTTTTCTTTGCTAAAAGGTATAAAGCTGCTTTTACAGAATGTTGCCAAGCTGCT | |
| | GATAAAGCTGCCTGCCTGTTGCCAAAGCTCGATGAACTTCGGGATGAAGGGAAGGCT | |
| | TCGTCTGCCAAACAGAGACTCAAGTGTGCCAGTCTCCAAAAATTTGGAGAAAGAGCT | |
| | TTCAAAGCATGGGCAGTAGCTCGCCTGAGCCAGAGATTTCCCAAAGCTGAGTTTGCA | |
| | GAAGTTTCCAAGTTAGTGACAGATCTTACCAAAGTCCACACGGAATGCTGCCATGGA | |
| | GATCTGCTTGAATGTGCTGATGACAGGGCGGACCTTGCCAAGTATATCTGTGAAAAT | |
| | CAAGATTCGATCTCCAGTAAACTGAAGGAATGCTGTGAAAAACCTCTGTTGGAAAAA | |
| | TCCCACTGCATTGCCGAAGTGGAAAATGATGAGATGCCTGCTGACTTGCCTTCATTA | |
| | GCTGCTGATTTTGTTGAAAGTAAGGATGTTTGCAAAAACTATGCTGAGGCAAAGGAT | |
| | GTCTTCCTGGGCATGTTTTTGTATGAATATGCAAGAAGGCATCCTGATTACTCTGTC | |
| | GTGCTGCTGCTGAGACTTGCCAAGACATATGAAACCACTCTAGAGAAGTGCTGTGCC | |
| | GCTGCAGATCCTCATGAATGCTATGCCAAAGTGTTCGATGAATTTAAACCTCTTGTG | |
| | GAAGAGCCTCAGAATTTAATCAAACAAAATTGTGAGCTTTTTGAGCAGCTTGGAGAG | |
| | TACAAATTCCAGAATGCGCTATTAGTTCGTTACACCAAGAAAGTACCCCAAGTGTCA | |
| | ACTCCAACTCTTGTAGAGGTCTCAAGAAACCTAGGAAAAGTGGGCAGCAAATGTTGT | |
| | AAACATCCTGAAGCAAAAAGAATGCCCTGTGCAGAAGACTATCTATCCGTGGTCCTG | |
| | AACCAGTTATGTGTGTTGCATGAGAAAACGCCAGTAAGTGACAGAGTCACCAAATGC | |
| | TGCACAGAATCCTTGGTGAACAGGCGACCATGCTTTTCAGCTCTGGAAGTCGATGAA | |
| | ACATACGTTCCCAAAGAGTTTAATGCTGAAACATTCACCTTCCATGCAGATATATGC | |
| | ACACTTTCTGAGAAGGAGAGACAAATCAAGAAACAAACTGCACTTGTTGAGCTCGTG | |
| | AAACACAAGCCCAAGGCAACAAAAGAGCAACTGAAAGCTGTTATGGATGATTTCGCA | |
| | GCTTTTGTAGAGAAGTGCTGCAAGGCTGACGATAAGGAGACCTGCTTTGCCGAGGAG | |
| | GGTAAAAAACTTGTTGCTGCAAGTCAAGCTGCCTTAGGCTTATGA | |
| KL958 | EPGDGAQTWARFSRPPAPEAAGLFQGTFPDGFLWAVGSAAYQTEGGWQQHGKGASIW | 66 |
| (C521S | DTFTHHPLAPPGDSRNASLPLGAPSPLQPATGDVASDSYNNVFRDTEALRELGVTHY | |
| C370S)-G4S- | RFSISWARVLPNGSAGVPNREGLRYYRRLLERLRELGVQPVVTLYHWDLPQRLQDAY | |
| HSA amino | GGWANRALADHFRDYAELCFRHFGGQVKYWITIDNPYVVAWHGYATGRLAPGIRGSP | |
| acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALSFGPTL | |
| | SFQLLDPHMKFRQLESPNLRQLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETGGGGSDAHKSEVAHRFKDLGEENFKALVLIAFAQYLQQCPFEDH | |
| | VKLVNEVTEFAKTCVADESAENCDKSLHTLFGDKLCTVATLRETYGEMADCCAKQEP | |
| | ERNECFLQHKDDNPNLPRLVRPEVDVMCTAFHDNEETFLKKYLYEIARRHPYFYAPE | |
| | LLFFAKRYKAAFTECCQAADKAACLLPKLDELRDEGKASSAKQRLKCASLQKFGERA | |
| | FKAWAVARLSQRFPKAEFAEVSKLVTDLTKVHTECCHGDLLECADDRADLAKYICEN | |
| | QDSISSKLKECCEKPLLEKSHCIAEVENDEMPADLPSLAADFVESKDVCKNYAEAKD | |
| | VFLGMFLYEYARRHPDYSVVLLLRLAKTYETTLEKCCAAADPHECYAKVFDEFKPLV | |
| | EEPQNLIKQNCELFEQLGEYKFQNALLVRYTKKVPQVSTPTLVEVSRNLGKVGSKCC | |
| | KHPEAKRMPCAEDYLSVVLNQLCVLHEKTPVSDRVTKCCTESLVNRRPCFSALEVDE | |
| | TYVPKEFNAETFTFHADICTLSEKERQIKKQTALVELVKHKPKATKEQLKAVMDDFA | |
| | AFVEKCCKADDKETCFAEEGKKLVAASQAALGL | |
| KL958 | GAGCCGGGCGACGGCGCGCAGACCTGGGCCCGTTTCTCGCGGCCTCCTGCCCCCGAG | 67 |
| (C521S)- | GCCGCGGGCCTCTTCCAGGGCACCTTCCCCGACGGCTTCCTCTGGGCCGTGGGCAGC | |
| PCL1-HSA | GCCGCCTACCAGACCGAGGGCGGCTGGCAGCAGCACGGCAAGGGTGCGTCCATCTGG | |
| nucleotide | GATACGTTCACCCACCACCCCCTGGCACCCCCGGGAGACTCCCGGAACGCCAGTCTG | |
| sequence | CCGTTGGGCGCCCCGTCGCCGCTGCAGCCCGCCACCGGGGACGTAGCCAGCGACAGC | |
| | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTGCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTTCTAGACTGCGGGCCTAC | |
| | CTGCTGCCTGCTCCACCTGCCCCTGGCAACGCCAGCGAGTCTGAGGAAGATAGAAGC | |
| | GCCGGCAGCGTGGAATCCCCTAGCGTCTCTAGCACCCATAGAGTGTCCGACCCCAAG | |
| | TTCCACCCCCTGCACAGCAAGATCATCATCATCAAGAAAGGCCACGCCAAGGACAGC | |
| | CAGCGGTACAAGGTGGACTACGAGAGCGATGCACACAAGAGTGAGGTTGCTCATCGG | |
| | TTTAAAGATTTGGGAGAAGAAAATTTCAAAGCCTTGGTGTTGATTGCCTTTGCTCAG | |
| | TATCTTCAGCAGTGTCCATTTGAAGATCATGTAAAATTAGTGAATGAAGTAACTGAA | |
| | TTTGCAAAAACATGTGTTGCTGATGAGTCAGCTGAAAATTGTGACAAATCACTTCAT | |
| | ACCCTTTTTGGAGACAAATTATGCACAGTTGCAACTCTTCGTGAAACCTATGGTGAA | |
| | ATGGCTGACTGCTGTGCAAAACAAGAACCTGAGAGAAATGAATGCTTCTTGCAACAC | |
| | AAAGATGACAACCCAAACCTCCCCCGATTGGTGAGACCAGAGGTTGATGTGATGTGC | |
| | ACTGCTTTTCATGACAATGAAGAGACATTTTTGAAAAAATACTTATATGAAATTGCC | |
| | AGAAGACATCCTTACTTTTATGCCCCGGAACTCCTTTTCTTTGCTAAAAGGTATAAA | |
| | GCTGCTTTTACAGAATGTTGCCAAGCTGCTGATAAAGCTGCCTGCCTGTTGCCAAAG | |
| | CTCGATGAACTTCGGGATGAAGGGAAGGCTTCGTCTGCCAAACAGAGACTCAAGTGT | |
| | GCCAGTCTCCAAAAATTTGGAGAAAGAGCTTTCAAAGCATGGGCAGTAGCTCGCCTG | |
| | AGCCAGAGATTTCCCAAAGCTGAGTTTGCAGAAGTTTCCAAGTTAGTGACAGATCTT | |
| | ACCAAAGTCCACACGGAATGCTGCCATGGAGATCTGCTTGAATGTGCTGATGACAGG | |
| | GCGGACCTTGCCAAGTATATCTGTGAAAATCAAGATTCGATCTCCAGTAAACTGAAG | |
| | GAATGCTGTGAAAAACCTCTGTTGGAAAAATCCCACTGCATTGCCGAAGTGGAAAAT | |
| | GATGAGATGCCTGCTGACTTGCCTTCATTAGCTGCTGATTTTGTTGAAAGTAAGGAT | |
| | GTTTGCAAAAACTATGCTGAGGCAAAGGATGTCTTCCTGGGCATGTTTTTGTATGAA | |
| | TATGCAAGAAGGCATCCTGATTACTCTGTCGTGCTGCTGCTGAGACTTGCCAAGACA | |
| | TATGAAACCACTCTAGAGAAGTGCTGTGCCGCTGCAGATCCTCATGAATGCTATGCC | |
| | AAAGTGTTCGATGAATTTAAACCTCTTGTGGAAGAGCCTCAGAATTTAATCAAACAA | |
| | AATTGTGAGCTTTTTGAGCAGCTTGGAGAGTACAAATTCCAGAATGCGCTATTAGTT | |
| | CGTTACACCAAGAAAGTACCCCAAGTGTCAACTCCAACTCTTGTAGAGGTCTCAAGA | |
| | AACCTAGGAAAAGTGGGCAGCAAATGTTGTAAACATCCTGAAGCAAAAAGAATGCCC | |
| | TGTGCAGAAGACTATCTATCCGTGGTCCTGAACCAGTTATGTGTGTTGCATGAGAAA | |
| | ACGCCAGTAAGTGACAGAGTCACCAAATGCTGCACAGAATCCTTGGTGAACAGGCGA | |
| | CCATGCTTTTCAGCTCTGGAAGTCGATGAAACATACGTTCCCAAAGAGTTTAATGCT | |
| | GAAACATTCACCTTCCATGCAGATATATGCACACTTTCTGAGAAGGAGAGACAAATC | |
| | AAGAAACAAACTGCACTTGTTGAGCTCGTGAAACACAAGCCCAAGGCAACAAAAGAG | |
| | CAACTGAAAGCTGTTATGGATGATTTCGCAGCTTTTGTAGAGAAGTGCTGCAAGGCT | |
| | GACGATAAGGAGACCTGCTTTGCCGAGGAGGGTAAAAAACTTGTTGCTGCAAGTCAA | |
| | GCTGCCTTAGGCTTATGA | |
| KL958 | EPGDGAQTWARFSRPPAPEAAGLFQGTFPDGFLWAVGSAAYQTEGGWQQHGKGASIW | 68 |
| (C521S)- | DTFTHHPLAPPGDSRNASLPLGAPSPLQPATGDVASDSYNNVFRDTEALRELGVTHY | |
| PCL1-HSA | RFSISWARVLPNGSAGVPNREGLRYYRRLLERLRELGVQPVVTLYHWDLPQRLQDAY | |
| amino acid | GGWANRALADHFRDYAELCFRHFGGQVKYWITIDNPYVVAWHGYATGRLAPGIRGSP | |
| sequence | RLGYLVAHNLLLAHAKVWHLYNTSFRPTQGGQVSIALSSHWINPRRMTDHSIKECQK | |
| | SLDFVLGWFAKPVFIDGDYPESMKNNLSSILPDFTESEKKFIKGTADFFALCFGPTL | |
| | SFQLLDPHMKFRQLESPNLRQLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETSRLRAYLLPAPPAPGNASESEEDRSAGSVESPSVSSTHRVSDPK | |
| | FHPLHSKIIIIKKGHAKDSQRYKVDYESDAHKSEVAHRFKDLGEENFKALVLIAFAQ | |
| | YLQQCPFEDHVKLVNEVTEFAKTCVADESAENCDKSLHTLFGDKLCTVATLRETYGE | |
| | MADCCAKQEPERNECFLQHKDDNPNLPRLVRPEVDVMCTAFHDNEETFLKKYLYEIA | |
| | RRHPYFYAPELLFFAKRYKAAFTECCQAADKAACLLPKLDELRDEGKASSAKQRLKC | |
| | ASLQKFGERAFKAWAVARLSQRFPKAEFAEVSKLVTDLTKVHTECCHGDLLECADDR | |
| | ADLAKYICENQDSISSKLKECCEKPLLEKSHCIAEVENDEMPADLPSLAADFVESKD | |
| | VCKNYAEAKDVFLGMFLYEYARRHPDYSVVLLLRLAKTYETTLEKCCAAADPHECYA | |
| | KVFDEFKPLVEEPQNLIKQNCELFEQLGEYKFQNALLVRYTKKVPQVSTPTLVEVSR | |
| | NLGKVGSKCCKHPEAKRMPCAEDYLSVVLNQLCVLHEKTPVSDRVTKCCTESLVNRR | |
| | PCFSALEVDETYVPKEFNAETFTFHADICTLSEKERQIKKQTALVELVKHKPKATKE | |
| | QLKAVMDDFAAFVEKCCKADDKETCFAEEGKKLVAASQAALGL | |
| KL958 | GAGCCGGGCGACGGCGCGCAGACCTGGGCCCGTTTCTCGCGGCCTCCTGCCCCCGAG | 69 |
| (C521S | GCCGCGGGCCTCTTCCAGGGCACCTTCCCCGACGGCTTCCTCTGGGCCGTGGGCAGC | |
| C370S)- | GCCGCCTACCAGACCGAGGGCGGCTGGCAGCAGCACGGCAAGGGTGCGTCCATCTGG | |
| PCL1-HSA | GATACGTTCACCCACCACCCCCTGGCACCCCCGGGAGACTCCCGGAACGCCAGTCTG | |
| nucleotide | CCGTTGGGCGCCCCGTCGCCGCTGCAGCCCGCCACCGGGGACGTAGCCAGCGACAGC | |
| sequence | TACAACAACGTCTTCCGCGACACGGAGGCGCTGCGCGAGCTCGGGGTCACTCACTAC | |
| | CGCTTCTCCATCTCGTGGGCGCGAGTGCTCCCCAATGGCAGCGCGGGCGTCCCCAAC | |
| | CGCGAGGGGCTGCGCTACTACCGGCGCCTGCTGGAGCGGCTGCGGGAGCTGGGCGTG | |
| | CAGCCCGTGGTCACCCTGTACCACTGGGACCTGCCCCAGCGCCTGCAGGACGCCTAC | |
| | GGCGGCTGGGCCAACCGCGCCCTGGCCGACCACTTCAGGGATTACGCGGAGCTCTGC | |
| | TTCCGCCACTTCGGCGGTCAGGTCAAGTACTGGATCACCATCGACAACCCCTACGTG | |
| | GTGGCCTGGCACGGCTACGCCACCGGGCGCCTGGCCCCCGGCATCCGGGGCAGCCCG | |
| | CGGCTCGGGTACCTGGTGGCGCACAACCTCCTCCTGGCTCATGCCAAAGTCTGGCAT | |
| | CTCTACAATACTTCTTTCCGTCCCACTCAGGGAGGTCAGGTGTCCATTGCCCTAAGC | |
| | TCTCACTGGATCAATCCTCGAAGAATGACCGACCACAGCATCAAAGAATGTCAAAAA | |
| | TCTCTGGACTTTGTACTAGGTTGGTTTGCCAAACCCGTATTTATTGATGGTGACTAT | |
| | CCCGAGAGCATGAAGAATAACCTTTCATCTATTCTGCCTGATTTTACTGAATCTGAG | |
| | AAAAAGTTCATCAAAGGAACTGCTGACTTTTTTGCTCTTTCCTTTGGACCCACCTTG | |
| | AGTTTTCAACTTTTGGACCCTCACATGAAGTTCCGCCAATTGGAATCTCCCAACCTG | |
| | AGGCAACTGCTTTCCTGGATTGACCTTGAATTTAACCATCCTCAAATATTTATTGTG | |
| | GAAAATGGCTGGTTTGTCTCAGGGACCACCAAGAGAGATGATGCCAAATATATGTAT | |
| | TACCTCAAAAAGTTCATCATGGAAACCTTAAAAGCCATCAAGCTGGATGGGGTGGAT | |
| | GTCATCGGGTATACCGCATGGTCCCTCATGGATGGTTTCGAGTGGCACAGAGGTTAC | |
| | AGCATCAGGCGTGGACTCTTCTATGTTGACTTTCTAAGCCAGGACAAGATGTTGTTG | |
| | CCAAAGTCTTCAGCCTTGTTCTACCAAAAGCTGATAGAGAAAAATGGCTTCCCTCCT | |
| | TTACCTGAAAATCAGCCCCTAGAAGGGACATTTCCCAGCGACTTTGCTTGGGGAGTT | |
| | GTTGACAACTACATTCAAGTAGATACCACTCTGTCTCAGTTTACCGACCTGAATGTT | |
| | TACCTGTGGGATGTCCACCACAGTAAAAGGCTTATTAAAGTGGATGGGGTTGTGACC | |
| | AAGAAGAGGAAATCCTACTGTGTTGACTTTGCTGCCATCCAGCCCCAGATCGCTTTA | |
| | CTCCAGGAAATGCACGTTACACATTTTCGCTTCTCCCTGGACTGGGCCCTGATTCTC | |
| | CCTCTGGGTAACCAGTCCCAGGTGAACCACACCATCCTGCAGTACTATCGCTGCATG | |
| | GCCAGCGAGCTTGTCCGTGTCAACATCACCCCAGTGGTGGCCCTGTGGCAGCCTATG | |
| | GCCCCGAACCAAGGACTGCCGCGCCTCCTGGCCAGGCAGGGCGCCTGGGAGAACCCC | |
| | TACACTGCCCTGGCCTTTGCAGAGTATGCCCGACTGTGCTTTCAAGAGCTCGGCCAT | |
| | CACGTCAAGCTTTGGATAACGATGAATGAGCCGTATACAAGGAATATGACATACAGT | |
| | GCTGGCCACAACCTTCTGAAGGCCCATGCCCTGGCTTGGCATGTGTACAATGAAAAG | |
| | TTTAGGCATGCTCAGAATGGGAAAATATCCATAGCCTTGCAGGCTGATTGGATAGAA | |
| | CCTGCCTGCCCTTTCTCCCAAAAGGACAAAGAGGTGGCTGAGAGAGTTTTGGAATTT | |
| | GACATTGGCTGGCTGGCTGAGCCCATTTTCGGCTCTGGAGATTATCCATGGGTGATG | |
| | AGGGACTGGCTGAACCAAAGAAACAATTTTCTTCTTCCTTATTTCACTGAAGATGAA | |
| | AAAAAGCTAATCCAGGGTACCTTTGACTTTTTGGCTTTAAGCCATTATACCACCATC | |
| | CTTGTAGACTCAGAAAAAGAAGATCCAATAAAATACAATGATTACCTAGAAGTGCAA | |
| | GAAATGACCGACATCACGTGGCTCAACTCCCCCAGTCAGGTGGCGGTAGTGCCCTGG | |
| | GGGTTGCGCAAAGTGCTGAACTGGCTGAAGTTCAAGTACGGAGACCTCCCCATGTAC | |
| | ATAATATCCAATGGAATCGATGACGGGCTGCATGCTGAGGACGACCAGCTGAGGGTG | |
| | TATTATATGCAGAATTACATAAACGAAGCTCTCAAAGCCCACATACTGGATGGTATC | |
| | AATCTTTGCGGATACTTTGCTTATTCGTTTAACGACCGCACAGCTCCGAGGTTTGGC | |
| | CTCTATCGTTATGCTGCAGATCAGTTTGAGCCCAAGGCATCCATGAAACATTACAGG | |
| | AAAATTATTGACAGCAATGGTTTCCCGGGCCCAGAAACTTCTAGACTGCGGGCCTAC | |
| | CTGCTGCCTGCTCCACCTGCCCCTGGCAACGCCAGCGAGTCTGAGGAAGATAGAAGC | |
| | GCCGGCAGCGTGGAATCCCCTAGCGTCTCTAGCACCCATAGAGTGTCCGACCCCAAG | |
| | TTCCACCCCCTGCACAGCAAGATCATCATCATCAAGAAAGGCCACGCCAAGGACAGC | |
| | CAGCGGTACAAGGTGGACTACGAGAGCGATGCACACAAGAGTGAGGTTGCTCATCGG | |
| | TTTAAAGATTTGGGAGAAGAAAATTTCAAAGCCTTGGTGTTGATTGCCTTTGCTCAG | |
| | TATCTTCAGCAGTGTCCATTTGAAGATCATGTAAAATTAGTGAATGAAGTAACTGAA | |
| | TTTGCAAAAACATGTGTTGCTGATGAGTCAGCTGAAAATTGTGACAAATCACTTCAT | |
| | ACCCTTTTTGGAGACAAATTATGCACAGTTGCAACTCTTCGTGAAACCTATGGTGAA | |
| | ATGGCTGACTGCTGTGCAAAACAAGAACCTGAGAGAAATGAATGCTTCTTGCAACAC | |
| | AAAGATGACAACCCAAACCTCCCCCGATTGGTGAGACCAGAGGTTGATGTGATGTGC | |
| | ACTGCTTTTCATGACAATGAAGAGACATTTTTGAAAAAATACTTATATGAAATTGCC | |
| | AGAAGACATCCTTACTTTTATGCCCCGGAACTCCTTTTCTTTGCTAAAAGGTATAAA | |
| | GCTGCTTTTACAGAATGTTGCCAAGCTGCTGATAAAGCTGCCTGCCTGTTGCCAAAG | |
| | CTCGATGAACTTCGGGATGAAGGGAAGGCTTCGTCTGCCAAACAGAGACTCAAGTGT | |
| | GCCAGTCTCCAAAAATTTGGAGAAAGAGCTTTCAAAGCATGGGCAGTAGCTCGCCTG | |
| | AGCCAGAGATTTCCCAAAGCTGAGTTTGCAGAAGTTTCCAAGTTAGTGACAGATCTT | |
| | ACCAAAGTCCACACGGAATGCTGCCATGGAGATCTGCTTGAATGTGCTGATGACAGG | |
| | GCGGACCTTGCCAAGTATATCTGTGAAAATCAAGATTCGATCTCCAGTAAACTGAAG | |
| | GAATGCTGTGAAAAACCTCTGTTGGAAAAATCCCACTGCATTGCCGAAGTGGAAAAT | |
| | GATGAGATGCCTGCTGACTTGCCTTCATTAGCTGCTGATTTTGTTGAAAGTAAGGAT | |
| | GTTTGCAAAAACTATGCTGAGGCAAAGGATGTCTTCCTGGGCATGTTTTTGTATGAA | |
| | TATGCAAGAAGGCATCCTGATTACTCTGTCGTGCTGCTGCTGAGACTTGCCAAGACA | |
| | TATGAAACCACTCTAGAGAAGTGCTGTGCCGCTGCAGATCCTCATGAATGCTATGCC | |
| | AAAGTGTTCGATGAATTTAAACCTCTTGTGGAAGAGCCTCAGAATTTAATCAAACAA | |
| | AATTGTGAGCTTTTTGAGCAGCTTGGAGAGTACAAATTCCAGAATGCGCTATTAGTT | |
| | CGTTACACCAAGAAAGTACCCCAAGTGTCAACTCCAACTCTTGTAGAGGTCTCAAGA | |
| | AACCTAGGAAAAGTGGGCAGCAAATGTTGTAAACATCCTGAAGCAAAAAGAATGCCC | |
| | TGTGCAGAAGACTATCTATCCGTGGTCCTGAACCAGTTATGTGTGTTGCATGAGAAA | |
| | ACGCCAGTAAGTGACAGAGTCACCAAATGCTGCACAGAATCCTTGGTGAACAGGCGA | |
| | CCATGCTTTTCAGCTCTGGAAGTCGATGAAACATACGTTCCCAAAGAGTTTAATGCT | |
| | GAAACATTCACCTTCCATGCAGATATATGCACACTTTCTGAGAAGGAGAGACAAATC | |
| | AAGAAACAAACTGCACTTGTTGAGCTCGTGAAACACAAGCCCAAGGCAACAAAAGAG | |
| | CAACTGAAAGCTGTTATGGATGATTTCGCAGCTTTTGTAGAGAAGTGCTGCAAGGCT | |
| | GACGATAAGGAGACCTGCTTTGCCGAGGAGGGTAAAAAACTTGTTGCTGCAAGTCAA | |
| | GCTGCCTTAGGCTTATGA | |
| KL958 | EPGDGAQTWARFSRPPAPEAAGLFQGTFPDGFLWAVGSAAYQTEGGWQQHGKGASIW | 70 |
| (C521S | DTFTHHPLAPPGDSRNASLPLGAPSPLQPATGDVASDSYNNVFRDTEALRELGVTHY | |
| C370S)- | RFSISWARVLPNGSAGVPNREGLRYYRRLLERLRELGVQPVVTLYHWDLPQRLQDAY | |
| PCL1-HSA | GGWANRALADHFRDYAELCFRHFGGQVKYWITIDNPYVVAWHGYATGRLAPGIRGSP | |
| amino acid | RLGYLVAHNLLLAHAKVWHLYNTSFRPTQGGQVSIALSSHWINPRRMTDHSIKECQK | |
| sequence | SLDFVLGWFAKPVFIDGDYPESMKNNLSSILPDFTESEKKFIKGTADFFALSFGPTL | |
| | SFQLLDPHMKFRQLESPNLRQLLSWIDLEFNHPQIFIVENGWFVSGTTKRDDAKYMY | |
| | YLKKFIMETLKAIKLDGVDVIGYTAWSLMDGFEWHRGYSIRRGLFYVDFLSQDKMLL | |
| | PKSSALFYQKLIEKNGFPPLPENQPLEGTFPSDFAWGVVDNYIQVDTTLSQFTDLNV | |
| | YLWDVHHSKRLIKVDGVVTKKRKSYCVDFAAIQPQIALLQEMHVTHFRFSLDWALIL | |
| | PLGNQSQVNHTILQYYRCMASELVRVNITPVVALWQPMAPNQGLPRLLARQGAWENP | |
| | YTALAFAEYARLCFQELGHHVKLWITMNEPYTRNMTYSAGHNLLKAHALAWHVYNEK | |
| | FRHAQNGKISIALQADWIEPACPFSQKDKEVAERVLEFDIGWLAEPIFGSGDYPWVM | |
| | RDWLNQRNNFLLPYFTEDEKKLIQGTFDFLALSHYTTILVDSEKEDPIKYNDYLEVQ | |
| | EMTDITWLNSPSQVAVVPWGLRKVLNWLKFKYGDLPMYIISNGIDDGLHAEDDQLRV | |
| | YYMQNYINEALKAHILDGINLCGYFAYSFNDRTAPRFGLYRYAADQFEPKASMKHYR | |
| | KIIDSNGFPGPETSRLRAYLLPAPPAPGNASESEEDRSAGSVESPSVSSTHRVSDPK | |
| | FHPLHSKIIIIKKGHAKDSQRYKVDYESDAHKSEVAHRFKDLGEENFKALVLIAFAQ | |
| | YLQQCPFEDHVKLVNEVTEFAKTCVADESAENCDKSLHTLFGDKLCTVATLRETYGE | |
| | MADCCAKQEPERNECFLQHKDDNPNLPRLVRPEVDVMCTAFHDNEETFLKKYLYEIA | |
| | RRHPYFYAPELLFFAKRYKAAFTECCQAADKAACLLPKLDELRDEGKASSAKQRLKC | |
| | ASLQKFGERAFKAWAVARLSQRFPKAEFAEVSKLVTDLTKVHTECCHGDLLECADDR | |
| | ADLAKYICENQDSISSKLKECCEKPLLEKSHCIAEVENDEMPADLPSLAADFVESKD | |
| | VCKNYAEAKDVFLGMFLYEYARRHPDYSVVLLLRLAKTYETTLEKCCAAADPHECYA | |
| | KVFDEFKPLVEEPQNLIKQNCELFEQLGEYKFQNALLVRYTKKVPQVSTPTLVEVSR | |
| | NLGKVGSKCCKHPEAKRMPCAEDYLSVVLNQLCVLHEKTPVSDRVTKCCTESLVNRR | |
| | PCFSALEVDETYVPKEFNAETFTFHADICTLSEKERQIKKQTALVELVKHKPKATKE | |
| | QLKAVMDDFAAFVEKCCKADDKETCFAEEGKKLVAASQAALGL | |

### 9.1.2. Purification of KL Polypeptide Constructs

### 9.1.2.1. Affinity Column Purification

For some evaluations, His- and HSA-tagged KL polypeptides were purified with Ni-NTA Agarose (Qiagen) or CaptureSelect™ Human Albumin Affinity Matrix (ThermoFisher). First the Ni-NTA and Albumin Affinity Matrix columns were equilibrated with 5 column volumes (CV) of equilibration buffer. Next, sterile filtered supernatant containing KL polypeptides was loaded over the pre-equilibrated column at a flow rate of 2.0 mL/min. Any non-specifically bound substances were washed out the columns using a wash buffer for 5CV at a flow rate of 2.0 mL/min. Finally, the affinity bound KL proteins were eluted from the column with 1CV elution buffer and further purified with SEC. The buffer compositions for His and HSA affinity columns are set forth in Table E2 below.

TABLE E2
Affinity Column Buffer Compositions

| Column | | | |
|---|---|---|---|
| Type | Buffer Name | Buffer Composition | Buffer pH |
| HSA | Equilibration | 50 mM Tris, 150 mM NaCl | 8 |
| | Wash | 50 mM Tris, 500 mM NaCl | 8 |
| | Elution | 100 mM Glycine | 3 |
| His | Equilibration | 50 mM Tris, 150 mM NaCl | 8 |
| | Wash | 50 mM Tris, 500 mM NaCl, | 8 |
| | | 20 mM Imidazole | |
| | Elution | 50 mM Tris, 500 mM NaCl, | 8 |
| | | 250 mM Imidazole | |

### 9.1.2.2. Twin-Strep Purification

Twin-Strep-tagged KL polypeptides were purified with Strep-Tactin® XT resin (IBA). First, the column was equilibrated with 5 column volumes (CV) of PBS buffer (pH7.4). Next, sterile filtered supernatant containing KL polypeptides was loaded over the pre-equilibrated column at a flow rate of 2.0 mL/min. Any non-specifically bound substances were washed out the columns using PBS for 5CV at a flow rate of 2.0 mL/min. 1CV PBS and HRV-3C protease (SigmaAldrich, 1:100) were added to the column, incubated at 4° C. for 24 hours, and the flow-through was harvested. Next, the column was washed with 3CV PBS and the resulting flow-through was harvested. Both flow-throughs were combined and further purified with SEC.

In some cases, Twin-Strep-tagged KL polypeptides were purified using StrepTrap XT® (Cytiva). First, the column was equilibrated with 5 column volumes (CV) of binding buffer (100 mM Tris-HCl, 150 mM NaCl; 1 mM EDTA, pH 8). Next, sterile filtered supernatant containing KL polypeptides was loaded over the pre-equilibrated column at a flow rate of 2.0 mL/min. Any non-specifically bound substances were washed out the columns using PBS for 5CV at a flow rate of 2.0 mL/min. Next, the column was eluted with 2 column volume (CV) of elution buffer (100 mM Tris-HCl, 150 mM NaCl; 1 mM EDTA, 50 mM Biotin, pH 8). The elution further purified with SEC.

### 9.1.2.3. Modified High Salt Purification

A CM volume of 500 mL was collected from CHO cell lines expressing KL polypeptide constructs. CaptureSelect™ HSA Affinity Resin (ThermoFisher) in PBS was utilized for binding overnight at 4° C. KL polypeptides were eluted from the resin with a high salt elution buffer (2.0 M MgCl2 in Tris-HCl, pH 7.4). Aggregated and HMW proteins and other contaminations were removed with SEC.

### 9.1.2.4. Modified Ion Exchange Purification

A three-step modified ion exchange (IEX) purification was utilized to capture the KL polypeptides from large volume samples. In step one, sterile filtered supernatant containing KL polypeptides were loaded onto Q-Sepharose column. The column was washed with 20 mM PBS pH6.5. Proteins were eluted with NaCl gradient (50 mM to 2M). SDS-PAGE gels were used for guidance with the fraction collection. In the next step, CaptureSelect™ Human Albumin Affinity Matrix was used to specifically capture the HSA-tagged KL polypeptides and remove the contaminants. Proteins were eluted with Pierce™ Gentle Ag/Ab Elution Buffer, pH 6.6 (ThermoFisher). KL polypeptides were further purified with SEC.

### 9.1.3. Pharmacokinetic Analysis of KL Polypeptides

To assess the pharmacokinetic properties of KL polypeptides in plasma, adult C57BL/6J mice were subcutaneously injected with a single dose of a KL polypeptide or a control antibody diluted in saline. Blood samples were collected at 0, 2, 8, 24, 48, 72, and 96 hours as well as at day 6 and 14 after dosing. Plasma was isolated by centrifuging the samples.

9.1.4. pERK HTRF Assay

Phospho-ERK (PERK) homogeneous time-resolved fluorescence-based (HTRF) assay utilizes ERK signaling as a readout of FGF receptor activity, triggered by co-binding of FGF23 and alpha-Klotho.

NIH3T3 cells (ATCC) in culture were washed twice with 10 mL Ca²⁺ and Mg²⁺-free PBS. 4 mL of TrypLE™ (ThermoFisher) was added to washed cells, which were then incubated at 37° C., 5% CO₂ for 5 minutes. 10 mL of PBS was added to the cells, and cell clumps were broken up by repeated pipetting. The dissociated cell mixture was transferred to a 50 ml conical tube and centrifuged at 1,000 rpm for 5 minutes. The supernatant was removed, and the cells were resuspended in 1 mL assay medium (0.5% BSA in OptiMEM) by repeated pipetting. Next, 10 mL of assay medium was added to the resuspended cells, and cells were counted by Auto T4. Cells were diluted to 2.0×10⁵ cells/mL in assay medium, seeded onto 96-well plates at 20,000 cells/well, and incubated overnight at 37° C., 5% CO₂.

The next day, the media were replaced with 50 μL of assay medium/well and plates were incubated at 37° C., 5% CO₂ for 2 hours. KL polypeptides were diluted to 80 nM in assay medium and further diluted 1:2. FGF23 and anti-pERK antibodies were diluted in assay medium to a concentration of 40 nM and 400 nM, respectively. Wells were pretreated with antibodies for 10 minutes. KL polypeptide dilutions and FGF23 were added to corresponding wells and plates were incubated at 37° C., 5% CO₂ for 15 minutes. The supernatant in each well was replaced with 30 μL NP40 lysis buffer containing a protease and phosphatase inhibitor cocktail. HTRF detection steps were carried out by following manufacturer's advanced phospho-ERK (Thr202/Tyr204) cellular HTRF kit protocol (CiSBio).

### 9.1.5. SRE/ERK Luciferase Assay

HEK293.FGFR1KO.hFGFR1c.Sre-Luc or HEK293.delR1.FGFR1c.SRE reporter lines were plated, kept overnight in DMEM+10% FBS, and treated with a KL polypeptide of interest (in the presence or absence of FGF23) for 5 hours in DMEM or DMEM+10% FBS.

### 9.1.6. 3T3 Cell Proliferation Assay

3T3 cells were maintained in culture according to suppliers' recommendations. For the assay, the cells were seeded in 96-well plates in culture medium and cultured for 24-48 hours at 37° C., 5% CO₂ until use. Next, the cells were treated with a KL polypeptide or a control substance in culture medium. Cell proliferation was monitored using live-cell imaging (IncuCyte®) every three hours for 66 hours and was expressed as percent phase confluence. Untreated cells were used as controls.

### 9.1.7. Protease-Mediated In Vitro Cleavage of KL Polypeptide Constructs

KL polypeptide constructs with protease-cleavable linkers were cleaved enzymatically either by incubating the constructs with kallikrein 2 (KLK2) or matrix metalloproteinase 7 (MMP7) enzymes. For enzymatic cleavage with KLK2, protein constructs were incubated with KLK2 enzyme in reaction buffer (1×TBS+0.05% Brij-35) for 3 or 18 hours at 37° C. For enzymatic cleavage with MMP7, protein constructs were incubated with MMP7 at an MMP7: KL construct molar ratio of 4:1 in reaction buffer for one hour at 37° C.

### 9.1.8. Cisplatin-Induced Acute Kidney Injury Model

C57BL/6 mice were randomly assigned into treatment groups. Uninephroectomy was performed in all mice seven days (D-7) before induction of acute kidney injury with a single IP injection of 20 mg/kg cisplatin or vehicle on Day 0 (DO). Starting on the day before the induction of acute kidney injury (D-1), mice in KL polypeptide treatment groups received subcutaneous injections of 30 mg/kg KL958 (C370S C521S)-PCL1-HSA daily for four days and mice in control group have received 25 mg/kg of an hIgG4 isotype antibody as a size control, which was delivered in a single subcutaneous injection on D-1. Blood, bone and kidney tissues were collected on Day 4 (D4) for further analyses.

### 9.2. Example 1: Sequence Engineering of KL Polypeptides

In order to engineer the KL amino acid sequence for efficient production of bioactive KL polypeptides, one or more modifications were introduced to the full-length hKL sequence shown in FIG. 3, as described in Section 9.1.1. Resulting polypeptides were purified using His affinity columns followed by SEC as described in Section 9.1.2.1 and the bioactivity of the purified KL polypeptides was assessed using pERK HTRF assay as described in Section 9.1.4.

Four distinct KL peptide sequences, KL981, KL958, KL958 C521S, and KL958 C521S C910S were engineered as follows: KL981 was generated by replacing its native signal sequence corresponding to the first 33 amino acids with an mROR1 signal sequence (SS). KL958 was generated by truncating KL981 at the C-terminal to remove the C-terminal loop. KL958 C521S was generated by replacing the Cys residue at 521 of KL958 with a Ser. KL958 C521S C910S was generated by replacing the Cys residue at 910 of KL958 C521S with a Ser. A C-terminal His-tag was added to all four constructs.

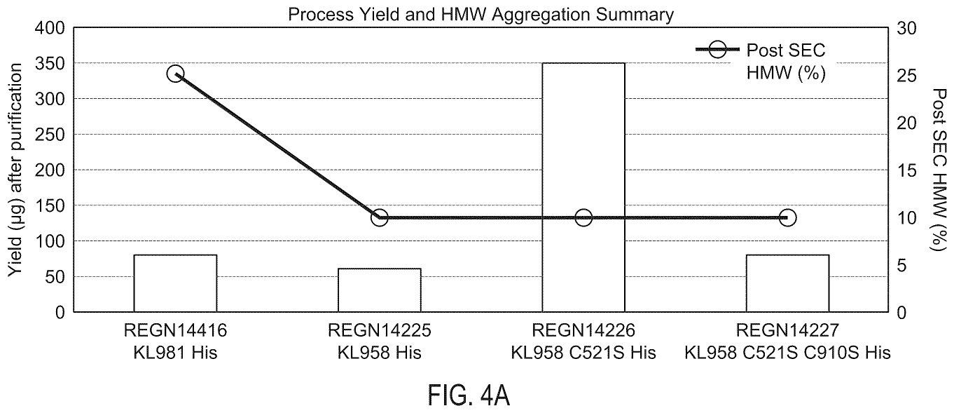

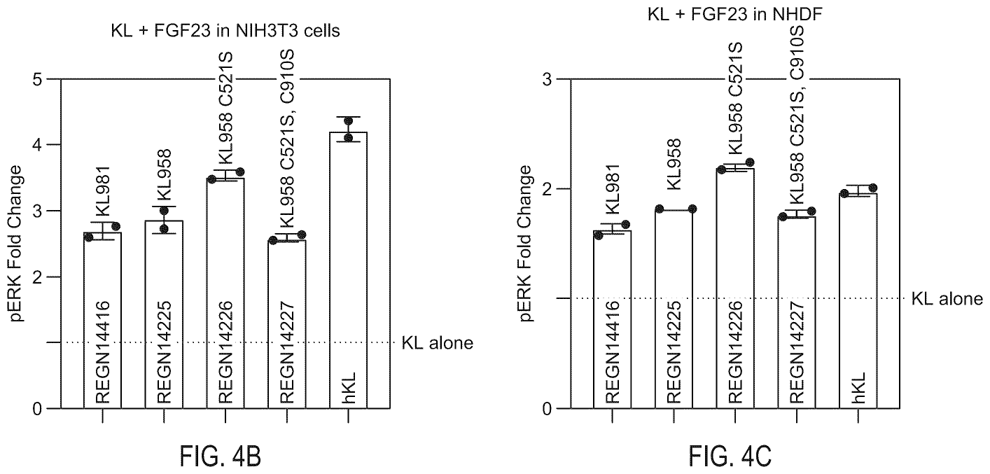

First, the yield and purity of each construct were evaluated. KL958 C521S was associated with ˜5-7 times higher yield than the other three constructs (FIG. 4A). The constructs truncated at the C-termini had a lower levels of high molecular weight species (HMW) than KL981 (FIG. 4A).

To determine whether sequence engineering could affect KL bioactivity, changes in phospho-ERK (PERK) levels were measured in NIH3T3 and NHDF cells upon KL and FGF23 co-treatment and quantified as fold changes in pERK relative to KL alone treatment. A positive control KL protein (hKL) was used for comparison. All four sequence-engineered constructs were associated with increased pERK fold change in NIH373 and NHDF cells (FIGS. 4B and 4C, respectively). Among the four engineered constructs, KL958 C521S displayed the highest pERK fold change in both cell lines and surpassed the pERK fold change observed with the positive control hKL protein.

In sum, C-terminal truncation combined with the C521S mutation not only resulted in relatively high yield of KL polypeptide with low levels of HMW, but it was also associated with enhanced bioactivity.

### 9.3. Example 2: Effect of Purification pH on KL Polypeptide Yield and Activity

In order to assess the effect of different affinity columns on the yield and activity of KL polypeptides, a double-tagged new construct, KL958 (C521S C910S)-HSA-His, was designed and produced as described in Section 9.1.1. Resulting polypeptides were purified using His or HSA affinity columns followed by SEC as described in Section 9.1.2.1 and the bioactivity of the purified KL polypeptides was assessed using PERK HTRF assay as described in Section 9.1.4.

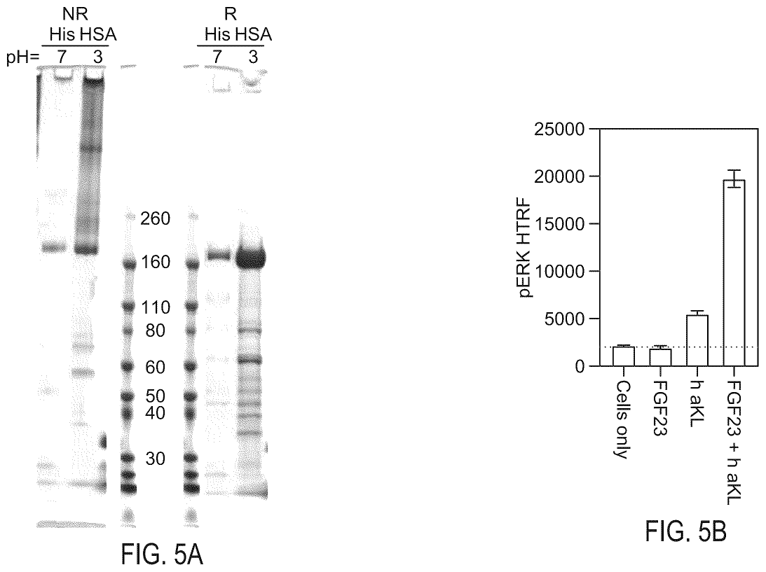

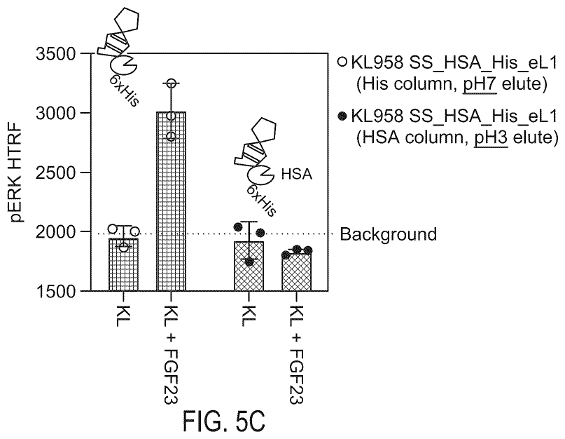

The yield and purity of the double-tagged KL polypeptide KL958 (C521S C910S)-HSA-His was evaluated with SDS-PAGE under non-reducing (NR) and reducing (R) conditions by loading 10 μL of purified polypeptide sample per well. Although HSA affinity column purification achieved a visibly higher yield (i.e., a much thicker band at ˜160 kDa under reducing conditions), it was associated with high molecular weight aggregates (i.e., a smear above ˜160 kDa under non-reducing conditions) (FIG. 5A) and displayed no activity, as the pERK HTRF signal of KL+FGF23 co-treatment was lower than the cells-only background signal (FIGS. 5B and 5C). Purification of the same construct with a His-affinity column resulted in a higher pERK HTRF signal in cells co-treated with FGF23 (FIG. 5C). Given that the elution buffer pH values differ, these results suggested that the acidic pH of the HSA elution buffer inactivated the purified KL polypeptide.

### 9.4. Example 3: Effect of Different C-Terminal Tags on Yield and Purity of KL Polypeptides

KL polypeptide constructs were designed by linking different C-terminal tags to REGN 14226 (KL958 C521S), and produced as described in Section 9.1.1. Resulting polypeptides were purified using Twin-Strep purification method as described in Section 9.1.2.2.

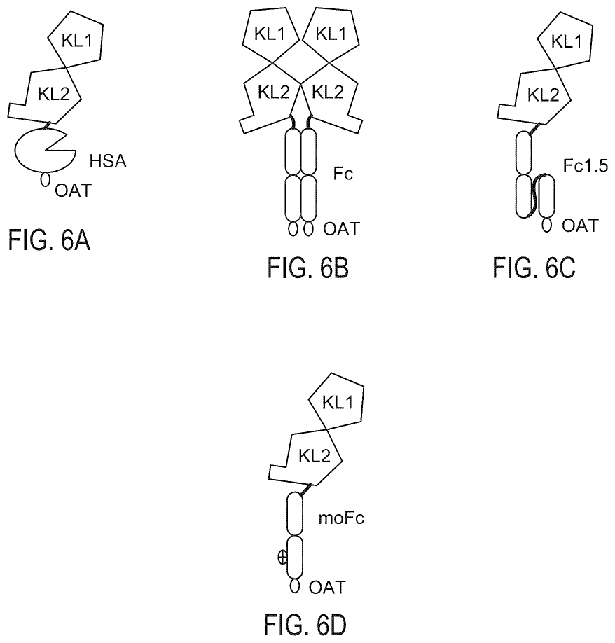

A set of four KL polypeptide constructs were utilized in this example. KL958 (C521S)-HSA comprised a C-terminal HSA and Twin-Strep-tag (FIG. 6A), KL958 (C521S)-Fc comprised a C-terminal Fc and Twin-Strep-tag (FIG. 6B); KL958 (C521S)-Fc1.5 comprised a C-terminal Fc1.5 and Twin-Strep-tag, wherein the Fc1.5 comprised one CH2 and two CH3 regions (FIG. 6C); and KL958 (C521S)-moFc comprised a C-terminal moFc and Twin-Strep-tag (FIG. 6D).

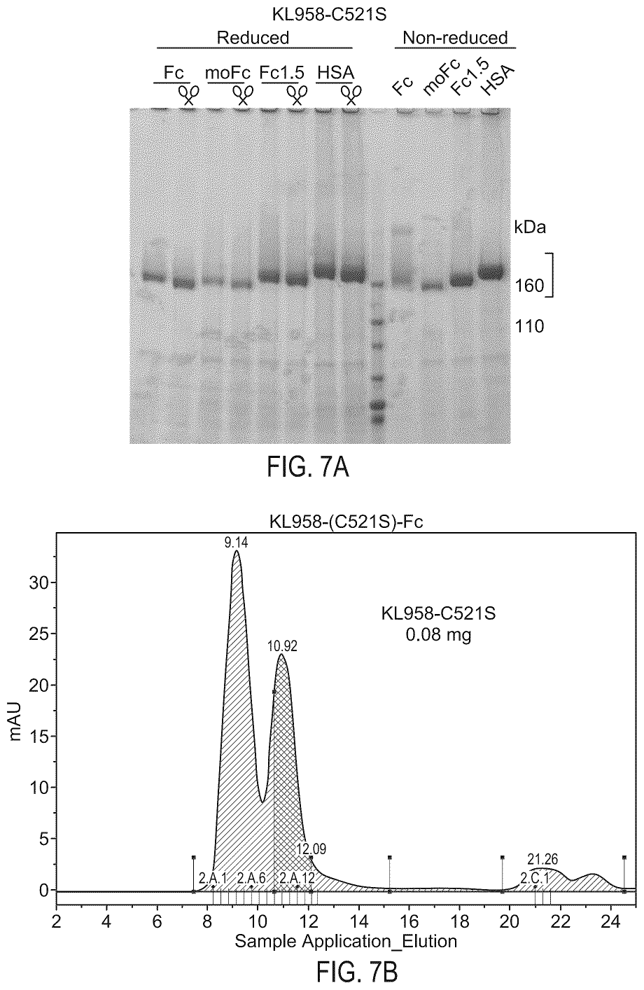

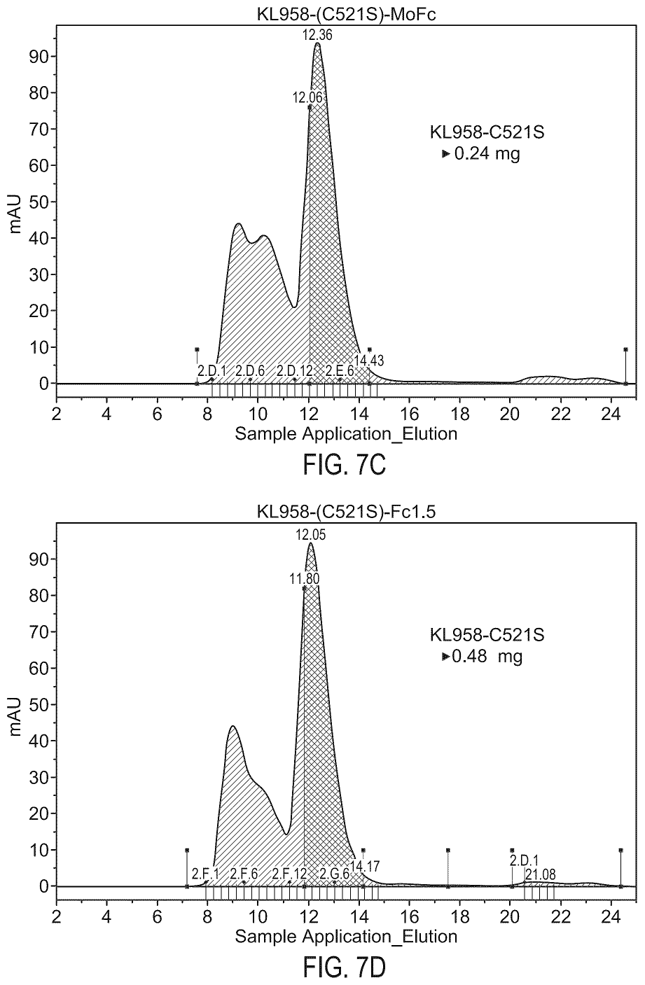

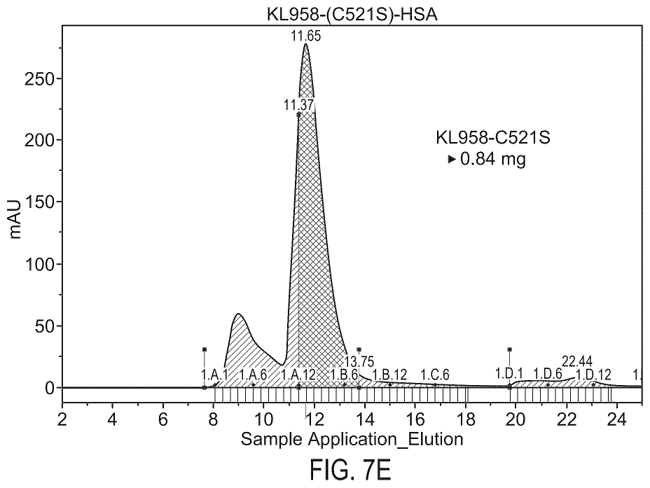

Following Twin-Strep purification, samples were treated with 3C protease to remove the Twin-Strep-tag. The yield and purity of the 3C-treated and untreated constructs were evaluated via SDS-PAGE under reducing and non-reducing conditions. 3C-protease treated and untreated KL polypeptide constructs resulted in slightly lower molecular weight bands with comparable thickness for each group, suggesting that the protease treatment successfully removed Twin-Strep-tag from each polypeptide construct (FIG. 7A). The highest yield was achieved with KL958 (C521S)-HSA and KL958 (C521S)-Fc1.5, whereas KL958 (C521S)-Fc was associated with high molecular weight aggregation (FIG. 7A).

The yield and purity of the KL polypeptide constructs comprising either an Fc, an Fc1.5, a moFc, or an HSA tag were also evaluated with SEC. Consistent with the SDS-PAGE results, the Fc-tagged KL polypeptide had the highest HMW peak, whereas the peak corresponding to the KL958 C521 polypeptide was relatively smaller (FIG. 7B). MoFc- and Fc1.5-tagged KI polypeptides had better yields than Fc-tagged polypeptide construct; however, the highest yield and lowest HMW levels were achieved with the HSA-tagged KL polypeptide construct (FIGS. 7C-7E).

### 9.5. Example 4: Enhancement of HSA-Tagged KL Polypeptide Purification

Experiments in Example 3 suggested that HSA-tagged KL polypeptide results in higher yields than KL polypeptides with other tags. Yet, the results of the experiments in Example 2 suggested that HSA affinity purification with a low pH elution buffer renders HSA-tagged KL polypeptides biologically inactive. The goal of the experiments in this present Example was to enhance the purification of biologically active KL958 (C521)-HSA polypeptides.

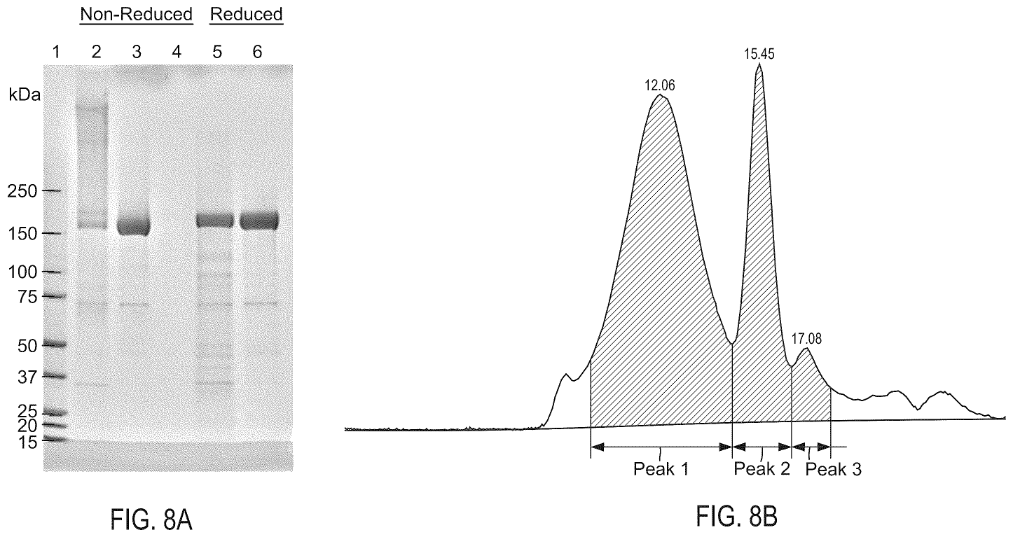

First, a modified high salt purification method was used as described in Section 9.1.2.3 by binding HSA-tagged KL polypeptides in a 500 ml of sample with an HSA Affinity resin, and eluting the proteins from the resin with a high salt buffer at pH 7.4. Next, HMW were removed with SEC, which accounted for approximately 75% of the total yield. SDS-PAGE was used to analyze the fractions that correspond to reduced and non-reduced HMW (peak 1 in FIG. 8B) and the eluted KL polypeptide (peak 2 in FIG. 8B). In non-reduced conditions, a thin band corresponding to the monomeric KL polypeptide was observed in the HMW lane, whereas in reduced conditions, the amount of monomeric KL polypeptide in the HMW lane was comparable to that of the eluted KL polypeptide lane (FIG. 8A), suggesting most the polypeptides in the HMW fraction were aggregates of KL polypeptides. The final yield of monomeric KL polypeptides purified with this method was 8 mg (16 mg/L).

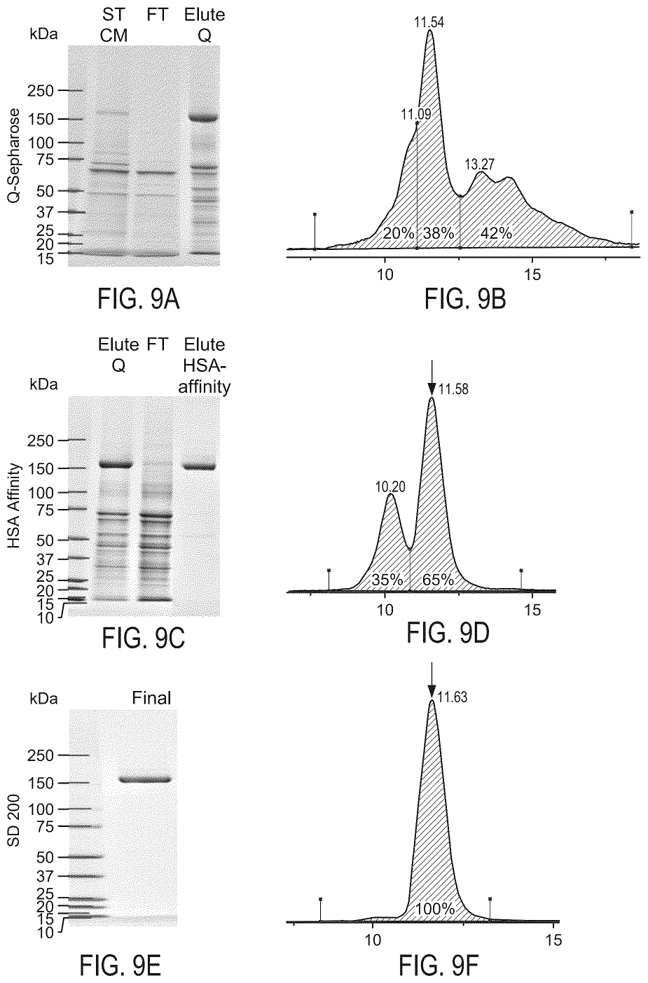

In order to further enhance the large scale purification of KL polypeptides, a modified ion exchange (IEX) purification method was used as described in Section 9.1.2.4, which involved three steps: a Q-Sepharose step to concentrate the KL polypeptides (FIGS. 9A and 9B), followed by capturing of KL polypeptides with an HSA affinity resin (FIGS. 9C and 9D), which was then followed by SD-200 step to further remove any remaining contaminants (FIGS. 9E and 9F). Purification of KL958 (C521)-HSA from a 25L sample with this approach resulted in approximately 5 mg/L monomeric KL polypeptides.

### 9.6. Example 5: Pharmacokinetic Profiles of His- and HSA-Tagged KL Polypeptides

Pharmacokinetic properties of His-tagged and HSA-tagged KL polypeptides were assessed in mice as described in Section 9.1.3. Briefly, mice received a single injection of one of the three doses of KL958 (C521)-HSA (3, 10, or 30 mg/kg) or 20 mg/kg KL958 (C521)-His. A positive control group of mice was dosed with a single injection of 3 mg/kg control mAb.

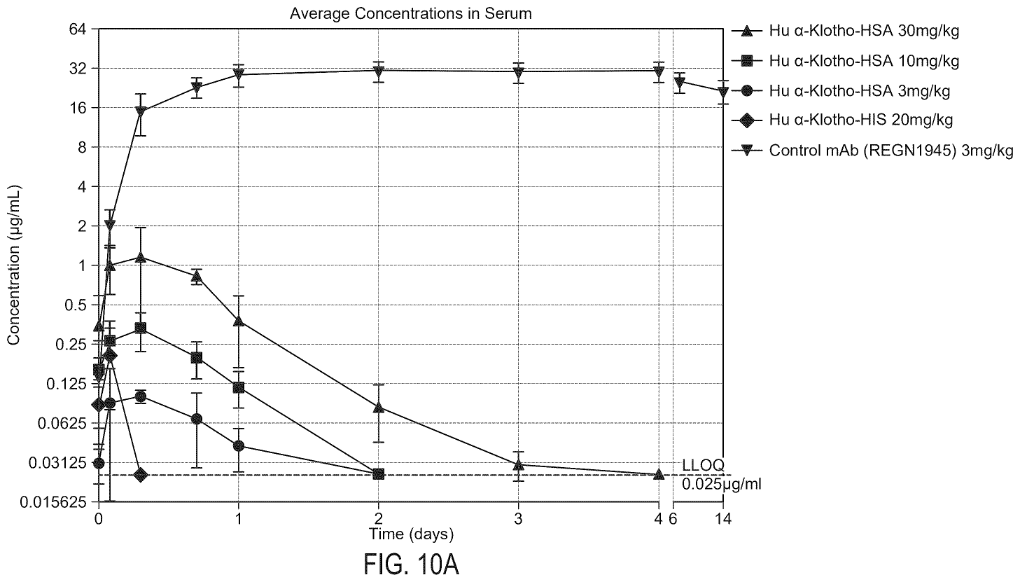

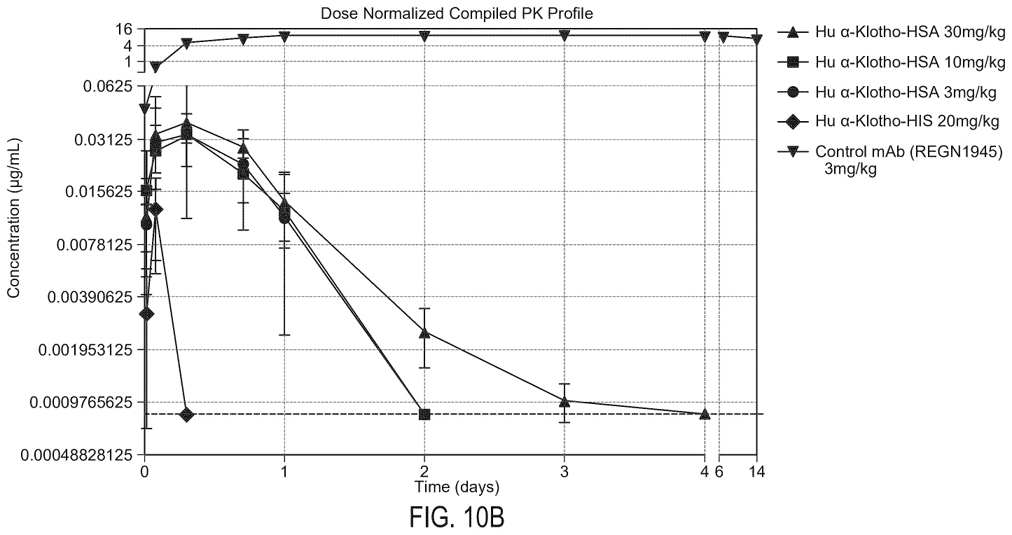

By 8 hours after dosing, KL958 (C521)-His was undetectable, suggesting that this polypeptide was cleared relatively rapidly. KL958 (C521)-HSA exhibited a slower clearance than the His-tagged KL polypeptide at every dose evaluated (FIG. 10A). Dose-normalization of the data revealed that although HSA-tagged KL polypeptide exhibited similar clearance with all three doses, clearance rates were slightly faster at 3 and 10 mg/kg after 24 hours (FIG. 10B).

### 9.7. Example 6: Effects of His- and HSA-Tagged KL Polypeptides on 3T3 Cell Proliferation

The proliferative effect of His-tagged and HSA-tagged KL polypeptides KL958 (C521)-His and KL958 (C521)-HSA was evaluated as described in Section 9.1.6.

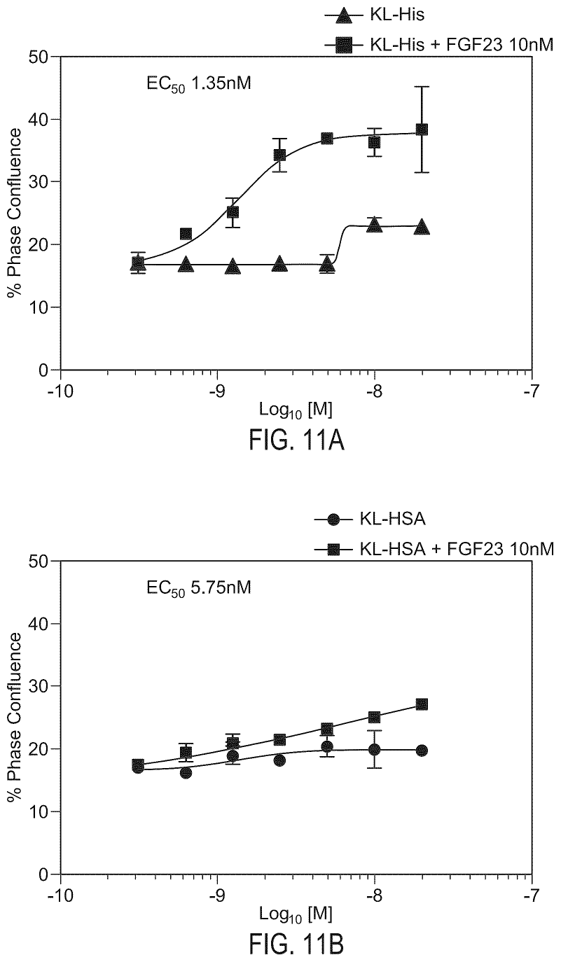

Both KL polypeptides promoted 3T3 cell proliferation when co-incubated with 10 nM FGF23. Yet, the potency of KL958 (C521)-His was greater than that of KL958 (C521)-HSA (FIGS. 11A and 11B). Next, the same concentration (20 nM) of KL958 (C521)-His and KL958 (C521)-HSA was added to cells with or without 10 nM FGF23. In the presence of FGF23, both KL polypeptides resulted in 3T3 cell proliferation, but the percent phase confluence was more pronounced with the KL958 (C521)-His construct (FIG. 11C).

### 9.8. Example 7: Differences in C-Terminal Truncation Affect the Activity of KL Polypeptides

The experiments in the previous Examples assessed the activity of KL958 polypeptides that were derived from the full-length human KL981 by truncating the 23 amino acids at the C-terminus. To determine whether a construct with a slightly different length would result in a construct with similar activity, a KL polypeptide with a 20 amino acid truncation at the C-terminus, KL961 (C521S)-HSA, was designed and produced as described in Section 9.1.1. The activity of KL958 (C521S)-HSA and KL961 (C521S)-HSA was measured as described in Section 9.1.5.

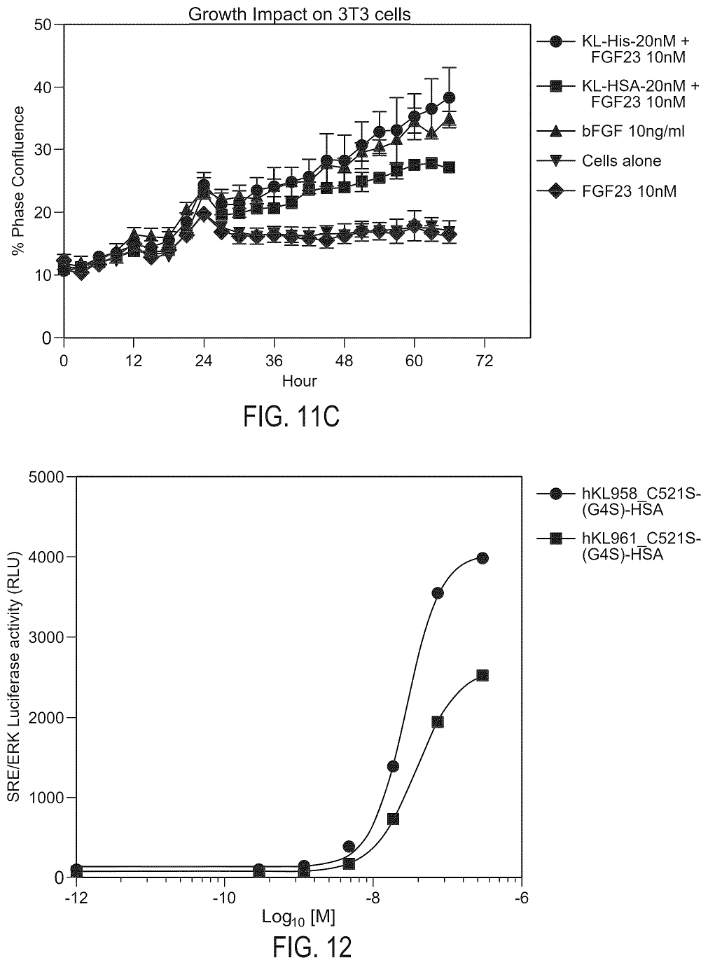

Both KL958 (C521S)-HSA and KL961 (C521S)-HSA displayed activity; however, the signal magnitude was higher with the KL958 (C521S)-HSA (FIG. 12). Similarly, the potency of KL958 (C521S)-HSA was higher with an EC50 value of 2.8E-08 M, which was approximately an order of magnitude higher than the EC50 value obtained with KL961 (C521S)-HSA.

### 9.9. Example 8: Targeted Sequence Engineering to Enhance KL Polypeptide Yield and Activity

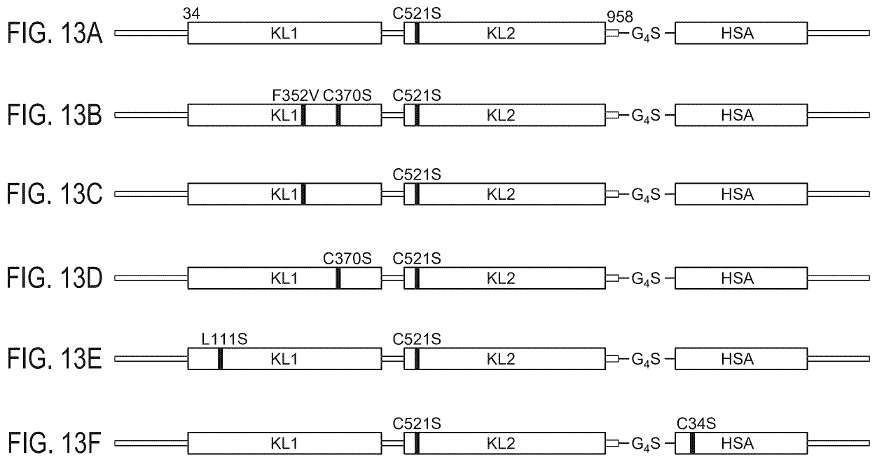

A new set of KL polypeptides were designed by introducing additional mutations to HSA-tagged KL958 C521S (FIGS. 13A-13F) and produced as described in Section 9.1.1 and purified as described in Section 9.1.2.2. The activity of the construct KL958 (C521S C370S)-G4S-HSA (FIG. 13D), was evaluated as described in Section 9.1.5. and compared to the activity of KL958 (C521)-G4S-HSA (FIG. 13A).

KL958 (C521S C370S)-G4S-HSA had a lower EC50 value and better yield relative to KL958 (C521)-G4S-HSA (Table E3).

TABLE E3
Targeted Sequence Engineering Associated
Changes in Potency and Expression

| | | Expression | | |
|---|---|---|---|---|
| | | level (rel. | | |
| | Yield | to KL958 | EC50 | EC50 |
| Construct | (mg/L) | (C521)-HSA) | (trial 1) | (trial 2) |
| KL958 (C521S)- | 4 | 1 | 11.9E−06 | 11.1E−06 |
| G₄S-HSA | | | | |
| KL958 (C521S | 8.1 | ~2 | 9.7E−6 | 9.8E−06 |
| C370S)-G₄S-HSA | | | | |

### 9.10. Example 9: Effect of Linker on KL Polypeptide Yield and Activity

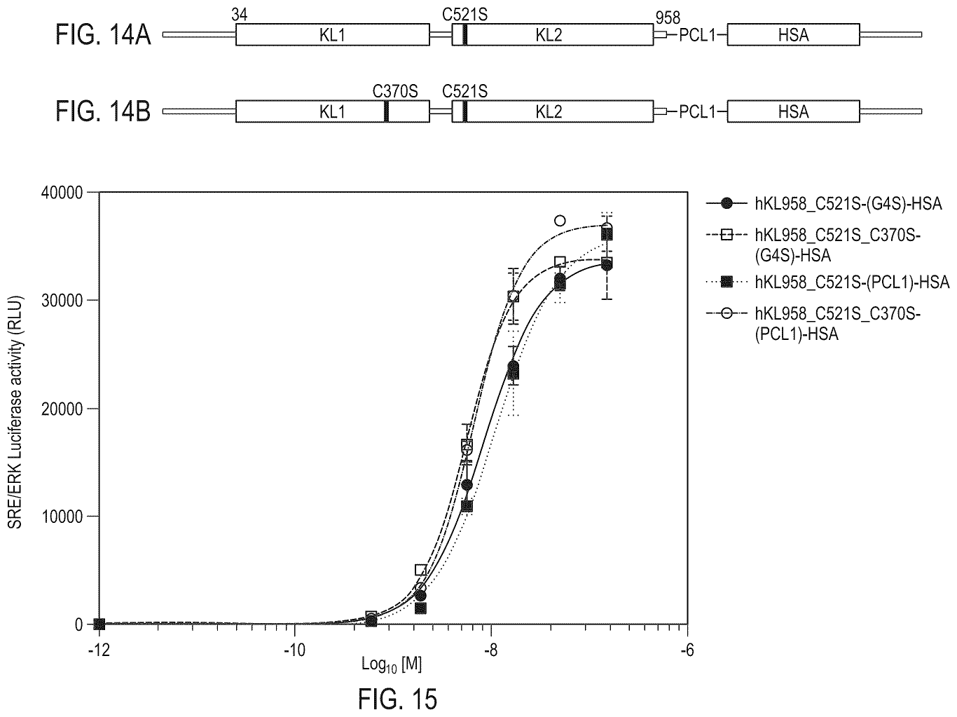

Two additional KL polypeptide constructs, KL958 (C521S)-PCL1-HSA (FIG. 14A) and KL958 (C521S C370S)-PCL1-HSA (FIG. 14B) were designed by replacing the G4S linkers with the PCL1 linker sequence in two KL polypeptide constructs, KL958 (C521S)-G4S-HSA and KL958 (C521S C370S)-G4S-HSA, respectively. The KL polypeptide constructs were produced and purified as described in Sections 9.1.1 and 9.1.2.2. The activity of the new constructs with PCL1 linkers and their G4S-linker comprising counterparts was evaluated as described in Section 9.1.5.

KL polypeptide constructs with both C521S and C370S mutations had better yields relative to those with only C521 mutations (Table E4). All four KL polypeptide constructs displayed comparable SRE-ERK luciferase activities and EC50 values (FIG. 15 and Table E4).

TABLE E4
Linker-Associated Changes in Potency and Expression

| | | Expression level | |
|---|---|---|---|
| | Yield | (rel. to KL958 | |
| Construct | (mg/L) | (C521)-HSA) | EC50 |
| KL958 (C521S)- | 4 | 1 | 8.3E−09 |
| G₄S-HSA | | | |
| KL958 (C521S | 8.1 | ~2 | 5.6E−09 |
| C370S)-G₄S-HSA | | | |
| KL958 (C521S)- | 3.4 | ~0.9 | 1.1E−08 |
| PCL1-HSA | | | |
| KL958 (C521S | 8.4 | ~2.1 | 6.7E−09 |
| C370S)-PCL1-HSA | | | |

### 9.11. Example 10: In Vitro Cleavage of KL Polypeptide Constructs

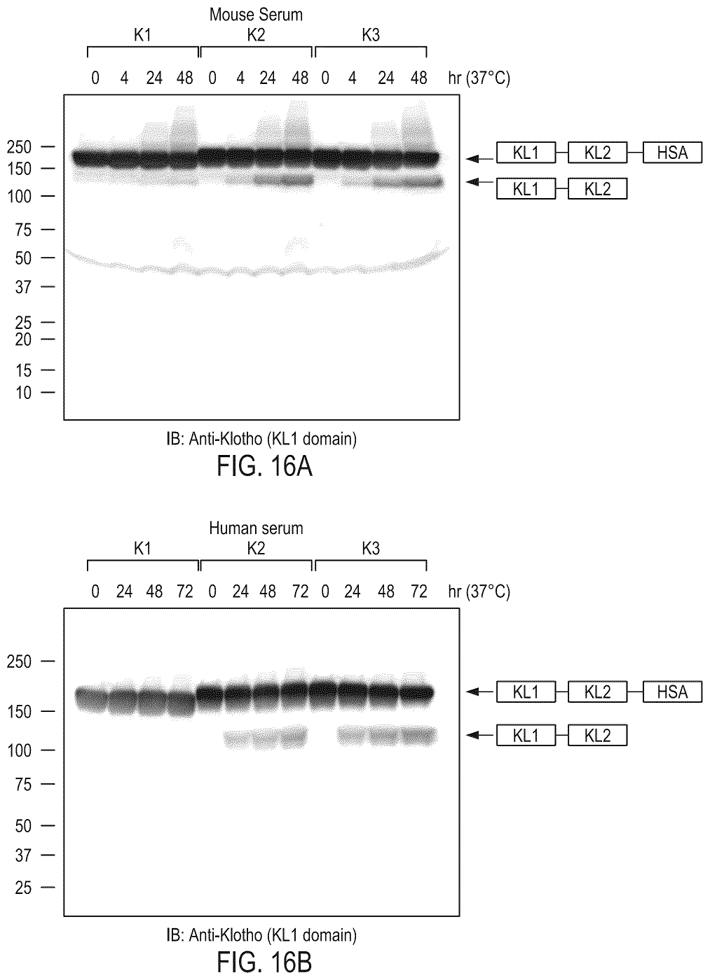

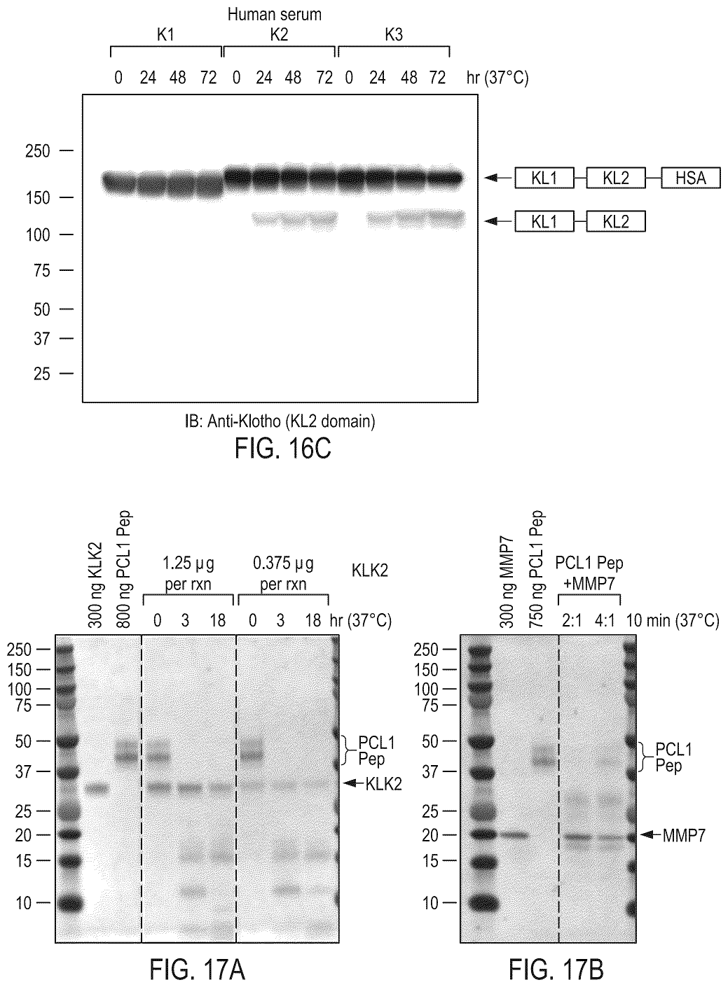

KL polypeptide constructs KL958 C521S-(G4S)-HSA (referred to as K1 in FIGS. 16-18), KL958 (C521S)-PCL1-HSA (referred to as K2 in FIGS. 16-18), and KL958 (C521S C370S)-PCL1-HSA (referred to as K3 in FIGS. 16-18), were incubated by adding 1 μg of KL peptide construct in 4 μL (˜75 μg) mouse or human serum for 4, 24, or 48 hours at 37° C. and assessed with western blot with anti-KL antibodies against KL1 or KL2 domains. An increase in cleaved KL polypeptide with longer incubations was observed with both KL958 (C521S)-PCL1-HSA and KL958 (C521S C370S)-PCL1-HSA in mouse serum (FIG. 16A) and human serum (FIGS. 16A-16C).

The ability of two proteases, kallikrein 2 (KLK2) and matrix metalloproteinase 7 (MMP7), to cleave the protease cleavable linker PCL1 was confirmed using a control peptide comprising PCL1 (PCL1 Pep) as described in Section 9.1.7 (FIGS. 17A and 17B, respectively).

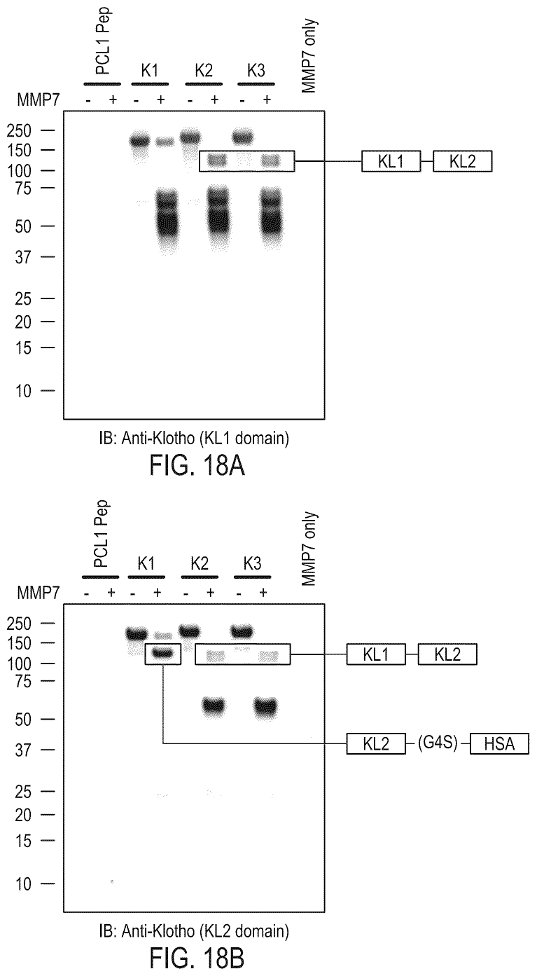

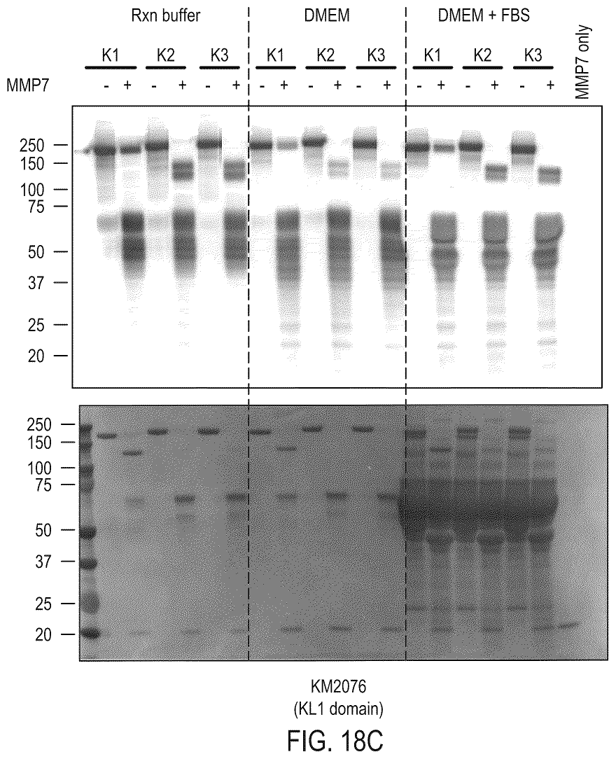

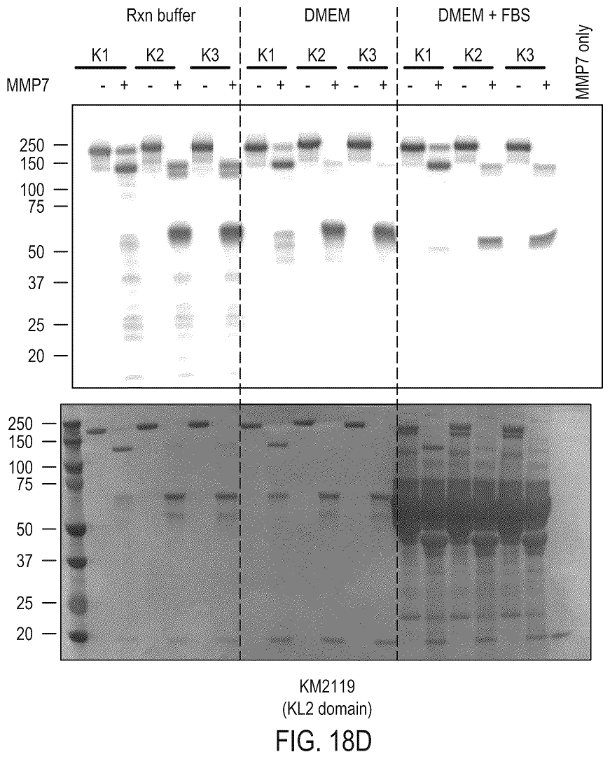

Cleavage of PCL1 by MMP7 was further assessed as described in Section 7.1.7, this time using KL958 (C521S)-PCL1-HSA and KL958 (C521S C370S)-PCL1-HSA (which comprise PCL1) and the control KL polypeptide construct KL958 C521S-(G4S)-HSA (which comprises a noncleavable G4S linker). When the cleavage reaction was carried out in reaction buffer, PCL1-containing KL polypeptides displayed cleavage of the linker, resulting in a shorter KL polypeptide in the presence of MMP7 (FIGS. 18A-18D). When DMEM was used instead of the reaction buffer, cleavage of the KL958 (C521S)-PCL1-HSA and KL958 (C521S C370S)-PCL1-HSA by MMP7 was reduced compared to reaction buffer. Addition of 10% FBS to DMEM increased cleavage of the PCL1-containing KL polypeptides compared to DMEM alone (FIGS. 18C and 18D).

### 9.12. Example 11: Effect of Protease Cleavage Conditions on the Activity of KL Polypeptides

The effect of MMP7 cleavage in DMEM with or without 10% FBS on the activity of the PCL1-containing KL polypeptide construct KL958 (C521S)-PCL 1-HSA relative to the activity of noncleavable linker-containing KL polypeptide construct KL958 (C521)-G4S-HSA incubated with MMP7 under the same conditions, was evaluated using HEK293.delR1.FGFR 1c.SRE reporter cells as described in Section 9.1.5.

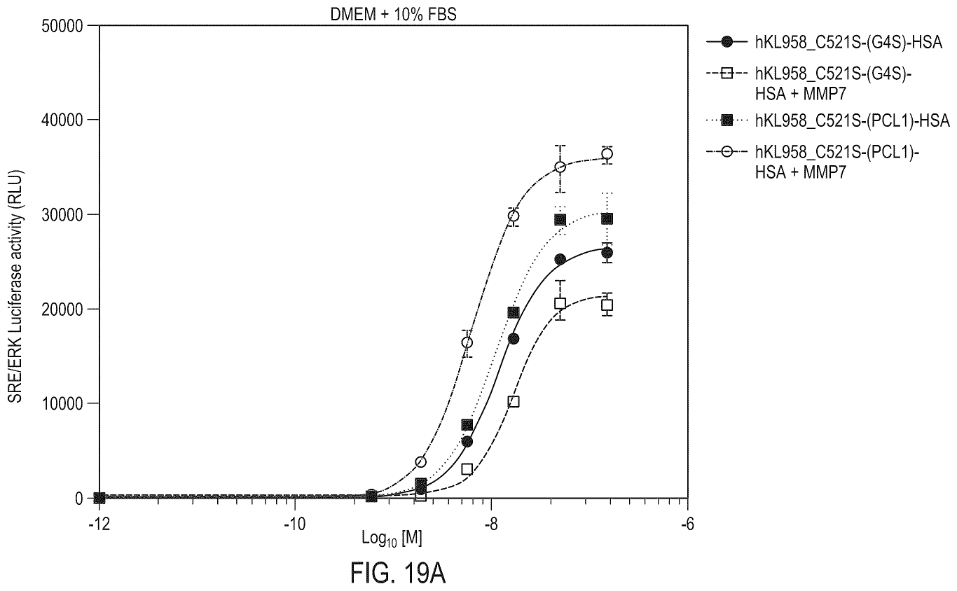

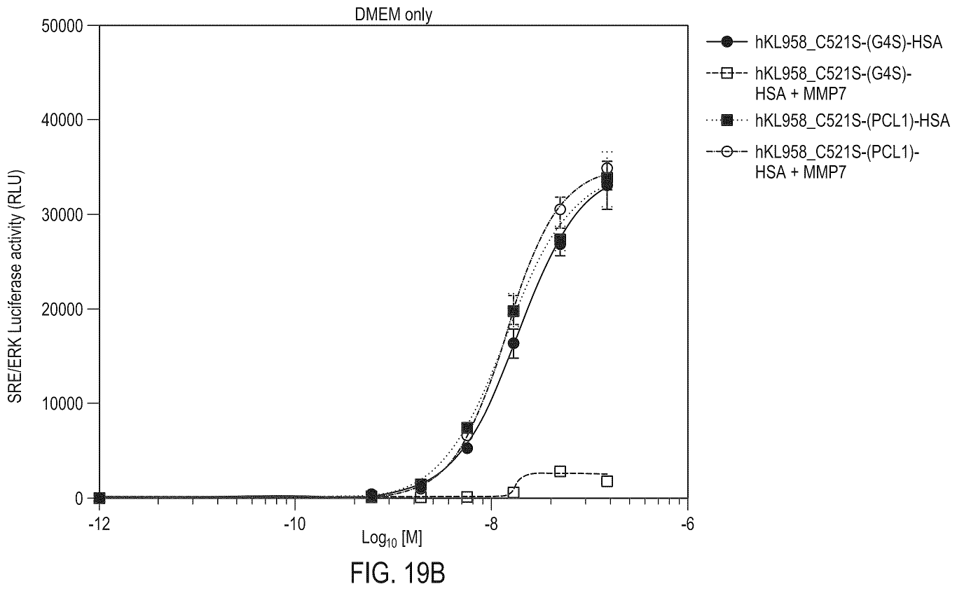

MMP7 cleavage resulted in a significant increase in the activity of the PCL1-containing KL polypeptide construct KL958 (C521S)-PCL1-HSA when the reaction was carried out in DMEM+10% FBS (FIG. 19A). This effect of MMP7 was reduced when carried out in DMEM only (FIG. 19B).

### 9.13. Example 12: Effects of PCL1-containing KL Polypeptides on 3T3 Cell Proliferation

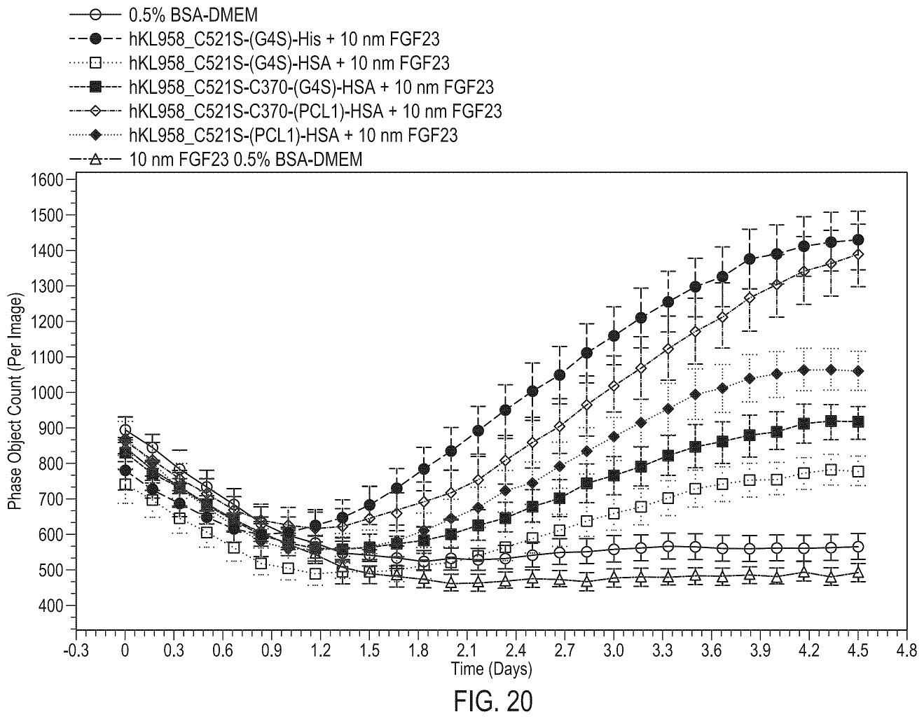

The proliferative effect of various KL polypeptides was evaluated as described in Section 9.1.6. All KL polypeptide constructs promoted varying levels of 373 cell proliferation when co-incubated with 10 nM FGF23, as shown in FIG. 20. Constructs labeled with “C370” in FIG. 20 contain a C370S mutation.

### 9.14. Example 13: Efficacy of an PCL1-containing KL Polypeptide in an Acute Kidney Injury Model

The KL polypeptide KL958 (C370S C521S)-PCL1-HSA was evaluated in a cisplatin-induced acute kidney injury model described in Section 9.1.8. Treatment groups are shown in Table E5.

TABLE E5

| Treatment | | | | Dose | |
|---|---|---|---|---|---|
| Group | Surgery | Model | Treatment | (mg/kg) | N |
| 1 | Uninephroectomy | Vehicle | Isotype | 25 | 6 |
| | | control | antibody | | |
| 2 | Uninephroectomy | Vehicle | KL polypeptide | 30 | 4 |
| | | control | | | |
| 3 | Uninephroectomy | Cisplatin | Isotype | 25 | 11 |
| | | | antibody | | |
| 4 | Uninephroectomy | Cisplatin | KL polypeptide | 30 | 11 |

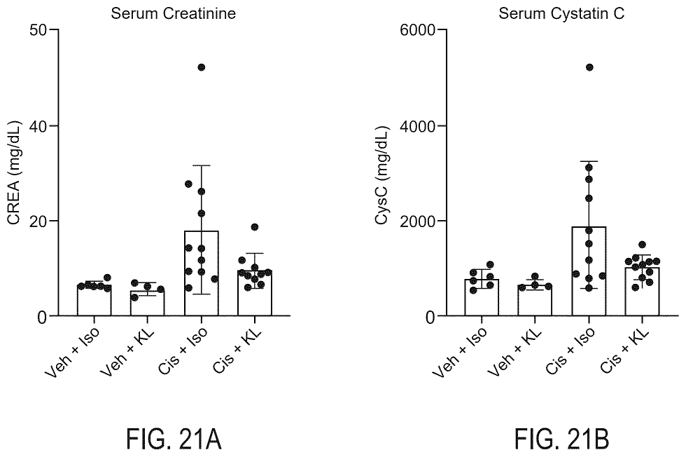

Kidney function in mice was assessed by determining the serum levels of creatinine and cystatin C. Mice that received cisplatin and were treated with isotype antibody had the highest levels of serum creatinine (FIG. 21A) and cystatin C (FIG. 21B), suggesting reduced kidney filtering. Mice that received cisplatin and were treated with KL958 (C370S C521S)-PCL1-HSA had levels of serum creatinine and cystatin C comparable to vehicle control mice (FIGS. 21A and 21B), suggesting that KL958 (C370S C521S)-PCL1-HSA was able to prevent cisplatin-induced reduction in kidney function.

1. A polypeptide comprising an alpha klotho moiety operably linked to a stabilization moiety via a protease cleavable linker.

2. The polypeptide of claim 1, wherein the alpha klotho moiety:
(a) comprises an alpha klotho KL2 domain having (i) at least about 80% sequence identity to SEQ ID NO:3 and (ii) an amino acid substitution at the position corresponding to amino acid C521 of SEQ ID NO:1; and
(b) lacks a cysteine at the amino acid corresponding to amino acid C970 of SEQ ID NO: 1, if present.

3. The polypeptide of claim 2, wherein the alpha klotho KL2 domain has at least about 99% sequence identity to SEQ ID NO:4.

4. (canceled)

5. The polypeptide of claim 2, wherein the alpha klotho moiety further comprises an alpha klotho KL1 domain having at least about 80% sequence identity to SEQ ID NO:9.

6. (canceled)

7. (canceled)

8. The polypeptide of claim 5, wherein the alpha klotho KL1 domain has at least about 99% sequence identity to the amino acid sequence of SEQ ID NO:10 or SEQ ID NO:71.

9.-11. (canceled)

12. The polypeptide of claim 5, wherein the alpha klotho KL1 domain comprises a cysteine to serine substitution at the position corresponding to position C370 of SEQ ID NO:1.

13. The polypeptide of claim 1, wherein the amino acid substitution at the position corresponding to amino acid C521 of SEQ ID NO: 1 is a cysteine to serine mutation.

14. The polypeptide of claim 1, wherein the alpha klotho moiety lacks an amino acid corresponding to amino acid C970 of SEQ ID NO: 1.

15.-18. (canceled)

19. The polypeptide of claim 1, wherein the alpha klotho moiety comprises a C-terminal deletion of at least 12 amino acids as compared to the amino acid sequence of SEQ ID NO: 13.

20.-23. (canceled)

24. The polypeptide of claim 1, wherein the alpha klotho moiety comprises a C-terminal deletion of between 20 and 23 amino acids as compared to the amino acid sequence of SEQ ID NO: 13.

25. The polypeptide of claim 1, wherein the alpha klotho moiety comprises or consists of an amino acid sequence having at least about 95% sequence identity to SEQ ID NO: 11 or SEQ ID NO: 12.

26.-28. (canceled)

29. The polypeptide of claim 1, wherein the alpha klotho moiety comprises or consists of an amino acid sequence having at least about 99.5% sequence identity to SEQ ID NO: 73 or SEQ ID NO:74.

30.-32. (canceled)

33. The polypeptide of claim 1, wherein the alpha klotho moiety comprises or consists of an amino acid sequence having at least about 95% sequence identity to SEQ ID NO: 15, SEQ ID NO: 16, or SEQ ID NO:76.

34.-37. (canceled)

38. The polypeptide of claim 1, wherein the alpha klotho moiety comprises or consists of the amino acid sequence of SEQ ID NO: 16.

39. The polypeptide of claim 1, wherein the alpha klotho moiety comprises or consists of the amino acid sequence of SEQ ID NO:76.

40. (canceled)

41. The polypeptide of claim 1, wherein the stabilization moiety is C-terminal to the alpha klotho moiety.

42. (canceled)

43. The polypeptide of claim 1, wherein the stabilization moiety is an albumin moiety.

44.-46. (canceled)

47. A polypeptide comprising:
(a) an amino acid sequence having at least 80% sequence identity to SEQ ID NO: 16, wherein the position corresponding to amino acid 488 of SEQ ID NO: 16 is not a cysteine, and which lacks an amino acid sequence having at least 80% sequence identity to SEQ ID NO:8;
(b) a protease cleavable linker; and
(c) a stabilization moiety.

48. The polypeptide of claim 47, wherein the position corresponding to amino acid 488 of SEQ ID NO: 16 is a serine.

49. The polypeptide of claim 47, wherein the amino acid sequence has at least 99% sequence identity to SEQ ID NO: 16.

50. (canceled)

51. The polypeptide of claim 47, wherein the amino acid sequence lacks the amino acid sequence of SEQ ID NO:8.

52. The polypeptide of claim 1, which comprises an amino acid sequence having at least 90% sequence identity to SEQ ID NO:68.

53. (canceled)

54. The polypeptide of claim 52, which comprises the amino acid sequence of SEQ ID NO:68.

55. The polypeptide of claim 1, which comprises an amino acid sequence having at least 90% sequence identity to SEQ ID NO:70.

56. (canceled)

57. The polypeptide of claim 55, which comprises the amino acid sequence of SEQ ID NO:70.

58. (canceled)

59. The polypeptide of claim 1, wherein the protease cleavable linker comprises the amino acid sequence of SEQ ID NO:90.

60.-62. (canceled)

63. A nucleic acid encoding the polypeptide of claim 1.

64. A host cell engineered to express the polypeptide of claim 1.

65. A method of producing a polypeptide of, comprising culturing the host cell of claim 64 and recovering the polypeptide expressed thereby.

66. (canceled)

67. A pharmaceutical composition comprising the polypeptide of claim 1 and an excipient.

68. A method of activating FGFR signaling in a cell, the method comprising contacting the cell with the polypeptide of claim 1.

69. The method of claim 68, wherein the cell is a kidney cell.

70.-76. (canceled)

77. A method of treating a subject suffering from an age-related condition, of preventing an age-related condition comprising, of treating a subject suffering from kidney disease, or of preventing kidney disease, the method comprising administering to the subject the polypeptide of claim 1.


SEQUENCE LISTING (440 sequences)

SEQ ID NO: 1 - protein, 1012 aa, Homo sapiens [sequence omitted]

SEQ ID NO: 2 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 3 - protein, 436 aa, Homo sapiens
LEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDS

SEQ ID NO: 4 - protein, 436 aa, synthetic construct
LEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDS

SEQ ID NO: 5 - protein, 467 aa, Homo sapiens
LEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKS

SEQ ID NO: 6 - protein, 467 aa, synthetic construct
LEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKS

SEQ ID NO: 7 - protein, 31 aa, Homo sapiens
NGFPGPETLERFCPEEFTVCTECSFFHTRKS

SEQ ID NO: 8 - protein, 23 aa, Homo sapiens
LERFCPEEFTVCTECSFFHTRKS

SEQ ID NO: 9 - protein, 473 aa, Homo sapiens
EPGDGAQTWARVSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGF

SEQ ID NO: 10 - protein, 473 aa, Homo sapiens
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGF

SEQ ID NO: 11 - protein, 917 aa, Homo sapiens
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDS

SEQ ID NO: 12 - protein, 917 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDS

SEQ ID NO: 13 - protein, 948 aa, Homo sapiens
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKS

SEQ ID NO: 14 - protein, 948 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKS

SEQ ID NO: 15 - protein, 925 aa, Homo sapiens
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPET

SEQ ID NO: 16 - protein, 925 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPET

SEQ ID NO: 17 - protein, 21 aa, Homo sapiens
LLAFIAFLFFASIISLSLIFY

SEQ ID NO: 18 - protein, 10 aa, Homo sapiens
YSKKGRRSYK

SEQ ID NO: 19 - protein, 585 aa, Homo sapiens [sequence omitted]

SEQ ID NO: 20 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 21 - protein, 8 aa, Homo sapiens
PPLPENQP

SEQ ID NO: 22 - protein, 8 aa, Homo sapiens
NGFPGPET

SEQ ID NO: 23 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 25 - protein, 217 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 26 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 28 - protein, 217 aa, synthetic construct
APELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 29 - protein, 217 aa, synthetic construct
APELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKGQCREPQVYTSPPSRDELTKNQVSLRCHVKGFYPSDIAVEWESNGQPENNYKTTKPVLDSDGSFFLYSKLTVDKSRWQQGNVFSCSVMHECLHNHYTQKSLSLSPGK

SEQ ID NO: 30 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTYPPSRDELTKNQVSLYCAVKGFYPSDIAVEWESNGQPENNYKTTRPVLDSDGSFRLMSALTVDKSRWQQGNVFSCSVMHEALHNHYTQKSLSLSPGK

SEQ ID NO: 31 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFELKSALTVDKSRWQQGNVFSCSVMHEALHNHYTQKSLSLSPGK

SEQ ID NO: 32 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 33 - protein, 359 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 354 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 349 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 344 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 339 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 334 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 329 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 324 aa, synthetic construct
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 2865 bp, synthetic construct
  source 1..2865: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctcaccatcaccatcaccattga

SEQ ID NO: 42 - protein, 954 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSHHHHHH

SEQ ID NO: 43 - DNA, 2796 bp, synthetic construct
  source 1..2796: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactcaccatcaccatcaccattga

SEQ ID NO: 44 - protein, 931 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETHHHHHH

SEQ ID NO: 45 - DNA, 2796 bp, synthetic construct
  source 1..2796: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactcaccatcaccatcaccattga

SEQ ID NO: 46 - protein, 931 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETHHHHHH

SEQ ID NO: 47 - DNA, 2796 bp, synthetic construct
  source 1..2796: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatcttagcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactcaccatcaccatcaccattga

SEQ ID NO: 48 - protein, 931 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLSGYFAYSFNDRTAPRFGLYRYAADQFEPKASMKHYRKIIDSNGFPGPETHHHHHH

SEQ ID NO: 49 - DNA, 4617 bp, synthetic construct
  source 1..4617: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaatttaccgtgtgtactgagtgcagtttttttcacacccgaaagtctggtggaggaggtagtgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatga

SEQ ID NO: 50 - protein, 1538 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 51 - DNA, 4548 bp, synthetic construct
  source 1..4548: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactggtggaggaggtagtgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatga

SEQ ID NO: 52 - protein, 1515 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 53 - DNA, 4551 bp, synthetic construct
  source 1..4551: mol_type = other DNA
gagcctggggacggcgctcagacctgggctcgtttttctcgtcctcctgctcctgaggctgctggcctcttccagggcacgttcccagacggctttttgtgggcggtgggttctgcggcctaccagacggaggggggttggcagcagcatggcaagggcgcctccatctgggatactttcacgcaccaccccctggctccgcctggcgattcccgcaacgcctccctgcccttaggagccccctctcccctgcagccggccaccggcgatgtagcgtccgatagctacaataacgtgttccgcgacaccgaggctttgagagagttgggcgttactcattaccgcttctctatcagttgggcccgcgtactgccgaacggttctgccggggtgccaaacagggaggggcttcgctactaccgccgcctgctggagcgtttacgggagctgggagtgcagcctgtcgttactctgtaccactgggacctcccgcagaggctgcaggacgcctatggaggctgggccaaccgcgcccttgcggaccattttcgggattacgcggaactgtgcttccgccacttcggtggccaggtgaagtattggatcaccattgacaatccgtatgtggtggcttggcatggatacgcgaccggtaggctggcccccggcatccgcgggagccctcggttgggctacctggtggcgcacaacctgctgttggcccacgctaaggtgtggcacctgtacaacacctcctttcgccccacccagggcggtcaggtgtccatcgccctcagctcccactggattaacccccggcgcatgaccgaccactccatcaaagaatgtcagaagtccctcgactttgtgctgggctggttcgccaagcccgtgtttattgacggtgactaccccgagtctatgaagaacaacctaagctcgatcctgcccgacttcacagagtctgagaaaaaatttattaagggcaccgcggacttcttcgcactctgctttggccctaccctgtcctttcagcttctggacccccacatgaaattcagacagctggagtcacccaacctgaggcagctgctgagttggatcgatctggaattcaatcaccctcagatcttcatcgtggagaatggatggttcgtctcaggcaccaccaagcgcgacgatgccaagtacatgtactatctgaagaaatttatcatggagacactcaaggcgatcaagctggacggcgtcgatgtgatcggttacaccgcttggtctttgatggatggcttcgaatggcaccgcggctactccatccgtcgcggactgttctacgtggatttcctgtctcaggacaaaatgctcctgccaaagtccagtgcactgttctatcagaagctaatcgagaaaaatggcttcccacctctgcctgagaaccagcccctcgaaggcaccttcccctcggacttcgcctggggcgtcgtggacaactatatacaagtggacaccacactttctcagtttactgacctgaacgtgtacctgtgggacgtgcaccactctaagcgacttatcaaggtggatggcgtggttactaagaagcgcaaatcatattgcgtggattttgcggccatccagcctcagatcgccctgctgcaggaaatgcacgtgacccactttcgattctctctcgactgggcactgattctgccacttggcaaccagagccaggtgaaccatacaatcctgcaatattacaggtgtatggccagcgagctggtgcgcgtgaacatcactccggtggtcgctctctggcaacccatggcccctaaccaaggcctgcctcgcctgctggctcgccagggtgcgtgggaaaacccgtacacggccctggccttcgccgagtacgcacgtctgtgcttccaggagctcggccaccacgtcaagttgtggatcaccatgaacgagccctacacccgtaatatgacctacagcgcaggccacaacctgttgaaggcccatgccctggcttggcacgtgtacaacgagaagttccgccacgcacagaacggtaagatctccatcgctcttcaggcggactggattgagccagcctgcccattctcgcagaaggacaaagaggtggccgaacgcgtcctcgaattcgacatcggttggctcgctgagcccatcttcggctccggcgactacccgtgggtgatgcgtgactggctgaaccagcgcaacaacttcctgctgccctatttcaccgaggacgagaagaaactgatacagggaaccttcgactttctagcactgtcgcactacacgacaatcctggtggacagcgagaaggaggacccgatcaagtacaacgactacctggaggtgcaggagatgaccgacatcacctggctgaactcgcccagccaggtcgccgtggttccgtggggcctgcgcaaggtactgaactggctgaagttcaagtacggcgacctgcccatgtatatcatctccaacgggattgacgatgggctacatgctgaggatgaccagctgcgggtgtactacatgcagaactacatcaatgaggctcttaaggctcacattctagacgggattaacttatcggggtacttcgcgtactccttcaacgaccgcacggccccgcgctttgggctctaccgctacgctgcggaccagttcgagccaaaagcgtccatgaagcactaccggaagataatcgacagcaatggcttccctggacccgagactgacgcacataagtccgaggtcgcccaccgcttcaaggacctgggcgaggagaatttcaaggccttggtcctcatcgccttcgcccagtatctgcagcagtgcccatttgaagatcacgtgaagctggtcaacgaggtgacggagttcgccaagacctgcgtggctgatgagagcgccgaaaactgcgataagagccttcacacactcttcggtgataagctgtgcaccgtggccaccctgcgcgagacgtacggcgagatggcagactgttgtgccaagcaggagcccgagagaaacgagtgctttctgcagcacaaagacgataatccaaacttgccgcgcctggtgcgtcccgaggtagatgtgatgtgcactgcgttccatgacaacgaggagactttcctgaagaagtatctctatgagatcgcccgccgccacccctatttctacgcgccggagctactttttttcgccaagcgttacaaggcggccttcaccgaatgctgtcaggctgccgacaaagctgcctgcctactgcctaagctggacgagctcagggacgagggcaaagcgagttctgctaagcagcgcctgaagtgcgcctccctgcagaagtttggggagcgcgctttcaaggcctgggcggtggcgcgcctgtcgcagcgttttcccaaagctgagttcgcggaggtgagcaaattggtaaccgacctgaccaaggtgcacactgaatgttgtcatggcgacctgctggagtgcgccgatgaccgcgccgacttagccaagtacatctgcgaaaatcaggactctatctcttctaagctcaaggaatgttgcgagaaacccctgctagagaagtcccactgtattgctgaggtcgagaacgacgagatgcccgccgacctgccctctctggccgccgactttgtcgagagcaaggacgtgtgcaagaactacgcggaggctaaggacgtgttcctcggcatgttcctgtacgagtacgcccgacggcaccccgactactccgtagtgcttttgctgcgtctggctaaaacctacgagactacccttgagaaatgttgtgcagctgcagacccgcatgagtgttacgccaaagtcttcgacgagttcaaacctctggtcgaagagcctcagaacctgatcaagcagaactgcgagctgttcgagcagctaggagagtacaagttccaaaatgctctgctggtgcgctacaccaagaaggtccctcaggtgtccaccccaaccctggtggaggtgtcgcgcaacctgggtaaggtgggctccaagtgttgcaagcacccggaggccaagcggatgccttgtgctgaggactacctgtccgtggtgttgaaccagctgtgcgtgttgcacgagaagactccggtgtccgatcgtgtgacgaaatgctgcactgagagtcttgtgaaccgcaggccttgcttttcagccctggaggtagacgagacttacgtgcccaaggagttcaacgctgagactttcacatttcacgccgacatctgcactcttagcgagaaggagcggcaaattaagaagcagacggcgctggttgaattagttaagcacaagccgaaggctaccaaggaacagctgaaggcggtgatggatgacttcgctgcattcgtcgagaaatgttgcaaagcggacgacaaggagacttgcttcgcagaggagggcaagaagctggtggctgcttcccaggccgccctggggctccatcaccatcatcaccactga

SEQ ID NO: 54 - protein, 1516 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLSGYFAYSFNDRTAPRFGLYRYAADQFEPKASMKHYRKIIDSNGFPGPETDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHHHHHH

SEQ ID NO: 55 - DNA, 3573 bp, synthetic construct
  source 1..3573: mol_type = other DNA
gagcctggggacggcgctcagacctgggctcgtttttctcgtcctcctgctcctgaggctgctggcctcttccagggcacgttcccagacggctttttgtgggcggtgggttctgcggcctaccagacggaggggggttggcagcagcatggcaagggcgcctccatctgggatactttcacgcaccaccccctggctccgcctggcgattcccgcaacgcctccctgcccttaggagccccctctcccctgcagccggccaccggcgatgtagcgtccgatagctacaataacgtgttccgcgacaccgaggctttgagagagttgggcgttactcattaccgcttctctatcagttgggcccgcgtactgccgaacggttctgccggggtgccaaacagggaggggcttcgctactaccgccgcctgctggagcgtttacgggagctgggagtgcagcctgtcgttactctgtaccactgggacctcccgcagaggctgcaggacgcctatggaggctgggccaaccgcgcccttgcggaccattttcgggattacgcggaactgtgcttccgccacttcggtggccaggtgaagtattggatcaccattgacaatccgtatgtggtggcttggcatggatacgcgaccggtaggctggcccccggcatccgcgggagccctcggttgggctacctggtggcgcacaacctgctgttggcccacgctaaggtgtggcacctgtacaacacctcctttcgccccacccagggcggtcaggtgtccatcgccctcagctcccactggattaacccccggcgcatgaccgaccactccatcaaagaatgtcagaagtccctcgactttgtgctgggctggttcgccaagcccgtgtttattgacggtgactaccccgagtctatgaagaacaacctaagctcgatcctgcccgacttcacagagtctgagaaaaaatttattaagggcaccgcggacttcttcgcactctgctttggccctaccctgtcctttcagcttctggacccccacatgaaattcagacagctggagtcacccaacctgaggcagctgctgagttggatcgatctggaattcaatcaccctcagatcttcatcgtggagaatggatggttcgtctcaggcaccaccaagcgcgacgatgccaagtacatgtactatctgaagaaatttatcatggagacactcaaggcgatcaagctggacggcgtcgatgtgatcggttacaccgcttggtctttgatggatggcttcgaatggcaccgcggctactccatccgtcgcggactgttctacgtggatttcctgtctcaggacaaaatgctcctgccaaagtccagtgcactgttctatcagaagctaatcgagaaaaatggcttcccacctctgcctgagaaccagcccctcgaaggcaccttcccctcggacttcgcctggggcgtcgtggacaactatatacaagtggacaccacactttctcagtttactgacctgaacgtgtacctgtgggacgtgcaccactctaagcgacttatcaaggtggatggcgtggttactaagaagcgcaaatcatattgcgtggattttgcggccatccagcctcagatcgccctgctgcaggaaatgcacgtgacccactttcgattctctctcgactgggcactgattctgccacttggcaaccagagccaggtgaaccatacaatcctgcaatattacaggtgtatggccagcgagctggtgcgcgtgaacatcactccggtggtcgctctctggcaacccatggcccctaaccaaggcctgcctcgcctgctggctcgccagggtgcgtgggaaaacccgtacacggccctggccttcgccgagtacgcacgtctgtgcttccaggagctcggccaccacgtcaagttgtggatcaccatgaacgagccctacacccgtaatatgacctacagcgcaggccacaacctgttgaaggcccatgccctggcttggcacgtgtacaacgagaagttccgccacgcacagaacggtaagatctccatcgctcttcaggcggactggattgagccagcctgcccattctcgcagaaggacaaagaggtggccgaacgcgtcctcgaatttgacatcggttggctcgctgagcccatcttcggctccggcgactacccgtgggtgatgcgtgactggctgaaccagcgcaacaacttcctgctgccctatttcaccgaggacgagaagaaactgatacagggaaccttcgactttctagcactgtcgcactacacgacaatcctggtggacagcgagaaggaggacccgatcaagtacaacgactacctggaggtgcaggagatgaccgacatcacctggctgaactcgcccagccaggtcgccgtggttccgtggggcctgcgcaaggtactgaactggctgaagttcaagtacggcgacctgcccatgtatatcatctccaacgggattgacgatgggctacatgctgaggatgaccagctgcgggtgtactacatgcagaactacatcaatgaggctcttaaggctcacattctagacgggattaacttatgcgggtacttcgcgtactccttcaacgaccgcacggccccgcgctttgggctctaccgctacgctgcggaccagttcgagccaaaagcgtccatgaagcactaccggaagataatcgacagcaatggcttccctggacccgagactggatccggaggcggttcggcccccgaggccgctggcggcccttccgtgttcctgttccccccaaagcccaaggacaccctgatgatctctcgcacccctgaggtgacctgcgtggtcgttgatgtgagccacgaagatccagaggttaagttcaactggtacgtggacggcgtcgaagtccacaatgccaagaccaagccgagggaggagcagtacaactccacgtaccgcgtggtgtccgttctaaccgtgcttcaccaggactggctaaacggcaaggagtacaagtgtaaggtgtcaaacaaggccctccctgcccccattgagaagaccatctcgaaggcgaagggacagccccgcgagcctcaggtctacacgctgcccccatcccgcgacgagctcactaagaaccaggtcagccttacttgcctggtgaagggcttttacccgtccgacatcgcggtagagtgggagagcaacggtcagccagagaataactacaagacgactcccccggtgctggacagtgacggctccttctttctgtattctaagctgacagtcgacaaaagccgctggcaacagggtaacgtgttctcgtgttctgtaatgcatgaggctctgcacaaccattacacccagaagagcctgtccctgtctcccggcaaaggttccctggaggtgctgttccaggggccaggcagcgcctggtcccacccccagttcgagaagggcggcggtagtggagggggcggatctggcggctcagcttggagccacccccagttcgaaaagtga

SEQ ID NO: 56 - protein, 1190 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETGSGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSLEVLFQGPGSAWSHPQFEKGGGSGGGGSGGSAWSHPQFEK

SEQ ID NO: 57 - DNA, 3573 bp, synthetic construct
  source 1..3573: mol_type = other DNA
gagcctggggacggcgctcagacctgggctcgtttttctcgtcctcctgctcctgaggctgctggcctcttccagggcacgttcccagacggctttttgtgggcggtgggttctgcggcctaccagacggaggggggttggcagcagcatggcaagggcgcctccatctgggatactttcacgcaccaccccctggctccgcctggcgattcccgcaacgcctccctgcccttaggagccccctctcccctgcagccggccaccggcgatgtagcgtccgatagctacaataacgtgttccgcgacaccgaggctttgagagagttgggcgttactcattaccgcttctctatcagttgggcccgcgtactgccgaacggttctgccggggtgccaaacagggaggggcttcgctactaccgccgcctgctggagcgtttacgggagctgggagtgcagcctgtcgttactctgtaccactgggacctcccgcagaggctgcaggacgcctatggaggctgggccaaccgcgcccttgcggaccattttcgggattacgcggaactgtgcttccgccacttcggtggccaggtgaagtattggatcaccattgacaatccgtatgtggtggcttggcatggatacgcgaccggtaggctggcccccggcatccgcgggagccctcggttgggctacctggtggcgcacaacctgctgttggcccacgctaaggtgtggcacctgtacaacacctcctttcgccccacccagggcggtcaggtgtccatcgccctcagctcccactggattaacccccggcgcatgaccgaccactccatcaaagaatgtcagaagtccctcgactttgtgctgggctggttcgccaagcccgtgtttattgacggtgactaccccgagtctatgaagaacaacctaagctcgatcctgcccgacttcacagagtctgagaaaaaatttattaagggcaccgcggacttcttcgcactctgctttggccctaccctgtcctttcagcttctggacccccacatgaaattcagacagctggagtcacccaacctgaggcagctgctgagttggatcgatctggaattcaatcaccctcagatcttcatcgtggagaatggatggttcgtctcaggcaccaccaagcgcgacgatgccaagtacatgtactatctgaagaaatttatcatggagacactcaaggcgatcaagctggacggcgtcgatgtgatcggttacaccgcttggtctttgatggatggcttcgaatggcaccgcggctactccatccgtcgcggactgttctacgtggatttcctgtctcaggacaaaatgctcctgccaaagtccagtgcactgttctatcagaagctaatcgagaaaaatggcttcccacctctgcctgagaaccagcccctcgaaggcaccttcccctcggacttcgcctggggcgtcgtggacaactatatacaagtggacaccacactttctcagtttactgacctgaacgtgtacctgtgggacgtgcaccactctaagcgacttatcaaggtggatggcgtggttactaagaagcgcaaatcatattgcgtggattttgcggccatccagcctcagatcgccctgctgcaggaaatgcacgtgacccactttcgattctctctcgactgggcactgattctgccacttggcaaccagagccaggtgaaccatacaatcctgcaatattacaggtgtatggccagcgagctggtgcgcgtgaacatcactccggtggtcgctctctggcaacccatggcccctaaccaaggcctgcctcgcctgctggctcgccagggtgcgtgggaaaacccgtacacggccctggccttcgccgagtacgcacgtctgtgcttccaggagctcggccaccacgtcaagttgtggatcaccatgaacgagccctacacccgtaatatgacctacagcgcaggccacaacctgttgaaggcccatgccctggcttggcacgtgtacaacgagaagttccgccacgcacagaacggtaagatctccatcgctcttcaggcggactggattgagccagcctgcccattctcgcagaaggacaaagaggtggccgaacgcgtcctcgaatttgacatcggttggctcgctgagcccatcttcggctccggcgactacccgtgggtgatgcgtgactggctgaaccagcgcaacaacttcctgctgccctatttcaccgaggacgagaagaaactgatacagggaaccttcgactttctagcactgtcgcactacacgacaatcctggtggacagcgagaaggaggacccgatcaagtacaacgactacctggaggtgcaggagatgaccgacatcacctggctgaactcgcccagccaggtcgccgtggttccgtggggcctgcgcaaggtactgaactggctgaagttcaagtacggcgacctgcccatgtatatcatctccaacgggattgacgatgggctacatgctgaggatgaccagctgcgggtgtactacatgcagaactacatcaatgaggctcttaaggctcacattctagacgggattaacttatgcgggtacttcgcgtactccttcaacgaccgcacggccccgcgctttgggctctaccgctacgctgcggaccagttcgagccaaaagcgtccatgaagcactaccggaagataatcgacagcaatggcttccctggacccgagactggatccggaggcggttcggcacctgaggccgccgggggtccctccgtgttcctgttcccacccaagcctaaggacaccctgatgatctcccgcactcctgaagtcacttgcgtggtggttgatgtaagtcatgaggacccggaggtgaaatttaattggtacgtggacggcgtggaggtgcacaacgccaagactaagccccgcgaggagcagtacaactccacctaccgggtcgtgtcggttcttacggtgctccaccaggactggctcaacggtaaggagtacaagtgcaaagtatctaacaaggcgctgcccgcccccattgagaaaaccatctctaaggctaagggacagccgcgcgaacctcaggtgtacacgagccctcccagccgcgacgagctgaccaagaaccaggtcagcctgaggtgtcacgtgaagggcttctatccatccgacatcgcggtggaatgggagagcaatggccagccagagaacaactacaagaccacaaagccggtgctagattctgatggctccttcttcctgtattccaagctgactgtggacaagtcccgttggcaacagggcaacgtcttttcatgctctgtgatgcacgaggctttgcacaaccattacacccagaagagcctttcgctgtcccccggcaaaggcagcctggaagtgctgttccagggacctggcagcgcctggagccaccctcagtttgagaagggcggcggaagcggcggaggaggctctggcggcagcgcctggtcccacccccagttcgagaagtga

SEQ ID NO: 58 - protein, 1190 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETGSGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGKGSLEVLFQGPGSAWSHPQFEKGGGSGGGGSGGSAWSHPQFEK

SEQ ID NO: 59 - DNA, 3954 bp, synthetic construct
  source 1..3954: mol_type = other DNA
gagcctggggacggcgctcagacctgggctcgtttttctcgtcctcctgctcctgaggctgctggcctcttccagggcacgttcccagacggctttttgtgggcggtgggttctgcggcctaccagacggaggggggttggcagcagcatggcaagggcgcctccatctgggatactttcacgcaccaccccctggctccgcctggcgattcccgcaacgcctccctgcccttaggagccccctctcccctgcagccggccaccggcgatgtagcgtccgatagctacaataacgtgttccgcgacaccgaggctttgagagagttgggcgttactcattaccgcttctctatcagttgggcccgcgtactgccgaacggttctgccggggtgccaaacagggaggggcttcgctactaccgccgcctgctggagcgtttacgggagctgggagtgcagcctgtcgttactctgtaccactgggacctcccgcagaggctgcaggacgcctatggaggctgggccaaccgcgcccttgcggaccattttcgggattacgcggaactgtgcttccgccacttcggtggccaggtgaagtattggatcaccattgacaatccgtatgtggtggcttggcatggatacgcgaccggtaggctggcccccggcatccgcgggagccctcggttgggctacctggtggcgcacaacctgctgttggcccacgctaaggtgtggcacctgtacaacacctcctttcgccccacccagggcggtcaggtgtccatcgccctcagctcccactggattaacccccggcgcatgaccgaccactccatcaaagaatgtcagaagtccctcgactttgtgctgggctggttcgccaagcccgtgtttattgacggtgactaccccgagtctatgaagaacaacctaagctcgatcctgcccgacttcacagagtctgagaaaaaatttattaagggcaccgcggacttcttcgcactctgctttggccctaccctgtcctttcagcttctggacccccacatgaaattcagacagctggagtcacccaacctgaggcagctgctgagttggatcgatctggaattcaatcaccctcagatcttcatcgtggagaatggatggttcgtctcaggcaccaccaagcgcgacgatgccaagtacatgtactatctgaagaaatttatcatggagacactcaaggcgatcaagctggacggcgtcgatgtgatcggttacaccgcttggtctttgatggatggcttcgaatggcaccgcggctactccatccgtcgcggactgttctacgtggatttcctgtctcaggacaaaatgctcctgccaaagtccagtgcactgttctatcagaagctaatcgagaaaaatggcttcccacctctgcctgagaaccagcccctcgaaggcaccttcccctcggacttcgcctggggcgtcgtggacaactatatacaagtggacaccacactttctcagtttactgacctgaacgtgtacctgtgggacgtgcaccactctaagcgacttatcaaggtggatggcgtggttactaagaagcgcaaatcatattgcgtggattttgcggccatccagcctcagatcgccctgctgcaggaaatgcacgtgacccactttcgattctctctcgactgggcactgattctgccacttggcaaccagagccaggtgaaccatacaatcctgcaatattacaggtgtatggccagcgagctggtgcgcgtgaacatcactccggtggtcgctctctggcaacccatggcccctaaccaaggcctgcctcgcctgctggctcgccagggtgcgtgggaaaacccgtacacggccctggccttcgccgagtacgcacgtctgtgcttccaggagctcggccaccacgtcaagttgtggatcaccatgaacgagccctacacccgtaatatgacctacagcgcaggccacaacctgttgaaggcccatgccctggcttggcacgtgtacaacgagaagttccgccacgcacagaacggtaagatctccatcgctcttcaggcggactggattgagccagcctgcccattctcgcagaaggacaaagaggtggccgaacgcgtcctcgaatttgacatcggttggctcgctgagcccatcttcggctccggcgactacccgtgggtgatgcgtgactggctgaaccagcgcaacaacttcctgctgccctatttcaccgaggacgagaagaaactgatacagggaaccttcgactttctagcactgtcgcactacacgacaatcctggtggacagcgagaaggaggacccgatcaagtacaacgactacctggaggtgcaggagatgaccgacatcacctggctgaactcgcccagccaggtcgccgtggttccgtggggcctgcgcaaggtactgaactggctgaagttcaagtacggcgacctgcccatgtatatcatctccaacgggattgacgatgggctacatgctgaggatgaccagctgcgggtgtactacatgcagaactacatcaatgaggctcttaaggctcacattctagacgggattaacttatgcgggtacttcgcgtactccttcaacgaccgcacggccccgcgctttgggctctaccgctacgctgcggaccagttcgagccaaaagcgtccatgaagcactaccggaagataatcgacagcaatggcttccctggacccgagactggatccggaggcggttcggcccccgaggccgctggcggcccttccgtgttcctgttccccccaaagcccaaggacaccctgatgatctctcgcacccctgaggtgacctgcgtggtcgttgatgtgagccacgaagatccagaggttaagttcaactggtacgtggacggcgtcgaagtccacaatgccaagaccaagccgagggaggagcagtacaactccacgtaccgcgtggtgtccgttctaaccgtgcttcaccaggactggctaaacggcaaggagtacaagtgtaaggtgtcaaacaaggccctccctgcccccattgagaagaccatctcgaaggcgaagggacagccccgcgagcctcaggtctacacgctgcccccatcccgcgacgagctcactaagaaccaggtcagccttacttgcctggtgaagggcttttacccgtccgacatcgcggtagagtgggagagcaacggtcagccagagaataactacaagacgactcccccggtgctggacagtgacggctccttctttctgtattctaagctgacagtcgacaaaagccgctggcaacagggtaacgtgttctcgtgttctgtaatgcatgaggctctgcacaaccattacacccagaagagcctgtccctgtctcccggcaaaggaggcggcgggtccgggggcggcggttcgggcggtggtggctccgggggtggaggttctggacagccgcgtgaacctcaggtgtacacccttcccccctctcgggacgagctcaccaaaaaccaggtgtccctgacctgcctggtaaagggcttctatccgtccgacatcgccgtggagtgggagagtaatgggcagccggagaacaactataaaaccacacctcccgtgctggacagcgatggctccttcttcctgtactccaagttgactgtcgataaatcgcgatggcagcagggcaacgtgttttcttgctctgtgatgcacgaagcactgcacaaccactacacccagaagtctttgtccctgagcccggggaaaggttccctggaggtgctgttccaggggccaggcagcgcctggtcccacccccagttcgagaagggcggcggtagtggagggggcggatctggcggctcagcttggagccacccccagttcgaaaagtga

SEQ ID NO: 60 - protein, 1317 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETGSGGGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSLEVLFQGPGSAWSHPQFEKGGGSGGGGSGGSAWSHPQFEK

SEQ ID NO: 61 - DNA, 8681 bp, synthetic construct
  source 1..8681: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactggtggaggaggtagtgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctatggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactggtggaggaggtagtgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttactggaagtgctgttccagggacctggcagcgcctggagccaccctcagtttgagaagggcggcggaagcggcggaggaggctctggcggcagcgcctggtcccacccccagttcgagaagtga

SEQ ID NO: 62 - protein, 1558 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGSLEVLFQGPGSAWSHPQFEKGGGSGGGGSGGSAWSHPQFEK

SEQ ID NO: 63 - DNA, 4548 bp, synthetic construct; identical to SEQ ID NO: 51
  source 1..4548: mol_type = other DNA

SEQ ID NO: 64 - protein, 1515 aa, synthetic construct; identical to SEQ ID NO: 52

SEQ ID NO: 65 - DNA, 4548 bp, synthetic construct
  source 1..4548: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttcctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactggtggaggaggtagtgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatga

SEQ ID NO: 66 - protein, 1515 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 67 - DNA, 4749 bp, synthetic construct
  source 1..4749: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaacttctagactgcgggcctacctgctgcctgctccacctgcccctggcaacgccagcgagtctgaggaagatagaagcgccggcagcgtggaatcccctagcgtctctagcacccatagagtgtccgaccccaagttccaccccctgcacagcaagatcatcatcatcaagaaaggccacgccaaggacagccagcggtacaaggtggactacgagagcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatga

SEQ ID NO: 68 - protein, 1582 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETSRLRAYLLPAPPAPGNASESEEDRSAGSVESPSVSSTHRVSDPKFHPLHSKIIIIKKGHAKDSQRYKVDYESDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 69 - DNA, 4749 bp, synthetic construct
  source 1..4749: mol_type = other DNA
gagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttcctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttcccagcgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaacttctagactgcgggcctacctgctgcctgctccacctgcccctggcaacgccagcgagtctgaggaagatagaagcgccggcagcgtggaatcccctagcgtctctagcacccatagagtgtccgaccccaagttccaccccctgcacagcaagatcatcatcatcaagaaaggccacgccaaggacagccagcggtacaaggtggactacgagagcgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatga

SEQ ID NO: 70 - protein, 1582 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETSRLRAYLLPAPPAPGNASESEEDRSAGSVESPSVSSTHRVSDPKFHPLHSKIIIIKKGHAKDSQRYKVDYESDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 71 - protein, 473 aa, synthetic construct
EPGDGAQTWARVSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGF

SEQ ID NO: 72 - protein, 473 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGF

SEQ ID NO: 73 - protein, 917 aa, synthetic construct; identical to SEQ ID NO: 12

SEQ ID NO: 74 - protein, 917 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDS

SEQ ID NO: 75 - protein, 948 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKS

SEQ ID NO: 76 - protein, 925 aa, synthetic construct
EPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPSDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPET

SEQ ID NO: 77-89 - skipped (13 entries)

SEQ ID NO: 90 - protein, 72 aa, synthetic construct
SRLRAYLLPAPPAPGNASESEEDRSAGSVESPSVSSTHRVSDPKFHPLHSKIIIIKKGHAKDSQRYKVDYES

SEQ ID NO: 91 - protein, 35 aa, synthetic construct
GGGISSGLLSGRSDNHGGGISSGLLSGRSDNHGGS

SEQ ID NO: 92 - protein, 70 aa, synthetic construct
GGGISSGLLSGRSDNHGGGISSGLLSGRSDNHGGSGGGISSGLLSGRSDNHGGGISSGLLSGRSDNHGGS

SEQ ID NO: 93 - protein, 35 aa, synthetic construct
GGSGGSIPVSLRSGGGISSGLLSGRSDNHGGSGGS

SEQ ID NO: 94 - protein, 35 aa, synthetic construct
GGSGGSVPLSLYSGGGISSGLLSGRSDNHGGSGGS

SEQ ID NO: 95 - protein, 35 aa, synthetic construct
GGSHPVGLLARGGGHPVGLLARGGGHPVGLLARGS

SEQ ID NO: 96 - protein, 36 aa, synthetic construct
GGSHPVGLLARGGGHPVGLLARGGSGRSAGGSGRSA

SEQ ID NO: 97 - protein, 18 aa, synthetic construct
AVGLLAPPGGLSGRSANI

SEQ ID NO: 98 - protein, 18 aa, synthetic construct
AVGLLAPPGGLSGRSANP

SEQ ID NO: 99 - protein, 18 aa, synthetic construct
AVGLLAPPGGLSGRSDDH

SEQ ID NO: 100 - protein, 18 aa, synthetic construct
AVGLLAPPGGLSGRSDIH

SEQ ID NO: 101 - protein, 18 aa, synthetic construct
AVGLLAPPGGLSGRSDNH

SEQ ID NO: 102 - protein, 18 aa, synthetic construct
AVGLLAPPGGLSGRSDNI

SEQ ID NO: 103 - protein, 18 aa, synthetic construct
AVGLLAPPGGLSGRSDNP

SEQ ID NO: 104 - protein, 18 aa, synthetic construct
AVGLLAPPGGLSGRSDQH

SEQ ID NO: 105 - protein, 18 aa, synthetic construct
AVGLLAPPGGLSGRSDTH

SEQ ID NO: 106 - protein, 18 aa, synthetic construct
AVGLLAPPGGLSGRSDYH

SEQ ID NO: 107 - protein, 22 aa, synthetic construct
AVGLLAPPGGTSTSGRSANPRG

SEQ ID NO: 108 - protein, 17 aa, synthetic construct
AVGLLAPPSGRSANPRG

SEQ ID NO: 109 - protein, 18 aa, synthetic construct
AVGLLAPPTSGRSANPRG

SEQ ID NO: 110 - protein, 30 aa, synthetic construct
GGALFKSSFPGPAGLYAQPLAQKLKSSGGK

SEQ ID NO: 111 - protein, 33 aa, synthetic construct
GGGGSGGGGSGGGGSFVGGTGGGGSGGGGSGGS

SEQ ID NO: 112 - protein, 34 aa, synthetic construct
GGGGSGGGGSGGGGSISSGLLSGRSDNHGGSGGS

SEQ ID NO: 113 - protein, 34 aa, synthetic construct
GGGGSGGGGSGGGGSVPLSLYSGGGSGGSGGSGS

SEQ ID NO: 114 - protein, 35 aa, synthetic construct
GGGGSGGGGSGPLGLWSQGGGGSGGGGSGGGGSGG

SEQ ID NO: 115 - protein, 33 aa, synthetic construct
GGGGSGGGGSKKAAPGGGGSGGGGSGGGGSGGS

SEQ ID NO: 116 - protein, 33 aa, synthetic construct
GGGGSGGGGSKKAAPVNGGGGGSGGGGSGGGGS

SEQ ID NO: 117 - protein, 33 aa, synthetic construct
GGGGSGGGGSPMAKKGGGGSGGGGSGGGGSGGS

SEQ ID NO: 118 - protein, 33 aa, synthetic construct
GGGGSGGGGSPMAKKVNGGGGGSGGGGSGGGGS

SEQ ID NO: 119 - protein, 33 aa, synthetic construct
GGGGSGGGGSQARAKGGGGSGGGGSGGGGSGGS

SEQ ID NO: 120 - protein, 33 aa, synthetic construct
GGGGSGGGGSQARAKVNGGGGGSGGGGSGGGGS

SEQ ID NO: 121 - protein, 33 aa, synthetic construct
GGGGSGGGGSRQARVVNGGGGGSGGGGSGGGGS

SEQ ID NO: 122 - protein, 41 aa, synthetic construct
GGGGSGGGGSRQARVVNGGGGGSVPLSLYSGGGGGSGGGGS

SEQ ID NO: 123 - protein, 36 aa, synthetic construct
GGGGSGGGGSRQARVVNSVPLSLYSGGGGGSGGGGS

SEQ ID NO: 124 - protein, 33 aa, synthetic construct
GGGGSGGGGSVHMPLGFLGPGGGGSGGGGSGGS

SEQ ID NO: 125 - protein, 28 aa, synthetic construct
GGGGSVHMPLGFLGPGRSRGSFPGGGGS

SEQ ID NO: 126 - protein, 33 aa, synthetic construct
GGGGSVHMPLGFLGPPMAKKGGGGSGGGGSGGS

SEQ ID NO: 127 - protein, 32 aa, synthetic construct
GGGGSVHMPLGFLGPRQARVVNGGGGSGGGGS

SEQ ID NO: 128 - protein, 34 aa, synthetic construct
GGGGSVHMPLGFLGPRQARVVNGGGGSGGGGSGG

SEQ ID NO: 129 - protein, 30 aa, synthetic construct
GGPLAQKLKSSALFKSSFPGPAGLYAQGGR

SEQ ID NO: 130 - protein, 19 aa, synthetic construct
GLSGRSDNHGGAVGLLAPP

SEQ ID NO: 131 - protein, 21 aa, synthetic construct
GLSGRSDNHGGVHMPLGFLGP

SEQ ID NO: 132 - protein, 13 aa, synthetic construct
ISSGLLSGRSANI

SEQ ID NO: 133 - protein, 13 aa, synthetic construct
ISSGLLSGRSANP

SEQ ID NO: 134 - protein, 15 aa, synthetic construct
ISSGLLSGRSANPRG

SEQ ID NO: 135 - protein, 13 aa, synthetic construct
ISSGLLSGRSDDH

SEQ ID NO: 136 - protein, 13 aa, synthetic construct
ISSGLLSGRSDIH

SEQ ID NO: 137 - protein, 13 aa, synthetic construct
ISSGLLSGRSDNH

SEQ ID NO: 138 - protein, 13 aa, synthetic construct
ISSGLLSGRSDNI

SEQ ID NO: 139 - protein, 13 aa, synthetic construct
ISSGLLSGRSDNP

SEQ ID NO: 140 - protein, 13 aa, synthetic construct
ISSGLLSGRSDQH

SEQ ID NO: 141 - protein, 13 aa, synthetic construct
ISSGLLSGRSDTH

SEQ ID NO: 142 - protein, 13 aa, synthetic construct
ISSGLLSGRSDYH

SEQ ID NO: 143 - protein, 13 aa, synthetic construct
ISSGLLSGRSGNH

SEQ ID NO: 144 - protein, 22 aa, synthetic construct
ISSGLLSSGGSGGSLSGRSDNH

SEQ ID NO: 145 - protein, 22 aa, synthetic construct
ISSGLLSSGGSGGSLSGRSGNH

SEQ ID NO: 146 - protein, 30 aa, synthetic construct
KGGPGGPAGIGPLAQRLRSSALFKSSFPGR

SEQ ID NO: 147 - protein, 30 aa, synthetic construct
KSGPGGPAGIGALFFSSPPLAQKLKSSGGR

SEQ ID NO: 148 - protein, 18 aa, synthetic construct
LSGRSDNHGGAVGLLAPP

SEQ ID NO: 149 - protein, 22 aa, synthetic construct
LSGRSDNHGGSGGSISSGLLSS

SEQ ID NO: 150 - protein, 22 aa, synthetic construct
LSGRSDNHGGSGGSQNQALRMA

SEQ ID NO: 151 - protein, 20 aa, synthetic construct
LSGRSDNHGGVHMPLGFLGP

SEQ ID NO: 152 - protein, 22 aa, synthetic construct
LSGRSGNHGGSGGSISSGLLSS

SEQ ID NO: 153 - protein, 22 aa, synthetic construct
LSGRSGNHGGSGGSQNQALRMA

SEQ ID NO: 154 - protein, 22 aa, synthetic construct
QNQALRMAGGSGGSLSGRSDNH

SEQ ID NO: 155 - protein, 22 aa, synthetic construct
QNQALRMAGGSGGSLSGRSGNH

SEQ ID NO: 156 - protein, 30 aa, synthetic construct
RGGALFKSSFPLAQKLKSSGPAGLYAQGGK

SEQ ID NO: 157 - protein, 30 aa, synthetic construct
RGGGPAGLYAQPLAQKLKSSALFKSSFPGG

SEQ ID NO: 158 - protein, 30 aa, synthetic construct
SGGFPRSGGSFNPRTFGSKRKRRGSRGGGG

SEQ ID NO: 159 - protein, 30 aa, synthetic construct
SGPLAQKLKSSGPAGLYAQALFKSSFPGSK

SEQ ID NO: 160 - protein, 22 aa, synthetic construct
TSTSGRSANPRGGGAVGLLAPP

SEQ ID NO: 161 - protein, 24 aa, synthetic construct
TSTSGRSANPRGGGVHMPLGFLGP

SEQ ID NO: 162 - protein, 20 aa, synthetic construct
VHMPLGFLGPGGLSGRSDNH

SEQ ID NO: 163 - protein, 24 aa, synthetic construct
VHMPLGFLGPGGTSTSGRSANPRG

SEQ ID NO: 164 - protein, 21 aa, synthetic construct
SGRSAGGGSGRSAGGGSGRSA

SEQ ID NO: 165 - protein, 35 aa, synthetic construct
HPVGLLARGGGHPVGLLARGGGSGRSAGGGSGRSA

SEQ ID NO: 166 - protein, 8 aa, synthetic construct
GPLGVRGK

SEQ ID NO: 167 - protein, 8 aa, synthetic construct
HPVGLLAR

SEQ ID NO: 168 - protein, 8 aa, synthetic construct
GPQGIAGQ

SEQ ID NO: 169 - protein, 8 aa, synthetic construct
VPMSMRGG

SEQ ID NO: 170 - protein, 8 aa, synthetic construct
IPVSLRSG

SEQ ID NO: 171 - protein, 8 aa, synthetic construct
RPFSMIMG

SEQ ID NO: 172 - protein, 8 aa, synthetic construct
VPLSLTMG

SEQ ID NO: 173 - protein, 8 aa, synthetic construct
VPLSLYSG

SEQ ID NO: 174 - protein, 8 aa, synthetic construct
IPESLRAG

SEQ ID NO: 175 - protein, 23 aa, synthetic construct
VPLSLYSGGGISSGLLSGRSDNH

SEQ ID NO: 176 - protein, 20 aa, synthetic construct
GGGISSGLLSGRSDNHGGGS

SEQ ID NO: 177 - protein, 15 aa, synthetic construct
GGGHPVGLLARGGGS

SEQ ID NO: 178 - protein, 36 aa, synthetic construct
GGGSGGGSGGGGISSGLLSGRSDNHGGGSGGGSGGS

SEQ ID NO: 179 - protein, 36 aa, synthetic construct
GGGGISSGLLSGRSDNHGGGISSGLLSGRSDNHGGS

SEQ ID NO: 180 - protein, 36 aa, synthetic construct
GGGSGGSIPVSLRSGGGISSGLLSGRSDNHGGSGGS

SEQ ID NO: 181 - protein, 36 aa, synthetic construct
GGGSGGSVPLSLYSGGGISSGLLSGRSDNHGGSGGS

SEQ ID NO: 182 - protein, 36 aa, synthetic construct
GGGSHPVGLLARGGGHPVGLLARGGGHPVGLLARGS

SEQ ID NO: 183 - protein, 36 aa, synthetic construct
GGGSHPVGLLARGGGHPVGLLARGGSGRSAGGSGRS

SEQ ID NO: 184 - protein, 15 aa, synthetic construct
GISSGLLSGRSDNHG

SEQ ID NO: 185 - protein, 21 aa, synthetic construct
GGGSISSGLLSGRSDNHGGGS

SEQ ID NO: 186 - protein, 6 aa, synthetic construct
HHHHHH

SEQ ID NO: 187 - protein, 28 aa, synthetic construct
WSHPQFEKGGGSGGGSGGSAWSHPQFEK

SEQ ID NO: 188 - protein, 5 aa, synthetic construct
GGGGS

SEQ ID NO: 189 - protein, 40 aa, synthetic construct
GGGGSGGGGSGGGGSGGGGSGGGGSGGGGSGGGGSGGGGS

SEQ ID NO: 190 - protein, 35 aa, synthetic construct
GGGGSGGGGSGGGGSGGGGSGGGGSGGGGSGGGGS

SEQ ID NO: 191 - protein, 30 aa, synthetic construct
GGGGSGGGGSGGGGSGGGGSGGGGSGGGGS

SEQ ID NO: 192 - protein, 25 aa, synthetic construct
GGGGSGGGGSGGGGSGGGGSGGGGS

SEQ ID NO: 193 - protein, 20 aa, synthetic construct
GGGGSGGGGSGGGGSGGGGS

SEQ ID NO: 194 - protein, 15 aa, synthetic construct
GGGGSGGGGSGGGGS

SEQ ID NO: 195 - protein, 10 aa, synthetic construct
GGGGSGGGGS

SEQ ID NO: 196 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 198 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 199 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 200 - protein, 11 aa, synthetic construct
  VARIANT 1..10: note = This region may encompass 1-10 residues
GGGGGGGGGGS

SEQ ID NO: 201 - protein, 11 aa, synthetic construct
  VARIANT 2..11: note = This region may encompass 1-10 residues
SGGGGGGGGGG

SEQ ID NO: 202 - protein, 4 aa, synthetic construct
GGGG

SEQ ID NO: 203 - protein, 5 aa, synthetic construct
GGGGG

SEQ ID NO: 204 - protein, 6 aa, synthetic construct
GGGGGG

SEQ ID NO: 205 - protein, 7 aa, synthetic construct
GGGGGGG

SEQ ID NO: 206 - protein, 8 aa, synthetic construct
GGGGGGGG

SEQ ID NO: 207 - protein, 9 aa, synthetic construct
GGGGGGGGG

SEQ ID NO: 208 - protein, 4 aa, synthetic construct
GGGS

SEQ ID NO: 209 - protein, 5 aa, synthetic construct
GSGGS

SEQ ID NO: 210 - protein, 5 aa, synthetic construct
AAAGG

SEQ ID NO: 211 - protein, 5 aa, synthetic construct
ADAAP

SEQ ID NO: 212 - protein, 11 aa, synthetic construct
ADAAPTVSIFP

SEQ ID NO: 213 - protein, 12 aa, synthetic construct
ADAAPTVSIFPP

SEQ ID NO: 214 - protein, 6 aa, synthetic construct
AKTTAP

SEQ ID NO: 215 - protein, 13 aa, synthetic construct
AKTTAPSVYPLAP

SEQ ID NO: 216 - protein, 17 aa, synthetic construct
AKTTPKLEEGEFSEARV

SEQ ID NO: 217 - protein, 9 aa, synthetic construct
AKTTPKLGG

SEQ ID NO: 218 - protein, 6 aa, synthetic construct
AKTTPP

SEQ ID NO: 219 - protein, 13 aa, synthetic construct
AKTTPPSVTPLAP

SEQ ID NO: 220 - protein, 6 aa, synthetic construct
ASTKGP

SEQ ID NO: 221 - protein, 26 aa, synthetic construct
ASTKGPSVFPLAPASTKGPSVFPLAP

SEQ ID NO: 222 - protein, 14 aa, synthetic construct
EGKSSGSGSESKST

SEQ ID NO: 223 - protein, 15 aa, synthetic construct
GEGESGEGESGEGES

SEQ ID NO: 224 - protein, 20 aa, synthetic construct
GEGESGEGESGEGESGEGES

SEQ ID NO: 225 - protein, 15 aa, synthetic construct
GEGGSGEGGSGEGGS

SEQ ID NO: 226 - protein, 15 aa, synthetic construct
GENKVEYAPALMALS

SEQ ID NO: 227 - protein, 15 aa, synthetic construct
GGEGSGGEGSGGEGS

SEQ ID NO: 228 - protein, 15 aa, synthetic construct
GGGESGGEGSGEGGS

SEQ ID NO: 229 - protein, 15 aa, synthetic construct
GGGESGGGESGGGES

SEQ ID NO: 230 - protein, 15 aa, synthetic construct
GGGKSGGGKSGGGKS

SEQ ID NO: 231 - protein, 15 aa, synthetic construct
GGGKSGGKGSGKGGS

SEQ ID NO: 232 - protein, 5 aa, synthetic construct
GGGSG

SEQ ID NO: 233 - protein, 15 aa, synthetic construct
GGKGSGGKGSGGKGS

SEQ ID NO: 234 - protein, 4 aa, synthetic construct
GGSG

SEQ ID NO: 235 - protein, 5 aa, synthetic construct
GGSGG

SEQ ID NO: 236 - protein, 9 aa, synthetic construct
GGSGGGGSG

SEQ ID NO: 237 - protein, 13 aa, synthetic construct
GGSGGGGSGGGGS

SEQ ID NO: 238 - protein, 15 aa, synthetic construct
GHEAAAVMQVQYPAS

SEQ ID NO: 239 - protein, 15 aa, synthetic construct
GKGGSGKGGSGKGGS

SEQ ID NO: 240 - protein, 15 aa, synthetic construct
GKGKSGKGKSGKGKS

SEQ ID NO: 241 - protein, 20 aa, synthetic construct
GKGKSGKGKSGKGKSGKGKS

SEQ ID NO: 242 - protein, 15 aa, synthetic construct
GKPGSGKPGSGKPGS

SEQ ID NO: 243 - protein, 21 aa, synthetic construct
GKPGSGKPGSGKPGSGKPSGS

SEQ ID NO: 244 - protein, 15 aa, synthetic construct
GPAKELTPLKEAKVS

SEQ ID NO: 245 - protein, 12 aa, synthetic construct
GSAGSAAGSGEF

SEQ ID NO: 246 - protein, 5 aa, synthetic construct
GSGGG

SEQ ID NO: 247 - protein, 5 aa, synthetic construct
GSGSG

SEQ ID NO: 248 - protein, 4 aa, synthetic construct
GSSG

SEQ ID NO: 249 - protein, 10 aa, synthetic construct
GSSGGSGGSG

SEQ ID NO: 250 - protein, 11 aa, synthetic construct
GSSGGSGGSGG

SEQ ID NO: 251 - protein, 12 aa, synthetic construct
GSSGGSGGSGGS

SEQ ID NO: 252 - protein, 13 aa, synthetic construct
GSSGGSGGSGGSG

SEQ ID NO: 253 - protein, 16 aa, synthetic construct
GSSGGSGGSGGSGGGS

SEQ ID NO: 254 - protein, 11 aa, synthetic construct
GSSGGSGGSGS

SEQ ID NO: 255 - protein, 5 aa, synthetic construct
GSSGT

SEQ ID NO: 256 - protein, 5 aa, synthetic construct
GSSSG

SEQ ID NO: 257 - protein, 18 aa, synthetic construct
GSTSGSGKPGSGEGSTKG

SEQ ID NO: 258 - protein, 19 aa, synthetic construct
GTAAAGAGAAGGAAAGAAG

SEQ ID NO: 259 - protein, 19 aa, synthetic construct
GTSGSSGSGSGGSGSGGGG

SEQ ID NO: 260 - protein, 14 aa, synthetic construct
IRPRAIGGSKPRVA

SEQ ID NO: 261 - protein, 18 aa, synthetic construct
KESGSVSSEQLAQFRSLD

SEQ ID NO: 262 - protein, 15 aa, synthetic construct
KTTPKLEEGEFSEAR

SEQ ID NO: 263 - protein, 19 aa, synthetic construct
PRGASKSGSASQTGSAPGS

SEQ ID NO: 264 - protein, 6 aa, synthetic construct
QPKAAP

SEQ ID NO: 265 - protein, 13 aa, synthetic construct
QPKAAPSVTLFPP

SEQ ID NO: 266 - protein, 27 aa, synthetic construct
RADAAAAGGGGSGGGGSGGGGSGGGGS

SEQ ID NO: 267 - protein, 12 aa, synthetic construct
RADAAAAGGPGS

SEQ ID NO: 268 - protein, 6 aa, synthetic construct
RADAAP

SEQ ID NO: 269 - protein, 9 aa, synthetic construct
RADAAPTVS

SEQ ID NO: 270 - protein, 6 aa, synthetic construct
SAKTTP

SEQ ID NO: 271 - protein, 18 aa, synthetic construct
SAKTTPKLEEGEFSEARV

SEQ ID NO: 272 - protein, 10 aa, synthetic construct
SAKTTPKLGG

SEQ ID NO: 273 - protein, 14 aa, synthetic construct
STAGDTHLGGEDFD

SEQ ID NO: 274 - protein, 5 aa, synthetic construct
TVAAP

SEQ ID NO: 275 - protein, 12 aa, synthetic construct
TVAAPSVFIFPP

SEQ ID NO: 276 - protein, 24 aa, synthetic construct
TVAAPSVFIFPPTVAAPSVFIFPP

SEQ ID NO: 277 - protein, 35 aa, synthetic construct
GGGGSLALGPGGGGGSLALGPGGGGGSLALGPGGS

SEQ ID NO: 278 - protein, 46 aa, synthetic construct
AGSGNSSGSGGSGGSGNSSGSGGSPVPSTPPTPSPSTPPTPSPSAS

SEQ ID NO: 279 - protein, 7 aa, synthetic construct
GGGGSAS

SEQ ID NO: 280 - protein, 12 aa, synthetic construct
GGGGSGGGGSAS

SEQ ID NO: 281 - protein, 17 aa, synthetic construct
GGGGSGGGGSGGGGSAS

SEQ ID NO: 282 - protein, 27 aa, synthetic construct
GGGGSGGGGSGGGGSGGGGSGGGGSAS

SEQ ID NO: 283 - protein, 32 aa, synthetic construct
GGGGSGGGGSGGGGSGGGGSGGGGSGGGGSAS

SEQ ID NO: 284 - protein, 37 aa, synthetic construct
AGGGSGGGGSGGGGSGGGGSGGGGSGGGGSGGGGSAS

SEQ ID NO: 285 - protein, 42 aa, synthetic construct
AGGGSGGGGSGGGGSGGGGSGGGGSGGGGSGGGGSGGGGSAS

SEQ ID NO: 286 - protein, 47 aa, synthetic construct
AGGGSGGGGSGGGGSGGGGSGGGGSGGGGSGGGGSGGGGSGGGGSAS

SEQ ID NO: 287 - protein, 40 aa, synthetic construct
DEDEDEDEDEDEDEDERPLALWRSDRDRDRDRDRDRDRDR

SEQ ID NO: 288 - protein, 8 aa, synthetic construct
AARGPAIH

SEQ ID NO: 289 - protein, 8 aa, synthetic construct
AAYHLVSQ

SEQ ID NO: 290 - protein, 8 aa, synthetic construct
AGLGISST

SEQ ID NO: 291 - protein, 8 aa, synthetic construct
AGLGVVER

SEQ ID NO: 292 - protein, 4 aa, synthetic construct
ALAL

SEQ ID NO: 293 - protein, 8 aa, synthetic construct
ALFFSSPP

SEQ ID NO: 294 - protein, 8 aa, synthetic construct
ALFKSSFP

SEQ ID NO: 295 - protein, 7 aa, synthetic construct
ALLLALL

SEQ ID NO: 296 - protein, 8 aa, synthetic construct
AQFVLTEG

SEQ ID NO: 297 - protein, 8 aa, synthetic construct
AQNLLGMV

SEQ ID NO: 298 - protein, 8 aa, synthetic construct
AVGLLAPP

SEQ ID NO: 299 - protein, 4 aa, synthetic construct
DAFK

SEQ ID NO: 300 - protein, 4 aa, synthetic construct
DEVD

SEQ ID NO: 301 - protein, 5 aa, synthetic construct
DEVDP

SEQ ID NO: 302 - protein, 6 aa, synthetic construct
DPRSFL

SEQ ID NO: 303 - protein, 8 aa, synthetic construct
DVAQFVLT

SEQ ID NO: 304 - protein, 4 aa, synthetic construct
DVLK

SEQ ID NO: 305 - protein, 8 aa, synthetic construct
DWLYWPGI

SEQ ID NO: 306 - protein, 7 aa, synthetic construct
EDDDDKA

SEQ ID NO: 307 - protein, 7 aa, synthetic construct
  SITE 3: note = Citrulline
  SITE 5: note = Homophenylalanine
EPXGXYL

SEQ ID NO: 308 - protein, 8 aa, synthetic construct
EPQALAMS

SEQ ID NO: 309 - protein, 8 aa, synthetic construct
ESLPVVAV

SEQ ID NO: 310 - protein, 8 aa, synthetic construct
ESPAYYTA

SEQ ID NO: 311 - protein, 10 aa, synthetic construct
FPRPLGITGL

SEQ ID NO: 312 - protein, 8 aa, synthetic construct
FRLLDWQW

SEQ ID NO: 313 - protein, 4 aa, synthetic construct
GFLG

SEQ ID NO: 314 - protein, 10 aa, synthetic construct
GGAANLVRGG

SEQ ID NO: 315 - protein, 5 aa, synthetic construct
GGGRR

SEQ ID NO: 316 - protein, 8 aa, synthetic construct
GGPRGLPG

SEQ ID NO: 317 - protein, 10 aa, synthetic construct
GGQPSGMWGW

SEQ ID NO: 318 - protein, 7 aa, synthetic construct
GGSIDGR

SEQ ID NO: 319 - protein, 8 aa, synthetic construct
GGWHTGRN

SEQ ID NO: 320 - protein, 5 aa, synthetic construct
GIAGQ

SEQ ID NO: 321 - protein, 6 aa, synthetic construct
GKAFRR

SEQ ID NO: 322 - protein, 8 aa, synthetic construct
GPAGLYAQ

SEQ ID NO: 323 - protein, 8 aa, synthetic construct
GPAGMKGL

SEQ ID NO: 324 - protein, 8 aa, synthetic construct
GPEGLRVG

SEQ ID NO: 325 - protein, 8 aa, synthetic construct
GPLGIAGI

SEQ ID NO: 326 - protein, 7 aa, synthetic construct
GPLGVRG

SEQ ID NO: 327 - protein, 8 aa, synthetic construct
GPQGLLGA

SEQ ID NO: 328 - protein, 6 aa, synthetic construct
GPRSFG

SEQ ID NO: 329 - protein, 7 aa, synthetic construct
GPRSFGL

SEQ ID NO: 330 - protein, 8 aa, synthetic construct
GPSHLVLT

SEQ ID NO: 331 - protein, 10 aa, synthetic construct
GVSQNYPIVG

SEQ ID NO: 332 - protein, 10 aa, synthetic construct
GVVQASCRLA

SEQ ID NO: 333 - protein, 6 aa, synthetic construct
GWEHDG

SEQ ID NO: 334 - protein, 6 aa, synthetic construct
HSSKLQ

SEQ ID NO: 335 - protein, 9 aa, synthetic construct
HSSKLQEDA

SEQ ID NO: 336 - protein, 7 aa, synthetic construct
HSSKLQL

SEQ ID NO: 337 - protein, 8 aa, synthetic construct
HTGRSGAL

SEQ ID NO: 338 - protein, 4 aa, synthetic construct
IDGR

SEQ ID NO: 339 - protein, 4 aa, synthetic construct
IEGR

SEQ ID NO: 340 - protein, 8 aa, synthetic construct
ILPRSPAF

SEQ ID NO: 341 - protein, 5 aa, synthetic construct
ISSGL

SEQ ID NO: 342 - protein, 6 aa, synthetic construct
ISSGLL

SEQ ID NO: 343 - protein, 7 aa, synthetic construct
ISSGLLS

SEQ ID NO: 344 - protein, 8 aa, synthetic construct
ISSGLLSS

SEQ ID NO: 345 - protein, 7 aa, synthetic construct
ISSGLSS

SEQ ID NO: 346 - protein, 8 aa, synthetic construct
KGSGDVEG

SEQ ID NO: 347 - protein, 9 aa, synthetic construct
KQEQNPGST

SEQ ID NO: 348 - protein, 8 aa, synthetic construct
KRALGLPG

SEQ ID NO: 349 - protein, 8 aa, synthetic construct
LAAPLGLL

SEQ ID NO: 350 - protein, 9 aa, synthetic construct
LAPLGLQRR

SEQ ID NO: 351 - protein, 8 aa, synthetic construct
LAQKLKSS

SEQ ID NO: 352 - protein, 8 aa, synthetic construct
LAQRLRSS

SEQ ID NO: 353 - protein, 5 aa, synthetic construct
LEATA

SEQ ID NO: 354 - protein, 8 aa, synthetic construct
LKAAPRWA

SEQ ID NO: 355 - protein, 8 aa, synthetic construct
LLAPSHRA

SEQ ID NO: 356 - protein, 8 aa, synthetic construct
LPGGLSPW

SEQ ID NO: 357 - protein, 8 aa, synthetic construct
LSGRSANI

SEQ ID NO: 358 - protein, 8 aa, synthetic construct
LSGRSANP

SEQ ID NO: 359 - protein, 8 aa, synthetic construct
LSGRSDDH

SEQ ID NO: 360 - protein, 8 aa, synthetic construct
LSGRSDIH

SEQ ID NO: 361 - protein, 8 aa, synthetic construct
LSGRSDNH

SEQ ID NO: 362 - protein, 8 aa, synthetic construct
LSGRSDNI

SEQ ID NO: 363 - protein, 8 aa, synthetic construct
LSGRSDNP

SEQ ID NO: 364 - protein, 8 aa, synthetic construct
LSGRSDQG

SEQ ID NO: 365 - protein, 8 aa, synthetic construct
LSGRSDQH

SEQ ID NO: 366 - protein, 8 aa, synthetic construct
LSGRSDTH

SEQ ID NO: 367 - protein, 8 aa, synthetic construct
LSGRSDYH

SEQ ID NO: 368 - protein, 8 aa, synthetic construct
LSGRSGNH

SEQ ID NO: 369 - protein, 10 aa, synthetic construct
LVLASSSFGY

SEQ ID NO: 370 - protein, 8 aa, synthetic construct
MDAFLESS

SEQ ID NO: 371 - protein, 8 aa, synthetic construct
MGLFSEAG

SEQ ID NO: 372 - protein, 8 aa, synthetic construct
MIAPVAYR

SEQ ID NO: 373 - protein, 8 aa, synthetic construct
MVLGRSLL

SEQ ID NO: 374 - protein, 12 aa, synthetic construct
NTLSGRSENHSG

SEQ ID NO: 375 - protein, 12 aa, synthetic construct
NTLSGRSGNHGS

SEQ ID NO: 376 - protein, 8 aa, synthetic construct
PAGLWLDP

SEQ ID NO: 377 - protein, 8 aa, synthetic construct
PGGPAGIG

SEQ ID NO: 378 - protein, 5 aa, synthetic construct
  SITE 3: note = S-Ethylcysteine
PICFF

SEQ ID NO: 379 - protein, 6 aa, synthetic construct
  SITE 4: note = S-Methylcysteine
PLGCAG

SEQ ID NO: 380 - protein, 4 aa, synthetic construct
PLGL

SEQ ID NO: 381 - protein, 6 aa, synthetic construct
PLGLAG

SEQ ID NO: 382 - protein, 6 aa, synthetic construct
  VARIANT 6: note = Any amino acid
PLGLAX

SEQ ID NO: 383 - protein, 6 aa, synthetic construct
PLGLWA

SEQ ID NO: 384 - protein, 7 aa, synthetic construct
PLGLWSQ

SEQ ID NO: 385 - protein, 8 aa, synthetic construct
PLTGRSGG

SEQ ID NO: 386 - protein, 5 aa, synthetic construct
PMAKK

SEQ ID NO: 387 - protein, 6 aa, synthetic construct
PPRSFL

SEQ ID NO: 388 - protein, 8 aa, synthetic construct
PRFRIIGG

SEQ ID NO: 389 - protein, 8 aa, synthetic construct
PVGYTSSL

SEQ ID NO: 390 - protein, 8 aa, synthetic construct
PVQPIGPQ

SEQ ID NO: 391 - protein, 8 aa, synthetic construct
QALAMSAI

SEQ ID NO: 392 - protein, 8 aa, synthetic construct
QGRAITFI

SEQ ID NO: 393 - protein, 8 aa, synthetic construct
QNQALRMA

SEQ ID NO: 394 - protein, 4 aa, synthetic construct
RGPA

SEQ ID NO: 395 - protein, 8 aa, synthetic construct
RGPAFNPM

SEQ ID NO: 396 - protein, 8 aa, synthetic construct
RGPATPIM

SEQ ID NO: 397 - protein, 14 aa, synthetic construct
RKSSIIIRMRDVVL

SEQ ID NO: 398 - protein, 7 aa, synthetic construct
RLQLKAC

SEQ ID NO: 399 - protein, 6 aa, synthetic construct
RLQLKL

SEQ ID NO: 400 - protein, 8 aa, synthetic construct
RMHLRSLG

SEQ ID NO: 401 - protein, 8 aa, synthetic construct
RPSPMWAY

SEQ ID NO: 402 - protein, 8 aa, synthetic construct
RQARVVNG

SEQ ID NO: 403 - protein, 8 aa, synthetic construct
SAGFSLPA

SEQ ID NO: 404 - protein, 8 aa, synthetic construct
SAPAVESE

SEQ ID NO: 405 - protein, 8 aa, synthetic construct
SARGPSRW

SEQ ID NO: 406 - protein, 9 aa, synthetic construct
SGEPAYYTA

SEQ ID NO: 407 - protein, 8 aa, synthetic construct
SGGPLGVR

SEQ ID NO: 408 - protein, 10 aa, synthetic construct
SGRIGFLRTA

SEQ ID NO: 409 - protein, 5 aa, synthetic construct
SGRSA

SEQ ID NO: 410 - protein, 9 aa, synthetic construct
SGRSANPRG

SEQ ID NO: 411 - protein, 8 aa, synthetic construct
SMLRSMPL

SEQ ID NO: 412 - protein, 8 aa, synthetic construct
SPLPLRVP

SEQ ID NO: 413 - protein, 8 aa, synthetic construct
SPLTGRSG

SEQ ID NO: 414 - protein, 8 aa, synthetic construct
SPRSIMLA

SEQ ID NO: 415 - protein, 8 aa, synthetic construct
SSRGPAYL

SEQ ID NO: 416 - protein, 9 aa, synthetic construct
SSRHRRALD

SEQ ID NO: 417 - protein, 15 aa, synthetic construct
SSSFDKGKYKKGDDA

SEQ ID NO: 418 - protein, 15 aa, synthetic construct
SSSFDKGKYKRGDDA

SEQ ID NO: 419 - protein, 8 aa, synthetic construct
STFPFGMF

SEQ ID NO: 420 - protein, 8 aa, synthetic construct
TARGPSFK

SEQ ID NO: 421 - protein, 8 aa, synthetic construct
TGRGPSWV

SEQ ID NO: 422 - protein, 8 aa, synthetic construct
TSGRSANP

SEQ ID NO: 423 - protein, 12 aa, synthetic construct
TSTSGRSANPRG

SEQ ID NO: 424 - protein, 8 aa, synthetic construct
VAGRSMRP

SEQ ID NO: 425 - protein, 8 aa, synthetic construct
VAQFVLTE

SEQ ID NO: 426 - protein, 10 aa, synthetic construct
VHMPLGFLGP

SEQ ID NO: 427 - protein, 8 aa, synthetic construct
VVPEGRRS

SEQ ID NO: 428 - protein, 8 aa, synthetic construct
WATPRPMR

SEQ ID NO: 429 - protein, 8 aa, synthetic construct
YGAGLGVV

SEQ ID NO: 430 - protein, 6 aa, synthetic construct
GGSGGS

SEQ ID NO: 431 - protein, 11 aa, synthetic construct
GGSGGSGGSGS

SEQ ID NO: 432 - protein, 6 aa, synthetic construct
QGQSGQ

SEQ ID NO: 433 - protein, 7 aa, synthetic construct
QGQSGQG

SEQ ID NO: 434 - protein, 6 aa, synthetic construct
QGQSGS

SEQ ID NO: 435 - protein, 4 aa, synthetic construct
QSGQ

SEQ ID NO: 436 - protein, 5 aa, synthetic construct
QSGQG

SEQ ID NO: 437 - protein, 4 aa, synthetic construct
QSGS

SEQ ID NO: 438 - protein, 4 aa, synthetic construct
SGQG

SEQ ID NO: 439 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 440 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK